(19) United States
(12) Patent Application Publication
(10) Pub. No.: US 2004/0043959 A1
(43) Pub. Date: Mar. 4, 2004

(54) Combination therapies for treating methylthioadenosine phosphorylase deficient cells

(76) Inventors: Laura A. Bloom, San Diego, CA (US); Theodore J. Boritzki, San Diego, CA (US); Richard Ogden, San Diego, CA (US); Donald Skalitzky, San Diego, CA (US); Pei-Pei Kung, San Diego, CA (US); Luke Zehnder, San Diego, CA (US); Leslie Kuhn, Haslett, MI (US); Jerry Jialun Meng, San Diego, CA (US)

Correspondence Address: Wendy Lei Hsu, Agouron Pharmaceuticals, Inc., Legal Division, Patent Department, 10777 Science Center Drive, San Diego, CA, 92121, US

(21) Appl. No.: 10/367,366

(22) Filed: Feb. 14, 2003

Related U.S. Application Data
(60) Provisional application No. 60/361,645, filed on Mar. 4, 2002.
(60) Provisional application No. 60/432,275, filed on Dec. 9, 2002.

(51) Int. Cl. A61K 31/7076; A61K 31/52

(52) U.S. Cl. 514/46; 514/263.23

(57) ABSTRACT

The present invention is directed to combination therapies for treating cell proliferative disorders associated with methylthioadenosine phosphorylase (MTAP) deficient cells in a mammal. The combination therapies selectively kill MTAP-deficient cells by administering an inhibitor of de novo inosinate synthesis and administering an anti-toxicity agent, wherein the inhibitors of de novo inosinate synthesis are inhibitors of glycinamide ribonucleotide formyltransferase (“GARFT”) and/or aminoinidazolecarboximide ribonucleotide formyltransferase (“AICARFT”), and the anti-toxicity agent is an MTAP substrate (e.g. methylthioadenosine or “MTA”), a precursor of MTA, an analog of an MTA precursor or a prodrug of an MTAP substrate.

### CROSS-REFERENCE TO RELATED APPLICATIONS

This application claims the benefit of U.S. Provisional Application Serial No. 60/361,645 filed Mar. 4, 2002, and U.S. Provisional Application Serial No. 60/432,275 filed Dec. 9, 2002, which are hereby incorporated by reference in their entireties.

### FIELD OF THE INVENTION

This invention relates to combination therapies for treating cell proliferative disorders in methylthioadenosine phosphorylase (“MTAP”) deficient cells in a mammal. The combination therapies selectively kill MTAP-deficient cells when an inhibitor of de novo inosinate synthesis is administered with an anti-toxicity agent. More particularly, this invention relates to combination therapies comprising an inhibitor of de novo inosinate synthesis selected from inhibitors of glycinamide ribonucleotide formyltransferase (“GARFT”), aminoinidazolecarboximide ribonucleotide formyltransferase (“AICARFT”), or both, and an anti-toxicity agent selected from MTAP substrates, precursors of methylthioadenosine (“MTA”), analogs of MTA precursors, or prodrugs of MTAP substrates.

### BACKGROUND OF THE INVENTION

Methylthioadenosine phosphorylase (“MTAP”) is an enzyme involved in the metabolism of polyamines and purines. Although MTAP is present in all healthy cells, certain cancers are known to have an incidence of MTAP-deficiency. See, e.g., Fitchen et al., “Methylthioadenosine phosphorylase deficiency in human leukemias and solid tumors,” Cancer Res., 46: 5409-5412,(1986); Nobori et al., “Methylthioadenosine phosphrylase deficiency in human non-small cell lung cancers,” Cancer Res., 53: 1098-1101 (1993).

As shown in FIG. 1, adenosine 5′-triphosphate (“ATP”) production relies on the salvage or synthesis of adenylate (“AMP”). In healthy, MTAP-competent cells, AMP is produced primarily through one of two ways: (1) the de novo synthesis of the intermediate inosinate (“IMP”; i.e., the de novo pathway), or (2) through the MTAP-mediated salvage pathway. In contrast, in MTAP-deficient cells, AMP production proceeds primarily through the de novo pathway, while the MTAP salvage pathway is closed. Accordingly, when the de novo pathway is also turned off, MTAP-deficient cells are expected to be selectively killed. The MTAP-deficient nature of certain cancers therefore provides an opportunity to design therapies that selectively kill MTAP-deficient cells by preventing toxicity in MTAP-competent cells.

Several attempts have been made to selectively target cancers deficient in MTAP in mammals by inhibiting the de novo pathway. One attempt employed the inhibitor L-alanosine, the L isomer of an antibiotic obtained from Streptomyces alanosinicus, which blocks the conversion of IMP to AMP by inhibition of adenylosuccinate synthetase. See, e.g., Batova et al., “Use of Alanosine as a Methylthioadenosine Phosphorylase-Selective Therapy for T-cell Acute Lymphoblastic Leukemia In vitro”, Cancer Research 59: 1492-1497 (1999); WO99/20791; U.S. Pat. No. 5,840,505. L-alanosine failed in its early antitumor clinical trials. Those early trials, however, did not identify or differentiate patients whose cancers were MTAP-deficient. Further clinical trials have been initiated.

Other inhibitors of de novo AMP synthesis have been discovered and studied for antitumor activity. Blockage of earlier steps in the de novo AMP synthesis pathway, i.e., blockage of de novo IMP synthesis, was investigated using the IMP synthesis inhibitor dideazatetrahydrofolate (“lometrexol” or “DDATHF”). In initial clinical trials, administration of lometrexol resulted in severe, delayed toxicities. Alati et al. asserted that lometrexol's severe toxicity was attributable to lower folate levels in human plasma as compared to mice. (Alati et al. “Augmentation of the Therapeutic Activity of Lometrexol [6-R)t, 10-Dideazatetrahydrofolate] by Oral Folic Acid,” Cancer Res. 56: 2331-2335 (1996)). Similar toxicity problems have been encountered with LY309887, an even more potent IMP synthesis inhibitor than lometrexol. Worzalla, et al., “Antitumor Therapeutic Index of LY309887 is Improved With Increased Folic Acid Supplementation in Mice Maintained on a Folate Deficient Diet,” Proc. AACR 37: 0197-016X (1996).

Lometrexol and LY309887 relied predominantly on the membrane folate binding protein (“mFBP”) for transport into cells. As mentioned above, administration of lometrexol and LY309887 resulted in markedly high toxicity in mammals with relatively lower circulating folate levels (e.g. humans, when compared to mice). It has been suggested that the undesirable toxicity of these inhibitors, particularly in mammals with lower circulating folate levels, is related to their high affinity for the mFBP, which is unregulated during times of folate deficiency. See Antony, “The Biological Chemistry of Folate Receptors,” Blood, 79: 2807-2820 (1992); see also Pizzorno et al., “5,10-Dideazatetrahydrofolic Acid (DDATHF) Transport in CCRF-CEM and MA104 Cell Lines, ” J. Biol. Chemistry, 268: 1017-1023 (1993). These toxicity problems have led to the use of folate supplementation in later clinical trials with inhibitors of GARFT.

Since MTAP provides a salvage pathway for AMP production (and therefore ATP production), administration of a substrate for MTAP, e.g., methylthioadenosine (“MTA”), along with a de novo AMP inhibitor, was expected to counteract the toxicity of the inhibitor in MTAP-competent (i.e., healthy) cells but not in MTAP-deficient (i.e., cancer) cells. This theory has been extensively studied by combination of MTA with L-alanosine. See, e.g., Batova et al., “Use of Alanosine as a Methylthioadenosine Phosphorylase-Selective Therapy for T-cell Acute Lymphoblastic Leukemia In vitro”, Cancer Research 59: 1492-1497 (1999); Batova et al., “Frequent Deletion in the Methylthioadenosine Phosphorylase Gene in T-Cell Acute Lymphoblastic Leukemia: Strategies for Enzyme-Targeted Therapy,” Blood, 88: 3083-3090 (1996); WO99/20791; U.S. Pat. No. 5,840,505; European Patent Publication No. 0974362A1. As described above, L-alanosine acts to inhibit the conversion of IMP to AMP, after the de novo synthesis of IMP.

The L-alanosine studies described above assert that blockage of earlier steps in the de novo AMP synthesis pathway, i.e. blockage of de novo IMP synthesis, would result in inhibition of not only AMP synthesis, but guanylate synthesis as well, and would thus prevent MTA from selectively rescuing MTAP-competent cells. Hori et al, “Methylthioadenosine Phosphorylase cDNA Transfection Alters Sensitivity to Depletion of Purine and Methionine in A549 Lung Cancer Cells”, Cancer Research, 56, 5656 (1996). This hypothesis was borne out by experiments involving the simultaneous in vitro administration of MTA with either lometrexol or with methotrexate. Lometrexol is an inhibitor of glycinamide ribonucleotide formyltransferase (“GARFT”), whereas methotrexate is primarily a dihydrofolate reductase inhibitor that also inhibits GARFT and aminoinidazolecarboximide ribonucleotide formyltransferase (“AICARFT”). For both lometrexol and methotrexate, simultaneous administration of MTA with the drug did not completely restore cell growth at therapeutically desirable concentrations of the inhibitors. See Hori et al, Cancer Res., 56, 5656 (1996).

There is a need for effective combination therapies for treating cell-proliferative disorders having incidence of MTAP-deficiency.

### SUMMARY OF THE INVENTION

This invention relates to a method of selectively killing methylthioadenosine phosphorylase (MTAP)-deficient cells of a mammal by administering a therapeutically effective amount of an inhibitor of glycinamide ribonucleotide formyltransferase (“GARFT”) and/or aminoimidazolecarboximide ribonucleotide formyltransferase (“AICARFT”), and administering an anti-toxicity agent in an amount effective to increase the maximally tolerated dose of the inhibitor, wherein the anti-toxicity agent is administered during and after administration of the inhibitor. Preferably, the anti-toxicity agent is selected from the group consisting of MTAP substrates and prodrugs of MTAP substrates, or combinations thereof.

In one embodiment, the anti-toxicity agent is an analog of MTA having Formula X, wherein R₄₁, R₄₂, R₄₃, R₄₄ and R₄₅ are as defined below:

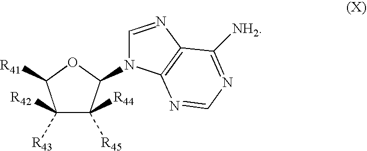

Alternatively, the an-toxicity agent is a prodrug of MTA having Formula XI, wherein Rₘ and Rₙ are as defined below:

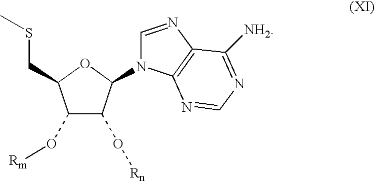

In a preferred embodiment of the invention, the combination therapy includes one or more inhibitors of GARFT and/or AICARFT which are derivatives of 5-thia or 5-selenopyrimidinonyl compounds containing a glutamic acid moiety. In this embodiment, the 5-thia or 5-selenopyrmidinonyl compounds containing a glutamic acid moiety have the Formula I, wherein A, Z, R₁, R₂ and R₃ are as defined herein below:

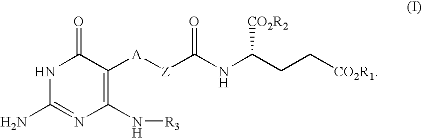

Preferably, the combination therapy comprises GARFT inhibitors having Formula VII, and the tautomers and steroisomers thereof, wherein L, M, T, R₂₀ and R₂₁ are as defined herein below:

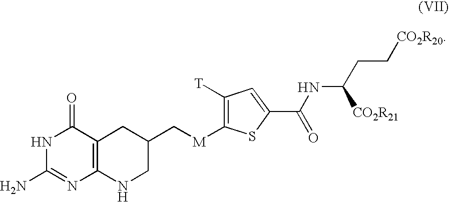

Most preferably, the GARFT inhibitor is a compound having the chemical structure:

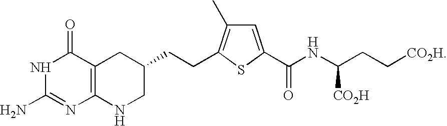

In another embodiment, the inhibitors of de novo inosinate synthesis are inhibitors specific to GARFT and are preferably GARFT inhibitors having a glutamic acid or ester moiety as defined in Formula IV, wherein n, D, M, Ar, R₂₀ and R₂₁ as defined herein below:

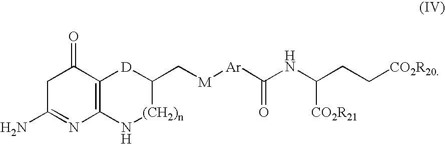

Alternatively, the present invention includes combination therapy with inhibitors specific to AICARFT and are preferably AICARFT inhibitors having a glutamate or ester moiety as defined in Formula VIII, wherein A, W, R₁, R₂ and R₃ as defined herein below.

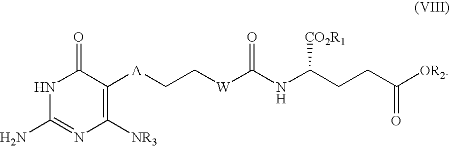

Additional inhibitors specific to AICARFT are also disclosed below.

This combination therapy is administered to a mammal in need thereof. Preferably, the mammal is a human and the anti-toxicity agent is administered to the mammal parenterally or orally. In a further preferred embodiment, the anti-toxicity agent is administered during and after each dose of the inhibitor. In another embodiment the anti-toxicity agent is administered to the mammal by multiple bolus or pump dosing, or by slow release formulations. In a most preferred embodiment, the method is used to treat a cell proliferative disorder selected from the group comprising lung cancer, leukemia, glioma, urothelial cancer, colon cancer, breast cancer, prostate cancer, pancreatic cancer, skin cancer, head and neck cancer.

The present invention is alternatively directed to a combination therapy wherein the inhibitor of GARFT and/or AICARFT does not have a high binding affinity to a membrane binding folate protein (mFBP). Preferably, the inhibitor is predominantly transported into cells by a reduced folate carrier protein. In a further preferred embodiment, the inhibitor is an inhibitor of GARFT having Formula VII. More preferably, the inhibitor is a compound having the chemical structure:

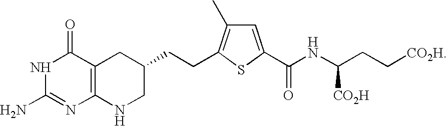

### BRIEF DESCRIPTION OF THE DRAWINGS

FIG. 1 is a chart depicting the intracellular metabolic pathway for production and salvage of adenylate (AMP).

FIG. 2 is a chart depicting the de novo inosinate (IMP) synthesis pathway.

FIG. 3 is a graph indicating the growth inhibition of MTAP-competent SK-MES-1 non-small cell lung cancer cells treated with varying concentrations of Compound 7 alone or with a combination therapy of Compound 7 and 10 μM MTA, as performed in Example 3(A) below.

FIG. 4 is a table indicating the magnitude of in vitro selective reversal of Compound 7 growth inhibition in MTAP-competent versus MTAP-deficient cells treated with Compound 7 and MTA, as in Example 3(A) below.

FIG. 5a is a chart depicting the in vitro cytotoxicity of BxPC-3 cells transfected with the MTAP gene when treated with varying concentrations of Compound 7 either alone or in combination with 50 μM MTA or 50 μM dcSAMe, as in Example 3(B) below.

FIG. 5b is a chart depicting the in vitro cytotoxicity of MTAP-deficient BxPC-3 treated with varying concentrations of Compound 7 in combination with either 50 μM MTA or 50 μM dcSAMe, as in Example 3(B) below.

FIG. 6 is a table indicating the selective reduction of Compound 7 cytoxicity by MTA in isogenic pairs of MTAP-competent and MTAP-deficient cell lines.

FIG. 7 is a table showing the reduced growth inhibition of combination therapy using either Compound 1 or Compound 3, in combination with MTA, in MTAP-competent NCI-H460 cells, as described in Example 3(C) below.

FIG. 8 is a graph showing the reduction in Compound 7 cytotoxicity in cells with MTA exposure for varying periods of time.

FIG. 9 is a graph depicting the decreased weight loss induced by Compound 7 in mice treated with doses of MTA.

FIG. 10 is a graph depicting the antitumour activity of Compound 7 when administered with and without MTA, in mice bearing BxPC-3 xenograft tumors.

### DETAILED DESCRIPTION OF THE INVENTION AND ITS PREFERRED EMBODIMENTS

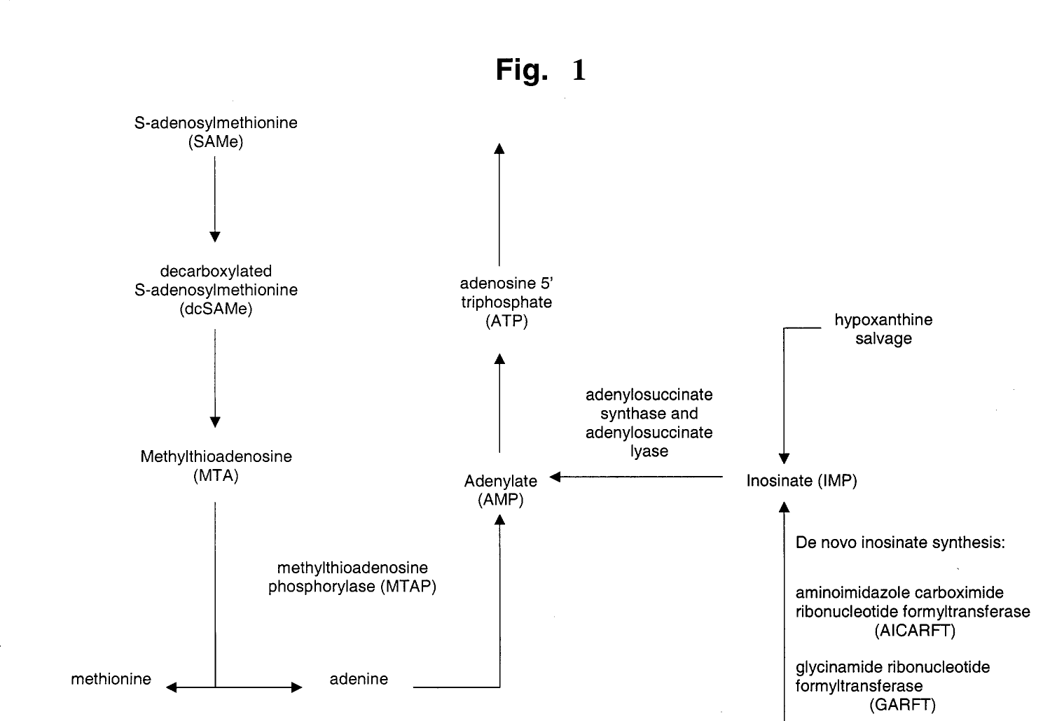

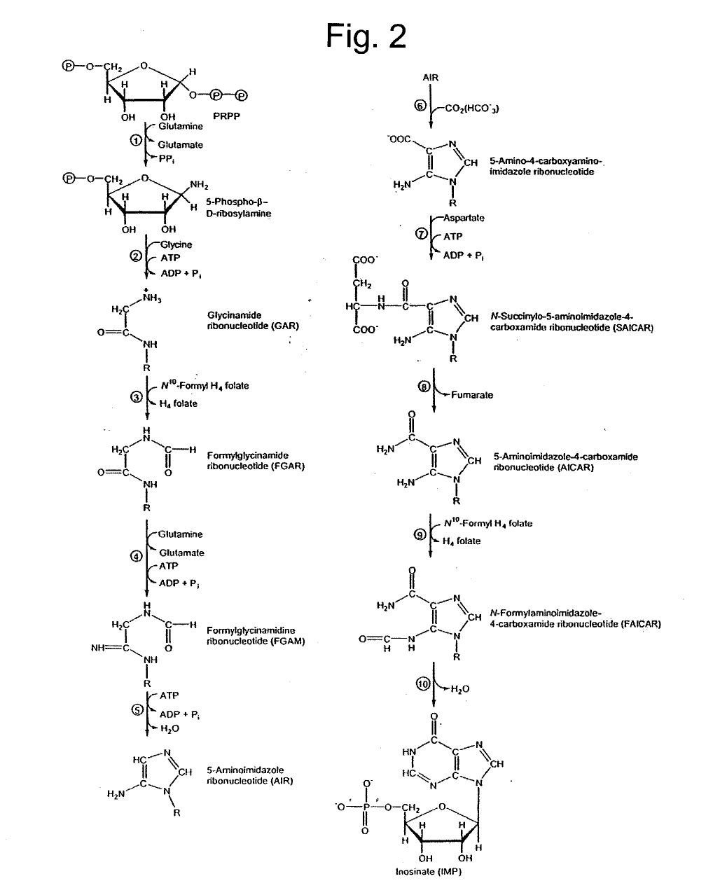

A chart depicting the role of methylthioadenosine phosphorylase (“MTAP”) in relation to the salvage of adenine in the metabolism of healthy cells in mammals is provided in FIG. 1. As depicted in the chart, there are two routes by which adenylate (“AMP”) is produced, by salvage of adenine via methylthioadenosine (“MTA”) and its precursors, and by de novo AMP synthesis via production of inosinate (“IMP”). It has been theorized that tumor cells, due to a high demand for nucleic acid synthesis and genetic alterations in salvage pathway enzymes, tend to make purines by the de novo pathway. In particular, MTAP-deficient cells are unable to cleave MTA into adenine, and are consequently unable to produce AMP via MTAP-mediated adenine salvage. Cells lacking MTAP are particularly reliant on de novo purine synthesis, and are therefore peculiarly vulnerable to disruptions to the de novo pathway. Therefore, MTAP-deficient cells rely on production of AMP via production of inosinate (“IMP”). Referring to FIG. 2, IMP is in turn produced by one of two, pathways, by salvage of hypoxanthine, or by de novo IMP synthesis. Hypoxanthine salvage alone is inadequate to provide a sufficient supply of IMP.

As used herein, “de novo IMP synthesis” refers to the process by which IMP is produced from the starting point of 5-phosphoribosyl-1-pyrophosphate (“PRPP”), as illustrated in FIG. 2. The starting point is the formation of 5′-phospho-β-D-ribosylamine from PRPP by glutamine PRPP amidotransferase (step 1), followed by conversion to glycinamide ribonucleotide (“GAR”) by GAR synthetase (step 2). GAR is then formylated to N-formylglycinamidine ribonucleotide (“FGAR”) by GAR formyltransferase (“GARFT”) (step 3). Synthesis continues with the formation of N-formylglycinamidine ribonucleotide (“FGAM”) by FGAR amidotransferase (step 4), followed by successive formation of 5-aminoimidazolecarboximide ribonucleotide (“AIR”) by AIR synthetase (step 5), 5-Amino-4-carboxyaminoimidazole ribonucleotide by AIR carboxylase (step 6), N-succinylo-5-aminoimidazole-4-carboxamide ribonucleotide (“SAICAR”) by SAICAR synthetase (step 7), 5-aminoimidazole-4-carboxamide ribonucleotide (“AICAR”) by adenylosuccinate lyase (also known as SAICAR lyase) (step 8), and N-Formylaminoimidazole-4-carboxamide ribonucleotide (“FAICAR”) by AICAR transformylase (“AICARFT”) (step 9). Finally, dehydration and ring closure of FAICAR (step 10) leads to production of IMP, which goes on to become either AMP or guanylate monophosphate (“GMP”). A decrease in cellular levels of IMP therefore causes a decrease in the pools along the GMP pathway as well as the AMP pathway.

I. Inhibitors of De Novo IMP Synthesis

As used herein, the term “inhibitor” includes, in its various grammatical forms (e.g., “inhibit”, “inhibition”, “inhibiting”, etc.), an agent, typically a molecule or compound, capable of disrupting and/or eliminating the activity of an enzymatic target involved in the synthesis of a target product. For example, an “inhibitor of de novo IMP synthesis” includes an agent capable of disrupting and/or eliminating the activity of at least one enzymatic target in de novo IMP synthesis, as described above with reference to FIG. 2. An inhibitor of de novo IMP synthesis may have multiple enzymatic targets. When the inhibitor has multiple enzymatic targets, the inhibitor preferably works predominantly through inhibition of one or more targets on the de novo IMP synthesis pathway. In particular, the inhibitors of the present invention preferably inhibit the enzymes glycinamide ribonucleotide formyltransferase (“GARFT”) and/or aminoimidazolecarboximide ribonucleotide formyltransferase (“AICARFT”). The inhibitors of the present invention also include specific inhibitors which have relative specificity or selectivity for inhibiting only one target enzyme on the de novo IMP synthesis pathway, e.g., an inhibitor specific to GARFT.

In one embodiment, the inhibitors of de novo IMP synthesis include inhibitors of GARFT, AICARFT or both, which are derivatives of 5-thia or 5-selenopyrimidinonyl compounds containing a glutamic acid moiety. GARFT and/or AICARFT inhibitors which are derivatives of 5-thia or 5-selenopyrimidinonyl compounds, their intermediates and methods of making the same, are disclosed in U.S. Pat. Nos. 5,739,141; 6,207,670; 5,945,427; and 5,726,312, the disclosures of which are incorporated by reference herein.

In another embodiment, the inhibitor of de novo IMP synthesis is a compound of the Formula I:

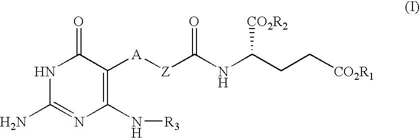

wherein:

A represents sulfur or selenium;

Z represents: a) a noncyclic spacer which separates A from the carbonyl carbon of the amido group by 1 to 10 atoms, said atoms being independently selected from carbon, oxygen, sulfur; nitrogen and phosphorus, said spacer being unsubstituted or substituted with one or more suitable substituents; b) a cycloalkyl, heterocycloalkyl, aryl or heteroaryl diradical, said diradical being unsubstituted or substituted with one or more suitable substituents c) a combination of at least one of said noncyclic spacers and at least one of said diradicals, wherein when said non-cyclic spacer is bonded directly to A, said non-cyclic spacer separates A from one of said diradicals by 1 to about 10 atoms, and further wherein when said non-cyclic spacer is bonded directly to the carbonyl carbon of the amido group, said non-cyclic spacer separates the carbonyl carbon of the amido group from one of said diradicals by 1 to about 10 atoms;

R₁ and R₂ represent, independently, hydro, C₁ to C₆ alkyl, or a readily hydrolyzable group; and

R₃ represents hydro or a cyclic C₁ to C₆ alkyl or cycloalkyl group unsubstituted or substituted by one or more halo, hydroxyl or amino.

In one embodiment of Formula I, the moiety Z is represented by Q-X—Ar wherein:

Q represents a C₁-C₅ alkenyl, or a C₂-C₅ alkenylene or alkynylene radical, unsubstituted or substituted by one or more substitutents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, a cycloalkyl, heterocycloalkyl, aryl or heteroaryl ring;

X represents a methylene, monocyclic cycloalkyl, heterocycloalkyl, aryl or heteroaryl ring, sulfur, oxygen or amino radical, unsubstituted or substituted by one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring; and

Ar represents a monocyclic or bicyclic cycloalkyl, heterocycloalkyl, aryl or heteroaryl ring, wherein Ar may be fused to the monocyclic cycloalkyl, heterocycloalkyl, aryl or heteroaryl ring of X, said Ar is unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring.

The term “alkyl” refers to a straight- or branched-chain, saturated or partially unsaturated, alkyl group having from 1 to about 12 carbon atoms, preferably from 1 to about 6 carbon atoms in the chain. Exemplary alkyl groups include methyl (Me, which also may be structurally depicted by/), ethyl (Et), n-propyl, isopropyl, butyl, isobutyl, sec-butyl, tert-butyl (tBu), pentyl, isopentyl, tert-pentyl, hexyl, isohexyl, and the like.

The term “heteroalkyl” refers to a straight- or branched-chain, saturated or partially unsaturated alkyl group having from 2 to about 12 atoms, and preferably from 2 to about 6 atoms, in the chain, one or more of which is a heteroatom selected from S, O, and N. Exemplary heteroalkyls include alkyl ethers, secondary and tertiary alkyl amines, alkyl sulfides, and the like.

The term “alkenyl” refers to a straight- or branched-chain alkenyl group having from 2 to about 12 carbon atoms, preferably from 2 to about 6 carbon atoms, in the chain. Illustrative alkenyl groups include prop-2-enyl, but-2-enyl, but-3-enyl, 2-methylprop-2-enyl, hex-2-enyl, ethenyl, pentenyl, and the like.

The term “alkynyl” refers to a straight- or branched-chain alkynyl group having from 2 to about 12 carbon atoms, and preferably from 2 to about 6 carbon atoms, in the chain. Illustrative alkynyl groups include prop-2-ynyl, but-2-ynyl, but-3-ynyl, 2-methylbut-2-ynyl, hex-2-ynyl, ethynyl, propynyl, pentynyl and the like.

The term “aryl” (Ar) refers to a monocyclic, or fused or spiro polycyclic, aromatic carbocycle (ring structure having ring atoms that are all carbon) having from to about 12 ring atoms, and preferably from 3 to about 8 ring atoms, per ring. Illustrative examples, of aryl groups include the following moieties:

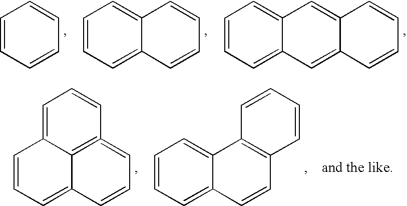

The term “heteroaryl” (heteroAr) refers to a monocyclic, or fused or spiro polycyclic, aromatic heterocycle (ring structure having ring atoms selected from carbon atoms as well as nitrogen, oxygen, and sulfur heteroatoms) having from 3 to about 12 ring atoms, and preferably from 3 to about 8 ring atoms, per ring. Illustrative examples of heteraryl groups include the following moieties:

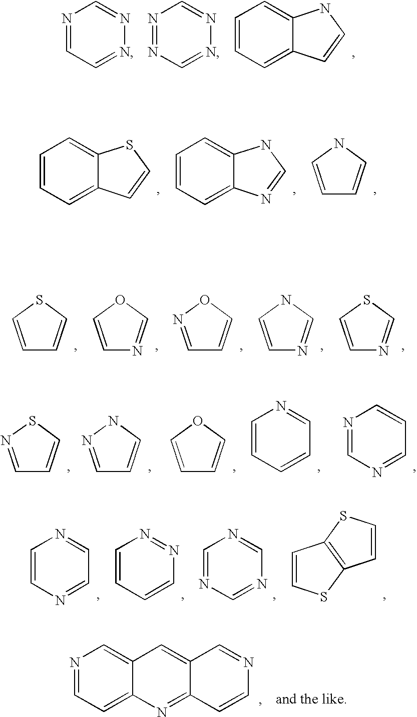

The term “cycloalkyl” refers to a saturated or partially saturated, monocyclic or fused or spiro polycyclic, carbocycle having from 3 to 12 ring atoms, and preferably from 3 to about 8 ring atoms, per ring. Illustrative examples of cycloalkyl groups include the following moieties:

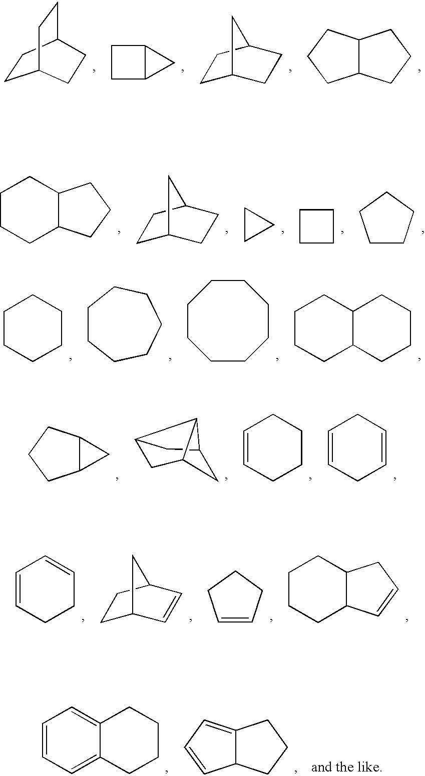

A “heterocycloalkyl” refers to a monocyclic, or fused or spiro polycyclic, ring structure that is saturated or partially saturated and has from 3 to about 12 ring atoms, and preferably from 3 to about 8 ring atoms, per ring selected from C atoms and N, O, and S heteroatoms. Illustrative examples of heterocycloalkyl groups include:

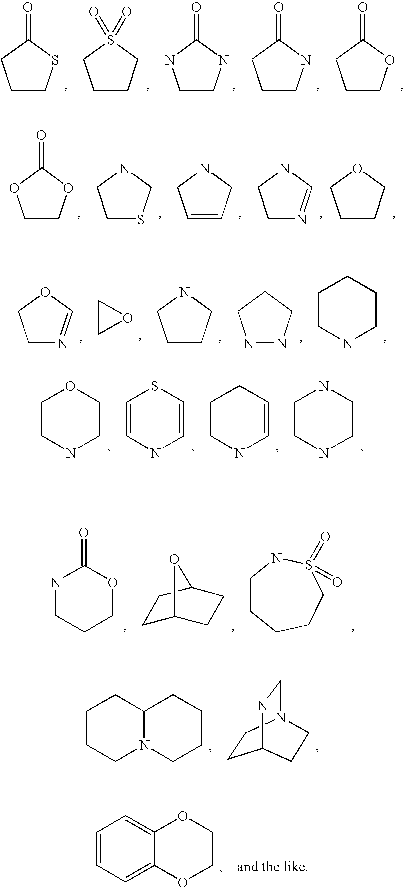

The term “halogen” represents chlorine, fluorine, bromine or iodine. The term “halo” represents chloro, fluoro, bromo or iodo. An “amino” group is intended to mean the radical —NH₂. A “mercapto” group is intended to mean the radical —SH. An “acyl” group is intended to mean any carboxylic acid, aldehyde, ester, ketone of the formula —C(O)H, —C(O)OH, —C(O)Rₜ, —C(O)ORₜ wherein Rₜ is any alkyl, alkenyl, alkynyl, heteroalkyl, cycloalkyl, heterocycloalkyl, aryl, or heteroaryl. Examples of acyl groups include, but are not limited to, formaldehyde, benzaldehyde, dimethyl ketone, acetone, diketone, peroxide, acetic acid, benzoic acid, ethyl acetate, peroxyacid, acid anhydride, and the like.

An “alkoxy group” is intended to mean the radical —ORₐ, where Rₐ is an alkyl group. Exemplary alkoxy groups include methoxy, ethoxy, and propoxy. “Lower alkoxy” refers to alkoxy groups wherein the alkyl portion has 1 to 4 carbon atoms.

An “hydrolyzable group” is intended to mean any group which can be hydrolyzed in an aqueous medium, either acidic or alkaline, to its free carboxylate form by means known in the art. An exemplary hydrolysable group is the glutamic acid dialkyl diester which can be hydrolyzed to either the free glutamic acid or the glutamate salt. Preferred hydrolysable ester groups include C₁-C₆ alkyl, hydroxyalkyl, alkylaryl and aralkyl.

In accordance with a convention used in the art,

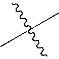

is used in structural formulae herein to depict the bond that is the point of attachment of the moiety or substituent to the core or backbone structure. Where chiral carbons are included in chemical structures, unless a particular orientation is depicted, both stereoisomeric forms are intended to be encompassed. Further, the specific inhibitors of the present invention may exist as single stereoisomers, racemates, and/or mixtures of enantiomers and/or diastereomers. All such single stereoisomers, racemates, and mixtures thereof are intended to be within the broad scope of the present invention. The chemical formulae referred to herein may exhibit the phenomenon of tautomerism. Although the structural formulae depict one of the possible tautomeric forms, it should be understood that the invention nonetheless encompasses all tautomeric forms.

The term “substituted” means that the specified group or moiety bears one or more substituents. The term “unsubstituted” means that the specified group bears no substituents. The term “substituent” or “suitable substituent” is intended to mean any suitable substituent that may be recognized or selected, such as through routine testing, by those skilled in the art. Unless expressly indicated otherwise, illustrative examples of suitable substituents include alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—OR_{c}, —(CH₂)_{z}—CN where z is 0-4, halo, —OH, —O—Rₐ—O—R_{b}, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NR_{d}Rₑ, —O—CO—NR_{d}Rₑ, —NR_{c}—CO—NR_{d}Rₑ, —NR_{c}—CO—Rₑ—NR_{c}—CO₂—ORₑ, —CO—NR_{c}—CO—R_{d}, —O—SO₂—Rₑ, —O—SO—R_{c}, —O—S—R_{c}, —S—CO—R_{c}, —SO—CO—OR_{c}, —SO₂—CO—OR_{c}, —O—SO₃, —NR_{c}—SR_{d}, —NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂—R_{c}, —SO₂—NR_{d}Rₑ, —SO—NR_{d}Rₑ, —S—NR_{d}Rₑ, —NR_{d}—CSO₂—R_{d}, —NR_{c—CSO—R}_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, hydroxyl, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group; and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —(CH₂)_{z}—CN where z is 0-4, halo, haloalkyl, haloaryl, —OH, keto (═O), —N—OH, NR_{c}—OR_{c}, —NR_{d}Rₑ, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d}, and Rₑ are as defined above.

In another embodiment of Formula I, the inhibitors are compounds having Formula II:

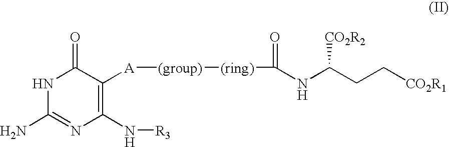

wherein:

A represents sulfur or selenium;

(group) represents a non-cyclic spacer which separates A from (ring) by 1 to 5 atoms, said atoms being independently selected from carbon, oxygen, sulfur, nitrogen and phosphorus, said spacer being unsubstituted or substituted by one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring;

(ring) represents a cycloalkyl, heterocycloalkyl, aryl or heteroaryl ring, unsubstituted or substituted with or more substituents selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring;

R₁ and R₂ represent, independently, hydro, C₁ to C₆ alkyl, or a readily hydrolyzable group; and

R₃ represents hydro or a C₁ to C₆ alkyl or cycloalkyl group unsubstituted or substituted by one or more halo, hydroxyl or amino.

Preferred species of Formula II are compounds having the following chemical structures:

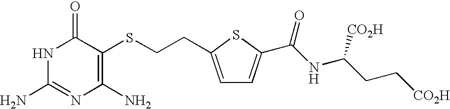

(Compound 1: N-[5-(2[(2,6-diamino-4(3H)-oxopyrimidin5yl)thio]ethyl)thieno-2-yl]-L-glutamic acid); and

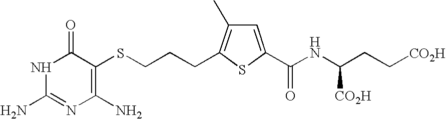

(Compound 2: N-[5-(3-[(2,6-diamino-4(3H)-oxopyrimidin-5yl)thio]propyl)-4-methyl-thieno-2-yl/-L-glutamic acid).

In yet another embodiment of Formula I, the inhibitors are compounds having Formula III:

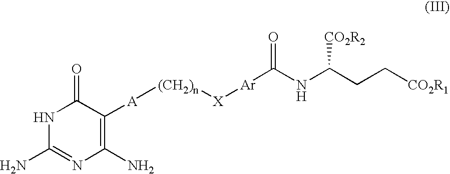

wherein:

n is an integer from 0 to 5;

A represents sulfur or selenium;

X represents a diradical of methylene, a monocyclic cycloalkyl, heterocycloalkyl, aryl or heteroaryl ring, oxygen, sulfur or an amine;

Ar represents an aromatic diradical wherein Ar can form a fused bicyclic ring system with said ring of X; and

R₁ and R₂, represent, independently, hydro or C₁-C₆ alkyl.

In an alternative embodiment, the inhibitors of de novo IMP synthesis include inhibitors of GARFT having a glutamic acid or ester moiety. GARFT inhibitors having a glutamic acid or ester moiety, their intermediates and methods of making thereof are disclosed in U.S. Pat. Nos. 5,723,607; 5,641,771; 5,639,749; 5,639,747; 5,610,319; 5,641,774; 5,625,061; and 5,594,139; the disclosures of which are hereby incorporated by reference in their entireties. In particular, GARFT inhibitors having a glutamic acid or ester moiety include compounds having the Formula IV:

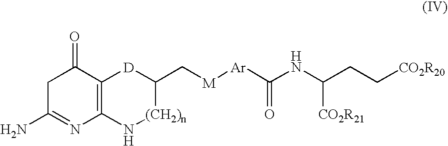

wherein:

n represents an integer from 0 to 2;

D represents sulfur, CH₂, oxygen, NH or selenium, provided that when n is 0, D is not CH₂, and when n is 1, D is not CH₂ or NH;

M represents sulfur, oxygen, or a diradical of C₁-C₃ alkane, C₂-C₃ alkene, C₂-C₃ alkyne, or amine, wherein M is unsubstituted or substituted by one or more suitable substituents;

Ar represents a diradical of a cycloalkyl, heterocycloalkyl, aryl or heteroaryl ring system, said Ar is unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring; and

R₂₀ and R₂₁ represent, independently, hydro or a moiety that forms, together with the attached CO₂, a readily hydrolyzable ester group.

In one embodiment of Formula IV, the inhibitors are compounds having the Formula V:

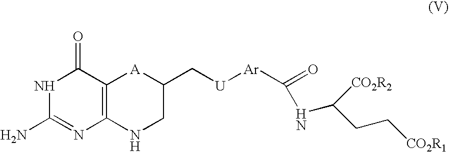

wherein:

A represents sulfur or selenium;

U represents CH₂, sulfur, oxygen or NH;

Ar represents a diradical of a cycloalkyl, heterocycloalkyl, aryl or heteroaryl ring system, said Ar is unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring; and

R₂₀ and R₂₁ represent, independently, hydro or a moiety that forms, together with the attached CO₂, a readily hydrolyzable ester group.

In another embodiment of Formula IV, the inhibitors are compounds having the Formula VI:

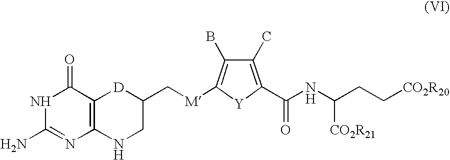

wherein:

D represents oxygen, sulfur or selenium;

M′ represents sulfur, oxygen, or a diradical of C₁-C₃ alkene, C₂-C₃ alkene, C₂-C₃ alkyne, or amine, said M′ is unsubstituted or substituted by one or more suitable substituents;

Y represents O, S or NH;

B represents hydro or halo;

C represents hydro or halo or an unsubstituted or substituted C₁-C₆ alkyl; and

R₂₀ and R₂₁ represent independently hydro or a moiety that forms, together with the attached CO₂, a readily hydrozyable ester group.

One preferred species of GARFT inhibitor of Formula VI is a compound having the chemical structure:

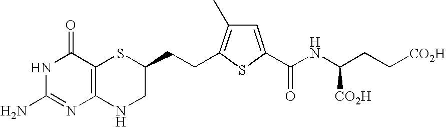

(Compound 3: 4-[2-(2-Amino-4-oxo-4,6,7,8-tetraydro-3H-pyrimido[5,4-b][1,4]thiazin-6-yl)(R)-ethyl]-3-methyl-2-thienoyl-5-amino-L-glutamic acid).

In another alternative embodiment of the invention, the inhibitors of de novo IMP synthesis are inhibitors specific to GARFT having the Formula VII:

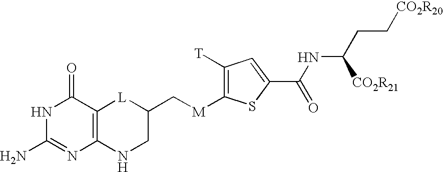

wherein L represents sulfur, CH₂ or selenium;

M represents a sulfur, oxygen, or a diradical of C₁-C₃ alkane, C₂-C₃ alkene, C₂-C₃ alkyne, or amine, wherein M is unsubstituted or substituted by one or more suitable substituents;

T represents C₁-C₆ alkyl; C₂-C₆ alkenyl; C₂-C₆ alkynyl; —C(O)E, wherein E represents hydro, C₁-C₃ alkyl, C₂-C₃ alkenyl, C₂-C₃ alkynyl, OC₁-C₃ alkoxy, or NR₁₀R₁₁, wherein R₁₀ and R₁₁ represent independently hydro, C₁-C₃ alkyl, C₂-C₃ alkenyl, C₂-C₃ alkynyl; or NR₁₀R₁₁, wherein R₁₀ and R₁₁ represent independently hydro, C₁-C₃ alkyl, C₂-C₃ alkenyl, C₂-C₃ alkynyl, hydroxyl; nitro; SR₁₂, wherein R₁₂ is hydro, C₁-C₆ alkyl, C₂-C₆ alkenyl, C₂-C₆ alkynyl, cyano; or O(C₁-C₃) alkyl; and

R₂₀ and R₂₁ are each independently hydro or a moiety that forms, together with the attached CO₂, a readily hydrolyzable ester group.

GARFT inhibitors having Formula VII, and the tautomers and stereoisomers thereof, are capable of particularly low binding affinities to mFBP. These inhibitors are capable of having mFBP disassociation constants that are at least thirty five times greater than lometrexol and are disclosed in U.S. Pat. Nos. 5,646,141 and 5,608,082, the disclosures of which are hereby incorporated by reference in their entireties.

Preferred species of a GARFT inhibitor of Formula VII are compounds having the following chemical structures:

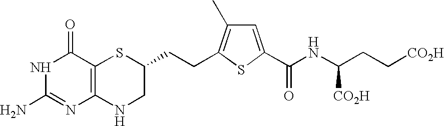

(Compound 4: 4-[2-(2-Amino-4-oxo-4,6,7,8-tetraydro-3H-pyrimido[5,4-b][1,4]thiazin-6-yl)-(R)-ethyl]-3-methyl-2-thienoyl-5-amino-L-glutamic acid),

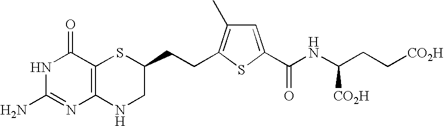

(Compound 5: 4-[2-(2-Amino-4-oxo-4,6,7,8-tetrahydro-3H-pyrimido[5,4-b][1,4]thiazin-6-yl)-(S)-ethyl]-3-methyl-2-thienoyl-5-amino-L-glutamic acid), and

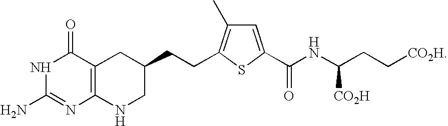

(Compound 6: N-(5-[2-(2-amino-4(3H)-oxo-5,6,7,8-tetrahydropyrido[2,3-d]pyrimidin-6-yl)-(R)-ethyl]-4-methylthieno-2-yl)-L-glutamic acid).

A more preferred species of a GARFT inhibitor having the formula VII, and which has limited binding affinity to mFBP, is a compound having the chemical structure:

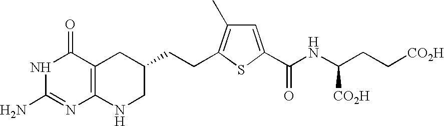

(Compound 7: N-(5-[2-(2-amino-4(3H)-oxo-5,6,7,8-tetrahydropyrido[2,3-d]pyrimidin-6-yl)-(S)-ethyl]-4-methylthieno-2-yl)-L-glutamic acid).

In another alternate embodiment, the inhibitors of de novo IMP synthesis include inhibitors specific to AICARFT which also have a glutamate or ester moiety. AICARFT inhibitors having a glutamate or ester moiety, their intermediates and methods of making the same are disclosed in U.S. Pat. Nos. 5,739,141; 6,207,670; 5,945,427; and 5,726,312, the disclosures of which are hereby incorporated by reference in their entireties. In particular, AICARFT inhibitors having a glutamate or ester moiety include compounds having the Formula VIII:

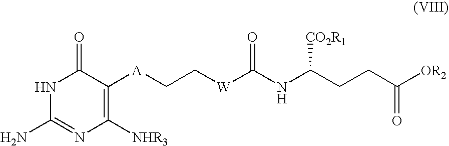

wherein:

A represents sulfur or selenium;

W represents an unsubstituted phenylene or thinylene diradical;

R₁ and R₂ represent, independently, hydro, C₁ to C₆ alkyl, or other readily hydrolyzable group; and

R₃ represents hydro or a C₁-C₆ alkyl or cycloalkyl group, unsubstituted or substituted by one or more halogen, hydroxyl or amino groups.

Additional AICARFT inhibitors useful in the present invention are disclosed in International Publication No. WO13688, the disclosure of which is hereby incorporated by reference in its entirety. In particular, the disclosed AICARFT inhibitors are compounds having the Formula IX:

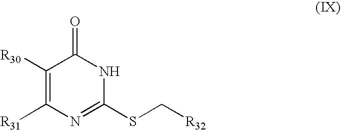

wherein:

R₃₀ represents hydro or CN;

R₃₁ represent phenyl or thienyl, unsubstituted or substituted with phenyl, phenoxy, thienyl, tetrazolyl, or 4-morpholinyl; and

R₃₂ is phenyl substituted with —SO₂NR₃₃R₃₄ or —NR₃₃SO₂R₃₄, unsubstituted or substituted with C₁-C₄ alkyl, C₁-C₄ alkoxy, or halo, wherein R₃₃ is H or C₁-C₄ alkyl and R₃₄ is C₁-C₄ alkyl, unsubstituted or substituted with heteroalkyl, aryl, heteroaryl, indolyl, or is

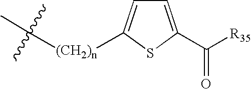

wherein n is an integer of from 1 to 4, R₃₅ is hydroxyl, C₁-C₄ alkoxy, or a glutamic-acid or glutamate-ester moiety linked through the amine functional group.

Preferred species of AICARFT inhibitors useful in the method of this invention include compounds having the following chemical structures:

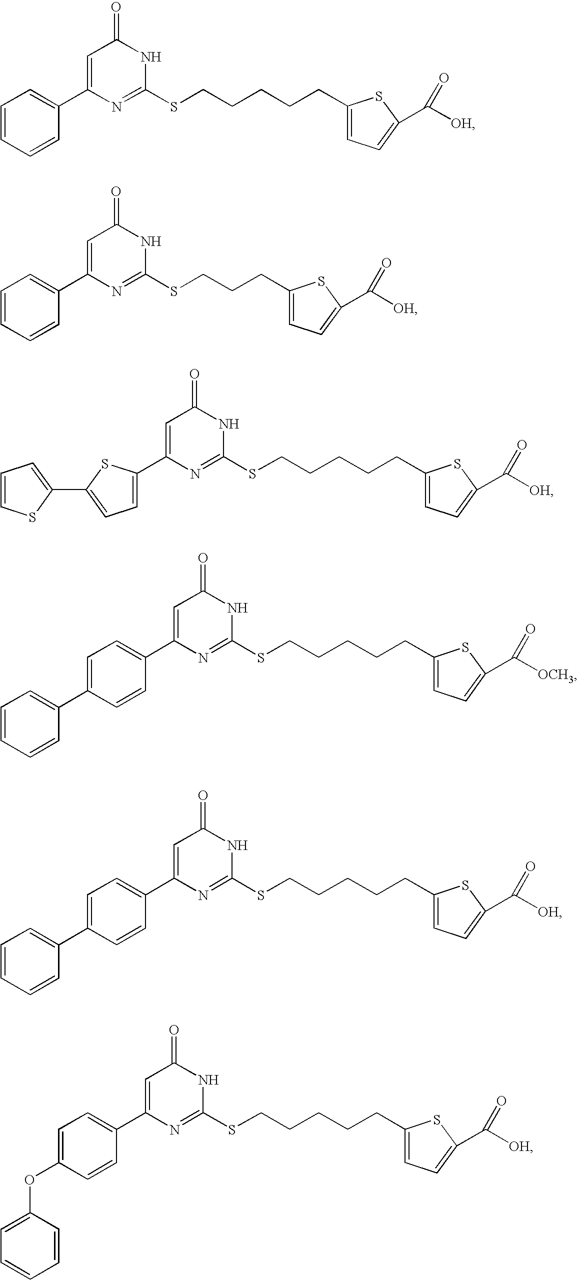

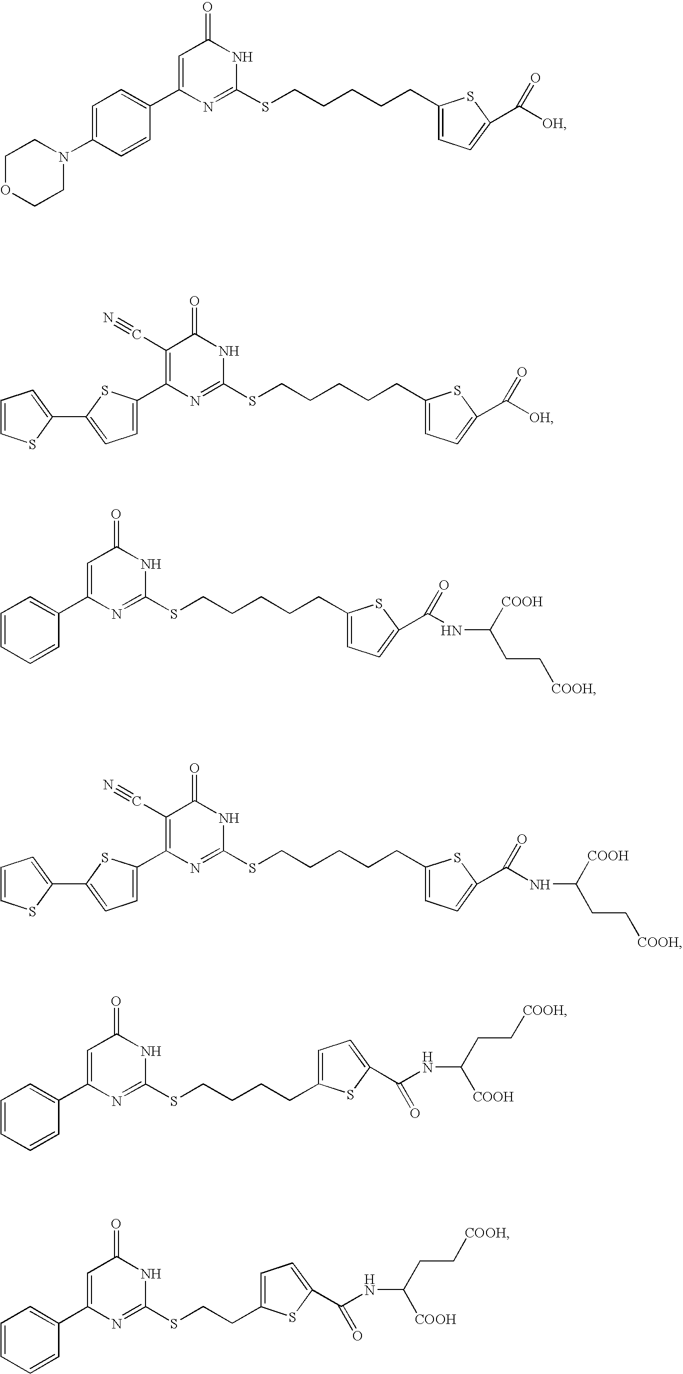

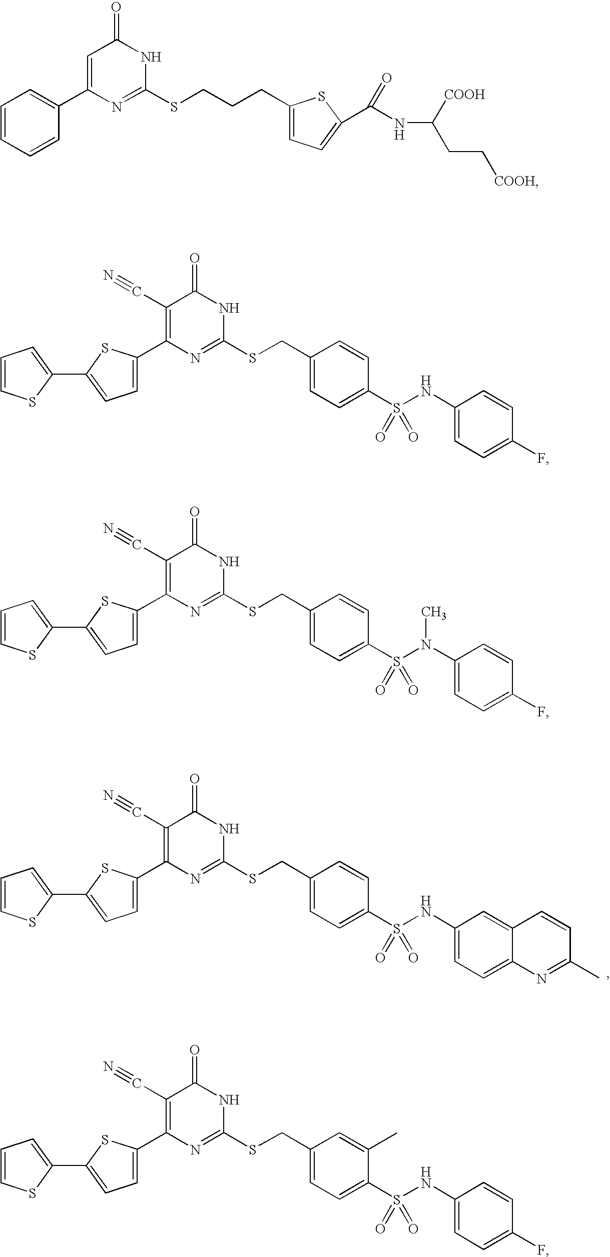

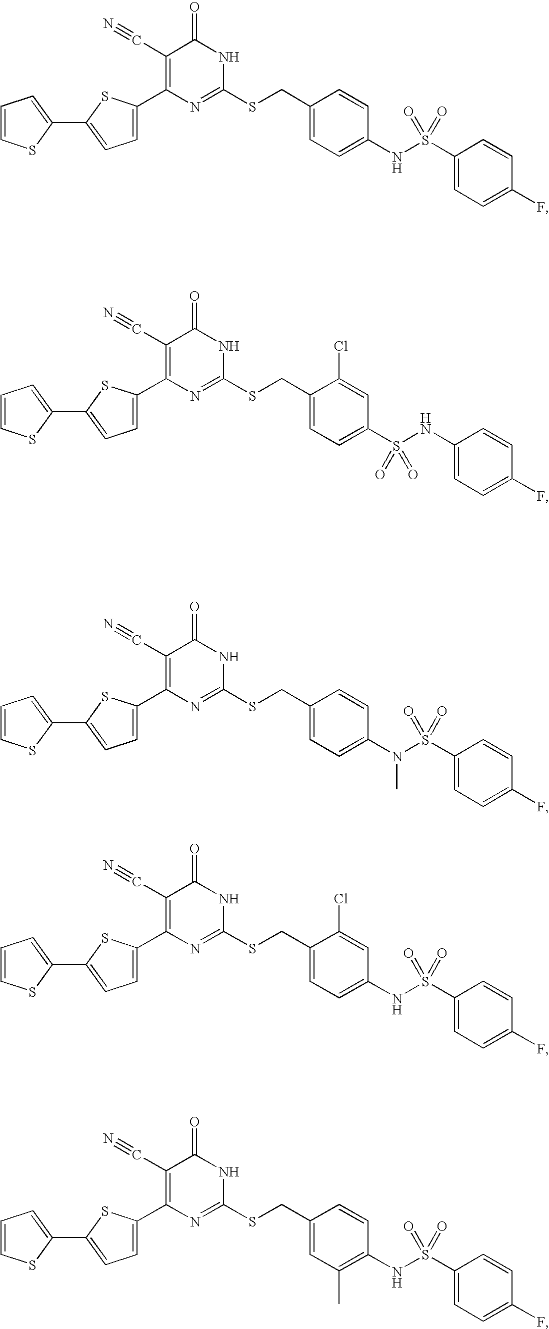

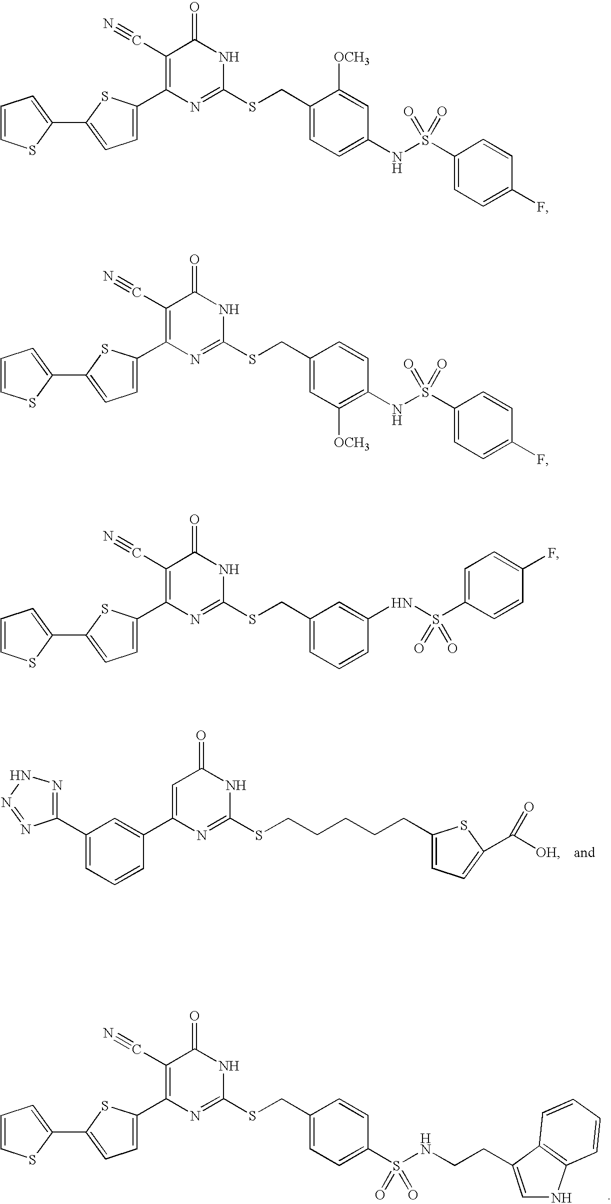

The inhibitors of de novo IMP synthesis useful in the methods of the present invention include any pharmaceutically acceptable salt, prodrug, solvate or pharmaceutically active metabolite thereof. As used herein, a “prodrug” is a compound that may be converted under physiological conditions or by solvolysis to the specified compound or to a pharmaceutically acceptable salt of such compound. An “active metabolite” is a pharmacologically active product produced through metabolism in the body of a specified compound or salt thereof. Prodrugs and active metabolites of a compound may be routinely identified using techniques known in the art. See, e.g., Bertolini et al., J. Med. Chem. (1997), 40:2011-2016; Shan et al., J. Pharm. Sci. (1997), 86 (7):765-767; Bagshawe, Drug Dev. Res. (1995), 34:220-230; Bodor, Advances in Drug Res. (1984), 13:224-331; Bundgaard, Design of Prodrugs (Elsevier Press 1985); Larsen, Design and Application of Prodrugs, Drug Design and Development (Krogsgaard-Larsen et al. eds., Harwood Academic Publishers, 1991); Dear et al., J. Chromatogr. B (2000), 748:281-293; Spraul et al., J. Pharmaceutical & Biomedical Analysis (1992), 10 (8):601-605; and Prox et al., Xenobiol. (1992), 3 (2):103-112. A “pharmaceutically acceptable salt” is intended to mean a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, (hydroxybutyrates, glycollates, tartrates, methane-sulfonates (mesylates), propanesulfonates, naphthalene-1-sulfonates, naphthalene-2-sulfonates, and mandelates. A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine.

In the case of compounds, salts, or solvates that are solids, it is understood by those skilled in the art that the useful inhibitor compounds, salts, and solvates of the invention may exist in different crystal forms, all of which are intended to be within the scope of the inhibitors of the present invention and their specified formulae. The inhibitor compounds according to the invention, as well as the pharmaceutically acceptable prodrugs, salts, solvates or pharmaceutically active metabolites thereof, may be incorporated into convenient dosage forms such as capsules, tablets or injectable preparations. Solid or liquid pharmaceutically acceptable carriers may also be employed. Solid carriers include starch, lactose, calcium sulphate dihydrate; terra alba, sucrose, talc, gelatin, agar, pectin, acacia, magnesium stearate and stearic acid. Liquid carriers include syrup, peanut oil, olive oil, saline solution and water, among other carriers well known in the art.

As mentioned above, the inhibitors of de novo IMP synthesis useful in the present invention are preferably capable of inhibiting GARFT and/or AICARFT and have a relative affinity that is higher for GARFT and/or AICARFT than for other enzymes in the de novo IMP synthesis pathway. More preferably, the inhibitors useful in the invention are specific to either GARFT or AICARFT, by having a relative affinity that is higher for either GARFT or AICARFT.

In a preferred embodiment, the inhibitors useful in the methods of the present invention do not have a high affinity to membrane folate binding protein (“mFBP”) and preferably have a disassociation constant to mFBP that is greater than lometrexol by at least a factor of about thirty-five. The disassociation constant to mFBP may be determined by using a competitive binding assay with mFBP, as described below. Accordingly, the inhibitors useful in the present invention are predominantly transported into cells by an alternate mechanism other than that involving mFBP, for example, via a reduced folate transport protein. The reduced folate transport protein has a preference for reduced folates but will transport a number of folic acid derivatives.

A. Determination of Inhibition Constants for Inhibitors of De Novo IMP Synthesis

The determination of inhibition constants for de novo IMP inhibitors may be conducted as per the assays disclosed in U.S. Pat. No. 5,646,141 or International Publication No. WO 13688, the disclosures of which are hereby incorporated by reference in their entireties. In particulars the inhibition constant can be determined by modifying the assay method of Young et al, Biochemistry 23 (1984) 3979-3986 or of Black et al, Anal. Biochem. 90 (1978) 397-401, the disclosures of which are also hereby incorporated by reference in their entireties. Generally, the reaction mixtures are designed to contain the catalytic domain of the human enzyme and its substrate (i.e., GARFT and GAR, or AICARFT and AICAR), the subject test inhibitor, and any necessary substrates (i.e. N¹⁰-formyl-5,8-dideazafolate). The reaction is initiated by addition of the enzyme and then monitored for an increase in absorbance at 298 nm at 25° C.

The inhibition constant (Kᵢ) can be determined from the dependence of the steady-state catalytic rate on inhibitor and substrate concentration. The type of inhibition observed is then analyzed for competitiveness with respect to any substrate of the target enzyme (e.g. N¹⁰-formyl H₄ folate or its analog, formyl-5,8-dideazafolate (“FDDF”), for GARFT and AICARFT inhibitors). The Michaelis constant Kₘ for N¹⁰-formyl H₄ folate or FDDF is then determined independently by the dependence of the catalytic rate on substrate concentration. Data for both the Kₘ and Kᵢ determinations are fitted by non-linear methods to the Michaelis equation, or the Michaelis equation for competitive inhibition, as appropriate. Data resulting from tight-binding inhibition is then analyzed and Kᵢ is determined by fitting the data to the tight-binding equation of Morrison, Biochem Biophys Acta 185 (1969), 269-286, using nonlinear methods.

B. Determination of Disassociation Constants for Human Membrane Folate Binding Protein

The dissociation constant (K_{d}) of the preferred inhibitors of the present invention for human membrane folate-binding protein (mFBP) can be determined in a competitive binding assay using mFBP prepared from cultured KB cells (human nasopharyngeal carcinoma cells) as disclosed in U.S. Pat. No. 5,646,141, the disclosures of which is hereby incorporated by reference in its entirety.

Human membrane folate binding protein can be obtained from KB cells by methods well known in the art. KB cells are washed, sonicated for cell lysis and centrifuged to form pelleted cells. The pellet can then be stripped of endogenous bound folate by resuspension in acidic buffer (KH₂PO₄—KOH and 2-mercaptoethanol) and centrifuged again. The pellet is then resuspended and the protein content quantitated using the Bradford method with bovine serum albumin (BSA) as standard.

Disassociation constants are determined by allowing, the test inhibitor to compete against ³H-folic acid for binding to mFBP. Reaction mixtures are designed to generally contain mFBP, ³H-folic acid, and various concentrations of the subject test inhibitor in acidic buffer (KH₂PO₄—KOH and 2-mercaptoethanol). The competition reaction is typically conducted at 25°. Because of the slow nature of release of bound ³H-folic acid, the test inhibitor may be prebound prior to addition of bound ³H-folic acid, after which the reaction should be allowed to equilibriate. The full reaction mixtures then should be drawn through nitrocellulose filters to isolate the cell membranes with bound ³H-folic acid. The trapped mFBP are then washed and measured by scintillation counting. The data can then be nonlinearly fitted as described above in determining Kᵢ. The mFBP K_{d} for ³H-folic acid, used for calculating the competitor K_{d}, can be obtained by directly titrating mFBP with ³H-folate. The mFBP K_{d} can then be used to calculate the competitor K_{d} by nonlinear fitting of the data to an equation for tight-binding K_{c}. Table 1 below provides the K_{d} values of several GARFT inhibitors using the assay described above.

TABLE 1

| GARFT Inhibitor | K_{d} (nM) to mFBP |
|---|---|
| Lometrexol | 0.019 |
| Compound 2 | 136 |
| Compound 3 | 0.0042 |
| Compound 4 | 1.0 |
| Compound 5 | 0.71 |
| Compound 7 | 290 |

II. Anti-Toxicity Agents

To reduce the toxicity of an IMP inhibitor on non-cancerous, MTAP-competent cells, an anti-toxicity agent is administered in combination with the inhibitor to provide a supply of adenine or AMP. The anti-toxicity agent comprises an MTAP substrate (e.g. methylthioadenosine or “MTA”), a precursor of MTA, an analog of an MTA precursor, a prodrug of an MTAP substrate, or a combination thereof. As used herein, an “MTAP substrate” refers to MTA or a synthetic analog of MTA, which is capable of providing a substrate for cleavage by MTAP for production of either adenine or AMP. MTA is represented by the chemical structure below:

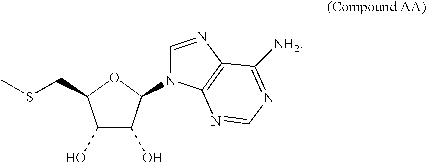

MTA can be prepared according to known methods as disclosed in Kikugawa et al. J. Med. Chem. 15, 387(1972) and Robins et al. Can. J. Chem. 69,1468 (1991). An alternate method of synthesizing MTA is provided in Example 2(A) below.

As used herein, an “analog of MTA” refers to any compound related to MTA in physical structure and which is capable of providing a cleavage site for MTAP. Synthetic analogs can be prepared to provide a substrate for cleavage by MTAP, which in turn provides adenine or AMP.

In one embodiment, the anti-toxicity agents of the present invention are analogs of MTA having the Formula X:

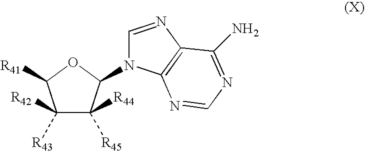

wherein

R₄₁ is selected from the group consisting of:

(a) —R_{g} wherein R_{g} represents a C₁-C₅ alkyl, C₂-C₅ alkenylene or alkynylene radical, unsubstituted or substituted by one or more substitutents independently selected from C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl;

(b) —R_{g}(Y)RₕRᵢ wherein R_{g} is as defined above, Y represents O, NH, S, or methylene; and Rₕ and Rᵢ represent, independently, (i) H; (ii) a C₁-C₉ alkyl, or a C₂-C₆ alkenyl or alkynyl, unsubstituted or substituted by one or more substitutents independently selected from C₁ to C₆ alkoxy; C₁ to C₆ alkoxy(C₁ to C₆)alkyl; C₂ to C₆ alkynyl; acyl; halo; amino; hydroxyl; nitro; mercapto; —NCOORₒ; —CONH₂; C(O)N(Rₒ)₂; C(O)Rₒ; or C(O)ORₒ, wherein Rₒ is selected from the group consisting of H, C₁-C₆ alkyl, C₂-C₆ heterocycloalkyl, cycloalkyl, heteroaryl, aryl, and amino, unsubstituted or substituted with C₁-C₆ alkyl, 2- to 6-membered heteroalkyl, heterocycloalkyl, cycloalkyl, C₁-C₆ boc-aminoalkyl; cycloalkyl, heterocycloalkyl, aryl or heteroaryl; or (iii) a monocyclic or bicyclic cycloalkyl, heterocycloalkyl, aryl or heteroaryl, unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl heteroaryl, —COORₒ, —NCORₒ wherein Rₒ is as defined above, 2 to 6 membered heteroalkyl, C₁ to C₆ alkyl-cycloalkyl, C₁ to C₆ alkyl-heterocycloalkyl, C₁ to C₆ alkyl-aryl or C₁ to C₆ alkyl-aryl;

(c) C(O)NRⱼRₖ wherein Rⱼ and Rₖ represent, independently, (i) H; or (ii) a C₁-C₆ alkyl, amino, C₁-C₆ haloalkyl, C₁-C₆ aminoalkyl, C₁-C₆ boc-aminoalkyl, C₁-C₆ cycloalkyl, C₁-C₆ alkenyl, C₂-C₆ alkenylene, C₂-C₆ alkynylene radical, wherein Rⱼ and Rₖ are optionally joined together to form, together with the nitrogen to which they are bound, a heterocycloalkyl or heteroaryl ring containing two to five carbon atoms and wherein the C(O)NRⱼRₖ group is further unsubstituted or substituted by one or more substitutents independently selected from —C(O)Rₒ, —C(O)ORₒ wherein Rₒ is as defined above, C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl; or

(d) C(O)ORₕ wherein Rₕ is as defined above;

R₄₂ and R₄₄ represent, independently, H or OH; and

R₄₃ and R₄₅ represent, independently, H, OH, amino or halo;

where any of the cycloalkyl, heterocycloalkyl, aryl, heteroaryl moieties present in the above may be further substituted with one or more additional substituents independently selected from the group consisting of nitro, amino, —(CH₂)_{z}—CN where z is 0-4, halo, haloalkyl, haloaryl, hydroxyl, keto, C₁ to C₆ alkyl, C₂ to C₆ alkenyl, C₂ to C₆ alkynyl, heteroalkyl, unsubstituted cycloalkyl, unsubstituted heterocycloalkyl, unsubstituted aryl or unsubstituted heteroaryl;

and salts or solvates thereof.

In another embodiment, the anti-toxicity agents of the present invention are analogs of MTA having the Formula XII:

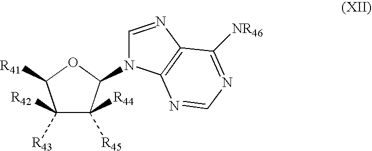

wherein R₄₆ represents (i) H; (ii) a C₁-C₉ alkyl, or a C₂-C₆ alkenyl or alkynyl, unsubstituted or substituted by one or more substitutents independently selected from C₁ to C₆ alkoxy; C₁ to C₆ alkoxy(C₁ to C₆)alkyl; C₂ to C₆ alkynyl; acyl; halo; amino; hydroxyl; nitro; mercapto; cycloalkyl, heterocycloalkyl, aryl or heteroaryl; or (iii) a monocyclic or bicyclic cycloalkyl, heterocycloalkyl, aryl or heteroaryl, unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, imercapto, cycloalkyl, heterocycloalkyl, aryl or heteroaryl; and wherein R₄₁, R₄₂, R₄₃, R₄₄ and R₄₅ are as described above.

MTA analogs can be prepared via literature methods. The 5′ thio analogs of adenosine can be prepared from 5′-chloro-5′-deoxyadenosine (Kikugawa et al. J. Med. Chem. 15, 387 (1972) and M. J. Robins et. al. Can. J. Chem. 69, 1468 (1991)), including 5′-deoxy 5′-methythioadenosine (Kikugawa et al.), 5′-deoxy 5′-ethylthioadenosine (Kikugawa et al.), 5′-deoxy 5′-phenylthioadenosine(Kikugawa et. al. and M. J. Robins et al.), 5′-deoxy 5′-hydroxyethylthioadenosine (Kikugawa et. al.), 5′-iso-butylthio 5′-deoxyadenosine (Craig and Moffatt Nucleosides Nucleotides 5, 399 (1986)), 3-adenosin-5′-ylsulfanyl-propionic acid (Hildesheim et al. Biochimie (1972), 54, 431), S-tert-butyl-5′-thio-adenosine (Kuhn et al. Chem. Ber. (1965), 98, 1699), S-butyl-5′-thio-adenosine (Hildesheim et al.), S-(2-amino-ethyl)-5′-thio-adenosine (Hildesheim et al), S-pyridin-2-yl-5′-thio-adenosine (Nakagawa et al. Tetrahedron Letter (1975), 17, 1409.-a different synthesis method), S-benzyl-5′-thio-adenosine (Kikugawa et al.), S-phenethyl-5′-thio-adenosine (Anderson et al. J. Med. Chem. (1981), 24, 1271.), S-methylbutyl-5′thio-adenosine (Vedel, M. Biochem. Biophysical Res. Comm. (1981) 99(4), 1316-25, Other preferred species of 5′ adenosine analogs of MTA can also be prepared via literature methods, including 5′-cyclohexylamino-5′-deoxyadenosine (Murayama, A. et. al. J. Org. Chem. (1971), 36, 3029.), 5′-morpholin-4-yl-5′-deoxyadenosine (Vuilhorgne, M. et. al. Hetercycles (1978), 11, 495.), 5′-dimethylamino-5′-deoxyadenosine (Morr, M. et. al. J. Chem. Res. Miniprint (1981), 4, 1153.), O^{5′}-methyl-adenosine (Smith, C. G. et al. J. Med. Chem. (1995), 38(12) 2259.), O⁵′-benzyl-adenosine (Chan, L. et al. Tetrahedron (1990), 46(1), 151.) and 1-(6-amino-purin-9-yl)-β-D-ribo-1,5,6-trideoxy-heptofuranuronic acid ethyl ester (Montgomery et al. J. Heterocycl. Chem. (1974), 11, 211.). 5′-Deoxyadenosine is commercially available from Sigma-Aldrich Corporation and can be prepared by methods disclosed in Robins et al, (1991).

The adenosine-5′-carboxamide derivative can be prepared from 2′,3′-O-isopropylideneadenosine-5′-carboxylic acid (Harmon et. al. Chem. Ind. (London) 1141 (1969); Harper and Hampton J. Org. Chem. 35, 1688(1970); Singh Tetrahedron Lett. 33, 2307 (1992)) using a variation of the method described by S. Wnuk J. Med. Chem. 39,4162 (1996):

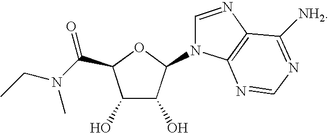

In addition, the adenosine-5′-carboxylic acid sodium salt (Prasad et. al. J. Med. Chem. 19, 1180 (1976)) can be prepared from adenosine-5′-carboxylic acid (R. E. Harmon et. al. Chem. Ind. (London) 1141 (1969); Harper and Hampton J. Org. Chem. 35, 1688 (1970); Singh Tetrahedron Lett. 33, 2307 (1992)) and NaOH:

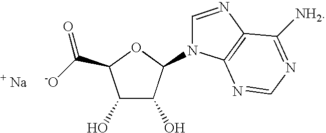

Additional species of MTA analogs of Formula X are compounds having the following chemical structures:

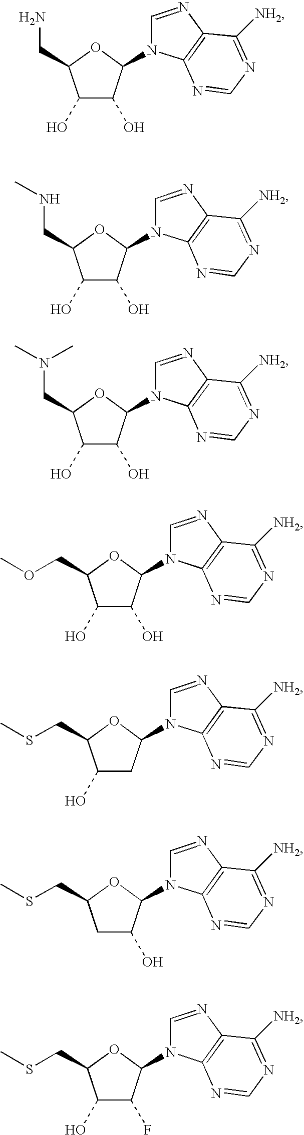

and

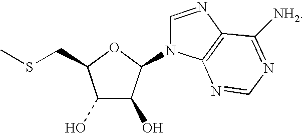

The latter four compounds can be made via literature methods (Montgomery et. Al. J. Med. Chem. 17, 1197 (1974); Gavagnin and Sodano, Nucleosides & Nucleotides 8, 1319 (1989); Allart et al., Nucleosides & Nucleotides 18, 857 (1999)).

Preferably, the anti-toxicity agents are MTAP substrates or prodrugs producing MTAP substrates which have a Km less than 150 times (330 μM) that of MTA. More preferably, the anti-toxicity agent is an MTAP substrate or prodrug thereof which has a Km less than 50 times (110 μM) that of MTA.

Other preferred anti-toxicity agents include MTAP substrates, or prodrugs thereof, which have a Kcat/Km ratio that is greater than 0.05 s^{-1.} μM⁻¹. More preferably the anti-toxicity agents are MTAP substrates or prodrugs thereof having a Kcat/Km ratio that is greater than 0.01 s^{-1.} μM⁻¹.

Examples 2(B), 2(D), 2(E), 2(F) and 2(G) below provides synthetic schemes for the synthesis of MTAP substrates.

In healthy cells, natural precursors of MTA will be converted to MTA for action by MTAP. As used herein, a “precursor” is a compound from which a target compound is formed via, one or a number of biochemical reactions that occur in vivo. A “precursor of MTA” is, therefore, an intermediate which occurs in vivo in the formation of MTA. For example, precursors of MTA include S-adenosylmethionine (“SAMe”) or decarboxylated S-adenosylmethionine (“dcSAMe” or “dSAM”). SAMe and dcSAMe, respectively, are described by the compounds BB and CC below:

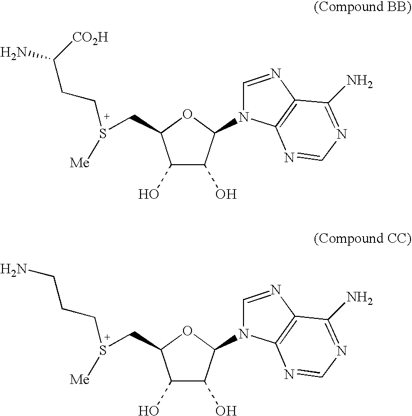

In addition, synthetic analogs of MTA precursors can be prepared. As used herein, an “analog of an MTA precursor” refers to a compound related in physical structure to an MTA precursor, e.g., SAMe or dcSAMe, and which in vivo acts as an intermediate in the formation of an MTAP substrate.

Prodrugs of MTAP substrates are also useful in the invention as anti-toxicity agents. Prodrugs may be designed to improve physicochemical or pharmacological characteristics of the MTAP substrate. For example a prodrug of a MTAP substrate may have functional groups added to increase its solubility and/or bioavailability. Prodrugs of MTAP substrates which are more soluble than MTA are disclosed, for example, in J. Org. Chem. (1994) 49(3): 544-555, the disclosures of which are hereby incorporated by reference in its entirety.

In the present invention, preferred prodrugs of MTAP substrates include carbamates, esters, phosphates, and diamino acid esters of MTA or of MTA analogs. Additional prodrugs can be prepared by those skilled in the, art. For example, the 2′, 3′-diacetate derivatives of 5′-deoxy 5′-methylthioadenosine (J. R. Sufrin et. al. J. Med. Chem. 32, 997 (1989)), 5′-deoxy 5′-ethylthioadenosine and 5′-iso-butylthio 5′-deoxyadenosine can be prepared according to the methods described in J. Org. Chem. 59, 544 (1994):

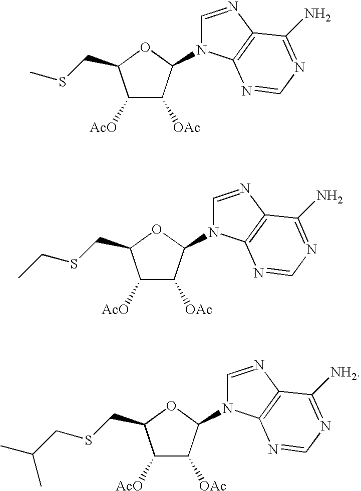

See also, e.g., Bertolini et al., J. Med. Chem. (1997), 40:2011-2016; Shan et al., J. Pharm. Sci. (1997), 86 (7):765-767; Bagshawe, Drug Dev. Res. (1995), 34:220-230; Bodor, Advances in Drug Res. (1984), 13:224-331; Bundgaard, Design of Prodrugs (Elsevier Press 1985); Larsen, Design and Application of Prodrugs, Drug Design and Development (Krogsgaard-Larsen et al. eds., Harwood Academic Publishers, 1991); Dear et al., J. Chromatogr. B (2000), 748:281-293; Spraul et al., J. Pharmaceutical & Biomedical Analysis (1992), 10 (8):601-605; and Prox et al., Xenobiol. (1992), 3 (2):103-112.

In one embodiment, the anti-toxicity agents of the present invention are prodrugs of MTAP substrates having the Formula XI:

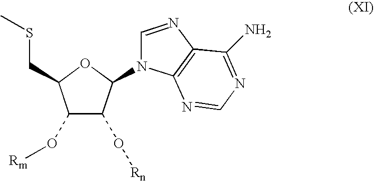

wherein

Rₘ and Rₙ are, independently, selected from the group consisting of H; a phosphate or a sodium salt thereof; C(O)N(Rₒ)₂; C(O)Rₒ; or C(O)ORₒ, wherein Rₒ is selected from the group consisting of H, C₁-C₆ alkyl, C₂-C₆ heterocycloalkyl, cycloalkyl, heteroaryl, aryl, and amino, unsubstituted or substituted with C₁-C₆ alkyl, C₁-C₆ heteroalkyl, C₂-C₆ heterocycloalkyl, cycloalkyl, C₁-C₆ boc-aminoalkyl;

and solvates or salts thereof.

Rₘand Rₙ may each, independently, represent:

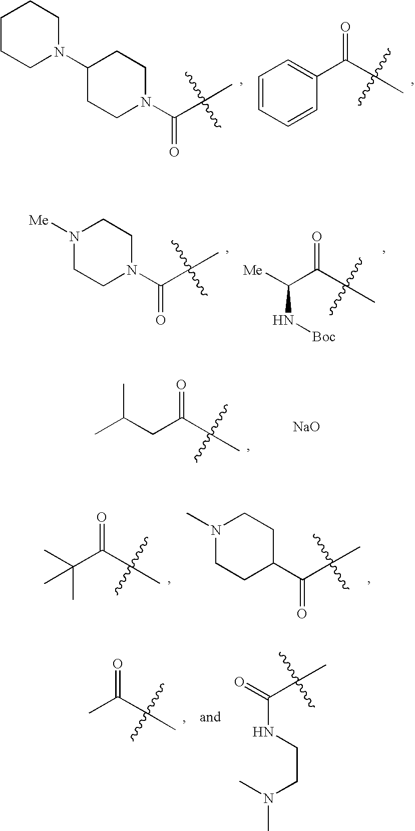

Additional prodrugs of MTAP substrates can be synthesized as shown in Example 2(C) below.

III. Identification of MTAP-Deficient Cells

The methods of the present invention are applicable to mammals having MTAP-deficient cells, preferably mammals having primary tumor cells lacking the MTAP gene product. As used herein, an “MTAP-deficient cell” is a cell incapable of producing a functional MTAP enzyme necessary for production of adenine through the salvage pathway of purine synthesis. Generally, the MTAP-deficient cells useful in the present invention have homozygous deletions of all or a part of the gene encoding MTAP, or have inactivations of the MTAP protein. These cells may be MTAP-deficient due to cellular changes including genetic changes, e.g. gene deletion or mutation, or by disruption of transcription, e.g. silencing of the gene promotor, and/or protein inactivation or degradation. The term “MTAP-deficient cells” also encompasses cells deficient of allelic variants or homologues of the MTAP-encoding gene, or cells lacking, adequate levels of functional MTAP protein to provide sufficient salvage of purines. Methods and assays for detecting the MTAP-deficient cells of a mammal are described below.

The present invention is directed to treating cell proliferative disorders which have incidence of MTAP deficiencies. Examples of cell proliferative disorders which have been associated with MTAP deficiency include, but are not limited to, breast cancer, pancreatic cancer, head and neck cancer, pancreatic cancer, colon cancer, prostrate cancer, melanoma or skin cancer, acute lymphoblastic leukemias, gliomas, osteosarcomas, non-small cell lung cancers and urothelial tumors (e.g., bladder cancer). Cancer cell samples should be assayed for MTAP deficiency as clinically indicated. Assays to assess MTAP-deficiency include those to assess gene status, transcription, and protein level or functionality. U.S. Pat. No. 5,840,505; U.S. Pat. No. 5,942,393 and International Publication No. WO99/20791 provide methods for the detection of MTAP deficient tumor cells, and are hereby incorporated by reference in their entireties.

A polynucleotide sequence of the human MTAP gene is on deposit with the American Type Culture Collection, Rockville, Md., as ATCC NM_{—}002451. The MTAP gene has been located on chromosome 9 at region p21. It is known that the MTAP homozygous deletion has also been correlated with homozygous deletion of the genes encoding p16 tumor suppressor and interferon-α. Detection of homozygous deletions of the p16 tumor suppressor and interferon-α genes may be an additional means to identify MTAP-deficient cells.

Table 2 below indicates the rate of MTAP deficiency, including those inferred based on rates of p16 deletion, in a sample of human primary cancers.

TABLE 2
MTAP Deletions in Human Primary Cancers

| | |
|---|---|
| Non-small cell lung cancer | 35-50% |
| Osteosarcoma | 30-40% |
| Leukemia (T-cell ALL) | 30-40% |
| Glioblastoma | 30-45% |
| Breast cancer | 0-15% |
| Prostate cancer | 0-20% |
| Pancreatic cancer | 50% |
| Melanoma | 10-20% |
| Bladder cancer | 25-40% |
| Head and Neck cancer | ˜30% |

To identify patients whose cell-proliferative disorders are MTAP-deficient, a number of methods known in the art may be employed. These methods include, but not are not limited to, hybridization assays for homozygous deletion of the MTAP gene (see, e.g., Sambrook, J., Fritsh, E. F., and Maniatis, T. Molecular Cloning: A Laboratory Manual. 2^{nd,} ed, Cold Spring Harbor Laboratory, Cold Spring Harbor Laboratory Press, Cold Spring Harbor, N.Y. (1989), and Current Protocols in Molecular Biology, eds. Ausubel et al, John Wiley & Sons (1992)). For example, it is convenient to assess the presence of MTAP-encoding DNA or cDNA can be determined by Southern analysis, in which total DNA from a cell or tissue sample is extracted and hybridized with a labeled probe (i.e. a complementary nucleic acid molecules), and the probe is detected. The label can be a radioisotope, a fluorescent compound, an enzyme or an enzyme co-factor. MTAP encoding nucleic acid can also be detected and/or quantified using PCR methods, gel electrophoresis, column chromatography, and immunohistochemistry, as would be known to those skilled in the art.

Other methodologies for identifying patients with an MTAP-deficient disorder involve detection of no transcribed polynucleotide, e.g., RNA extraction from a cell or tissue sample, followed by hybridization of a labeled probe (i.e., a complementary nucleic acid molecule) specific for the target MTAP RNA to the extracted RNA and detection of the probe (i.e. Northern blotting). The label can be a radioisotope, a fluorescent compound, an enzyme, or an enzyme co-factor. The MTAP protein can also be detected using antibody screening methods, such as Western blot analysis. Another method for identifying patients with an MTAP-deficient disorder is by screening for MTAP enzymatic activity in cell or tissue samples.

An assay for MTAP-deficient cells can comprise an assay for homozygous deletions of the MTAP-encoding gene, or for lack of mRNA and/or MTAP protein. See U.S. Pat. No. 5,942,393, which is hereby incorporated by reference in its entirety. Because identification of homozygous deletions of the MTAP-encoding gene involves the detection of low, if any, quantities of MTAP, amplification may be desirable to increase sensitivity. Detection of the MTAP-encoding gene would thus involve the use of a probe/primer in a polymerase chain reaction (PCR), such as anchor PCR or RACE PCR, or, alternatively, in a ligation chain reaction (LCR) (see, e.g., U.S. Pat. Nos. 4,683,195; 4,683,202 Landegran et al (1988) Science 241:1077-1080; and Nakazawa et al. (1994) Proc. Mail. Acad. Sci. USA 91:360-364, each of which is hereby incorporated by reference in its entirety). PCR and/or LCR may be desirable to use as a preliminary amplification step in conjunction with any of the techniques used for detecting deletion of the MTAP gene. Alternative amplification methods for amplifying any present MTAP-encoding polynucleotides include self sustained sequence replication (Guatelli, J C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques known to those of skill in the art.

Preferably, the MTAP-deficient cell samples are obtained by biopsy or surgical extraction of portions of tumor tissue from the mammalian host. More preferably, the cell samples are free of healthy cells which may contaminate the sample by providing false positives.

IV. Administration of the Inhibitor of De Novo IMP Synthesis and Anti-Toxicity Agent

Once a mammal in need of treatment has been identified as possessing MTAP-deficient cells, the mammal may be treated with a therapeutically effective dosage of an inhibitor of de novo IMP synthesis and an antitoxicity agent in an amount effective to increase the maximally tolerated dose of such inhibitor. It is also within the scope of the invention that more than one inhibitor may be concurrently administered in the present invention. While rodent subjects are provided in the examples of the present invention (Examples 4 and 5), combination therapy of the present invention may ultimately be applicable to human patients as well. Analysis of the toxicity of other mammals may also be obtained using obvious variants of the techniques outlined below.

The methods of the present invention are suitable for all mammals independent of circulating folate levels. See Alati et al. “Augmentation of the Therapeutic Activity of Lometrexol [6-R)t, 10-Dideazatetrahydrofolate] by Oral Folic Acid, Cancer Res. 56: 2331-2335 (1996). The present invention is therefore advantageous in that folic acid supplementation is not required.

Therapeutic efficacy and toxicity of the combinations of inhibitor and anti-toxicity agent can be determined by standard pre-clinical and clinical procedures in cell cultures, experimental animals or human patients. Therapeutically effective dosages of the compounds include pharmaceutical dosage units comprising an effective amount of the active compound.

A “therapeutically effective amount” of an inhibitor of de novo IMP synthesis means an amount sufficient to inhibit the de novo purine pathways and derive the beneficial effects therefrom. With reference to these standards, a determination of therapeutically effective dosages for the IMP inhibitors to be used in the invention may be readily made by those of ordinary skill in the oncological art.

In the present invention the anti-toxicity agent is administered in a dosage amount effective to decrease the toxicity of the inhibitor. In regards to in vitro cell culture experiments, a decrease in toxicity can be determined by detecting an increase in the IC₅₀, i.e., the concentration of inhibitor needed to inhibit cell growth or induce cell death by 50%. In mammals, ad erease intoxicity can be determined by detecting an increase in the maximally tolerated dose. As used in the present invention, a dose of an anti-toxicity, agent usefull in this invention contains at least “an amount effective to increase the maximally tolerated dose” of the inhibitor. A “maximally tolerated dose” as used herein, refers to the highest dose that is considered tolerable, as determined against accepted pre-clinical and clinical standards. Toxicity studies can be designed to determine the inhibitor's maximally tolerated dose (“MTD”). In experimental animal studies, the MTD can be defined as the LD₅₀ or by other statistically useful standards, e.g, as the amount causing no more than 20% weight loss and no toxic deaths (see, e.g., Example 4 below). In clinical studies, the MTD can be determined as that dose at which fewer than one third of patients suffer dose limiting toxicity, which is in turn defined by pertinent clinical standards (e.g., by a grade 4 thrombocytopenia or a grade 3 anemia). See National Cancer Institute's cancer therapy evaluation program for common toxicity criteria; and Mani, Sridhar and Ratain, Mark J., New Phase I Trial Methodology, Seminars in Oncology, vol. 24, 253-261 (1997), the disclosures of which are hereby incorporated by reference in their entireties.

The dose ratio between toxic and therapeutic effects is the therapeutic index. The therapeutic index can be expressed as the ratio of maximally tolerated dose over the minimum therapeutically effective dose. In the present invention, combination therapies which increase the therapeutic index are preferred.

Data obtained from cell culture assays and animal studies can be used in formulating a range of dosages and schedules of administration for the inhibitor and anti-toxicity agent when used in humans. The dosage of such inhibitor compounds preferably yields a circulating plasma concentration that lies within a range that includes the therapeutically effective amount of the inhibitor but below the amount that causes dose-limiting toxicity. Consequently, the dosage of any anti-toxicity agent preferably yields a circulating plasma concentration that lies within a range that includes the amount effective to increase the dosage of inhibitor which causes dose-limiting toxicity. The dosage may vary depending upon the form employed and the route of administration utilized. For any inhibitor compound used in the methods of the invention, the therapeutically effective plasma concentration can be estimated initially from cell culture data, as shown in Example 3 below. Such information can be used to more accurately determine useful doses in humans. Levels in plasma may be measured, for example, by mass spectrometry. An exemplary initial dose of the inhibitor or anti-toxicity agent for a mammalian host comprises an amount of up to two grams per square meter of body surface area of the host, preferably one gram, and more preferably, about 700 milligrams or less, per square meter of the animal's body surface area.

The present invention provides that the anti-toxicity agent is administered during and after administration of the inhibitor such that the effects of the agent persist throughout the period of inhibitor activity for sufficient cell survival and viability of the organism. Administration of the anti-toxicity agent may be performed by any suitable method, including but not limited to, during and after each dose of the inhibitor, by multiple bolus or pump dosing, or by slow release formulations. In one aspect, the anti-toxicity agent is administered such that the effects of the agent persist for a period concurrent with the presence of the inhibitor. The in vivo presence of the inhibitor can be determined using pharmacokinetic indicators as determined by one skilled in the art, e.g., direct measurement of the presence of inhibitor in plasma or tissues. In another aspect, the anti-toxicity agent is administered such that the effects of the agent persist until inhibitor activity has substantially ceased, as determined by using pharmacodynamic indicators, e.g., as purine nucleoside levels in plasma. As shown in Example 4 below, the anti-toxicity agent increased the MTD of the inhibitor compound in mice when it was administered for an additional 4 days after the last dose of the inhibitor. Example 3(D) further demonstrates that cytotoxicity decreased most dramatically in cell culture samples when administration with the anti-toxicity agent was prolonged long after dosing with the inhibitor compound was terminated.

The agents of the invention, both the IMP inhibitors and the anti-toxicity agent, may be independently administered by any clinically acceptable means to a mammal, e.g. a human patient, in need thereof. Clincally acceptable means for administering a dose include topically, for example, as an ointment or a cream orally, including as a mouthwash; rectally, for example as a suppository; parenterally or infusion; or continuously by intravaginal, intranasal, intrabronchial intraaural or intraocular infusion. Preferably, the agents of the invention are administered orally or parenterally.

Preferred embodiments of the invention are illustrated by the examples set forth below. It will be understood, that the examples do not limit the scope of the invention, which is defined by the appended claims. Standard abbreviations are used throughout the Examples, such as “μl” for microliter, “hr” for hour and “mg” for milligram.

### EXAMPLE 1

Syntheses of Compounds 6 and 7

Compound 6: N-(5-[2-(2-amino-4(3H)-oxo-5,6,7,8-tetrahydropyrido[2,3-d]pyrimidin-6-yl)-(R)-ethyl]-4-methylthieno-2-yl)-L-glutamic acid

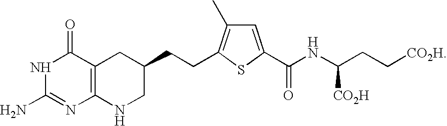

Compound 7: N-(5-[2-(2-amino-4(3H)-oxo-5,6,7,8-tetrahydropyrido[2,3-d]pyrimidin 6-yl)-(S)-ethyl]-4-methylthieno-2-yl)-L-glutamic acid

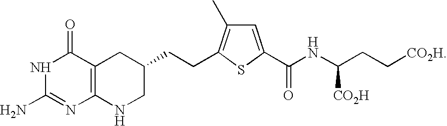

### EXAMPLE 1(A)

Synthesis route for Compounds 6 and 7

In one method, compounds 6 and 7 were synthesized by the following process.

Step 1: 5-bromo-4-methylthiophene-2-carboxylic acid

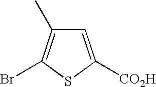

This compound was prepared according to M. Nemec, Collection Czechoslov. Chem. Commun., vol. 39 (1974), 3527.

Step 2: 6-ethynyl-2-(pivaloylamino)-4(3H)-oxopyrido [2,3-d]pyrimidine

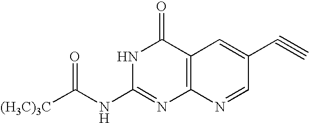

This compound was prepared according to E. C. Taylor & G. S. K. Wong, J. Org. Chem., vol. 54 (1989), 3618.

Step 3: Diethyl N-(5-bromo-4-methylthieno-2-yl)-L-glutamate

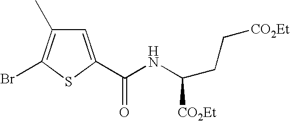

To a stirred solution of 5-bromo-4-methylthiophene-2-carboxylic acid (3.32 g, 15 mmol), 1-hydroxybenzotriazole (2.24 g, 16.6 mmol), L-glutamic acid diethyl ester hydrochloride (3.98 g, 16.6 mmol) and diisopropylethylamine (2.9 ml, 2.15 g, 16.6 mmol) in dimethylformamide (DMF) (40 ml) was added 1-(3-dimethylaminopropyl)-3-ethylcarbodiimide hydrochloride (3.18 g, 16.6 mmol). The resulting solution was stirred under argon at ambient temperature for 18 hours, poured into brine (300 ml), diluted with water (100 ml) and extracted with ether (3×120 ml). The combined organic extracts were washed with water (150 ml), dried over MgSO₄ and concentrated in vacuo to give a brown gum, which was purified by flash chromatography. Elution with hexane: EtOAc (2:1) provided the product as an orange oil (5.05 g, 83% yield). Analyses indicated that the product was diethyl N-(5-bromo-4-methylthieno-2-yl) glutamate. NMR(CDCl₃) δ:7.22 (1H, s), 6.86 (1H, d, J=7.5 Hz), 4.69 (1H, ddd, J=4.8, 7.5, 9.4 Hz), 4.23 (2H, q, J=7.1 Hz), 4.12 (2H, q, J=7.1 Hz), 2.55−2.39 (2H, m), 2.35−2.22 (1H, m), 2.19 (3H, s), 2.17−2.04 (1H, m), 1.29 (3H, t, J=7.1 Hz), 1.23 (3H, t, J=7.1 Hz). Anal. (C₁₅H₂₀ NO₅ SBr) C,H,N,S,Br.

Step 4: Diethyl N-(5-[(2-[pivaloylamino]-4(3H)-oxopyrido[2,3-d]pyrimidin-6-yl) ethynyl]-4-methylthieno-2-yl) glutamate:

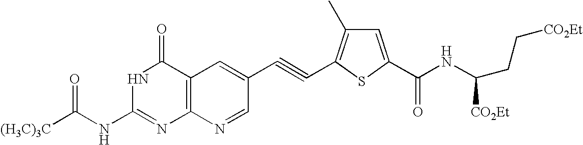

To a stirred solution of diethyl N-(5-bromo-4-methylthieno-2-yl) glutamate (4.21 g, 10.4 mmol) in acetonitrile (55 ml) under an argon atmosphere were added bis (triphenylphosphine) palladium chloride (702 mg, 1.0 mmol), cuprous iodide (200 mg, 1.1 mmol), triethylamine (1.5 ml, 1.09 g, 10.8 mmol) and 6-ethynyl-2-(pivaloylamino)-4(3H)-oxopyrido[2,3-d]pyrimidine (5.68 g, 21 mmol). The resultant suspension was heated at reflux for 6 hours. After cooling to room temperature, the crude reaction mixture was filtered and the precipitate was washed with acetonitrile (50 ml) and ethylacetate (EtOAc) (2×50 ml). The combined filtrates were concentrated in vacuo to give a brown resin, which was purified by flash chromatography. Elution with CH₂Cl₂:CH₃OH (49:1) provided the product as an orange solid (4.16 g, 67% yield). Analyses indicated that the product was diethyl N-(5-[(2-[pivaloylamino]-4(3H)-oxopyrido[2,3-d]pyrimidin-6-yl) ethynyl]-4-methylthieno-2-yl) glutamate. NMR (CDCl₃) δ:8.95 (1H, d, J=2.2 Hz), 8.59 (1H, d, J=2.2 Hz), 7.33 (1H, s), 7.03 (1H, d, J=7.4 Hz), 4.73 (1H, ddd, J=4.8, 7.4, 9.5 Hz), 4.24 (2H, q, J=7.1 Hz), 4.13 (2H, q, J=7.1 Hz), 2.55−2.41 (2H, m), 2.38 (3H, s), 2.35−2.24 (1H, m),2.19−2.05 (1H, m), 1.34 (9H, s), 1.30 (3H, t, J=7.1 Hz), 1.24 (3H, t, J=7.1 Hz). Anal. (C₂₉H₃₃N₅O₇S.0.75H₂O) C,H,N,S.

Step 5: Diethyl N-(5-[(2-[pivaloylamino]-4(3H)-oxopyrido[2,3,d] pyrimidin-6-yl) ethyl]-4-methylthieno-2-yl) glutamate

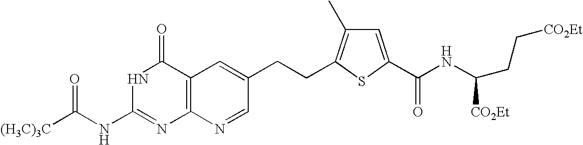

A suspension of diethyl N-(5-[(2-[pivaloylamino]-4(3H)-oxopyrido [2,3-d]pyrimidin-6-yl)ethyl]-4-methylthieno-2-yl) glutamate (959 mg, 1.6 mmol) and 10% Pd on carbon (1.5 g, 150% wt. eq.) in trifluoroacetic acid (30 ml) was shaken under 50 psi of H₂ for 22 hours. The crude reaction mixture was diluted with CH₂Cl₂, filtered through a pad of Celite (diatomaceous earth) and concentrated in vacuo. The residue obtained was dissolved in CH₂Cl₂ (120 ml), washed with saturated NaHCO₃ (2×100 ml), dried over Na₂SO₄ and concentrated in vacuo to give a brown gum, which was purified by flash chromatography. Elution with CH₂Cl₂:CH₃OH (49:1) provided the product as a yellow solid (772 mg, 80% yield). Analyses indicated that the product was diethyl N-(5-[(2-[pivaloylamino]-4(3H)-oxopyrido[2,3-d]pyrimidin-6-yl)ethyl]-4-methylthieno-2-yl) glutamate. NMR (CDCl.sub.3) δ:8.60 (1H, d, J=2.2 Hz), 8.49 (1H, broad), 8.32 (1H, d, J=2.2 Hz), 7.22 (1H, s), 6.78 (1H, d, J=7.5 Hz), 4.72 (1H, ddd, J=4.8, 7.5, 9.5 Hz), 4.23 (2H, q, J=7.1 Hz), 4.11 (2H, q, J=7.1 Hz), 3.12−3.00 (4H, m), 2.52−2.41 (2H, m), 2.37−2.22 (1H, m), 2.16−2.04 (1H, m), 2.02 (3H, s), 1.33 (9H, s), 1.29 (3H, t, J=7.1 Hz), 1.23 (3H, t, J=7.1 Hz). Anal. (C₂₉H₃₇N₅O₇S.0.5H₂O) C,H,N,S.

Step 6: Diethyl N-(5-[(2-[pivaloylamino]-4(3H)-oxo-5,6,7,8-tetrahydropyrido[2,3-d]pyrimidin-6-yl)-ethyl]-4-methylthieno-2-yl) glutamate

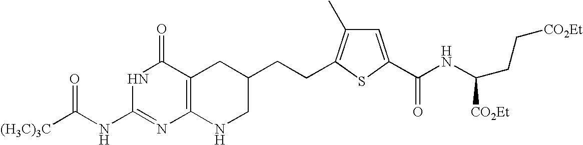

A suspension of diethyl N-(5-[(2-[pivaloylamino]-4(3H)-oxopyrido [2,3-d]pyrimidin-6-yl)ethyl]-4-methylthieno-2-yl) glutamate (32.2 g, 59 mmol), 10% Pt on carbon (25.12 g, 78% wt. eq.), 10% Pd on carbon (10.05 g, 30% wt. eq.) and PtO.sub.2 (10 g, 30% wt. eq.) in trifluoroacetic acid (170 ml) was shaken under 900 psi of H.sub.2 for 330 hours. The crude reaction mixture was diluted with CH₂Cl₂, filtered through a pad of Celite, and concentrated in vacuo. The residue obtained was dissolved if CH₂Cl₂ (600 ml), washed with saturated NaHCO₃ (2×400 ml), dried over Na₂SO₄, and concentrated in vacuo to give a brown resin, which was purified by flash chromatography. Elution with CH₂Cl_{2:}CH₃OH (24:1) provided initially an unreacted substrate (10.33 g, 32% yield) and then the product, yellow solid, as a mixture of diastereomers (4.06 g, 11% yield). Analyses indicated that the product was diethyl N-(5-[(2-[pivaloylamino]-4(3H)-oxo-5,6,7,8-tetrahydropyrido-[2,3-d]pyrimidin-6-yl)ethyl]-4-methylthieno-2-yl) glutamate. NMR (CDCl.sub.3) δ:7.24 (1H, s), 6.75 (1H, d, J=7.6 Hz), 5.57 (1H, broad), 4.72 (1H, ddd, J=4.8, 7.6, 12.6 Hz), 4.22 (2H, q, J=7.1 Hz), 4.11 (2H, q, J=7.1 Hz), 3.43−3.36 (1H, m), 3.06−2.98 (1H, m), 2.89−2.68 (3H, m), 2.52−2.40 (3H, m), 2.37−2.23 (1H, m), 2.15 (3H, s), 2.14−2.03 (1H, m), 1.94−1.83 (1H, m), 1.73−1.63 (2H, m), 1.32 (9H,s), 1.29 (3H, t, J=7.1 Hz), 1.23 (3H, t, J=7.1 Hz). Anal. (C₂₉H₄₁N₅O₇S.0.5H₂O) C,H,N,S.

This diastreomeric mixture was further purified by chiral-phase HPLC. Elution from a Chiralpak column with hexane:ethanol:diethylamine (70:30:0.15) at a temperature of 40° C. and a flow rate of 1.0 ml/minute provided the separate diastereomers as yellow solids (1.07 g and 1.34 g, respectively). The ¹H NMR spectra of the individual diastereomers were indistinguishable from each other and from the spectrum obtained for the mixture.

Step 7: N-(5-[2-(2-amino-4(3H)-oxo-5,6,7,8-tetrahydropyrido-[2,3-d]pyrimidin-6-(R)-yl) ethyl]-4-methylthieno-2-yl): glutamic acid (Compound 6):

A suspension of the slower-eluting diastereomer of diethyl N-(5-[(2-[pivaloylamino]-4(3H)-oxo-5,6,7,8-tetrahydrlpyrido[2,3-d]pyrimidin-6-yl)ethyl]-4- methylthieno-2- yl) glutamate (1.31 g, 2.2 mmol) in 2N NaOH (40 ml) was stirred at ambient temperature for 120 hours, then filtered to remove any remaining particulate matter. The filtrate was subsequently adjusted to pH 5.5 with 6N HCl. The precipitate that formed was collected by filtration and washed with water (2×10 ml) and ether (2×10 ml) to provide the product as a yellow solid (794 mg, 79% yield). Analyses indicated that the product was N-(5-[2-(2-amino-4(3H)-oxo-5,6,7,8-tetrahydropyrido[2,3-d]pyrimidin-6-yl)ethyl]-4-methylthieno-2-yl) glutamic acid. NMR (DMSO-d₆) δ:12.35 (2H, broad), 9.83 (1H, broad), 8.41 (1H, d, J=7.7 Hz), 7.57 (1H, s), 6.43 (1H, br s), 6.20 (2H, br s), 4.34−4.26 (1H, m), 3.29−3.19 (2H, m), 2.83−2.74 (3H, m), 2.32 (2H, t, J=7.3 Hz), 2.12 (3H, s), 2.08−2.00 (1H, m), 1.92−1.81 (2H, m), 1.68−1.49 (3H, m), Anal. (C₂₀H₂₅N₅O₆S0.8H₂O) C,H,N,S.

Step 8: N-(5-[2-(2-amino-4(3H)-oxo-5,6,7,8-tetrahydropyrido-[2,3-d]pyrimidin-6-(S)-yl) ethyl]-4-methylthieno-2-yl) glutamic acid (Compound 7):

A suspension of the faster-eluting diastereomer of diethyl N-(5-[(2-[pivaloylamino]-4(3H)-oxo-5,6,7,8-tetrahydropyrido[2,3-d]pyrimidin-6-yl)ethyl]-4-methylthieno-2-yl) glutamate (1.02 g, 1.7 mmol) in 2N NaOH (35 ml) was stirred at ambient temperature for 120 hours, then filtered to remove any remaining particulate matter. The filtrate was subsequently adjusted to pH 5.5 with 6N HCl. The precipitate that formed was collected by filtration and washed with water (2×10 ml) and ether (2×10 ml) to provide the product as a yellow solid (531 mg, 68% yield). Analyses indicated that the product was N-(5-[2-(2-amino-4(3H)-oxo-5,6,7,8-tetrahydropyrido[2,3-d]pyrimidin-6-yl)ethyl]-4-methylthieno-2-yl) glutamic acid. NMR (DMSO-d₆) δ:12.52 (2H, broad), 9.69 (1H, broad), 8.36 (1H, d, J=7.7 Hz), 7.56 (1H, s), 6.26 (1H, br s), 5.93 (2H, br s), 4.32−4.25 (1H, m), 3.24−3.16 (2H, m), 2.81−2.73 (3H, m), 2.31 (2H, t, J=7.2 Hz), 2.12 (3H, s), 2.07−1.98 (1H, m), 1.91−1.79 (2H, m), 1.65−1.48 (3H,m). Anal. (C₂₀H₂₅N₅O₆S.0.7H₂O) C,H,N,S.

Step 8: Crystallography of Compounds 6 and 7

The GART domain (residues 808-1010) of the trifunctional human GARS-AIRS-GART enzyme was purified according to the method described by Kan, C. C., et al., J. Protein Chem. 11:467-473, (1992). Following purification, GART was concentrated to 20 mg/mL in a buffer containing 25 mM Tris pH 7.0 and 1 mM DTT. Crystallization was done by hanging-drop vapor diffusion, mixing the protein and reservoir solution (38-44% MPD, 0.1 M Hepes, pH 7.2-7.6) in a 1:1 ratio, and equilibrating at 13° C. Crystals would typically grow within 3 days and measure 0.2×0.25×0.3 mm.

X-ray diffraction data were collected from ternary complex crystals of GART, GAR 1 and inhibitor at 4° C. using a San Diego Multiwire Systems 2-panel area detector and a Rigaku AFC-6R monochomatic Cu Kα X-ray source and goniostat (Table 3). The space group was determined to be P3₂21, with the cell constants shown below. The crystal structures of both compounds 6 and 7 complexes were solved by molecular replacement using MERLOT (Fitzgerald, P. M. D. MERLOT, an Integrated Package of Computer Programs for the Determination of Crystal Structures by Molecular Replacement. J. Appl. Crystallogr. 21:273-278 (1988)). The search model consisted of residues 1-209 from an E. coli GART ternary complex structure (Protein Data Bank accession number 1 cde). The highest peak in the cross rotation function (Crowther, R. A. The Fast Rotation Function. In The Molecular Replacement Method, 1972) was used in 3-dimensional translation functions (Crowther, R. A., et al., A method of Positioning a Known Molecule in an Unknown Crystal Structure. Acta Crystallogr. 23:544-548 (1967)), in search of Harker vectors. The top peak in all five searches (i.e. from one molecule to each of the five symmetry related molecules) produced a consistent set of vectors that positioned the model. After initial refinement with XPLOR (Brunger, A. T. X-PLOR Version 3.1: A System for X-ray Crystallography and NMR. New Haven, Conn. (1992)), density was seen for the substrate GAR 1 and the inhibitor. The final structures were obtained by manual model building in 2Fₒ−F_{c} and Fₒ−F_{c} election density maps followed by further refinement with XPLOR (Table 3).

TABLE 3
Summary of X-ray Data and Refinement for Compounds 6 and 7

| | 6 | 7 |
|---|---|---|
| Resolution (Å) | 10-2.3 | 10-3.2 |
| cell (a, Å) | 77.17 | 76.77 |
| cell (c, Å) | 102.67 | 101.45 |
| R_{merge} (%)^{a} | 6.51 | 12.75 |
| Total rels | 59522 | 25756 |
| Unique refls | 16606 | 6858 |
| R factor (%)^{b} | 17.8 | 17.1 |
| No. solvent | 65 | 62 |

^{a}R_{merge}: 100 × ΣₕΣᵢ|I(h)>|/ΣₕΣᵢI(h)I where I(h)i is the ith measurement of reflection h and I(h)i is the mean intensity from N measurements of reflection h.
^{b}R factor: Σ∥Fₒ|—|F_{c}∥/Σ|Fₒ|.
^{c}Average deviation from ideal values.

EXAMPLE 1(B)

Alternate Synthesis Route for Compound 7

Compound 7 can be synthesized by an alternate route, according to the following scheme.

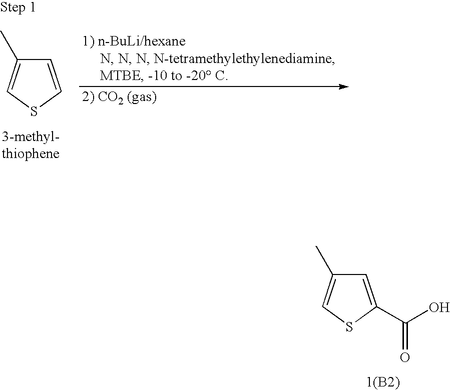

The synthesis begins with the regioselective lithiation at the 5′ position of commercially available 3-methylthiphene (La Porte Performance Chemicals, UK). Under argon, 4.4L MTBE and 800 mL N,N,N,N-tetramethylethylenediamine (“TMEDA”) was combined and cooled to −10° C. 2.10 L of 2.5 M n-BuLi was then added over 30-45 minutes and allow to equilibrate (10-20 min). Also under argon, 500 mL of 3-methylthiphene and 4.4 L MTBE was combined in a separate flask and cooled to −10° C. The n-BuLi-TMEDA was then added to the 3-methylthiphene/MTBE solution, while stirring at a temperature below 20° C. After warming the mixture to room temperature (2 hrs), the solution was then cooled to −10° C. and CO₂ was bubbled through. After purging with CO₂, the reaction mixture was quenched with 14 L water, and the organic phase was separated and extracted with NaOH. The aqueous extract was acidified to pH 2 with HCl. The precipitated product 1(B2) was then collected by filtration, washed twice with water and dried in vacuo at 60-65° C. The material thus obtained was an approximately 90/10 mixture of the desired product 4-methyl-2-thiphenecarboxylic acid 1(B2) and regioisomeric 3-methyl-2-thiphenecarboxylic acid (541 g; 3.81 mol; 66% yield of 1(B2)).

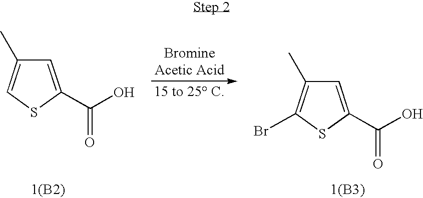

The product mixture containing 1(B2) was brominated with a solution of bromine in acetic acid (195 mL bromine in 2.8 L acetic acid), added to a stirred solution of 1(B2) over 1.5 hours. After 30 minutes the reaction mixture was quenched in 19 L water at room temperature with vigorous stirring. During quenching the desired product 5-bromo-4-methyl-2-thiophenecarboxylic acid 1(B3) precipitated out, and was collected by vacuum filtration, washed twice with water, and dried in vacuo at 65-70° C. The product was obtained as a single isomer by proton NMR (692 g; 3.13 mol; 82% yield). It appeared that the undesired isomer of 1(B2) was only partially brominated and that the unreacted materials and unwanted isomers remained in solution.

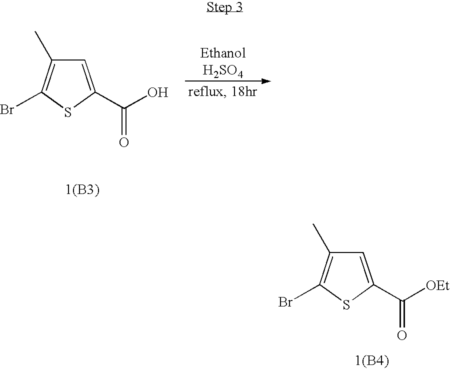

Fisher esterification of acid 1(B3) with ethanol and 1.8 equivalents of concentrated sulfuric acid provided ethyl ester 1(B4) as an oil, after an extractive work-up. 690 g of 1(B3) (in 7.4 L of EtOH) was combined with 270 mL H₂SO₄ and the reaction was refluxed under a calcium sulfate drying tube for 18 hours. After cooling to room temperature, the solution pH was adjusted to pH 8 with sodium bicarbonate and the resulting slurry was concentrated in vacuo to remove ethanol. Water was added and this mixture was extracted twice with 4 L of MTBE. Solvents were removed in vacuo to give 726 g of ethyl 5-bromo-4-methylthiphene-2-carboxylate 1(B4) as an oil (2.92 mol; 93% yield).

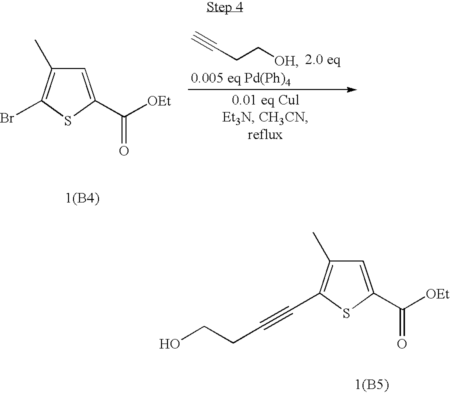

Under argon, the bromothiophene ester 1(B4) was combined with 3-butyn-1-ol (2 equivalents), triethylamine, and CH₃CN in the presence of catalytic tetrakis(triphenylphosphine)palladium and copper(I)iodide and warmed to 78-82° C. for 18 hours. The mixture was then cooled to about 50° C., diluted with water, and concentrated in vacuo to remove CH₃CN. The reaction mixture was then further diluted with 4 L ethyl acetate and 4 L water, and the aqueous phase was extracted further with 2 L additional ethyl acetate. After washing of the combined organic extract (2.5 L of 0.5 M aq HCl and 4 L water), the excess water was removed by azeotropic distillation with ethyl acetate and MTBE to provide the alkyne 1(B5) as a dark oil (1.7 kg; 85% yield).

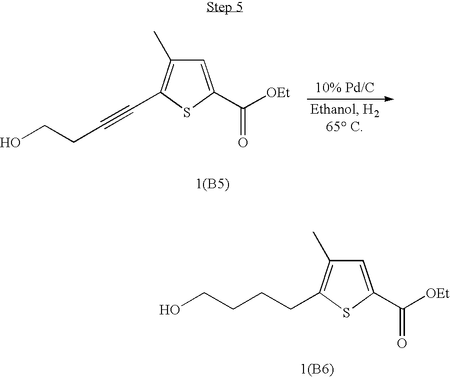

Alkyne 1(B5) was hydrogenated over a 10 day period to cleanly give alcohol 1(B6). 1.56 kg of alkyne 1(B5) was dissolved in 5 L ethanol and charged into a 19 L hydrogenator under nitrogen, followed by the addition of a slurry of Pd/C (100 g of 10% Pd/C in 350 mL, ethanol). The hydrogenator was pressurized to 50 psi with nitrogen and vented with stirring, for a total of 3 cycles, followed by an additional 3 cycles at 100 psi and period repressurization over 1-2 days. After slowing of hydrogen uptake, the reaction mixture was filtered through a 1 inch pad of Celite and subsequently recharged into the hydrogenator along with 100 g of fresh 10% Pd/C in ethanol. The recharging was repeated as described above four times, with 1.5-2 days between each recharge of catalyst. Upon complete consumption of any unsaturated species, the reaction was filtered through a Celite pad and dried in vacuo to yield ethyl 5-(4-hydroxbutyl)-3-methylthiphene-2-carboxylate 1(B6) (1.55 kg; 6.40 mol; 96% yield).

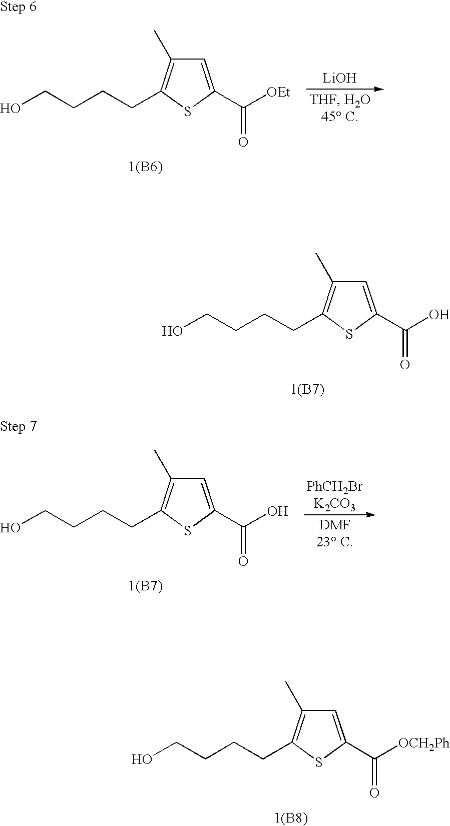

Saponification of ethyl ester 1(B6) yields alcohol-acid 1(B7), which undergoes benzylation with benzyl bromide to give alcohol-ester 1(B8). 306 g aqueous LiOH was added to a solution of ethyl ester 1(B6) (1.55 kg ethyl ester 1(B6)/6.5 L THF), and the mixture was warmed to 45° C. for 19 hrs. The reaction mixture was then cooled to 32° C. and diluted with 3 L MTBE. After phase separation and organic phase extraction (2×500 mL of 1 M NaOH), the aqueous phases were combined and washed twice with 1.5 L MTBE. The aqueous phase was acidified to pH 1 with HCl, and extracted three times with 2 L methylene chloride. The solvents were then removed in vacuo and water removed by azeotropic distillation with 2 L methylene chloride followed by 2 L MTBE to provide alcohol-acid 1(B7). 1.21 kg alcohol-acid 1(B7) and benzyl bromide (1 equivalent) were then dissolved in DMF (8 L), and 1.18 kg K₂CO₃ (1.5 equivalents) was added. After cooling the reaction temperature to 15° C., and then warming to room temperature overnight, water and MTBE were added. After phase separation, the aqueous phase was recharged into the 50 L extractor and the remaining inorganic salts were washed three times with MTBE, and all organic phases were combined for extraction of the aqueous phase. The organic extract was washed with aqueous sodium bicarbonate and water then evaporated in vacuo to provide benzyl ester 1(B8) (1.61 kg; 5.28 mol; 93% yield).

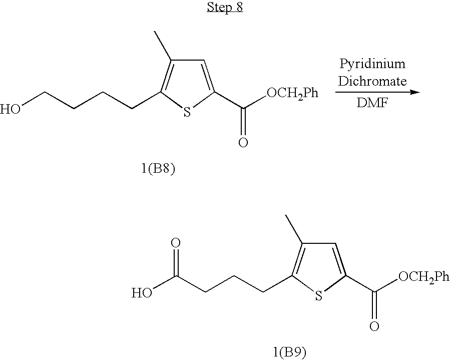

Alcohol 1(B8) was oxidized with four equivalents of pyridinium dichromate to give acid 1(B9). 5.5 kg of pyridinium dichromate was added in 500 g portions to a flask charged with 8 L DMF, and the solution was allowed to warm to 18° C. Alcohol 1(B8) (1.11 kg) was dissolved in 1.5 L DMF and added dropwise to the pyridium dichromate solution at a reaction temperature of 23-24° C. The reaction was allowed to warm to room temperature overnight, then was quenched into a 50 L extractor containing 18 L water, 8 L MTBE and 0.5 L methylene chloride). After phase separation, the aqueous phase was extracted twice with 4 L MTBE. The solid salts were combined with 4 L water and the resulting slurry was extracted with MTBE. The combined MTBE extract was then worked with 0.4 M HCl and water, and the product was back-extracted into aqueous sodium carbonate. After washing the aqueous phase with MTBE the pH was adjusted to 3-4 with HCl, and the product was extracted into MTBE. The MTBE extract was worked with water and washed and dried in vacuo to provide product 1(B9) (816 g; 2.56 mol; 70% yield).

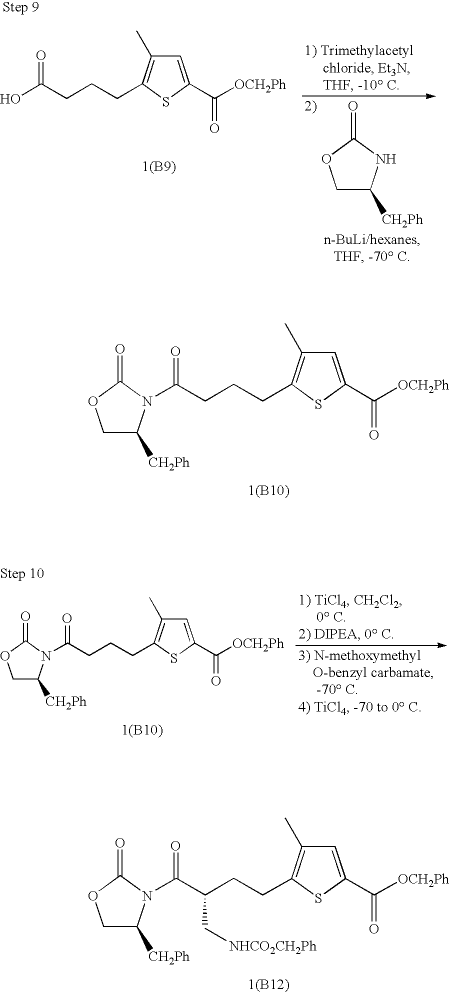

Acid 1(B9) is converted to the mixed pivaloyl anhydride 1(B10), which is immediately reacted with the lithiated benzyloxazolidinone chiral auxiliary to give acyloxazolidinone 1(B11). Triethylamine (214 mL) was added to a solution of carboxylic acid 1(B9) (423 g in 3.2 L MTBE) and the reaction was cooled to −16° C. Pivaloyl chloride was added and the reaction was stirred, then allowed to warm to room temperature. The slurry was filtered through a pad of Celite 545, rinsed with 3.2 L MTBE, and then cooled to −70° C.

In a separate flask, a 2.5 M solution of n-butyllithium in hexanes was added dropwise to a solution of (S)-4-benzyl-2-oxazolidinone (246.8 g in 3.2 L tetrahydrofuran) and cooled to −70° C. for 1 hr with stirring. The lithiated oxazolidinone was added to the mixed anhydride, and after one hour the reaction was quenched by the addition of 2 L of 2 M aq potassium hydrogen sulfate. After phase separation, the organic phase was washed with aqueous sodium bicarbonate, water and brine, and then dried in vacuo to remove solvents and water.

The first permanent chiral center was installed by the diastereoselective alkylation of the titanium enolate of acyloxazolidinone 1(B11) with O-benzyl N-methoxymethyl carbamate, to give CBZ protected amine 1(B12). Starting with a solution of acyloxazolidinone 1(B11) (884 g in 3.1 L methylene chlride), a 1 M solution of titanium tetrachloride in methylene chloride (1.05 equivalents) was added dropwise over 1.25 hours at 3-7° C. and stirred for an additional hour. Hunigs base (1.1 equivalents) was added dropwise, and the mixture stirred for 1 hr. The solution was cooled to −70° C. and then a solution of N-Methoxymethyl O-benzyl carbamate (1.25 equivalents) (453 g in 496 mL methylene chloride) was added. The O-benzyl N-methoxymethyl carbamate is obtained in two steps via known literature methods. Tetrahedron, 44: 5605-5614 (1998). After 30 minutes, 2.31 L of 1 M titanium tetrachloride in methylene chloride (1.25 equivalents) was added over 1.5 hr and the reaction was continued for 1 hour. The reaction was then placed in a 4° C. room for 16 hr, after which the reaction was quenched into a 50 L extractor containing a solution of water and ammonium chloride (1 kg NH₄CL in 8 L water). The flask then was rinsed with methylene chloride, the phases were separated, and the organic phase washed in aqueous ammonium chloride. The methylene chloride was removed in vacuo and the resulting product solidified overnight and was subsequently slurried in 3.8 L methanol. The product was collected by filtration and reslurried in methanol twice, before drying in vacuo, to give carbamate 1(B12) (714 g).

Preparation of N-Methoxymethyl O-Benzyl Carbamate

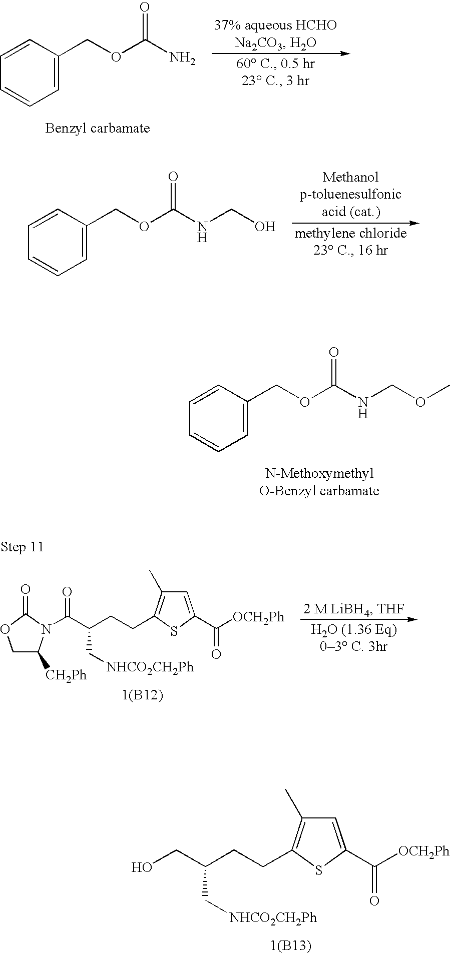

The chiral auxiliary was removed reductively to give alcohol 1(B13). A 2 M solution of lithium borohydride in THF (1.44 equivalents) was added dropwise to a solution of substrate 1(B12) (714 g in 2.0 L THF and 27.2 mL water). The reaction was stirred for 2.5 hours, and then quenched by dropwise addition of 3.0 L of 3 M aq HCl. The reaction was worked up by addition of 4 L methylene chloride, the phases were separated, and the organic phase was washed with 2 L saturated sodium bicarbonate solution. The organic solvents were removed in vacuo to give product 1(B13) (716 g) containing cleaved chiral auxiliary. (The chiral auxiliary is not removed during the workup and is carried on through the next two reactions.)

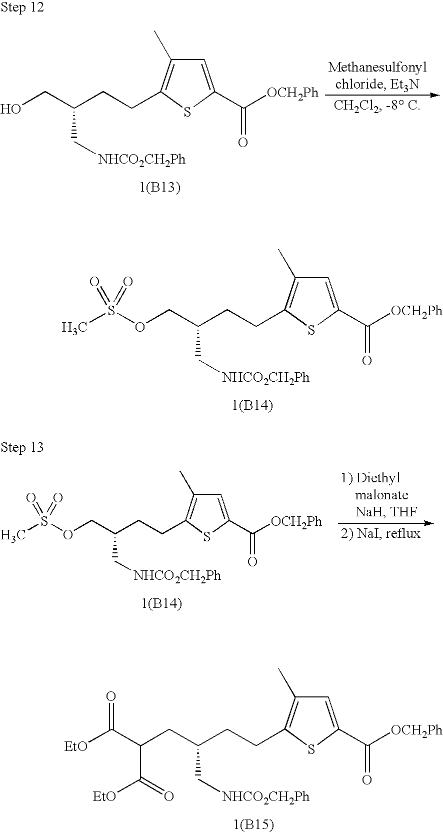

Treatment of alcohol 1(B13) with methanesulfonyl chloride provides mesylate 1(B14), which is reacted with sodio diethyl malonate in the presence of catalytic sodium iodide to give very crude tualonate 1(B15). Starting with a solution of alcohol 1(B13) (432 g in 2.60 L methylene chloride), triethylamine was added and the reaction cooled to −10.3° C., after which 86 mL methanesulfonyl chloride was added dropwise. After about 2.25 hours, the reaction was quenched by addition of 1 L of M aq HCl. The organic phase was separated, washed with aqueous sodium bicarbonate, and dried in vacuo to remove solvent and water to give mesylate 1(B14) as an oil (661 g). To a solution of the mesylate 1(B14) (580 g in 3.83 L THF) was then added a solution of sodium salt of diethyl malonate (340 mL diethyle malonate in 2 L THF, in a flask charged with 50 g sodium hydride). Sodium iodide (0.27 equivalents) was added and the reaction was heated at 62° C. until complete. The reaction was quenched into a mixture of 8 L MTBE and 4 L saturated aqueous sodium bicarbonate. After phase separation, the organic phase was washed with 3 L saturated aqueous sodium bicarbonate and evaporated in vacuo to give malonate 1(B15) (968 g), which was purified by chromatography on silica and eluted with hexane/methylene chloride (75/25).

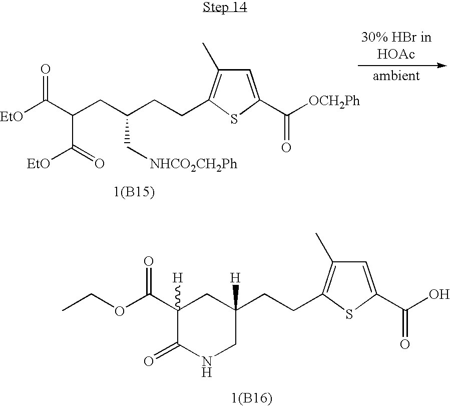

The carbonylbenzyloxy group of 1(B15) was removed from the amine, which then cyclized onto one of the carboethoxy groups to give a pyridinone ring system. At the same time, the benzyl ester was debenzylated to give the carboxylic acid 1(B16). After purification by chromotagraphy, 162.8 g of the malonate 1(B15) was treated with 30% HBr in acetic acid (86.5 g in 213 mL; 4 equivalents) at room temperature. After 15 hours, the reaction was poured into an extractor and buffered to a pH 8-9 by addition of sodium bicarbonate/potassium carbonate. After phase separation, the aqueous phase was washed with 2 L MTBE. The aqueous phase was then diluted with 1.5 L methylene chloride, adjusted to pH 1, and the organic phase was washed with water and aqueous sodium chloride. After drying over anhydrous magnesium sulfate, the methylene chloride solution of lactam 1(B16) was concentrated in vacuo to about 200 mL. The resulting slurry was left to stand at room temperature overnight. The solids were collected by filtration and dried in vacuo over night to provide the product 1(B16) (67.1 g).

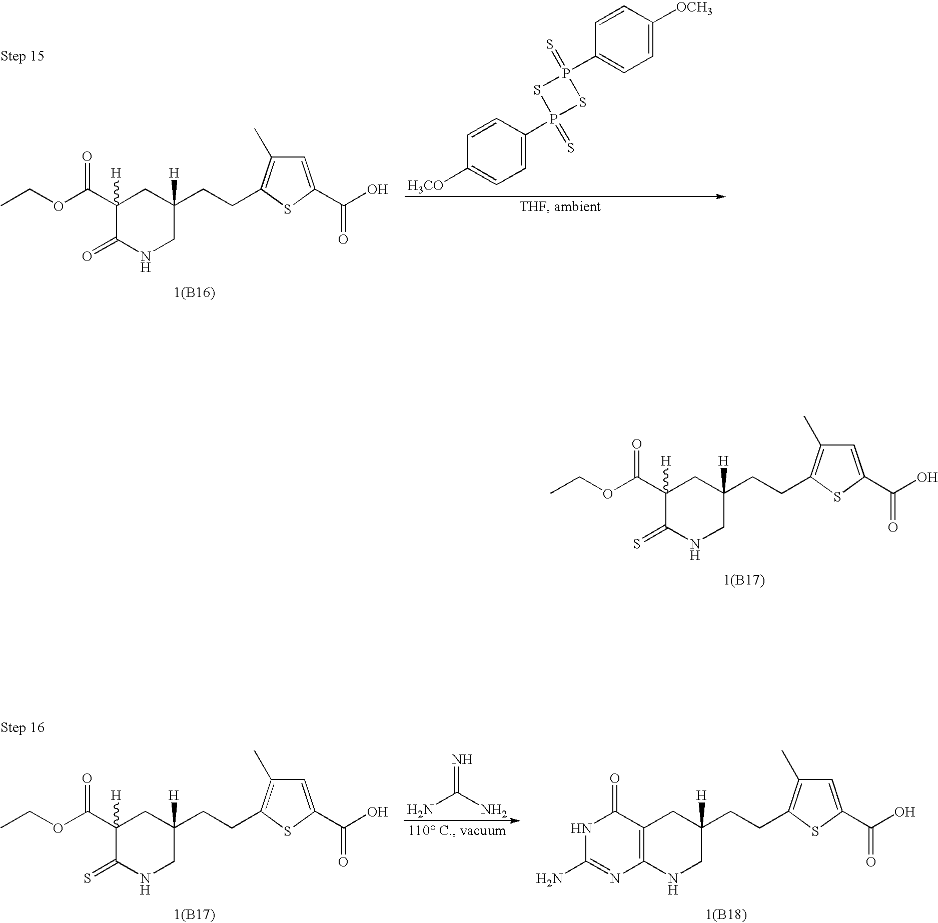

Reaction of lactam 1(B16) (53.5 g in 1.60 L THF, heated to 45° C. then re-cooled to 35° C.) with Lawesson's reagent (71.0 g; 1.12 equivalents) yielded the thiolactam 1(B17) over a period of about 21.5 hours. The reaction was quenched by dilution into 8 L methylene chloride, followed by 4 L water and 0.4 L saturated aqueous sodium chloride. The phases were split, and the organic phase was washed with 4 L water and 0.4 L saturated aqueous sodium chloride, and further evaporated in vacuo to provide thiolactam 1(B17) (estimated 56 g). No purification was performed at this point and the very crude thiolactam 1(B17) (along with all of the Lawesson's reagent by-products) was treated with neat guanidine under vacuum at 110° C. Cyclization in the melt provided pyrimidinone acid 1(B18). The crude product was dissolved in 700 ml water and the mixture was acidified with HCl to pH 5-6. The precipitated solid was collected by filtration. Acid 1(B18) was purified by slurry washing with acetone, and collection by filtration, followed by drying at 50° C. to give a crude material (45.34 g) that is pure enough for the next reaction.

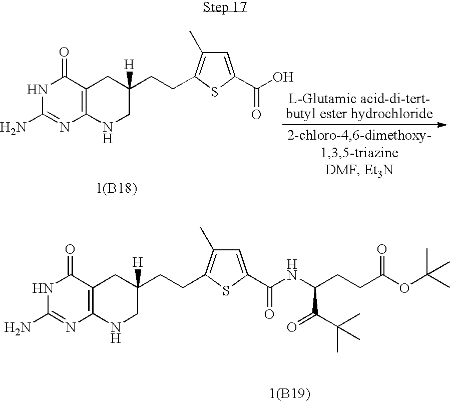

Coupling of 45.3 g of acid 1(B18) with di-t-butyl glutamate using the coupling agent, 2-chloro-4,6-dimethoxy-1,3,5-triazine (1.1 equivalents), yielded diethyl ester 1(B19). The coupling agent was added to a solution of acid 1(B18) (57.0 mL triethylamine and 698 mL DMF) at room temperature. The reaction was blanketed with argon and stirred for 1.5 hours. Di-t-butyl glutamate hydrochloride (1.1 equivalents) was added and stirring was continued for 24 hours. After filtration of solids, the filtrate was concentrated in vacuo to provide a yellow oil. The oil was dissolved in methylene chloride, washed with aqueous sodium bicarbonate, water and brine, and dried in vacuo. This material was then carefully purified by chromatography on silica (750 g) and elucted with methylene chloride/methanol (40:10) to provide di-t-butyl ester 1(B19).

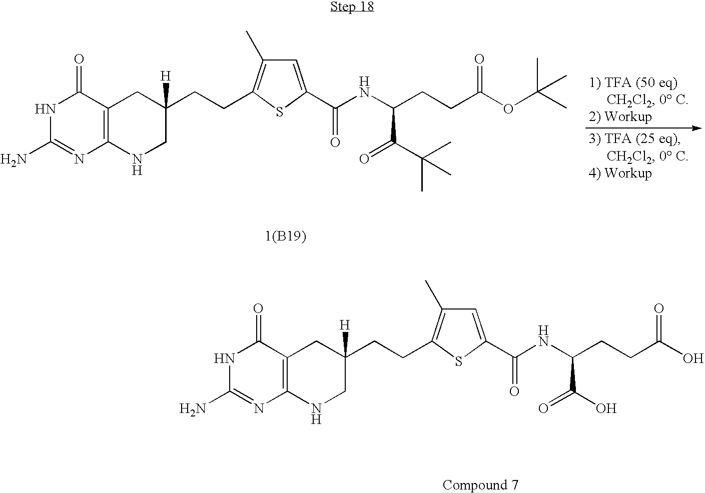

Final deprotection of di-t-butyl ester 1(B19) to give Compound 7 was accomplished as follows. A solution of purified di-t-butyl ester 1(B19) was treated with pre-chilled trifluoroacetic acid (50 equivalents) at 0° C. for 10-16 hours. All solvents were removed in vacuo at 0-3° C. The crude product was then dissolved in aqueous sodium bicarbonate, washed with methylene chloride, and obtained as a solid following acidification of the aqueous phase with HCl and collection by filtration. The solid thus obtained was treated with trifluoroacetic acid (25 equivalents) a second time as described above, and isolated in an identical manner, to give Compound 7 as a white solid. Two consecutive water re-slurries were carried out in order to free the desired compound from residual trifluoroacetic acid The product thus obtained exhibited diastereomeric purity of 99.8%;and an overall purity of>96%.

### EXAMPLE 2

Synthesis of Anti-Toxicity Agents

### EXAMPLE 2(A)

Synthesis of Methylthioadenosine (“MTA ”) (Compound AA)

Scheme I, which is depicted below, is useful for preparing MTA (Compound AA).

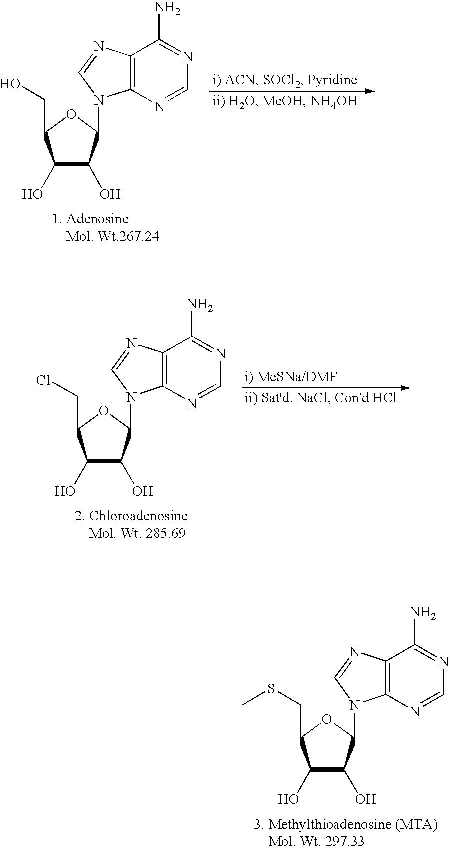

Step 1: Synthesis of Chloroadenosine

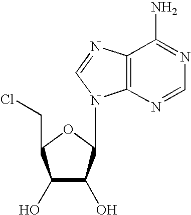

A 2-liter, 3-neck flask equipped with a mechanical stirrer and a temperature probe was charged with 400 mL of acetonitrile followed by adenosine (100 g, 0.374 mol). The resulting slurry was stirred while cooling to −8° C. with ice/acetone. The reaction was then charged with thionyl chloride (82 mL, 1.124 mol) over 5 minutes. The reaction was then charged with pyridine (6908 mL, 0.749 mol) dropwise over 40 minutes (the addition is exothermic). The ice bath was removed and the temperature was allowed to rise to room temperature while stirring for 18 hours. The product began to precipitate out of solution. After a total of 18 hours, the reaction was charged with water (600 mL) dropwise (the addition is exothermic). Acetonitrile was removed by vacuum distillation at 35° C. The reaction was then charged with methanol (350 mL). The reaction was stirred vigorously and charged dropwise with concentrated NH₄OH (225 mL). The addition was controlled to maintain the temperature below 40° C. The pH of the solution after addition was 9. The resulting solution was stirred for 1.5 hours, allowing it to cool to room temperature. After 1.5 hours, 200 mL of methanol was removed by vacuum distillation at 35° C. The resulting clear yellow solution was cooled to 0° C. for one hour and filtered. The resulting colorless solid was washed with, cold methanol (100 mL). Then dried at 40° C. under vacuum for 18 hours. The reaction afforded chloroadenosine as a colorless crystalline solid (98.9 g, 92.7%). The NMR¹H indicated that a very clean desired product with a small water peak was produced. ¹H NMR (DMSO-d6): 8.35 (1H), 8.17 (H), 7.32 (2H), 5.94 (d, J=5.7 Hz, 1H), 5.61 (d, J=6 Hz, 1H), 5.47 (d, J=5.1 Hz, 1H), 4.76 (dd, J=5.7 & 5.4 Hz, 1H), 4.23 (dd, J=5.1 Hz & 3.9 Hz, 1H), 4.10 (m, 1H), 3.35-3.98 (m, 2H).

Step 2: Synthesis of Methylthiodenosine

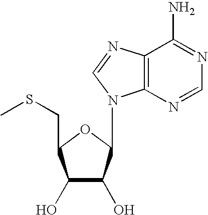

A 3-liter, 3-neck flask equipped with a mechanical stirrer and a temperature probe was charged with DMF (486 mL) followed by chloroadenosine (97.16 g, 0.341 mol). The resulting slurry was charged with NaSCH₃ (52.54 g, 0.75 mol), and the addition is exothermic. The reaction was then stirred with a mechanical stirrer for 18 hours. The reaction was charged with saturated brine (1500 mL) and the pH was adjusted to 7 with concentrated HCl (≈40 mL). The pH was monitored during addition with a pH probe. The resulting slurry was cooled to 0° C., stirred for one hour with a mechanical stirrer, and filtered. The colorless residue was triturated with water (500 mL) for one hour, filtered, and dried under vacuum for 18 hours at 40° C. A colorless solid of methylthioadenosine was produced (94.44 g, 93.3% yield from chloroadenosine; 86.5% yield from initial starting materials). The resulting MTA was 99% pure. ¹H NMR (DMSO-d6): 8.36 (1H), 8.16 (1H), 7.30 (2H), 5.90 (d, J=6.0 Hz, 1H), 5.51 (d, J=6 Hz, 1H), 5.33 (d, J=5.1 Hz, 1H), 4.76 (dd, J=6.0 & 5.4 Hz, 1H), 4.15 (dd, J=4.8 Hz & 3.9 Hz, 1H), 4.04 (m, 1H), 2.75-2.91 (m, 2H), and 2.52 (s, 3H).

### EXAMPLE 2(B)

Synthesis of Analogs of MTA

The preparation of 5′-adenosine analogs is illustrated in Scheme II:

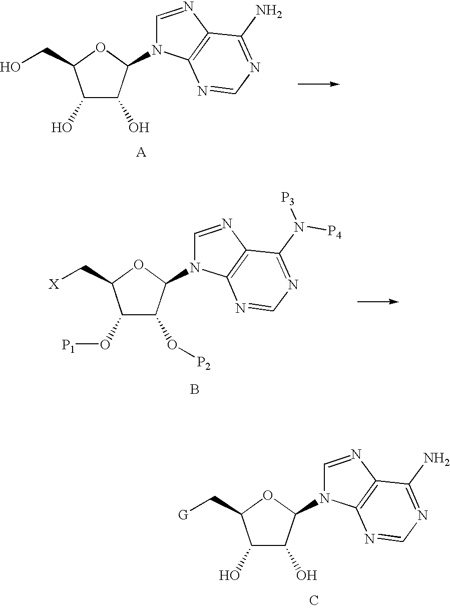

Starting with an adenosine A, the 5′ position is converted to an appropriate activated functionality X (with or without additional protecting groups P₁, P₂, P₃, P₄). For ether formation at the 5′ position, this group may be, but is not limited to a metal alkoxide. To incorporate thioethers, amines or simple reduction, the X functionality may be a leaving group such as chloride, bromide, triflate, tosylate, etc. In additon, the X group may be an aldehyde for incorporation of amine via reductive amination or carbon chain extension via Wittig olefination. After conversion to the intermediate to the desired 5′ substitution, the protecting groups (if applicable) are removed to give 5′ adenosine analogs of type C, which may be further transformed.

Scheme III shows the general method for conversion of intermediate B (X═OH) into 5′ carboxylate derivatives:

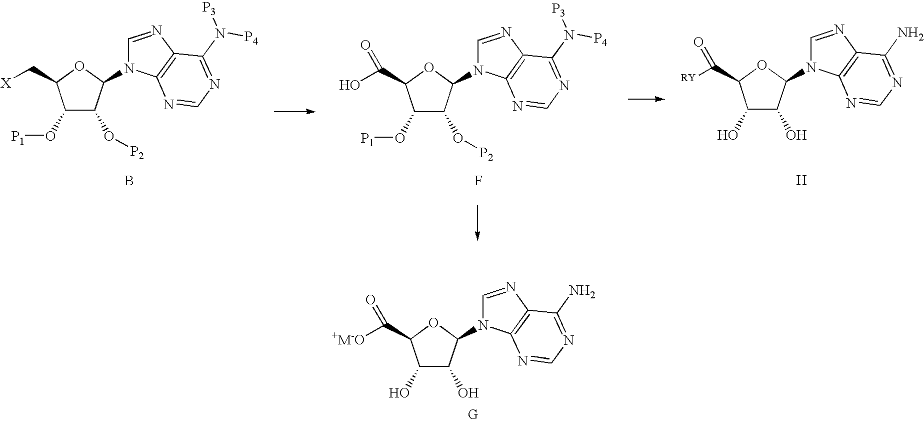

Oxidation of the 5′ hydroxyl group of compound B gives intermediate F. This compound can be further converted into either a carboxylate salt G or to carboxylic ester (Y═O) or carboxamide (Y═N) derivative H.

### EXAMPLE 2(B)(1)

(2S,3S,4R,5R)-5-(6-amino-9H-purin-9-yl)-N-ethyl-3,4-dihydroxy-N-methyltetrahydrofuran-2-carboxamide.

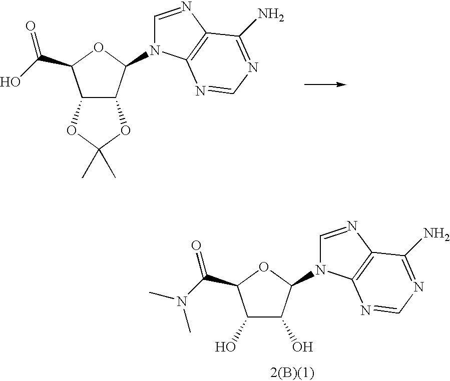

The title compound was prepared from 2′,3′-O-isopropylideneadenosine-5′-carboxylic acid (R. E. Harmon et. al. Chem. Ind. (London) 1141 (1969); P. J. Harper and A. Hampton J. Org. Chem. 35, 1688 (1970); A. K. Singh Tetrahedron Lett. 33, 2307 (1992)) and N-ethylmethylamine using a modification of the procedure of S. F. Wnuk et. al. (J. Med. Chem. 39, 4162 (1996)) as follows:

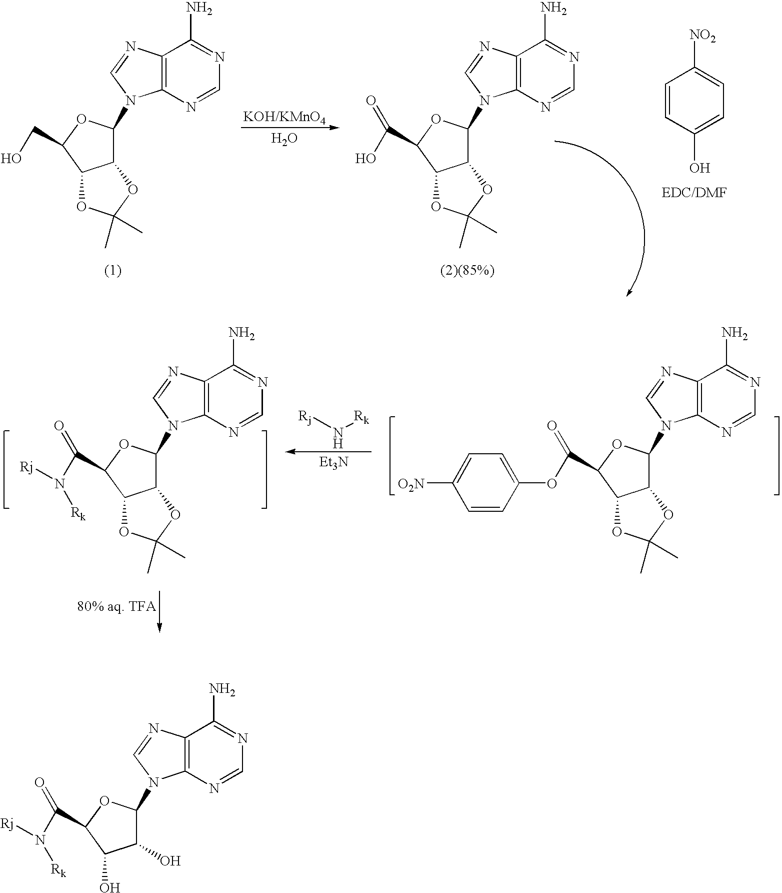

The reagents 1-[3-(dimethylamino)propyl]-3-ethylcarbodiimide hydrochloride and 4-nitrophenol were used to couple the two starting materials and the protecting group was removed with aqueous TFA (as described in the reference listed above) to give, after purification by silica gel column chromatography (eluted with 9:1 CH₂Cl₂:MeOH), 336 mg (57%) of product 2(B)(1) as white solid. mp: 86-90 ° C.; ¹H-NMR (DMSO-d₆) δ0.90-1.14 (m, 6H), 2.76 (s, 1H), 2.90 (s, 1H), 3.21-3.35 (m, 2H), 4.18 (br s, 1H), 4.37 (br s, 2H), 4.69-4.74 (dd, 1H, J=3.0, 2.3 Hz), 5.59 (br s, 1H), 5.94-5.96 (d, 1H, J=5.2 Hz), 7.29 (br s, 2H), 8.06 (s, 1H), 8.50-8.52 (d, 1H, J=7.5 Hz). LRMS (m/z) 323 (M+H)⁺]and 345 (M+Na)⁺. Anal. (C₁₃H₁₈N₆O₄-2.3 TFA) C,H,N.

### EXAMPLE 2(B)(2)

2-(6-Amino-purin-9-yl)-5-(4-fluoro-benzyloxymethyl-tetrahydro-furan-3,4-diol.

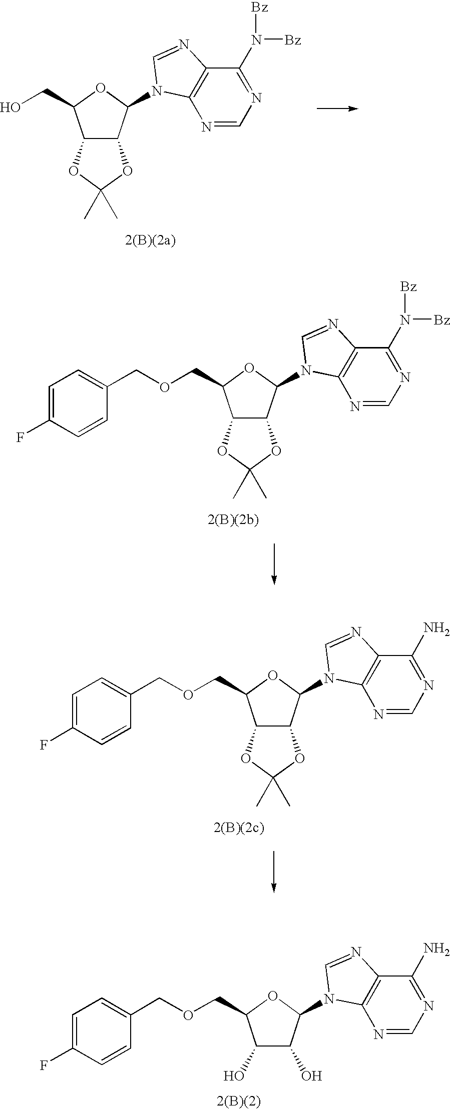

Intermediate 2(B)(2a): N-Benzoyl-N-{9-[6-(4-fluoro-benzyloxymethyl)-2,2-dimethyl-tetrahydro-furo-[3,4-d][1,3]dioxo-4-yl]-9H-purine-6-yl}-benzamide. To a solution of the starting reagent 2(B)(2a) (400 mg, 0.78 mmol) with nBu4N⁺I⁻ (15 mg, 0.04 mmol.) in 16 ml of THF was added NaH (47 mg, 1.16 mmol., 60% in mineral oil). After 30 min, 4-fluorobenzyl bromide (0.12 ml, 0.94 mmol) was added dropwise. The resulting mixture was stirred at room temperature overnight. The mixture was quenched with MeOH and neutralized with HOAc to pH7.0 and florisil (2.0 g) was added, then concentrated by vacuum. The residue was treated with CH₂Cl₂ and filtered off and washed well with CH₂Cl₂. The filtrate was extracted with 10% NaHSO₃ (30 ml), brine (30 ml). The organic layer was dried (Na₂SO₄), then concentrated by vacuum. The residue was purified by Dionex System (25%-95% MeCN:H₂O w 0.1% HOAc buffer) to collect desired fraction to afford intermediate 2(B)(2b) (114 mg, 0.18 mmol., 23% yield) as white solid. TLC: R_{f}=0.2 (Hexane:EtOAc/2:1). ¹H NMR (400 MHz, CHLOROFORM-D) □ ppm 1.31 (d, J=10.11 Hz, 3 H) 1.55 (d, J=7.07 Hz, 3 H) 4.36 (dd, J=11.62, 5.56 Hz, 1 H) 4.49 (m, 2 H) 5.04 (m, J=6.32, 3.54 Hz, 1 H) 5.39 (dd, J=6.44, 2.40 Hz, 2 H) 5.48 (m, J=1.26 Hz, 2 H) 5.99 (d, J=2.27 Hz, 1 H) 6.84 (m, 2 H) 7.08 (m, J=7.58, 7.58 Hz, 3 H) 7.35 (m, 5 H) 7.49 (t, J=7.45 Hz, 1 H) 7.87 (m, 3 H) 8.42 (s, 1 H). MS for C₃₄H₃₀FN₅O₆ (MW:623), m/e 624 (MH⁺).

Intermediate 2(B)(2c): 9-[6-(4-Fluoro-benzyloxymethyl-2,2-dimethyl-tetrahydro-furo-[3,4-d][1,3]dioxo-4-yl]-9H-purin-6-ylamine. To a solution of 2(B)(2b) (110 mg, 0.18 mmol.) in 2 ml of MeOH was added concentrate NH₄OH (2 ml). The resulting mixture was stirred at room temperature under N₂ for overnight. The reaction mixture was concentrated by vacuum. The residue was purified by Dionex System (5%-95% MeCN:H₂O w 0.1% HOAc) to collect desired fraction to afford intermediate 2(B)(2c) (47 mg, 0.11 mmol.,63% yield) as white solid. TLC: R_{f}<0.3 (CH₂Cl₂:EtOAc/2:1). ¹H NMR (400 MHz, CHLOROFORM-D) □ ppm 1.31 (s, 3 H) 1.58 (s, 3 H) 3.74 (m, 1 H) 3.91 (d, J=12.88 Hz, 1 H) 4.48 (s, 1 H) 4.75 (s, 2 H) 5.05 (d, J=5.81 Hz, 1 H) 5.14 (t, J=5.31 Hz, 1 H) 5.77 (d, J=5.05 Hz, 1 H) 6.16 (s, 1 H) 6.66 (s, 1 H) 6.95 (m, J=8.59, 8.59 Hz, 2 H) 7.27 (m, J=8.21, 5.43 Hz, 2 H) 7.71 (s, 1 H) 8.30 (s, 1 H). MS for C₂₀H₂₂FN₅O₄ (MW:415), m/e 416(MH⁺). The title compound 2(B)(2) was made as follows. The reaction mixture of 2(B)(2c) (45 mg, 0.11 mmol.) in 1.5 ml of HOAc and 1.5 ml of H₂O was heated at 70° C. for 8 hours. The mixture was concentrated by vacuum. The residue was purified by Dionex System (5%-95% MeCN:H₂O w 0.1% HOAc) to collect desired fraction to afford 2(B)(2) (35 mg, 0.1 mmol, 85% yield) as white solid. TLC: R_{f}=0.1 (CH₂Cl₂:MeOH/9:1). ¹H NMR (400 MHz, MeOD) □ ppm 3.66 (dd, J=12.63, 2.53 Hz, 1 H) 3.80 (m, 1 H) 4.09 (q, J=2.53 Hz, 1 H) 4.24 (dd, J=5.05, 2.53 Hz, 1 H) 4.66 (dd, J=6.44, 5.18 Hz, 1 H) 4.75 (m, 2 H) 5.87 (d, J=6.32 Hz, 1 H) 6.96 (m, 2 H) 7.32 (dd, J=8.59, 5.56 Hz, 2 H) 8.17 (d, J=9.85 Hz, 2 H). HRMS for C₁₇H₁₈FN₅O₄ (MW:375.35), m/e 376.1417 (MH⁺). EA Calcd for C₁₇H₁₈F N₅O₄.1.1H₂O: C 51.67, H 5.15, N 17.72. Found: C 51.76, H 4.96, N 17.33.

### EXAMPLE 2(B)(3)

2S,3R,4R,5R,)-2-(6-Amino-purin-9-yl)-5-(tert-butylamino-methyl)-tetrahydro-furan-3,4-diol

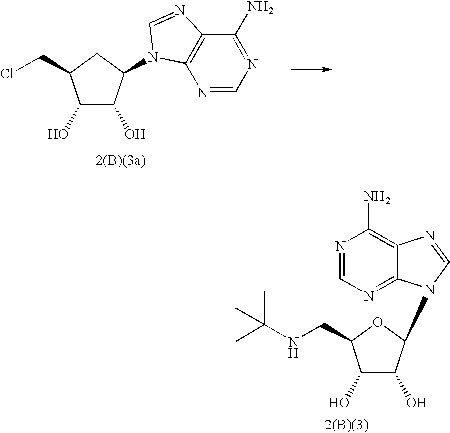

tert-Butylamine (1.5 mL, 15 mmol) was added to 2(B)(3a) (286 mg, 1.0 mmol) and the mixture was microwaved using Smithsynthesizer (150° C., 1 h). The resulting mixture was concentrated under reduced pressure to reduce the volume. The crude mixture was then purified by reverse phase HPLC (Dionex System; 100 →50% MeCN:H₂O) to afford Cc1 (120 mg, 37% yield) as a white foam. ¹H NMR (400 MHz, CD₃OD) δ ppm 1.24 (d, J=8.8 Hz, 9 H) 1.82 (s, 1 H) 3.42 (m, 1 H) 3.69 (s, 1 H) 4.18 (m, 1 H) 4.33 (m, 1 H) 4.41 (br. s., 1 H) 5.71 (s, 1 H) 5.76 (br. s., 1 H) 5.92 (d, J=5.1 Hz, 1 H) 7.31 (s, 1 H) 7.54 (m, 1 H) 8.11 (s, 1 H) 8.15 (s, 1 H). LCMS Calcd for C₁₄H₂₂N₆O₃ (MW:322), m/e 323 (MH⁺). Anal. Calcd. for C₁₄H₂₂ N₆O₃.1.4CH₃COOH.2.0H₂O C: 45.60, H: 7.20, N: 18.99. Found C: 45.47, H: 7.45, N: 18.62.

### EXAMPLE 2(B)(4)

(2S,3R,4R,5R)-2-(6-Amino-purin-9-yl)-5-phenylaminomethyl-tetrahydro-furan-3,4-diol

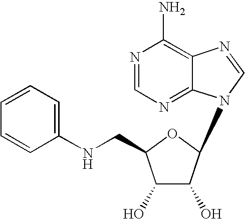

Compound 2(B)(4) was prepared and isolated by modifying the method described in Example 2(B)(3). ¹H NMR (400 MHz, CD₃OD) δ ppm 1.80 (s, 1 H) 3.39 (m, J=4.0 Hz, 2 H) 4.18 (m, J=4.0 Hz, 1 H) 4.24 (m, 1 H) 4.73 (m, 1 H) 5.86 (d, J=5.8 Hz, 1 H) 6.53 (t, J=7.2 Hz, 1 H) 6.63 (m, J=7.6 Hz, 2 H) 7.01 (m, 2 H) 8.08 (s, 1 H) 8.15 (s, 1 H). HRMS Calcd for C₁₆H₁₉N₆O₃ (M+H)=343.1519, observed MS=343.1516.

### EXAMPLE 2(B)(5)

2-(6-Amino-purin-9-yl)-5-dimethylaminomethyl-tetrahydro-furan-3,4-diol

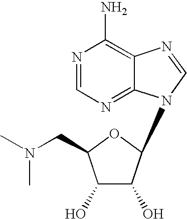

Compound 2(B)(5) was prepared and isolated by modifying the method described in Example 2(B)(3). ¹H NMR (400 MHz, CD₃OD) δ ppm 2.72 (s, 3 H) 2.88 (s, 3 H) 3.77 (s, 1 H) 4.25 (m, J=5.8 Hz, 1 H),4.36 (m, 2 H) 4.46 (m, 1 H) 4.52 (s 1 H) 5.89 (s, 1 H) 6.05 (d, J=5.6 Hz, 1 H) 7.66 (s, 1 H) 8.26 (s, 1 H) 8.28 (s, 1 H) HRMS Calcd for C₁₂H₁₉N₆O₃ (M+H)=295.1519, observed MS=295.1501.

### EXAMPLE 2(B)(6)

(2S,3R,4R,5R)-2-(6-Amino-purin-9-yl)-5-[(2-pyridin-2-yl-ethylamino)-methyl]-tetrahydro-furan-3,4-diol

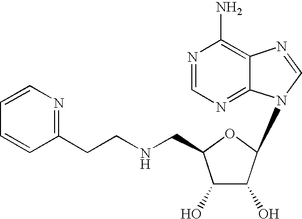

Compound 2(B)(6) was prepare and isolated by modifying the method described in Example 2(B)(3). ¹H NMR (300 MHz, CD₃OD) δ ppm 1.94 (m, 2 H) 2.77 (m, 1 H) 3.17 (t, J=6.8 Hz, 3 H) 3.36 (m, 4 H) 3.73 (m, 1 H) 4.43 (d, J=9.2 Hz, 1 H) 6.05 (d, J=5.7 Hz, 1 H) 7.36 (dd, J=14.3, 7.9 Hz, 2 H) 7.80 (m, 1 H) 8.07 (d, J=3.6 Hz, 1 H) 8.27 (d, J=8.1 Hz, 1 H) 8.55 (m, 1 H). HRMS Calcd for C₁₇H₂₁N₇O₃ (M+H)=372.1784, observed MS=372.1799.

### EXAMPLE 2(B)(7)

(2S,3R,4R,5R)-2-(6-Amino-purin-9-yl)-5-[(4-benzylamino)-methyl]-tetrahydro-furan-3,4-diol

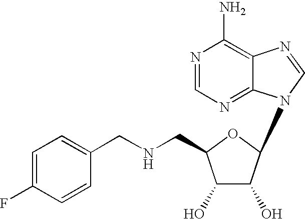

Compound 2(B)(7) was prepared and isolated by modifying the method described in Example 2(B)(3). ¹H NMR (300 MHz, CD₃OD) δ ppm 2.00 (s, 2 H) 3.38 (m, 2 H) 4.13 (s, 2 H) 4.23 (d, J=3.8 Hz, 2 H) 4.41 (m, 2 H) 4.66 (s, 1 H) 5.89 (s, 1 H) 6.03 (d, J=4.9 Hz, 1 H) 7.19 (m, 2 H) 7.51 (m, 2 H) 8.05 (d, J=2.6 Hz, 1 H) 8.25 (s, 1 H). HRMS Calcd for C₁₇H₁₉FN_{6l O}₃ (M+H)=3 75.1581, observed MS=375.1582.

### EXAMPLE 2(B)(8)

(2S,3R,4R,5R)-2-(6-Amino-purin-9-yl)-5-[(2-hydroxy-ethylamino)-methyl]-tetrahydro-furan,3,4-diol.

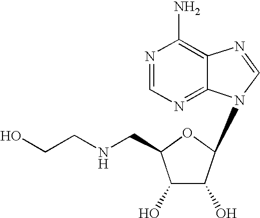

Compound 2(B)(8) was prepared and isolated by modifying the method described in Example 2(B)(3). ¹H NMR (400 MHz, CD₃OD) δ ppm 1.78 (s, 2 H) 2.69 (t, J=5.4 Hz, 1 H) 2.81 (t, J=5.3 Hz, 2 H) 3.24 (s, 2 H) 3.57 (m, 2 H) 4.11 (br. s., 1 H) 4.18 (m, J=4.8 Hz, 1 H) 4.70 (m, J=5.2 Hz, 2 H) 5.38 (s, 1 H) 5.86 (d, J=5.3 Hz, 1 H) 8.11 (s, 1 H) 8.16 (s, 1 H). HRMS Calcd for C₁₂H₁₈N₆O₄ (M+H)=311.1468, observed MS=311.1480.

### EXAMPLE 2(B)(9)

2-(6-Amino-purin-9-yl)-5-morpholin-yl-methyl-tetrahydro-furan-3,4-diol

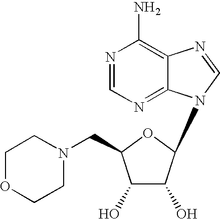

(Compound 2(B)(9) was prepared and isolated by modifying the method described in Example 2(B)(3). ¹H NMR (400 MHz, CD₃OD) δ ppm 1.72 (d, J=5.6 Hz, 2 H) 2.37 (m, 2 H) 2.57 (m, 2 H,) 2.93 (m, 2 H) 3.08 (m, 1 H) 3.45 (m, J=4.8, 4.8 Hz, 2 H) 3.61 (m, 2 H) 3.99 (m, 2 H) 4.07 (t, J=5.7 Hz, 1 H) 4.46 (m, 1 H) 5.75 (d, J=4.3 Hz, 1 H) 7.97 (s, 1 H) 8.07 (s, 1 H). HRMS Calcd for C₁₄H₂₀N₆O₄ (M+H)=337.1624, observed MS=337.1626. Anal. Calcd for C₁₄H₂₀N₆O₄.1.5CH₃COOH C: 46.50, H: 6.29, N: 19.14. Found C: 46.42, H: 6.85, N: 19.10.

### EXAMPLE 2(B)(10)

2-(6-Amino-purin-9-yl)-5-pyrrolidin-yl-methyl-tetrahydro-furan-3,4-diol.

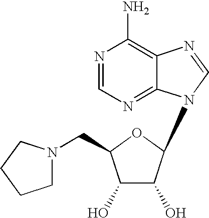

Compound 2(B)(10) was prepared and isolated by modifying the method described in Example 2(B)(3). ¹H NMR (400 MHz, CD₃OD) δ ppm 1.82 (m, 2 H) 2.93 (m, J=6.44, 6.44 Hz, 4 H) 3.13 (m, 2 H) 3.20 (m, 2 H) 3.24 (s, 1 H) 3.33 (m, J=13.0, 9.2 Hz, 2 H) 4.20 (m, 2 H) 4.71 (t, J=4.8 Hz, 1 H) 5.90 (d, J=4.8 Hz, 1 H) 8.12 (s, 1 H) 8.15 (s, 1 H). HRMS Calcd for C₁₄H₂₀N₆O₃ (M+H)=321.1675, observed MS 321.1662. Anal. Calcd for C₁₄H₂₀N₆O₃.1.0CH₃COOH.0.6CH₂Cl₂ C: 41.07, H: 6.48, N: 17.31. Found C: 41.11, H: 5.86, N: 17.61.

### EXAMPLE 2(B)(11)

2-(6-Amino-purin-9-yl)-5-cyclopentylaminomethyl-tetrahydro-furan-3,4-diol.

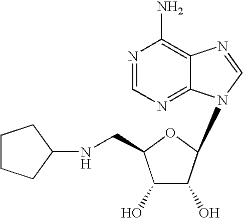

Compound 2(B)(11) was prepared and isolated by modifying the method described in Example 2(B)(3). ¹H NMR (400 MHz, CD₃OD) δ ppm 0.07 (m, 6 H) 0.30 (m, 2 H) 0.45 (m, 4 H) 1.87 (m, 2 H) 1.96 (m, 2 H) 2.19 (s, 1 H) 2.70 (m, 1 H) 2.78 (t, J=4.7 Hz, 1 H) 4.40 (d, J=5.1 Hz, 1 H) 6.61 (s, 1 H) 6.65 (s, 1 H). LCMS Calcd for C₁₅H₂₂N₆O₃ (M+H)=335, observed MS=335. Anal. Calcd for C₁₄H₂₂N₆O₃.2.2CH₃COOH.0.8C₆H₁₂ C: 51.84, H: 8.05, N: 14.99. Found C: 51.89, H: 8.46, N: 15.02.

### EXAMPLE 2(B)(12)

(2S,3R,4R,5R)-2-(6-amino-9H-purin-9-yl)-5-(phenoxymethyl)tetrahydrofuran-3,4-diol.

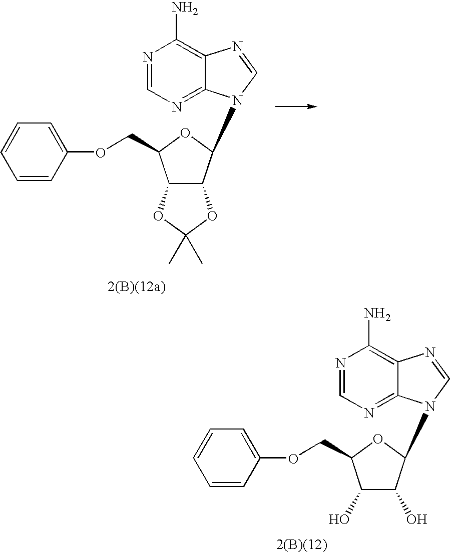

Intermediate 2(B)(12a): (2S,3R,4R,5R)-9-[2,2-dimethyl-6-(phenoxymethyl)tetrahydrofuro[3,4-d][1,3]dioxol-4-yl]-9H-purin-6-amine Triphenyl phosphine (641 mg, 2.44 mmol) and phenol (311 mg, 3.30 mmol) were added sequentially to a stirred solution of 2′, 3′-isopropylidene adenosine (500 mg, 1.63 mmol) in THF (15 mL). The reaction mixture was then put in an ice bath and diisopropyl azodicarboxylate (0.5 mL; 2.44 mmol) was added. The ice bath was removed and the mixture was stirred at room temperature for 12 h. The solvent was evaporated to give a brown-yellow oil residue. The residue was purified by silica gel chromatography (eluting with 80→100% EtOAc in hexanes) to give compound 2(B)(12a) as a white foam (152.8 mg; 0.4 mmol; 40% yield). ¹H NMR (400 MHz, CDCl₃) δ ppm 1.43 (s, 3 H) 1.67 (s, 3 H) 4.14 (dd, J=10.2, 4.7 Hz, 1 H) 4.27 (m, 1 H) 4.70 (m, 1 H) 5.18 (dd, J=6.1, 2.8 Hz, 1 H) 5.46 (dd, J=6.2, 2.1 Hz, 1 H) 6.24 (d, J=2.3 Hz, 1 H) 6.37 (m, 1 H) 6.80 (d, J=8.1 Hz, 1 H) 6.95 (t, J=7.5 Hz, 1 H) 7.26 (m, 1 H) 7.48 (m, 2 H) 7.68 (m, 1 H) 7.99 (s, 1 H) 8.37 (s, 1 H). Acetic acid (20 mL, 80% in H₂O) was added to compound 2(B)(12a) (153 mg, 0.4 mmol). The resulting solution was heated to 100° C. for 6 hrs. The reaction mixture was evaporated and was purified by silica gel chromatography (eluting with 28% MeOH, 2% H₂O in CH₂Cl₂) to give compound 2(B)(12) as a white foam (75.5 mg; 0.22 mmol; 40% yield); ¹H NMR (300 MHz, CD₃OD) □ ppm 4.13 (dd, J=10.7, 3.4 Hz, 1 H) 4.23 (d, J=3.2 Hz, 1 H) 4.29 (m, 1 H) 4.40 (t, J=4.9 Hz, 1 H) 4.63 (t, J=4.7 Hz, 1 H) 6.00 (d, J=4.5 Hz, 1 H) 6.85 (dd, J=12.7, 7.6 Hz, 3 H) 7.18 (m, 2 H) 8.10 (s, 1 H) 8.22 (s, 1 H). Anal. Calcd for C₁₆H₁₇N₅O₄.0.25H₂O. 2CH₃COOH C: 53.00, H: 5.31, N: 17.17. Found C: 52.82, H: 5.52, N: 17.29.

### EXAMPLE 2(B)(13)

(2S,3R,4R,5R)-2-(6-amino-9H-purin-9-yl)-5-[(pyridin-3-yloxy)methyl]tetrahydrofuran-3,4-diol.

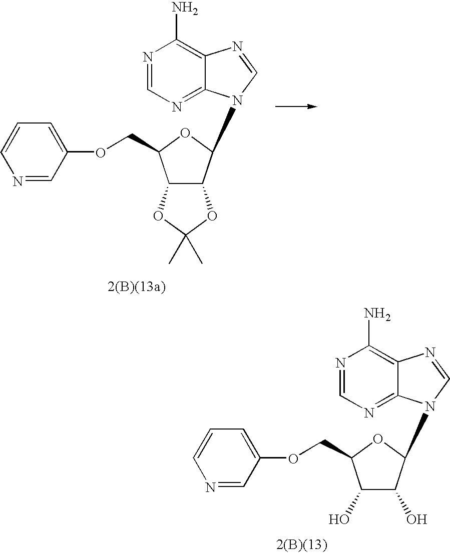

Compound 2(B)(13a) was prepared and isolated by modifying the method described in Example 2(B)(12), with the substitution of 3-hydroxypyridine for the phenol reagent. ¹H NMR (400 MHz, CDCl₃) δ ppm 1.39 (s, 3 H) 1.62 (s, 3 H) 4.17 (dd, J=10.1, 5.6 Hz, 1 H) 4.28 (m, 1 H) 4.64 (m, 1 H) 5.18 (dd, J=6.3, 3.3 Hz, 1 H) 5.48 (dd, J=6.3, 2.0 Hz, 1 H) 6.16 (d, J=2.0 Hz, 1 H) 6.27 (s, 2 H) 7.05 (ddd, J=8.4, 3.0, 1.3 Hz, 1 H) 7.13 (m, 1 H) 7.89 (s, 1 H) 8.19 (m, 2 H) 8.31 (s, 1 H).

Compound 2(B)(13) was prepared and isolated from intermediate 2(B)(13a) using the method described in Example.2(B)(12). Compound 2(B)(13): ¹H NMR (400 MHz, CD₃OD) δ ppm 4.30 (m, 3 H) 4.45 (t, J=4.9 Hz, 1 H) 4.70 (t, J=4.8 Hz, 1 H) 5.97 (d, J=4.6 Hz, 1 H) 7.23 (dd, J8.5, 4.7 Hz, 1 H) 7.36 (ddd, J=8.5, 2.8, 1.3 Hz, 1 H) 8.02 (d, J=4.3 Hz, 1 H) 8.08 (s, 1 H) 8.17 (s, 2 H). Anal. Calcd for C₁₅H₁₆N₆O₄.1.25H₂O.0.25CH₃COOH C: 48.75, H: 5.15,N: 22.01. Found C: 48.32, H: 5.12, N: 22.35.

### EXAMPLE 2(B)(14)

(2S,3R,4R,5R)-2-(6-amino-9H-purin-9-yl)-5-[(pyridin-2-yloxy)methyl]tetrahydrofuran-3,4-diol.

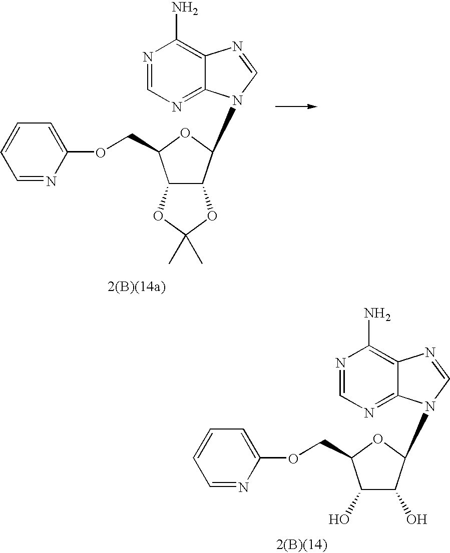

Compound 2(B)(14a) was prepared and isolated by modifying the method described in Example 2(B)(12), with the substitution of 2-hydroxypyridine for the phenol reagent. Intermediate 2(B)(14a): ¹H NMR (400 MHz, CDCl₃) δ ppm 1.37 (s, 3 H) 1.60 (s, 3 H) 4.46 (dd, J=1.6, 5.3 Hz, 1 H) 4.54 (m, 1 H) 4.68 (m, 1 H) 5.09 (dd, J=6.2, 2.9 Hz, 1 H) 5.44 (dd, J=6.2, 2.2 Hz, 1 H) 6.17 (d, J=2.0 Hz, 1 H) 6.41 (s, 2 H) 6.52 (d, J=8.3 Hz, 1 H) 6.80 (dd, J=6.3, 5.1 Hz, 1 H) 7.47 (m, 1 H) 7.94 (s, 1 H) 8.04 (dd, J=5.1, 1.0 Hz, 1 H) 8.32 (s, 1 H).

Compound 2(B)(14) was prepared and isolated from intermediate 2(B)(14a) using the method described in Example 2(B)(12). Compound 2(B)(12). ¹H NMR (400 MHz, CD₃OD) δ ppm 4.41 (q, J=4.2 Hz, 1 H) 4.48 (t, J=4.9 Hz, 1 H) 4.54 (m, 1 H) 4.61 (m, 1 H) 4.76 (t, J=4.9 Hz, 1 H) 6.08 (d, J=4.6 Hz, 1 H) 6.83 (d, J=8.3 Hz, 1 H) 6.95 (dd, J=6.7, 5.4 Hz, 1 H) 7.68 (m, 1 H) 8.12 (dd, J=5.1, 1.3 Hz, 1 H) 8.19 (s, 1 H) 8.31 (s, 1 H). Anal. Calcd for C₁₅H₁₆N₆O₄.0.75H₂O.0.5CH₃COOH C: 49.55, H: 5.07, N: 21.67. Found C: 49.85, H: 5.04, N: 21.74.

### EXAMPLE 2(B)(15)

(2S,3R,4R,5R)-2-(6-amino-9H-purin-9-yl)-5-[(4-methoxyphenoxy)methyl]tetrahydrofuran-3,4-diol.

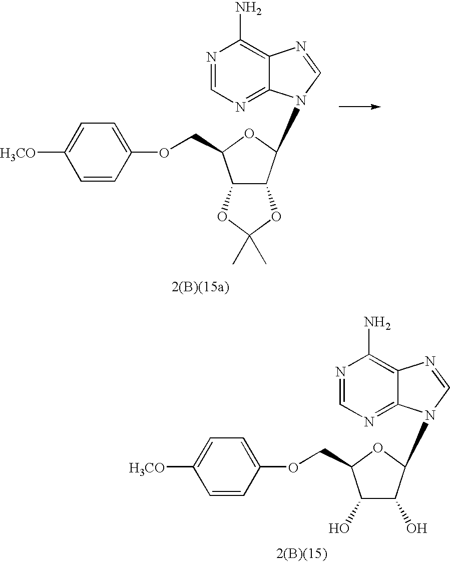

Compound 2(B)(15a) was prepared and isolated by modifying the method described in Example 2(B)(12), with the substitution of 4-methoxyphenol for the phenol reagent. Intermediate 2(B) (15a): ¹H NMR (400 MHz, CDCl₃) δ ppm 1.39 (s, 3 H) 1.63 (s, 3 H) 3.72 (s, 3 H) 4.06 (dd, J=10.2, 4.7. Hz, 1 H) 4.18 (m, 1 H) 4.65 (m, 1 H) 5.12 (dd, J=6.2, 2.7 Hz, 1 H) 5.41 (dd, J=6.1, 2.3 Hz, 1 H) 6.21 (m, 3 H) 6.73 (m, 3 H) 7.97 (s, 1 H) 8.34 (s, 1 H).

Compound 2(B)(15) was prepared and isolated from intermediate 2(B)(15a) using the method described in Example 2(B)(12). Compound 2(B)(15): ¹H NMR (400 MHz, DMSO-d₆) δ ppm 3.68 (s, 3 H) 4.11 (m, 1 H) 4.18 (m, 2 H) 4.30 (q, J=4.6 Hz, 1 H) 4.67 (m, 1 H) 5.38 (d, J=5.3 Hz, 1 H) 5.58 (d, J=5.8 Hz, 1 H) 5.94 (d, J=5.1 Hz, 1 H) 6.87 (m, 4 H) 7.30 (s, 2 H) 8.14 (s, 1 H) 8.33 (s, 1 H). Anal. Calcd for C₁₇H₁₉N₅O₅.0.5H₂O C: 53.40, H: 5.27, N: 18.32. Found C: 53.49, H: 5.33, N: 18.02.

### EXAMPLE 2(B)(16)

(2S,3R,4R,5R)-N-Benzoyl-N-{9-[2,2-dimethyl-6-((E)-styryl)-tetrahydro-furo[3,4-d][1,3]dioxol-4-yl]-9H-purin-6-yl}-benzamide

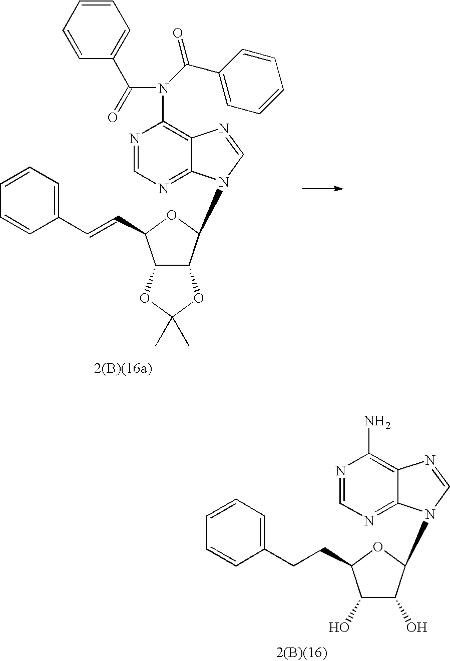

Intermediate 2(B)(16a) was prepared and isolated using the method disclosed in Montgomery et al., J. Heterocycl. Chem. 11, 211 (1974). Intermediate 2(B)(16a): ¹H NMR (300 MHz, CHLOROFORM-D) δ ppm 1.33 (s, 3 H) 1.59 (s, 3 H) 4.81 (dd, J=7.6, 3.1 Hz, 1 H) 4.98 (m, 1 H) 5.44 (m, 1 H) 5.63 (dd, J=11.5, 9.6 Hz, 1 H) 6.07 (d, J=1.9 Hz, 1 H) 6.12 (d, J=2.3 Hz, 1 H) 6.19 (dd, J=15.9, 7.6 Hz, 1 H) 6.59 (m, 1 H) 7.31 (m, 10 H) 7.78 (m, 4 H) 8.13 (m, 1 H) 8.63 (s, 1 H).

Compound 2(B)(16) was then prepared and isolated by modifying the method described in Montgomery et al, J. Heterocycl. Chem. 11, 211 (1974). ¹H NMR (400 MHz, DMSO-d₆) δ ppm 1.95 (m, 2 H) 2.59 (m, 1 H) 2.66 (dd, J=9.4, 5.6 Hz, 1 H) 3.84 (m, 1 H) 4.07 (q, J=4.7 Hz, 1 H) 4.71 (q, J=5.6 Hz, 1 H) 5.18 (d, J=5.1 Hz, 1 H) 5.42 (d, J=6.1 Hz, 1 H) 5.86 (d, J=5.6 Hz, 1 H) 7.21 (m, 5 H) 8.14 (s, 1 H) 8.34 (s, 1 H). Anal. Calcd for C₁₇H₁₉N₅O₃.1H₂O C: 56.82, H: 5.89, N: 19.49. Found C: 56.89, H: 5.70, N: 19.56.

### EXAMPLE 2(B)(17)

{[5-(6-Amino-purin-9-yl)-3,4-dihydroxy-tetrahydro-furan-2-carbonyl]-amino}-acetic acid methyl ester.

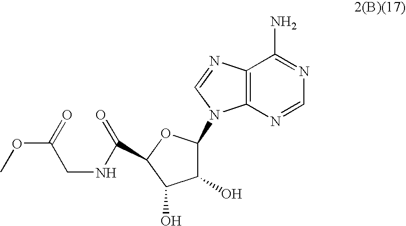

Compound 2(B)(17) was made by modification of the method described in Example 2(B)(1), with the addition of Glycine methylester*HCl (249 mg, 198 mmol) and Et₃N (0.5 ml, 3.3 mmol) in place of N-ethylmethylamine. 2(B)(17): ¹HNMR (300 MHz, DMSO-D6) δ ppm 1.20 (t, J=7.16 Hz, 2H) 4.03 (m, 3 H) 4.17 (d, J=4.52 Hz, 1 H) 4.42 (d, J=0.94 Hz, 1 H) 4.61 (m, J=7.82, 4.62 Hz, 2 H) 6.02 (d, J=7.91 Hz, 2 H) 7.78 (s, 2 H) 8.28 (s, 1 H) 8.45 (s, 1 H) 9.54 (s, 1 H). LCMS Calcd for C₁₃H₁₆N₆O₆ (M+H)=353, observed MS=353. EA calcd for C₁₃H₁₆N₆O₆*0.6TFA; C:40.54, H:3.98, N:19.98. Found C:40.98, H:4.40 N:19.38.

### EXAMPLE 2(B)(18)

{[5-(6-Amino-purin-9-yl)-3,4-dihydroxy-tetrahydro-furan-2-carbonyl]-amino}-3-phenyl-propionic acid methyl ester

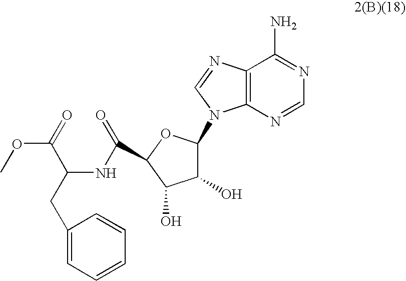

Compound 2(B)(18) was made by modification of the method described in Example 2(B)(1), with the addition of H-Phe-OMe*HCl (418 mg, 1.98 mmol) and Et₃N (0.5 ml, 3.3 mmol) in place of N-ethylmethylamine. 2(B)(18): ¹H NMR (300 MHz, DMSO-D6) δ ppm 3.38 (m, 3 H) 3.63 (m, 3 H) 4.25 (s, 1 H) 4.48 (m, 1 H) 4.88 (m, 1 H) 5.56 (d, J=6.78 Hz, 1 H) 5.76 (d, J=4.14 Hz, 1 H) 5.89 (m, J=8.29 Hz, 1 H) 7.23 (m, 5 H) 7.51 (s, 2 H) 8.13 (m, 1 H) 8.30 (m, 1 H) 9.55 (d, J=8.67 Hz, 1 H). LCMS Calcd for C₂₀H₂₂ N₆O₆ (M+H)=443, observed MS=443. EA calcd for C₂₀H₂₂N₆O₆*0.55TFA; C:50.26, H:4.51, N:16.67. Found C:50.56, H:4.94, N:16.14.

### EXAMPLE 2(B)(19)

5-(6-Amino-purin-9-yl)-3,4-dihydroxy-tetrahydro-furan-2-carbonylic acid (2-hydroxy-ethyl)-amide

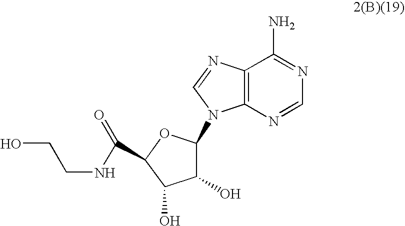

Compound 2(B)(18) was made by modification of the method described in Example 2(B)(1), with the addition of ethanolamine (0.12 ml, 1.92 mmol) in place of N-ethylmethylamine. 2(B)(19): ¹H NMR (300 MHz, DMSO-D6) δ ppm 3.23 (m, 2 H) 3.41 (m, 3H) 4.10 (m, J=4.14 Hz, 1 H) 4.29 (d, J=1.32 Hz, 1 H) 4.57 (m, J=2.83 Hz, 1 H) 5.52 (m, 1 H) 5.71 (m, 1 H) 5.92 (d, J=7.72 Hz, 1 H) 7.48 (s, 2 H) 8.18 (s, 1 H) 8.37 (s, 1 H) 8.92 (m, J=5.84 Hz, 1 H). LCMS Calcd for C₁₂H₁₆ N₆O₅ (M+H)=325, observed MS=325. EA calcd for C₁₂H₁₆N₆O₅*3.3TFA*1.0 CH₂Cl₂; C:29.97, H:2.73, N: 10.70. Found C:29.41, H:2.93, N:11.02.

### Example 2(C)

Synthesis of Prodrugs of MTAP Substrates

Scheme IV shows the conversion of intermediate C, from Scheme II above, to either symmetrically substituted prodrug D or unsymmetrically substituted prodrugs E and E′:

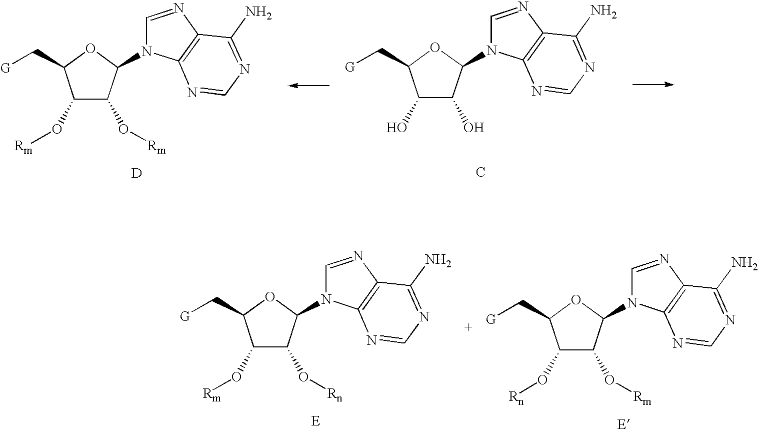

The capping groups Rₘ and Rₙ, may include, but are not limited to esters, carbonates, carbamates, ethers, phosphates and sulfonates. After introduction of the prodrug moiety, the compounds maybe further modified.

In particular, Scheme V shows the preparation of asymmetrically substituted prodrugs of 5′ adenosine analogs, starting from an appropriate 5′ substituted adenosine analog C as derived from Scheme II above (i.e., R═Me, Y═S, 5′-deoxy 5′-methythioadenosine; MTA):

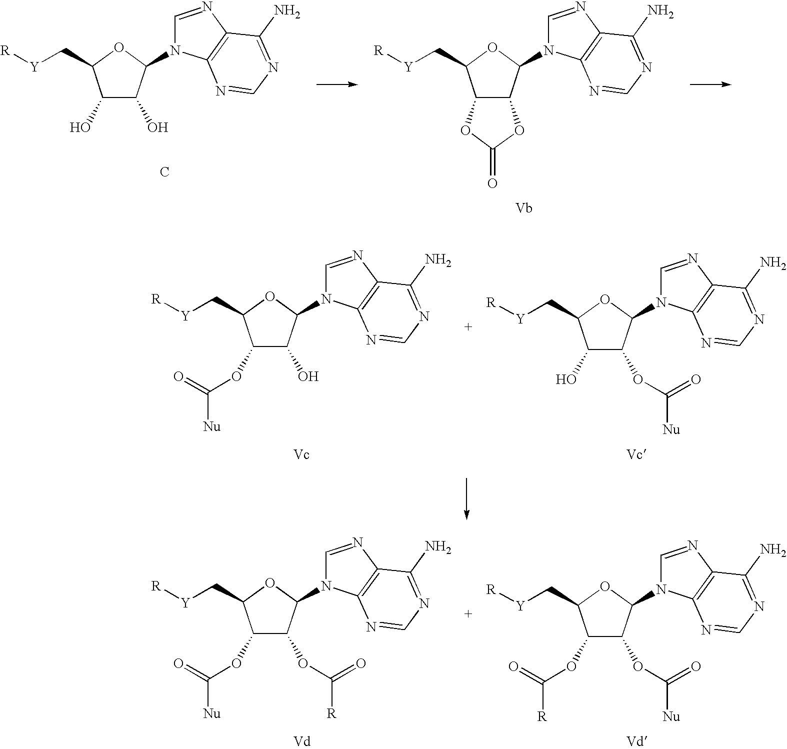

The diol C is converted to the cyclic carbonate Vb by treatment with 1,1′-carbonyldiimidazole (CDI) or a related reagent to give intermediate Vb. The cyclic carbonate is opened by treatment with a nucleophilic species, such as an amine, alcohol or thiol. The reaction is not regiospecific giving a mixture of two isomers, Vc and Vc′, which may rapidly interconvert. This mixture is not purified, but is treated with an acylating agent to cap the remaining free hydroxyl group and allow separation of the two isomeric final products, Vd and Vd′. The acylating groups may include, but are not limited to carboxylic acids, amino acids, carboxylic acid anhydrides, dialkyl dicarbonates (or pyrocarbonates), carbamyl chlorides, isocyantes, etc. Either the nucleophile utilized to open the cyclic carbonate or the subsequent acylating group may contain either an intact or masked solubilizing group. If necessary, the individual products Vd or Vd′ maybe further transformed to liberate the desired solubilizing group.

Alternatively, Scheme VI shows the preparation of symmetrically substituted prodrugs of 5′ adenosine analogs.

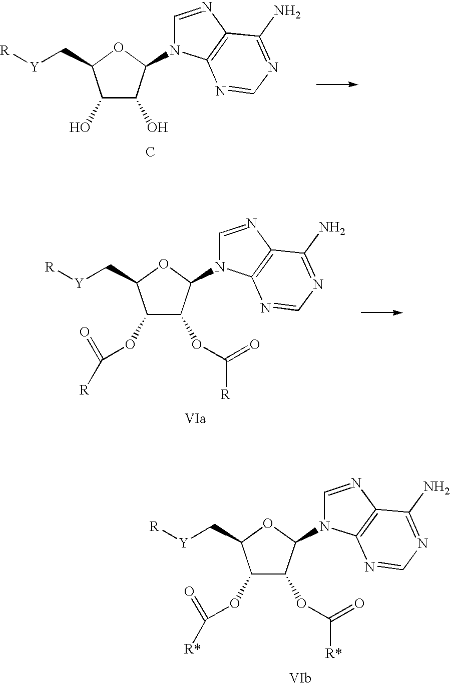

Starting from analog C, as derived from Scheme II above, both alcohols of the starting material are capped with the same acylating group the acylating group may include, but are not limited to carboxylic acids, amino acids, carboxylic acid anhydrides, dialkyl dicarbonates (or pyrocarbonates), carbamyl chlorides, isocyantes, etc. which contains either an intact or masked solubilizing group(R). If necessary, the compound VIa maybe further transformed to VIb in order liberate the desired solubilizing group (R*).

### EXAMPLES 2(C)(1) AND 2(C)(1′)

(2S,3S,4R,5R)-5-(6-amino-9H-purin-9-yl)-4-[(2,2-dimethylpropanoyl)oxy]-2-[(methylsulfanyl)methyl]tetrahydrofuran-3-yl-1,4′-bipiperidine-1′-carboxylate), and (2R,3R,4S,5S)-2-(6-amino-9H-purin-9-yl)-4-[(2,2-dimethylpropanoyl)oxy]-5-[(methylsulfanyl)methyl]tetrahydrofuran-3-yl 1,4′-bipiperidine-1′-carboxylate).

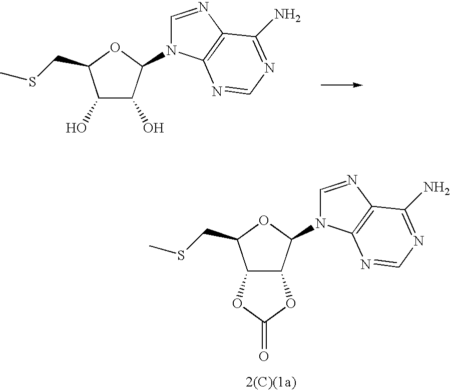

2(C)(1a): (3aR,4R,6S,6aS)-4-(6-amino-9H-purin-9-yl)-6-[(methylsulfanyl)methyl]tetrahydrofuro[3,4-d][1,3]dioxol-2-one.

To a solution of 5′-deoxy-5′-methylthioadenosine (13.4 g, 45.1 mmol) in DMF (250 mL) at 0° C., was added 1,1′-carbonyldiimidazole (8.50 g, 52.4 mmol) in one portion. After 1 h, the reaction was complete by HPLC, and the DMF was removed under vacuum. The resulting crude residue was dissolved in CHCl₃ and a minimal amount of i-PrOH. The organic layer was washed with a 4% aqueous solution of AcOH and then concentrated under vacuum. Azeatropic removal of excess acetic acid with heptane gave 2(C)(1a) as a white powder which was sufficiently pure to use without further purification (15.1 g, 100%). ¹H NMR (DMSO-d₆) δ:8.34 (1H, s), 8.18 (1H, s), 7.44 (2H, Br), 6.49 (1H, d, J=2.3 Hz), 6.05 (1H, dd, J=7.7 and 2.4 Hz), 5.48 (1H, dd, J=7.7 and 3.4 Hz), 4.56 (1H, dt, J=3.4 and 7.7 Hz), 2.78−2.71 (2H, m), 2.03 (3H, s). HPLC Rt=2.616 min. LRMS (m/z) 324 (M+H)⁺.

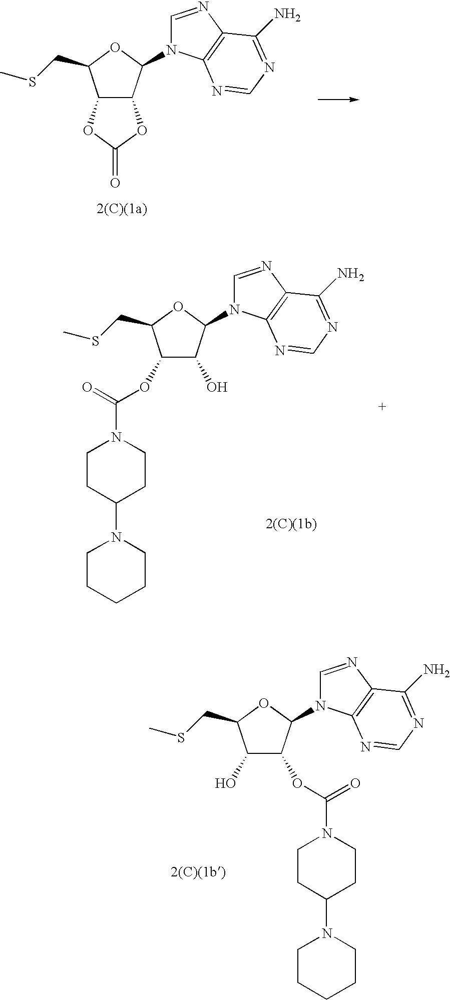

2(C)(1b): (2S,3S,4R,5R)-5-(6-amino-9H-purin-9-yl)-4-hydroxy-2-[(methylsulfanyl)methyl]tetrahydrofuran-3-yl 1,4′-bipiperidine-1′-carboxylate), and

2(C)(1b′): (2R,3R,4S,5S)-2-(6-amino-9H-purin-9-yl)-4-hydroxy-5-[(methylsulfanyl)methyl]tetrahydrofuran-3-yl 1,4′-bipiperidine-1′-carboxylate).

To a solution of 2(C)(1a) (3.18 g, 9.83 mmol) in DMF (40 mL) at room temperature (″rt) was added 4-piperidinopiperidine (6.06 g, 36.0 mmol). After 1.5 h at rt, the reaction wasp complete by HPLC, and the reaction mixture was split into four equal fractions. Each fraction was purified on a reverse phase column (Biotage Flash 40i System, Flash 40M cartridge, C-18, 10% MeOH/H₂O to 100% MeOH gradient) to give compounds 2(C)(1b) and 2(C)(1b′) in a 2.2:1 ratio, respectively. The individual regeoisomers were not isolated due to facile isomerization.

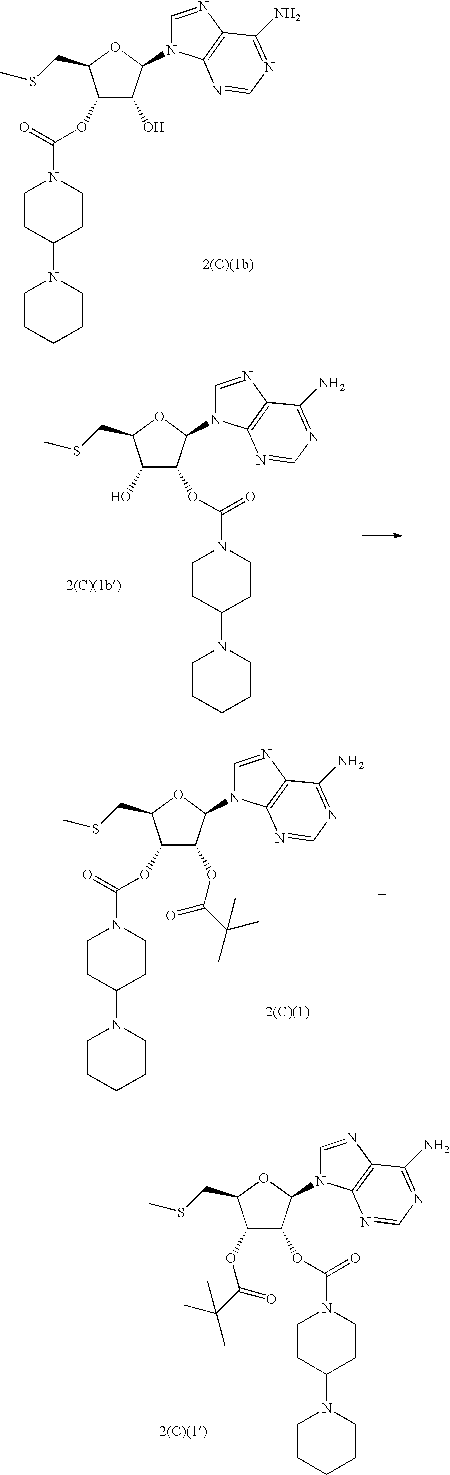

To a solution of 2(C)(1b) and 2(C)(1b′) (750 mg, 1.53 mmol) in CH₂Cl₂ (45 mL) at 0° C. was added trimethylacetic anhydride (1.0 mL, 4.9 mmol) and 4-dimethylaminopyridine (30 mg, 0.25 mmol), and the reaction mixture was warmed to rt. After 20 h, a 1:1 mixture of DMF and i-PrOH (3 mL) was added and the CH₂Cl₂ was removed under vacuum. The resulting solution was purified on semipreparative HPLC with a linear gradient elution of 20% A/80% B to 40% A/60% B over 30 min to give compounds 2(C)(1) and 2(C)(1′) as white powders (387 mg, 44% and 142 mg, 16% respectively). 2(C)(1): ¹H NMR (CDCl₃) δ:8.37 (1H, s), 8.07 (1H, s), 6.16 (1H, d, J=5.8 Hz), 5.88 (1H, t, J=5.6 Hz), 5.59 (2H, s), 5.53 (1H, s), 4.47 (1H, q, J=4.5 Hz), 4.22 (2H, m), 3.00 (2H, d, J=4.9 Hz), 2.92−2.69 (2H, m), 2.56−2.38 (5H, m), 2.17 (3H, s), 1.88−1.83 (2H, m), 1.77−1.70 (2H, m), 1.65−1.39 (6H, m), 1.14 and 1.15 (9H, 2s). HPLC Rt=3.318 min. LRMS (m/z) 576 (M+H)⁺. Anal. (C₂₇H₄₁N₇O₅S-0.25 H₂O) C, H, N, S. 2(C)(1′): (474 mg, 76%). ¹H NMR (CDCl₃) δ:8.38 (1H, s), 8.08 (1H, s), 6.20 (1H, d, J=5.6 Hz), 5.87−5.80 (1H, m), 5.60 (1H, dd, J=5.8 and 4.5 Hz), 5.54 (2H, s), 4.38 (1H, q, J=5.1 Hz), 4.15−4.11 (2H, m), 2.98 (2H, d, J=5.0 Hz), 2.83−2.67 (2H, m), 2.50−2.32 (5H, m), 2.16 (3H, s), 1.82−1.72 (2H, m), 1.61−1.52 (4H, m), 1.48−1.30 (4H, m), 1.26 and 1.24 (9H, 2s). HPLC Rt=3.512 min. LRMS (m/z ) 576 (M+H)⁺. Anal. (C₂₇H₄₁N₇O₅S-0.20 H₂O) C, H, N, S.

### EXAMPLES 2(C)(2) AND 2(C)(2′)

(2S,3S,4R,5R)-5-(6-amino-9H-purin-9-yl)-4-(isobutyryloxy)-2-[(methylthio)methyl]tetrahydrofuran-3-yl 1,4′-bipiperidine-1′-carboxylate, and (2R,3R,4S,5S)-2-(6-amino-9H-purin-9-yl)-4-(isobutyryloxy)-5-[(methylthio)methyl]tetrahydrofuran-3-yl 1,4′-bipiperidine-1′-carboxylate.

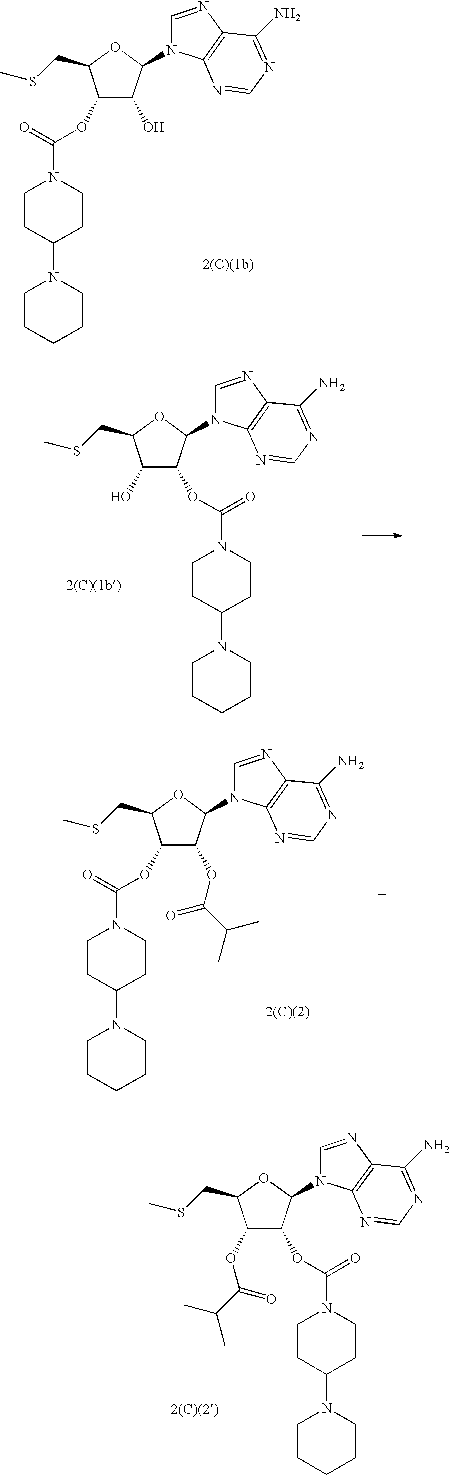

To a solution of alcohols 2(C)(1b) and 2(C)(1b′) (202 mg, 0.411 mmol) in CH₂Cl₂ (4 mL) at rt was added isobutyric acid (95.0 mg, 1.08 mmol), 1,3-dicyclohexylcarbodiimide (244 mg, 1.19 mmol), and 4-dimethylaminopyridine (3.2 mg, 0.026 mmol). After 24 h, the reaction was complete, and a 1:1 mixture of DMF and i-PrOH (1 mL) was added. The CH₂Cl₂ was removed under vacuum, leaving the DMF/i-PrOH solution which was purified by semipreparative HPLC with a linear gradient elution of 20% A/80% B to 40% A/60% B over 30 min to give the title compounds 2(C)(2) and 2(C)(2′) as white powders (83.9 mg, 36% and 22.0 mg, 10% respectively). 2(C)(2): ¹H NMR (CDCl₃) δ:8.38 (1H, s), 8.08 (1H, s), 6.18 (1H, d, J=6.0 Hz), 5.93 (1H, t, J=4.5 Hz), 5.58 (2H, s), 5.53 (1H, t, J=4.1 Hz), 4.46 (1H, q, J=4.9 Hz), 4.20 (2H, m), 3.00 (2H, d, J=5.1 Hz), 2.90−2.68 (2H, m), 2.60−2.38 (6H, m), 2.17 (3H, s), 1.87−1.83 (2H, m), 1.64−1.40 (8H, m), 1.19−1.10 (6H, m). HPLC Rt=3.322 min. LRMS (m/z) 562 (M+H)⁺. Anal. (C₂₆H₃₉N₇O₅S) C, H, N, S. 2(C)(2′): ¹H NMR (CDCl₃) δ:8.38 (1H, s), 8.08 (1H, s), 6.21 (1H, d, J=5.6 Hz), 5.85 (1H, t, J=5.3 Hz), 5.63−5.56 (3H, m), 4.40 (1H, q, J=4.7 Hz), 4.18−4.04 (2H, m), 2.97 (2H, d, J=5.2 Hz), 2.85−2.55 (3H, m), 2.51−2.31 (5H, m), 2.16 (3H, s), 1.84−1.80 (2H, m), 1.62−1.52 (4H, m), 1.48−1.3 (4H, m), 1.27−1.16 (6H, m). HPLC Rt=3.432 min. LRMS (m/z) 562 (M+H)⁺. Anal. (C₂₆H₃₉N₇O₅S-0.40 H₂O) C, H, N, S.

### EXAMPLES 2(C)(3) and 2(C)(3′)

(2S,3S,4R,5R)-5-(6-amino-9H-purin-9-yl)-4-({(2R)-2-[(tert-butoxycarbonyl)amino]propanoyl}oxy)-2-[(methylthio)methyl]tetrahydrofuran-3-yl 1,4′-bipiperidine-1′-carboxylate, and (2R,3R,4S,5S)-2-(6-amino-9H-purin-9-yl)-4-({(2R)-2-[(tert-butoxycarbonyl)amino]propanoyl}oxy)-5-[(methylthio)methyl]tetrahydrofuran-3-yl 1,4′-bipiperidine-1′-carboxylate.

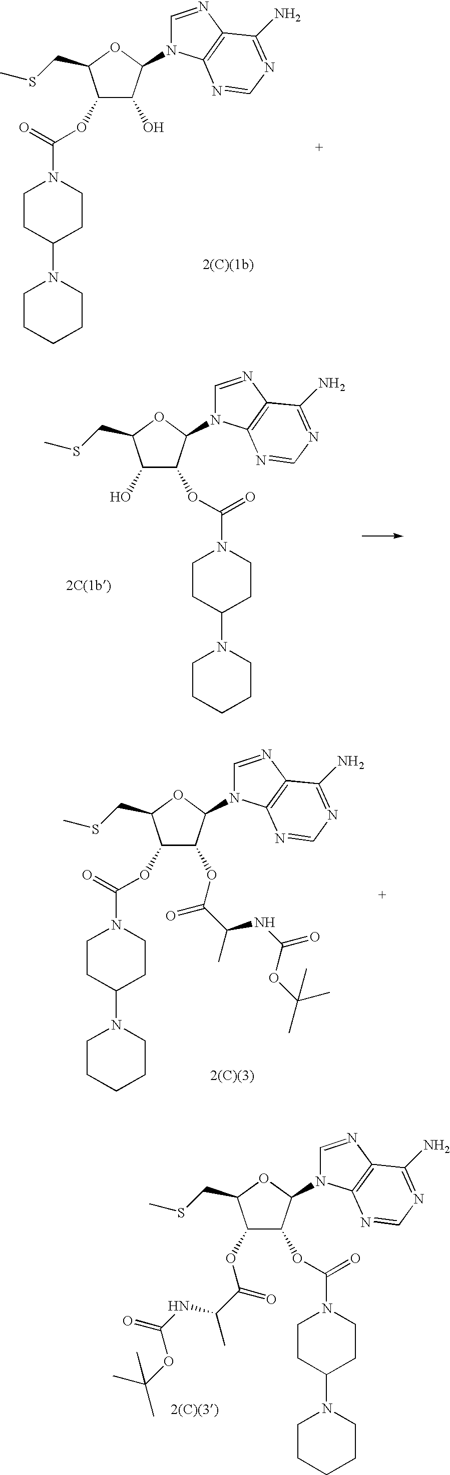

To a solution of alcohols 2(C)(1b) and 2(C)(1b′) (329 mg, 0.668 mmol) in CH₂Cl₂ (6.5 mL) at rt was added N-(tert-butoxycarbonyl)-L-alanine (329 mg, 1.74 mmol), 1,3-dicyclohexylcarbodiimide (400 mg, 1.94 mmol), and 4-dimethylaminopyridine (10 mg, 0.082 mmol). After 0.5 h, the reaction was complete, the precipitate was filtered, and a 1:1 mixture of DMF/i-PrOH (2 mL) was added to the filtrate. The CH₂Cl₂ was removed under vacuum, leaving the DMF/i-PrOH solution which was purified by semipreparative HPLC with a linear gradient elution of 15% A/85% B to 35% A/65% B over 30 min to give the title compounds 2(C)(3) and 2(C)(3′) as white powders (134 mg, 30% and 36.9 mg, 8% respectively). 2(C)(3): ¹H NMR (CDCl₃) δ:8.37 (1H, s), 8.01 (1H, s), 6.15 (1H, d, J=5.3 Hz), 6.09−6.02 (1H, m), 5.63−5.52 (3H, m), 4.44 (1H, q, J=5.1 H) 4.38−4.26 (1H, m), 4.25−4.12 (2H, m), 2.99 (2H, d, J=5.2 Hz), 2.93−2.67 (2H, m), 2.54−2.36 (5H, m), 2.15 (3H, s), 1.90−1.80 (2H, m), 1.64−1.54 (4H, m), 1.51−1.25 (16H, m). HPLC Rt=3.513 min. LRMS (m/z) 663 (M+H)⁺. Anal. (C₃₀H₄₆N₈O₇S) C, H, N, S. 2(C)(3′): ¹HNMR(CDCl₃) δ:8.37,(1H, s), 8.05 (1H, s), 6.17 (1H, d, J=5.4 Hz), 5.90 (1H, t, J=5.4 Hz), 5.70,(1H, t, J=4.8 Hz), 5.55 (2H, s), 4.41 (2H, q, J=4.9 Hz), 4.16−4.01 (2H, m), 2.97 (2H, d, J=5.1 Hz), 2.86−2.64 (2H, m), 2.53−2.30 (5H, m), 2.15 (3H, s); 1.85−1.72 (2H, m), 1.61−1.51 (4H, m), 1.50−1.38 (16H, m). HPLC Rt=3.642 min. LRMS (m/z) 663 (M+H)⁺. Anal. (C₃₀H₄₆N₈O₇S) C, H, N, S.

### EXAMPLES 2(C)(4) AND 2(C)(4′)

(2S,3S,4R,5R)-5-(6-amino-9H-purin-9-yl)-4-(benzoyloxy)-2-[(methylthio)methyl]tetrahydrofuran-3-yl 1,4′-bipiperidine-1′-carboxylate and (2R,3R,4S,5,)-2-(6-amino-9H-purin-9-yl)-4-(benzoyloxy)-5-[(methylthio)methyl]tetrahydrofuran-3-yl 1,4′-bipiperidine-1′-carboxylate.

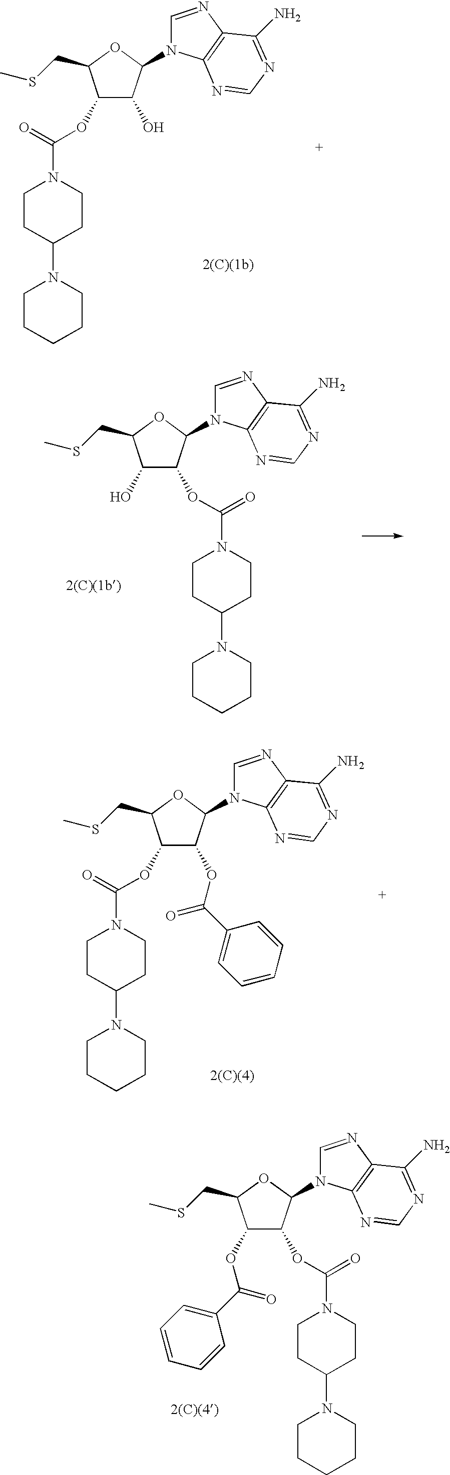

To a solution of alcohols 2(C)(1b) and 2(C)(1b′) (559 mg, 1.14 mmol) in CH₂Cl₂ (11 mL) at rt was added benzoic acid (250 mg, 2.05 mmol), 1,3-dicyclohexylcarbodiimide (469 mg, 2.27 mmol), and 4-dimethylaminopyridine (17 mg, 0.14 mmol). After 45 min., the reaction was complete, the precipitate was filtered, and a 3:1 mixture of DMF/i-PrOH (4 mL) was added to the filtrate. The CH₂Cl₂ was removed under vacuum, leaving the DMF/i-PrOH solution which was purified by semipreparative HPLC with a linear gradient elution of 20% A/80% B to 25% A/75% B over 30 min to give the title compounds 2(C)(4) and 2(C)(4′) as white powders (264 mg, 39% and 032.8 mg, 5% respectively). 2(C)(4): ¹H NMR (CDCl₃) δ:8.39 (1H, s), 8.13 (1H, s), 8.01 (2H, m), 7.59 (1H, t, J=7.5 Hz, 7.44 (2H, t, J=7.5 Hz), 6.37 (1H, d, J=5.3 Hz), 6.13 (1H, t, J=5.6 Hz), 5.67 (1H, t, J=5.1 Hz), 5.58 (2H, s), 4.54 (1H, q, J=4.7 Hz), 4.19−3.98 (2H, m), 3.06−3.03 (2H, m), 2.77=2.62 (2H, m), 2.52=2.27 (5H, m), 2.20 (3H, s), 1.82−1.71 (2H, m), 1.63−1.48 (4H, m), 1.48−1.24 (4H, m). HPLC Rt=3.483 min. LRMS (m/z) 596 (M+H)⁺. Anal. (C₂₉H₃₇N₇O₅S) C, H, N, S. 2(C)(4′): ¹H NMR (CDCl₃) δ:8.40 (1H, s), 8.11 (1H, s), 8.03-8.06 (2H, m), 7.63 (1H, t, J=7.6 Hz), 7.49 (2H, t, J=7.9 Hz), 6.28 (1H, d, J=5.6 Hz), 6.05−5.98 (1H, m), 5.90−5.84 (1H, m), 5.54 2H, s), 4.61 (1H, q, J=4.5 Hz), 4.13−3.88 (2H, m), 3.05 (2H, d, J=5.1 Hz), 2.68−2.53 (2H, m), 2.43−2.23 (5H, m), 2.19 (3H, s), 1.75−1.62 (2H, m), 1.58−1.47 (4H, m), 1.48−1.25 (4H, m). HPLC Rt=3.640 min. LRMS (m/z) 596 (M+H)⁺. Anal. (C₂₉H₃₇N₇O₅S-0.25 H₂O) C, H, N, S.

### EXAMPLES 2(C)(5) AND 2(C)(5′)

(2R,3R,4S,5S)-2-(6-amino-9H-purin-9-yl)-4-[({[2-(dimethylamino)ethyl]amino}carbonyl) oxy]-5-[(methylthio)methyl]tetrahydrofuran-3-yl pivalate and (2S,3S,4R,5R)-5-(6-amino-9H-purin-9-yl)-4-[({[2-(dimethylamino)ethyl]amino}carbonyl)oxy]-2-[(methylthio)methyl]tetrahydrofuran-3-yl pivalate.

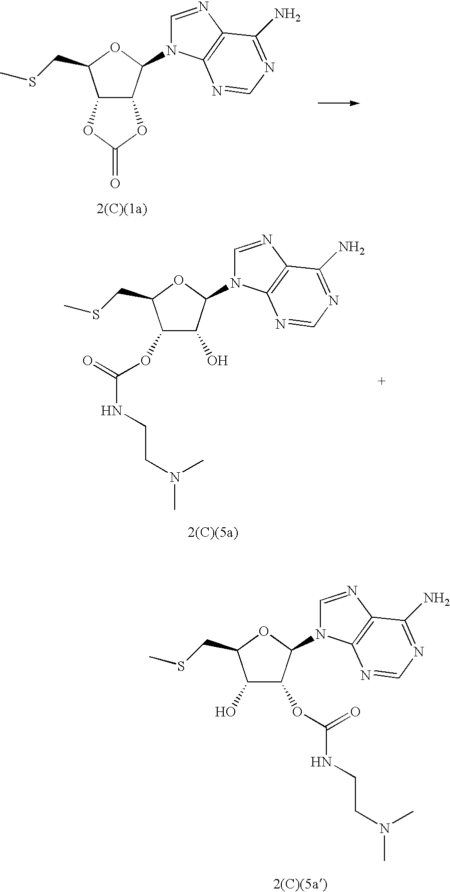

2(C)(5)(a) and 2(C)(5)(a′): (2S,3S,4R,5R)-5-(6-amino-9H-purin-9-yl)-4-hydroxy-2-[(methylthio)methyl]tetrahydrofuran-3-yl 2-(dimethylamino)ethylcarbamate, and (2R,3R,4S,5S)-2-(6-amino-9H-purin-9-yl)-4-hydroxy-5-[(methylthio)methyl]tetrahydrofuran-3-yl 2-(dimethylamino)ethylcarbamate.

To a solution of 2(C)(1a) (1.90 g, 5.88 mmol) in DMF (5 mL) at rt was added N,N-dimethylethylenediamine (803 mg, 9.11 mmol). After 20 min. at rt, the reaction was complete by HPLC. The reaction mixture was loaded directly on a reverse phase column (Biotage Flash 40i System, Flash 40M cartridge, C-18, 10% MeOH/H₂O to 100% MeOH gradient) to give the title compounds 2(C)(5a) and 2(C)(5a′) in a 1.9:1 ratio, respectively. As with intermediates 2(C)(1b) and 2(C)(1b′), the individual regeoisomers were not isolated due to facile isomerization.

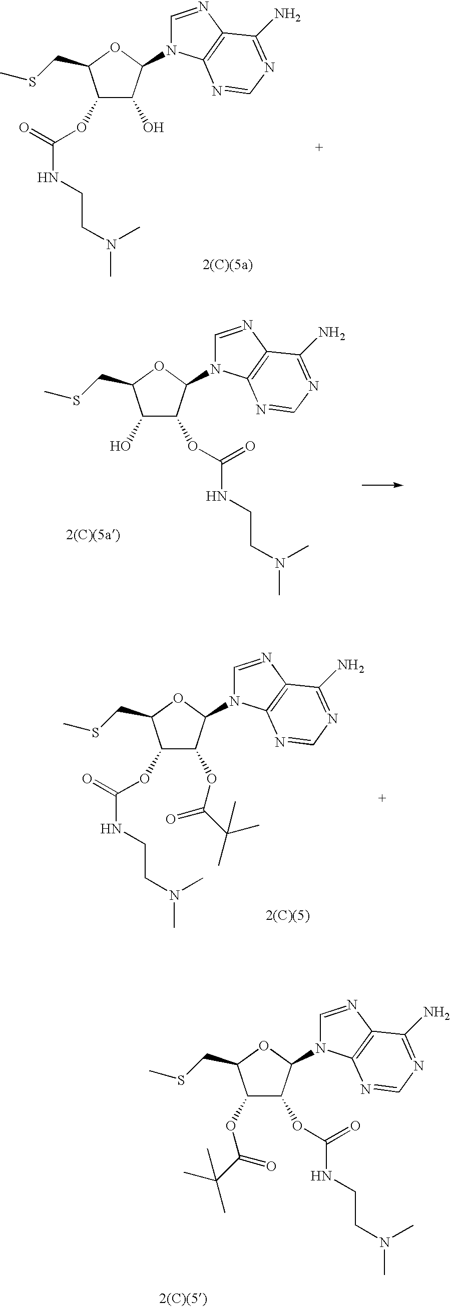

Alcohols 2(C)(5a) and 2(C)(5a′) (748 mg, 1.82 mmol) were aceylated and purified according the procedure given for Example 2(C)(1) and 2(C)(1′) to give the title compounds 2(C)(5) and 2(C)(5′) as white powders (243 mg, 27% and 128 mg, 14% respectively). Compound 2(C)(5):¹H NMR (CDCl₃) δ:8.37 (1H, s), 8.05 (1H, s), 6.16 (1H, d, J=5.7 Hz), 5.87 (1H, t, J=5.7 Hz), 5.67 (2H, s), 5.55 (1H, t, J=4.7 Hz), 5.51−5.44 (1H, m), 4.43 (1H, q, J=4.7 Hz), 3.31−3.21 (2H, m), 2.99−2.96 (2H, m), 2.41 (2H, q, J=4.4 Hz), 2.24 (6H, s), 2.17 (3H, s), 1.15 (9H, s). HPLC Rt=3.024 min. LRMS (m/z) 496 (M+H)⁺. Anal. (C₂₁H₃₃N₇O₅S) C, H, N, S. Compound 2(C)(5′): ¹H NMR (CDCl₃) δ:8.39 (1H, s), 8.07 (1H, s), 6.16 (1H, d, J=5.7 Hz), 5.86 (1H, t, J=5.8 Hz), 5.63−5.55 (3H, m), 5.42 (1H, t, J=5.1 Hz), 4.38 (1H, q, J=4.9 Hz), 3.19 (2H, q, J=5.7 Hz), 2.97 (2H, d, J=5.1 Hz), 2.37−2.33 (2H, m), 2.18 (6H, s), 2.16 (3H, s), 1.25 (9H, s). HPLC Rt=3.291 min. LRMS (m/z) 496 (M+H)⁺. Anal. (C₂₁H₃₃N₇O₅S) C, H, N, S.

### EXAMPLES 2(C)(6) AND 2(C)(6′)

(2R,3R,4S,5S)-2-(6-amino-9H-purin-9-yl)-4-[({[2-(dimethylamino)ethyl]amino}carbonyl) oxy]-5-[(methylthio)methyl]tetrahydrofuran-3-yl benzoate, and (2S,3S,4R,5R)-5-(6-amino-9H-purin-9-yl)-4-[({[2-(dimethylamino)ethyl]amino}carbonyl)oxy]-2-[(methylthio)methyl]tetrahydrofuran-3-yl benzoate.

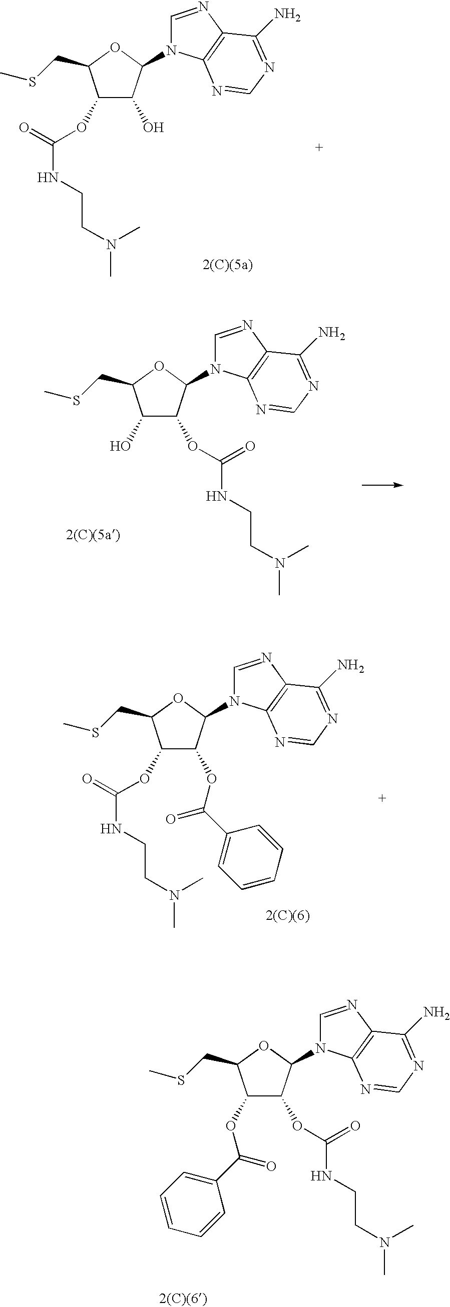

Alcohols 2(C)(5a) and 2(C)(5a′) (1.04 g, 2.52 mmol) were aceylated and purified according the procedure given for Example 2(C)(4) and 2(C)(4′) to give the title compounds 2(C)(6) and 2(C)(6′) as white powders (473 mg, 36% and 220 mg, 17% respectively). Compound 2(C)(6): ¹H NMR (CDCl₃) δ:8.39 (1H, s), 8.11 (1H, s), 7.92 (2H, d, J=7.5 Hz), 7.56 (1H, t, J=7.5 Hz), 7.40 (2H, t, J=7.5 Hz), 6.35 (1H, d, J=5.7 Hz), 6.18 (1H, t, J=5.6 Hz), 5.70−5.61 (3H, m), 5.57−5.49 (1H, m), 4.52 (1H, q, J=4.7 Hz), 3.23−3.16 (2H, m), 3.05−3.02 (2H, m), 2.34 (2H, q, J=5.8 Hz), 2.19 (3H, s), 2.18 (6H, s). HPLC Rt=3.090 min. LRMS (m/z) 516 (M+H)⁺. Anal. (C₂₃H₂₉N₇O₅S) C, H, N, S. Compound 2(C)(6′): ¹H NMR (CDCl₃) δ:8.40 (1H, s), 8.11−8.08 (3H, m), 7.62 (1H, t, J=7.3 Hz), 7.48 (2H, t, J=7.5 Hz), 6.28 (1H, d, J=5.9 Hz), 5.99 (1H, t, J=5.8 Hz), 5.87 (1H, t, J=4.1 Hz), 5.68 (2H, s), 5.45 (1H, t, J=4.7 Hz), 4.57 (1H, q, J=4.3 Hz), 3.13 (2H, q, J=5.5 Hz), 3.06 (2H, d, J=5.3 Hz), 2.32−2.23 (2H, m), 2.19 (3H, s), 2.12 (6H, s). HPLC Rt=3.348 min. LRMS (m/z) 516 (M+H)⁺. Anal. (C₂₃H₂₉N₇O₅S) C, H, N, S.

### EXAMPLE 2(C)(7)

(2R,3R,4S,5S)-2-(6-amino-9H-purin-9-yl)-4-{[(1-methylpiperidin-4-yl)carbonyl]oxy}-5-[(methylsulfanyl)methyl]tetrahydrofuran-3-yl 1-methylpiperidine-4-carboxylate.

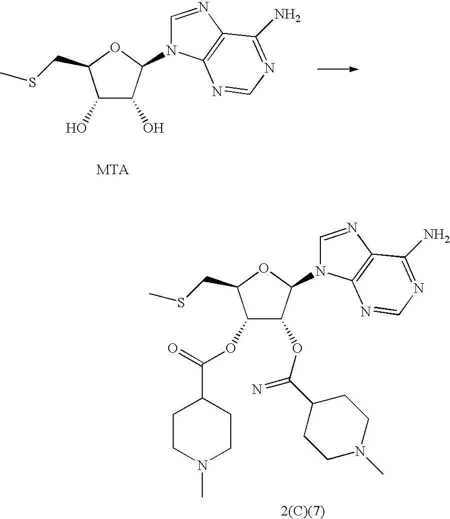

To a heterogeneous mixture of 5′-deoxy-5′-methylthioadenosine (MTA) (2.12 g, 7.13 mmol) in CH₂Cl₂ (100 mL) at rt was added 1,3-dicyclohexylcarbodiimide (4.85 g, 23.5 mmol) and 4-dimethylaminopyridine (174 mg, 1.43 mmol). After 16 h, the precipitate was removed by filtration, the filtrate was diluted with MeOH, and the CH₂Cl₂ was removed under vacuum. The resulting methanolic solution was purified on semipreparative HPLC with a linear gradient elution of 5% A/95% B to 12% A/88% B over 30 min to give B(1) as a white powder (207 mg, 5.3%). ¹H NMR (CDCl₃) δ:8.37 (1H, s), 8.03 (1H, s), 6.14 (1H, d, J=5.7 Hz), 5.98 (1H, t, J=5.6 Hz), 5.65 (1H, t, J=5.6 Hz), 5.64 (2H, s), 4.39 (1H, q, J=4.7 Hz), 2.98 (2H, d, J=5.0 Hz), 2.86−2.82 (2H, m), 2.78−2.72 (2H, m), 2.39−2.21 (2H, m), 2.29 (3H, s), 2.24 (3H, s), 2.16 (3H, s), 2.05−1.66 (12H, m). HPLC Rt=2.637 min. LRMS (m/z) 548 (M+H)⁺. Anal. (C₂₅H₃₇N₇O₅S-0.20 H₂O) C, H, N, S.

### EXAMPLES 2(C)(8) AND 2(C)(9)

(2R,3R,4S,5S)-4-(acetyloxy)-2-(6-amino-9H-purin-9-yl)-5-[(ethylsulfanyl)methyl]tetrahydrofuran-3-yl acetate, and (2R,3R,4S,5S)-4-(acetyloxy)-2-(6-amino-9H-purin-9-yl)-5-[(isobutylsulfanyl)methyl]tetrahydrofuran-3-yl acetate.

The following 2′, 3′-diacetate derivatives of 5′-deoxy 5′-alkylthioadenosine were prepared according to the method described by M. J. Robins et. al. J. Org. Chem. 59, 544 (1994).

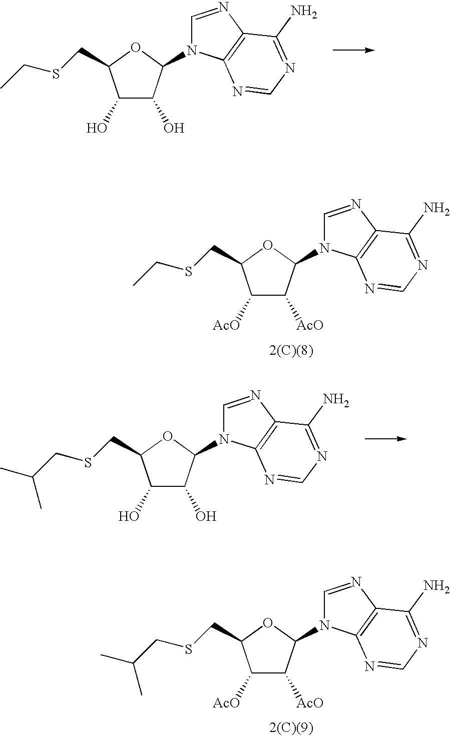

2(c)(8): ¹H NMR (DMSO-d₆) δ:1.14 (t, 3H, J=7.4 Hz), 2.04 (s, 3H), 2.15 (s, 3H), 2.54 (q, 2H, J=7.4 Hz), 2.95-3.10 (m, 2H), 4.31(dd, 1H, J=6.4, 6.0 Hz), 5.60 (dd, 1H, J=5.3, 4.3 Hz), 6.12-6.18 (m, 1H), 6.20-6.25 (m, 1H), 7.44 (s, 2H), 8.22 (s, 1H), 8.44 (s, 1H). LRMS (m/z) 395 (M+H)⁺ Anal. C₁₆H₂₁N₅O₅S-1.0 H₂O) C, H N, S. 2(c)(9): ¹H NMR (DMSO-d₆) δ:0.82 (t, 6H, J=7.0 Hz), 1.62-1.75 (m, 1H), 2.00 (s, 3H), 2.11 (s, 3H), 2.32-2.46 (m, 2H), 2.93-3.07 (m, 2H), 4.25-4.35 (m, 1H), 5.56 (t, 1H, J=4.4 Hz), 6.15-6.27 (m, 2H), 7.41 (s, 2H), 8.17 (s, 1H), 8.40 (s, 1H). LRMS (m/z) 423 (M+H)⁺. Anal. (C₁₈H₂₅N₅O₅S-0.5 H₂O) C,H,N,S.

### EXAMPLE 2(C)(10)

(2S,3S,4R,5R)-5-(6-amino-9H-purin-9-yl)-4-azido-2-[(methylthio)methyl]tetrahydrofuran-3-ol.

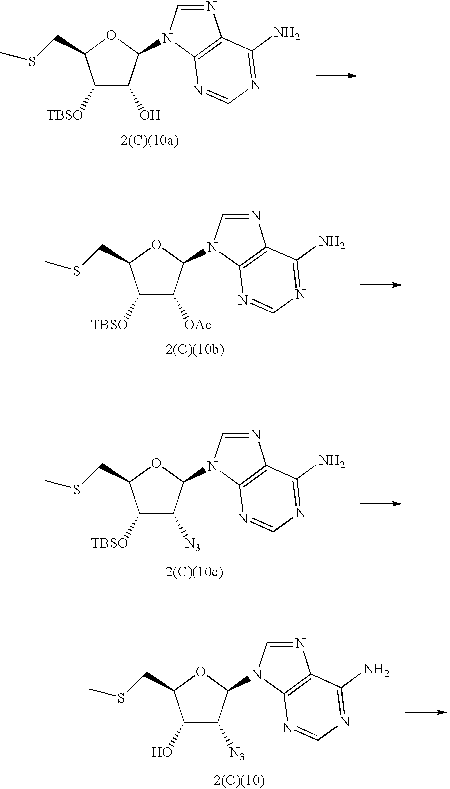

Intermediate 2(C)(10b): (2R,3S,4S,5S)-2-(6-amino-9H-purin-9-yl)-4-{[tert-butyl(dimethyl)silyl]oxy}-5-[(methylthio)methyl]tetrahydrofuran-3-yl hydrogen carbonate. To a solution of 2(C)(10a) (prepared via the method described by Gavagnin and Sodano. Nucleosides & Nucleotides, 8, 1319 (1989))(1.82 g, 4.42 mmol), pyridine (3 mL), and DMAP (1.78 g, 14.6 mmol) in CH₂Cl₂ (150 mL) at 0° C. was added triflic anhydride (1.42 g, 8.46 mmol) dropwise. After 1 h, the reaction mixture was poured into cold 1N NaHSO₄ and partitioned with CHCl₃. The organic layer was concentrated, and the resulting residue was redissolved in HMPA (20 mL), treated with NaOAc (2.99 g, 36.5 mmol), warmed to 40° C. for 1 h, and then stirred at rt for 16 h. The reaction mixture was then poured into H₂O and partitioned with CHCl₃. The organic layer was concentrated under vacuum, and the resulting residue was purified by reverse phase chromatography (Biotage Fash 40, C-18) eluting with a linear gradient of 5-60% acetonitrile in H₂O to give 2(C)(10b) as a white solid (0.437 g, 22%). LRMS (m/z) 454.(M+H)⁺.

Intermediate 2(C)(10c): 9-{(2R,3R,4S,5S)-3-azido-4-{[tert-butyl(dimethyl)silyl]oxy}-5-[(methylthio)methyl]tetrahydrofuran-2-yl}-9H-purin-6-amine. A solution of 2(C)(10b) (0.437 g, 0.964 mmol) in MeOH (30 mL) was saturated with NH₃(g). The removal of the acetate group was complete after 20 min, after which solvent and reagent were removed under vacuum to give the free alcohol as a yellow solid. This crude material was dissolved in CH₂Cl₂ (30 mL) at 0° C., to which was added pyridine (0.685 g, 8.65 mmol) and DMAP (0.391 g, 3.20 mmol), followed by dropwise addition of triflic anhydride (0.395 g, 2.35 mmol). After 3 h at 0° C., the reaction mixture was poured into cold 1N NaHSO₄, partitioned with CHCl₃ and the organic layer concentrated. The resulting crude triflate was dissolve in DMF (40 mL) and treated with NaN₃ (0.627 g, 9.65 mmol). After 16 h at rt, the DMF was removed under vacuum, and the residue was partially dissolved in CHCl₃ and washed with H₂O. The organic layer was concentrated to give intermediate 2(C)(10c) as a yellow oil. This material was used without any further purification. LRMS (m/z) 436 (M+H)⁺.

The title compound 2(C)(10) was prepared as follows. To a solution of 2(C)(10c) in THF (20 mL) at 0° C. was added TBAF (1M in THF, 1.5 mL, 1.5 mmol) dropwise. After 30 min at rt, AcOH (0.5 mL) and CH₂Cl₂ (50 mL) were added, and the reaction mixture was filtered through silicone treated filter paper (Whatman 1PS) and concentrated under vacuum. The resulting residue was purified on semipreparative reverse phase HPLC using water and acetonitrile (each containing 0.1% v/v acetic acid) as mobile phase to give the title compound 2(C)(10) as a white powder (103 mg, 18%). ¹H NMR (DMSO-d₆) δ:8.37 (1H, s), 8.17 (1H, s), 7.38 (2H, s), 6.16 (1H, s), 6.02 (1H, d, J=5.8 Hz), 4.88 (1H, t, J=5.7 Hz), 4.59 (1H, t, J=4.5 Hz), 4.06 (1H, q, J=5.8 Hz), 2.91 (1H, dd, J=13.9 and 5.7 Hz), 2.79 (1H, dd, J=16.4 and 7.0 Hz), 2.05 (3H, s). LRMS (m/z) 323 (M+H)⁺ Anal. (C₁₁H₁₄N₈O₂S-0.20 H₂O) C, H, N, S.

### EXAMPLE 2(C)(11)

(2S,3S,4R,5R)-4-amino-5-(6-amino-9H-purin-9-yl)-2-[(methylthio)methyl]tetrahydrofuran-3-ol.

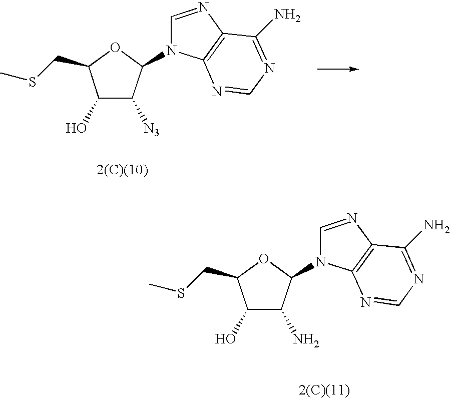

To a solution of example 2(C)(10) (0.480 g, 1.49 mmol) in pyridine (40 mL) at rt was added PPh₃ (0.586 g, 2.24 mmol). After 24 h, H₂O (5 mL) was added and the reaction stirred for an additional 60 h. The solvents were removed under vacuum, and the resulting residue was dissolved in H₂O and washed with Et₂O. The aqueous layer was concentrated under vacuum, and the resulting residue purified by reverse phase chromatography (Biotage Flash 40M, C-18) with a linear gradient elution of 5-10% acetonirile in H₂O to give the title compound 2(C)(11) as a white powder (176 mg, 40%). ¹H NMR (DMSO-d₆) δ:8.35 (1H, s), 8.14 (1H, s), 7.27 (2H, s), 5.72 (1H, d, J=7.8 Hz), 4.19−4.15 (1H, m), 4.10−4.02 (2H, m), 2.88 (1H, dd, J=13.9 and 6.8 Hz), 2.79 (1H, dd, J=13.6 and 6.6 Hz), 2.06 (3H, s). LRMS (m/z) 297 (M+H)⁺Anal. (C₁₁H₁₆N₆O₂S-0.40 H₂O) C, H, N, S.

EXAMPLE 2(C)(12)

(2S,3R,4R,5R)-5-(6-amino-9H-purin-9-yl)-4-chloro-2-[(methylthio)methyl]tetrahydrofuran-3-ol.

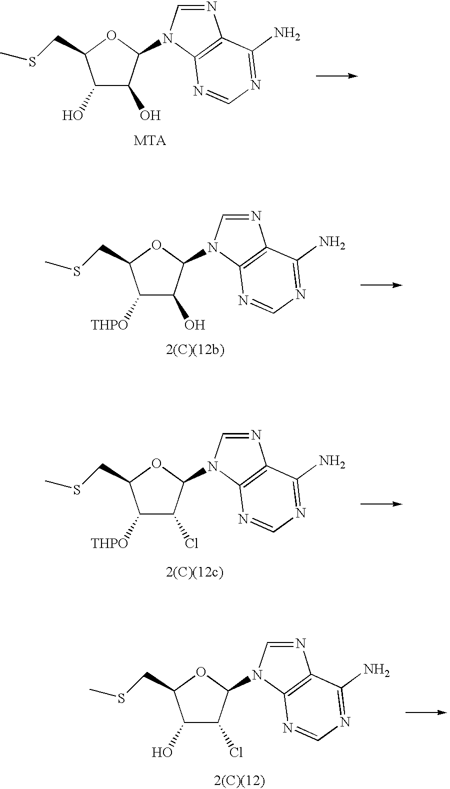

Intermediate 2(C)(12b): (2R,3S,4S,5S)-2-(6-amino-9H-purin-9-yl)-5-[(methylthio)methyl]-4-(tetrahydro-2H-pyran-2-yloxy)tetrahydrofuran-3-ol. To a solution of MTA [J. A. Montgomery et. al. J. Med. Chem. 17, 1197 (1974); Gavagnin and Sodano Nucleosides & Nucleotides 8, 1319 (1989)] (0.480 g, 1.661 mmol) in DMF (36 mL) was added dihydropyran (8 mL) and para-toluenesulfonic acid (0.450 g, 2.37 mmol). After 45 min at rt, sat. aq. NaHCO₃ (200 mL) was added and the aqueous solution was extracted with EtOAc. The organic layer was concentrated, and the residue chromatographed with acetone/CH₂Cl₂ (product elutes with 2:1) to give 2(C)(12b) as a white solid (0.413 g, 67%). LRMS (m/z) 382 (M+H)⁺.

Intermediate 2(C)(12c): 9-[(2R,3R,4R,5S)-3-chloro-5-[(methylthio)methyl]-4-(tetrahydro-2H-pyran-2-yloxy)tetrahydrofuran-2-yl]-9H-purin-6-amine. A solution of 2(C)(12b) (0.361 g, 0.946 mmol), pyridine (0.684 g, 8.65 mmol) and DMAP (0.381 g, 3.12 mmol) in CH₂Cl₂ (40 mL) at 0° C. was treated with triflic anhydride (0.395 g, 2.35 mmol) dropwise. After 2 h at 0° C., the reaction mixture was poured into cold 1N NaHSO₄, extracted with CHCl₃, and the organic layer concentrated. The resulting residue was dissolve in DMF (60 mL) and treated with tetrabutylammonium chloride-hydrate (0.526 g, 1.89 mmol). After 16 h at rt, the DMF was removed under vacuum and the resulting residue chromatographed with acetone/CH₂Cl₂ (product elutes with 1:1) to give 2(C)(12c) as a white solid (0.270 g, 71%). LRMS (m/z) 400 (M+H)⁺.

The title compound 2(C)(12) was prepared as follows. A solution of 2(C)(12c) (0.226 g, 0.565 mmol) in MeOH (20 mL) was treated with aq. 1N HCl (20 mL). After 1 h at rt, the reaction mixture was poured into H₂O, neutralized with NaHCO₃, extracted with CHCl₃, and concentrated. The resulting residue was purified by reverse phase chromatography (Biotage Flash 40M, C-18) with acetonitrile/H₂O (1:4) to give the title compound as a white powder (126 mg, 71%). ¹H NMR (DMSO-d₆) δ:8.41 (1H, s), 8.17 (1H, s), 7.39 (2H, s), 6.16 (1H, d, J=7.3 Hz), 6.11 (1H, d, J=5.1 Hz), 5.40−5.37 (1H, m), 4.39 (1H, q, J=2.8 Hz), 4.15 (1H, dt, J=6.6 and 2.8 Hz), 2.91 (1H, dd, J=13.9 and 6.3 Hz), 2.83 (1H, dd, J=13.9 and 6.8 Hz), 2.07 (3H, s). LRMS (m/z) 316 (M+H)⁺.

### EXAMPLE 2(D)

Synthesis of Purine Analogs of MTAP Substrates

The following examples illustrate methods to prepare MTA analogs at the 6′ position of the purine ring.

Scheme VII shows the method to prepare additional prodrugs of 5′ adenosine analogs. The prodrugs have been nitrogen substituted at the 6′ position of the purine ring. Starting from VIIa, the compound is acylated on all open positions (2′ and 3′ alcohol and N⁶ of the adenine ring) to give intermediate VIIb. The acylating group may include, but is not limited to carboxylic acids, amino acids, carboxylic acid anhydrides, etc. which contains either an intact or masked solubilizing group (R). Compound VIIb is typically not isolated, but rather immediately placed under hydrolysis conditions (i.e. NaOH or related reagents) to remove the esters to give VII. As necessary, VII may or may not be further treated in order liberate the desired solubilizing group.

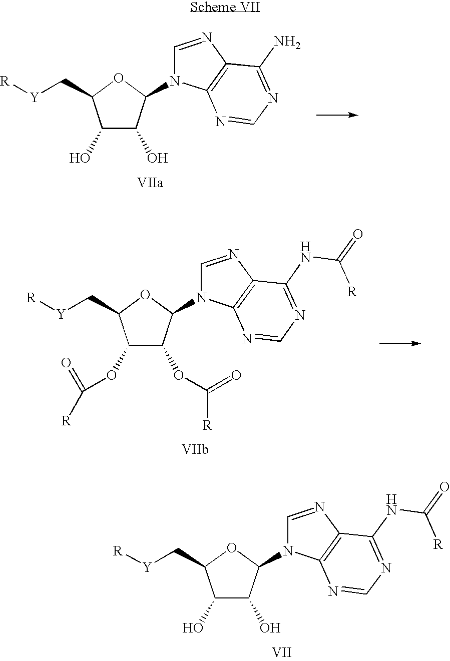

### EXAMPLE 2(D)(1)

N-(9-{(2R,3R,4S,5S)-3,4-dihydroxy-5-[(methylthio)methyl]tetrahydrofuran-2-yl}-9H-purin-6-yl)benzamide.

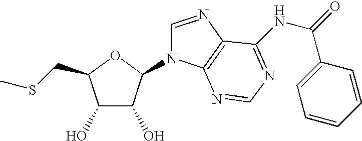

To a solution of MTA (1.12 g, 3.78 mmol) in pyridine (47 mL) was added benzoyl chloride (1.6 mL, 13.8 mmol) at rt. After 1 h, additional benzoyl chloride (0.4 mL, 3.45 mmol) was added and the reaction stirred for another hour before the pyridine was removed under vacuum. The resulting foam was dissolved in EtOH (35 mL) and THF (30 mL) and treated with 2N NaOH (26 mL). After 1h, the reaction was diluted with ice (100 mL) and pH=7 phosphate buffer (50 mL), and neutralized with 1N HCl. The aqueous solution was extracted with CHCl₃, concentrated, and the resulting solid triturated with CHCl₃/Et₂O to give the title compound as a white solid (1.32 g, 3.28 mmol). ¹H NMR (DMSO-d₆) δ:11.23 (1H, s), 8.78 (1H, s), 8.73 (1H, s), 8.05 (2H, d, J=7.2 Hz), 7.66 (1H, t, J=7.2 Hz), 7.56 (2H, t, J=8.1 Hz), 6.05 (1H, d, J=5.8 Hz), 5.62 (1H, d, J=6.0 Hz), 5.41 (1H, d, J=4.9 Hz), 4.83 (1H, q, J=5.3 Hz), 4.19 (1H, q, J=3.8 Hz), 4.17−4.06 (1H, m), 2.92 (1H, dd, J=13.9 and 5.8 Hz), 2.82 (1H, dd, J=13.9 and 6.8 Hz), 2.07 (3H, s). LRMS (m/z) 402 (M+H)⁺. Anal. (C₁₈H₁₉N₅O₄S) C, H, N, S.

### Example 2(D)(2)

5-[(9-{(2R,3R,4S,5S)-3,4-dihydroxy-5-[(methylthio)methyl]tetrahydrofuran-2-yl}-9H-purin-6-yl)amino]-5-oxopentanoic acid.

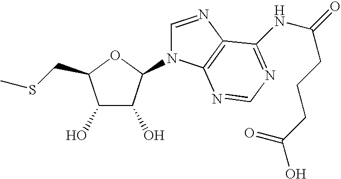

To a solution of MTA (1.07 g, 3.60 mmol) in pyridine (45 mL) was added ethyl glutarylchloride (2.3 mL, 14.6 mmol) at rt. After 16 h, the pyridine was removed under vacuum, and the resulting foam was redissolved in EtOH (35 mL) and THF (50 mL) and treated with 2N NaOH (40 mL). After 1 h at 0° C., the reaction was diluted with pH=7 phosphate buffer (50 mL) and neutralized with 1N HCl. The aqueous solution was extracted with CHCl₃, concentrated, and the resulting solid purified on semipreparative HPLC to give the title compound as a white solid (154 mg, 10%). ¹H NMR (DMSO-d₆) δ:10.72 (1H, s), 8.69 (1H, s), 8.67 (1H, s), 6.01 (1H, d, J=5.8 Hz), 5.62−5.56 (1H, m), 5.41−5.37 (1H, m), 4.82−4.75 (1H, m), 4.20−4.14 (1H, m), 4.10−4.03 (1H, m), 2.91 (1H, dd, J=13.9 and 5.8 Hz), 2.82 (1H, dd, J=13.9 and 6.8 Hz), 2.61 (2H, t, J=7.2 Hz), 2.30 (2H, t, J=7.4 Hz), 2.06 (3H, s), 1.87−1.77 (2H, m). LRMS (m/z) 412 (M+H)⁺. Anal. (C₁₆H₂₁N₅O₆S) C, H, N, S.

### EXAMPLE 2(D)(3)

6-[(9-{(2R,3R,4S,5S)-3,4-dihydroxy-5-[(methylthio)methyl]tetrahydrofuran-2-yl}-9H-purin-6-yl)amino]-6-oxohexanoic acid.

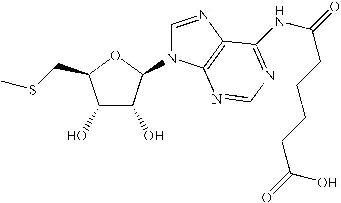

The title compound 2(D)(3) was prepared in a similar fashion to the previous example using adipoylchloride and MTA. ¹H NMR (DMSO-d₆) δ:12.02 (1H, br s), 10.70 (1H, s), 8.69 (1H, s), 8.67 (1H, s), 6.01 (1H, d, J=5.8 Hz), 5.63−5.55 (1H, m), 5.43−5.36 (1H, m), 4.79 (1H t, J=5.5 Hz), 4.21−4.14 (1H, m), 4.11−4.03 (1H, m), 2.91 (1H, dd, J=13.9 and 6.0 Hz) 2.80 (1H, dd, J=14.3 and 6.0 Hz), 2.57 (2H, t, J=6.6 Hz), 2.25 (2H, t, J=6.8 Hz), 2.06 (3H, s), 1.67−1.49 (4H, m). LRMS (m/z) 426 (M+H)⁺. Anal. (C₁₇H₂₃N₅O₆S-0.4 H2O) C, H, N, S.

### EXAMPLE 2(E)

Synthesis of Additional Adenosine Analogs of MTAP Substrates

Schemes VIII and IX outline the general methods to prepare adenosine analogs at the 5′ position of the sugar ring, where the 2′ position has already been modified. In scheme VIII, the sequence is begun with an appropriate intermediate that is already modified at the 2′ position (VIIIa). Conversion of the 5′ position into a leaving group (VIIIb; X═Cl) and subsequent displacement with a thiol gives the desired product VIIIc. The stereochemistry of the starting diol VIIIa is not specified and it may be either diastereomer.

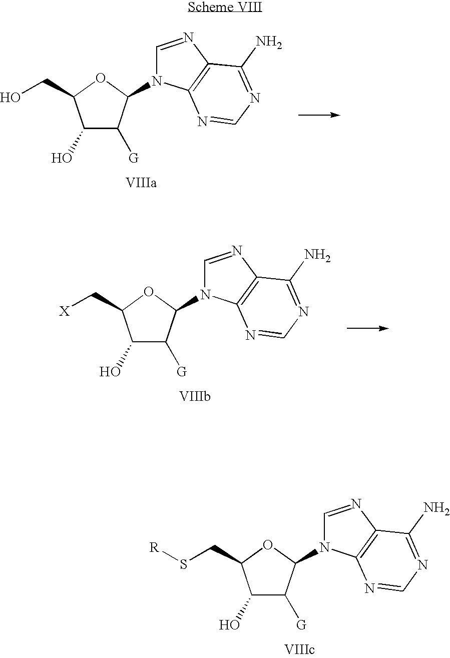

Alternatively, scheme IX illustrates a sequence wherein the 5′ position is already substituted with an appropriate thiol. Selective protection of the 3′ position gives the desired starting alcohol IXa. The free alcohol is converted to a leaving group (IXb; X=triflate (—OTf)), which is then displaced by a nucleophile (including, but not limited to azide, thiols, amines, alcohols, etc.). Following deprotection of the 3′ protecting group, the final products are obtained. Depending on the stereochemistry of the intermediates, it is possible to get both possible products, that is to say IXc or IXc′.

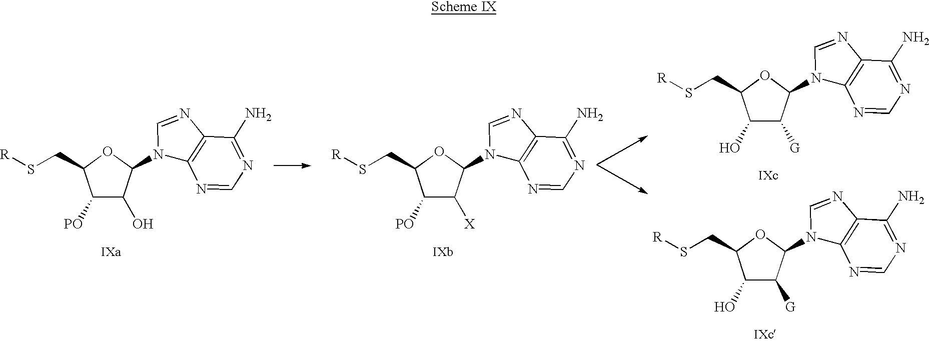

### Example 2(E)(1)

(2S,3R,4R,5R)-5-(6-amino-9H-purin-9-yl)-4-(methylthio)-2-[(methylthio)methyl]tetrahydrofuran-3-ol.

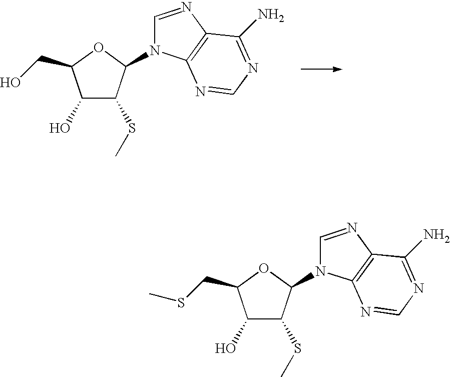

The title compound was prepared from S-methyl-2′-thio-adenosine (Robins et al. J. Amer. Chem. Soc. 1996, 46, 11341.; Fraser et al. J. Heterocycl. Chem. 1993, 5, 1277.; Montgomery, T. J. Heterocycl. Chem. 1979, 16, 353.; Ryan et al. J. Org. Chem. 1971, 36, 2646.) To a solution of S-methyl-2′-thio-adenosine (0.365 g, 1.23 mmol) in DMF (10 mL) and CCl₄ (2 mL) was added PPh₃ (0.322 g, 1.23 mmol). After 0.5 h at rt, the reaction was quenched with i-PrOH (10 mL), and the mixture was concentrated under vacuum. The resulting oil was redissolved in DMF (10 mL) and treated with NaSMe (0.222 g, 3.17 mmol). After 16 h at rt, the reaction mixture was concentrated under vacuum, and the resulting crude residue was purified on semipreparative HPLC with a linear gradient elution of 10% A/90% B to 30% A/70% B over 30 min to give the titled compound as a white powder (72.4 mg, 18%). ¹H NMR (DMSO-d₆) δ:8.43 (1H, s), 8.17 (1H, s), 7.35 (2H, s), 6.12 (1H, d, J=8.6 Hz), 5.89 (1H, bs), 4.35−4.24 (2H, m), 4.08 (1H, t, J=6.6 Hz), 2.90 (1H, dd, J=13.9 and 7.1 Hz), 2.82 (1H, dd, J=13.6 and 6.8 Hz), 2.08 (3H, s), 1.79 (3H, s). Anal. (C₁₂H₁₇N₅O₂S₂) C, H, N, S.

### EXAMPLE 2(E)(2)

(2S,3R,4R,5R)-5-(6-amino-9H-purin-9-yl)-4-(ethylthio)-2-[(methylthio)methyl]tetrahydrofuran-3-ol.

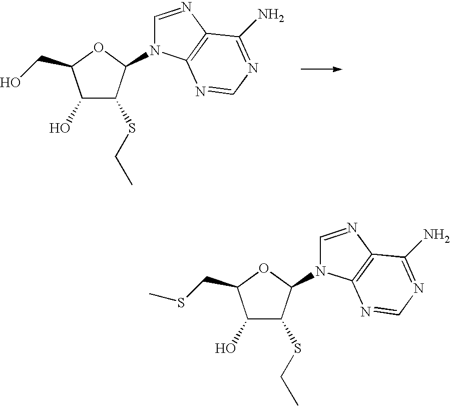

S-ethyl-2′-thio-adenosine was prepared in a similar fashion to that of S-methyl-2′-thio-adenosine (see references above) and was converted to the title compound using the procedure described for the example above. ¹H NMR (DMSO-d₆) δ:8.44 (1H, s), 8.16 (1H, s), 7.34 (2H, s), 6.07 (1H, d, J=8.8 Hz), 5.83 (1H, s), 4.39−4.36 (1H, m), 4.28−4.26 (1H, m), 4.08 (1H, t, J=6.8 Hz), 2.92 (1H, dd, J=13.9 and 7.3 Hz), 2.83 (1H, dd, J=13.6 and 6.8 Hz), 2.21 (2H, q, J=7.3 Hz), 2.07 (3H, s), 0.92 (3H, t, J=7.3 Hz). LRMS (m/z) 342 (M+H)⁺. Anal. (C₁₃H₁₉N₅O₂S₂-0.2 Hexanes) C, H, N, S.

### EXAMPLE 2(F)

Synthesis of Thiol Analogs of MTAP Substrates

The following examples were made using 5′-chloroadenosine as outlined in the procedure for Scheme I of Example 2(A), with substitution of the appropriate thiolate salt reagent in place of NaSCH₃. For those thiols where the thiolate salt was not commercially available, the anion was generated in situ using potassium t-butoxide.

### EXAMPLE 2(F)(1)

(2S,3S,4R,5R)-2-(6-amino-9H-purin-9-yl)-5-{[(4-chlorobenzyl)thio]methyl}tetrahydrofuran-3,4-diol.

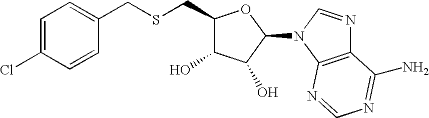

¹H-NMR (DMSO-d₆) δ:8.35 (1H, s), 8.15 (1H,s), 7.33−7.23 (6H, m), 5.89 (1H, d, J=5.2 Hz), 5.53 (1H, d, J=5.8 Hz), 5.33 (1H, d, J=5.2 Hz), 4.77−4.72 (1H, m), 4.20−4.15 (1H, m), 4.02−3.98 (1H; m), 3.73 (2H, s), 2.86−2.67 (2H, m). LRMS (m/z), 408 (M+H)⁺. Anal. (C₁₇H₁₈ClN₅O₃S) C, H, N, S.

### EXAMPLE 2(F)(2)

(2S,3S,4R,5R)-2-(6-amino-9H-purin-9-yl)-5-{[(3-hydroxypropyl)thio]methyl}tetrahydrofuran-3,4-diol.

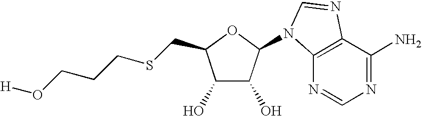

¹H-NMR (DMSO-d₆) δ:8.35 (1H, s), 8.15 (1H, s), 7.29 (2H, s), 5.89 (1H d, J=5.8 Hz), 5.49 (1H, s, J=6.2 Hz), 5.32 (1H, s, J=4.9 Hz), 4.78−4.73 (1H, m), 4.47−4.43 (1H, m), 4.17−4.12 (1H, m), 4.03−3.98 (1H, m), 3.43−3.37 (2H, m), 2.94−2.76 (1H, m), 2.57−2.52 (2H, m), 1.67−1.58 (2H, m). LRMS (m/z) 442 (M+H)⁺. Anal. (C₁₃H₁₉N₅O₄S-0.3 H₂O, 0.1 MeOH) C, H, N, S.

### EXAMPLE 2(F)(3)

(2S,3S,4R,5R)-2-(6-amino-9H-purin-9-yl)-5-[(pyrimidin-2-ylthio)methyl]tetrahydrofuran-3,4-diol.

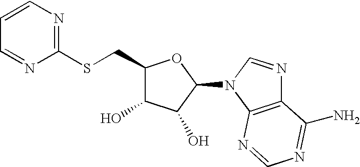

¹H-NMR (DMSO-d₆) δ:8.64 (2H, d, J=4.9 Hz), 8.37 (1H, s), 8.15 (1H, s), 7.30 (2H, s), 7.23 (1H, t, J=4.9 Hz), 5.90 (1H, d, J=6.2 Hz), 5.51 (1H, d, J=6.2 Hz), 5.39 (1H, d, J=4.7 Hz), 4.89−4.83(1H, m), 4.23−4.19 (1H, s), 4.15−4.10 (1H, s), 3.64−3.45 (1H, m). LRMS (m/z) 362 (M+H)⁺. Anal. (C₁₄H₁₅N₇O₃S-0.75 H₂O, 0.25 MeOH) C, H, N, S.

### EXAMPLE 2(F)(4)

(2S,3S,4R,5R)-2-(6-amino-9H-purin-9-yl)-5-{[(2-methylbutyl)thio]methyl}tetrahydrofuran-3,4-dio.

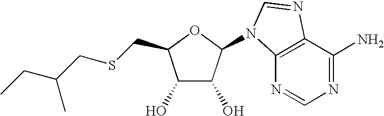

¹H-NMR (DMSO-d₆) δ:8.35 (1H, s); 8.15 (1H, s), 7.29 (2H, s), 5.88 (1H, d, J=4.7 Hz), 5.49 (1H, d, J=6.2 Hz), 5.29 (1H, d, J=4.5 Hz), 4.77 (br s, 1H), 4.15 (br s, 1H), 4.01 (br s, 1H), 2.91−2.81 (2H, m), 2.38−2.31 (1H, m), 1.48 (br s, 1H), 1.32 (br s, 1H), 1.10 (br s, 1H), 0.87−0.77 (6H, m). LRMS (m/z) 354 (M+H)⁺. Anal. (C₁₅H₂₃N₅O₃S-0.5 H₂O) C, H, N, S.

### EXAMPLE 2(F)(5)

(2S,3S,4R,5R)-2-(6-amino-9H-purin-9-yl)-5-{[(4-methyoxybenzyl)thio]methyl}tetrahydrofuran-3,4-diol.

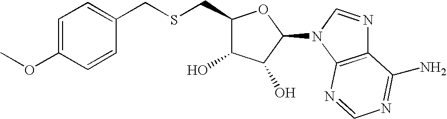

¹H-NMR (DMSO-d₆) δ8.35 (1H, s), 8.14 (1H, s), 7.31 (2H, s), 7.13 (2H, d, J=8.4 HZ), 6.81 (2H, d, J=8.4), 5.89 (1H, d, J=5.2 Hz), 5.51 (1H, d, J=6.0 Hz), 5.31 (1H, d, J=5.0), 4.77−4.71 (1H, m), 4.20−4.15 (1H, m), 4.04−3.98 (1H, m), 3.72 (3H, s), 3.68 (2H, s), 2.85−2.61 (2H, m). LRMS (m/z) 404 (M+H)⁺. Anal. (C₁₈H₂₁N₅O₄S-0.5 H₂O) C, H, N, S.

### EXAMPLE 2(F)(6)

(2S,3S,4R,5R)-2-(6-amino-9H-purin-9-yl)-5-[(quinolin-2-ylthio)methyl]tetrahydrofuran-3,4-diol.

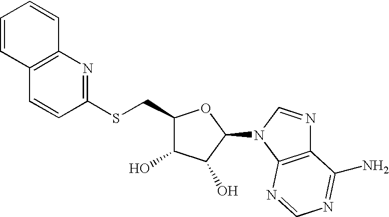

¹H-NMR (DMSO-d₆) δ:8.31 (1H, s), 8.09−8.06 (2H, m), 7.83−7.77 (2H, m), 7.65−7.59 (1H, m), 7.44−7.42 (1H, m), 7.31 (1H, d, J=8.6 Hz), 7.21 (2H, s), 5.82 (1H, d, J=6.4 Hz), 5.42 (1H, d, J=6.2 Hz), 5.28 (1H, d, J=4.9 Hz), 4.88−4.82 (1H, m), 4.17−4.08 (2H, m), 3.79−3.52 (2H, m). LRMS (m/z) 411 (M+H)⁺. Anal. (C₁₉H₁₈N₆O₃S) C, H, N, S.

### EXAMPLE 2(F)(7)

(2R,3R,4S,5S)-2-(6-amino-9H-purin-9-yl)-5-{[(3-methylphenyl)thio]methyl}tetrahydrofuran-3,4-diol.

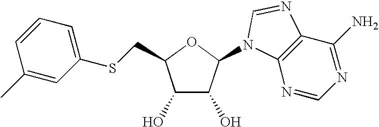

¹H NMR (DMSO-d₆) δ:8.34 (1H, s), 8.14 (1H, s), 7.30 (2H, s), 7.18−7.11 (3H, m), 6.98 (1H, d, J=7.1 Hz), 5.88 (1H, d, J=5.8 Hz), 5.51 (1H, d, J=6.3 Hz), 5.36 (1H, d, J=5.1 Hz), 4.81 (1H, q, J=5.8 Hz), 4.18 (1H, q, J=3.8 Hz), 3.98 (1H, q, J=3.8 Hz), 3.39 (1H, dd, J=13.9 and 6.1 Hz), 3.28 (1H, dd, J=13.9 and 6.06 Hz), 2.34 (3H, s). LRMS (m/z) 374 (M+H)⁺. Anal. (C₁₇H₁₉N₅O₃S-0.50 H₂O) C, H, N, S.

### EXAMPLE 2(F)(8)

(2R,3R,4S,5S)-2-(6-amino-9H-purin-9-yl)-5-{[(4-methylphenyl)thio]methyl}tetrahydrofuran-3,4-diol.

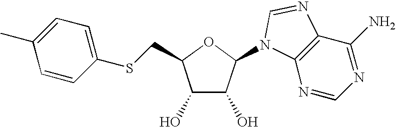

¹H NMR (DMSO-d₆) δ:8.34 (1H, s), 8.14 (1H, s), 7.30 (2H, s), 7.25 (2H, d, J=8.3 Hz), 7.11 (1H, d, J=8.3 Hz), 5.87 (1H, d, J=5.8 Hz), 5.50 (1H, d, J=6.3 Hz), 5.35 (1H, d, J=4.8 Hz), 4.80 (1H, q, J=6.1 Hz), 4.16 (1H, q, J=3.3 Hz), 3.96 (1H, m), 3.36 (1H, dd, J=13.9 and 6.06 Hz), 3.23 (1H, dd, J=13.9 and 7.06 Hz), 2.25 (3H, s). LRMS (m/z) 374 (M+H)⁺. Anal (C₁₇H₁₉N₅O₃S-0.70 H₂O) C, H, N, S.

### EXAMPLE 2(F)(9)

(2R,3R,4,5 S)-2-(6-amino-9H-purin-9-yl)-5-{[(2-methoxyphenyl)thio]methyl}tetrahydrofuran-3,4-diol.

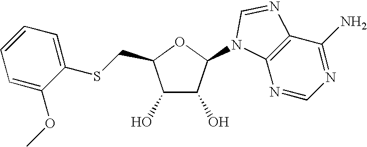

¹H NMR (DMSO-d₆) δ:8.35 (1H, s), 8.14 (1H, s), 7.29 (2H, s), 7.27 (1H, d, J=7.8 Hz), 7.17 (1H, t, J=7.6 Hz), 6.97 (d, 1H, J=8.1 Hz), 6.96 (t, 1H, J=7.3 Hz), 5.87 (1H, d, J=6.1 Hz), 5.50 (1H, d, J=6.1 Hz), 5.36 (1H, d, J=4.8 Hz), 4.82 (1H, q, J=5.3 Hz), 4.18 (1H, q, J=3.3 Hz), 4.00−3.95 (1H, m), 3.79 (s, 3H), 3.37−3.30 (1H, m), 3.22−3.15 (1H, m). LRMS (m/z) 390 (M+H)⁺. Anal. (C₁₇H₁₉N₅O₄S-0.50 H₂O) C, H, N, S.

### EXAMPLE 2(F)(10)

(2R,3R,4S,5S)-2-(6-amino-9H-purin-9-yl)-5-{[(3-methoxyphenyl)thio]methyl}tetrahydrofuran-3,4-diol.

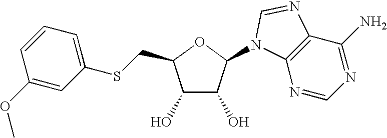

¹H NMR (DMSO-d₆) δ:8.34(1H, s), 8.14 (1H, s), 7.30 (2H, s), 7.19 (1H, t, J=7.8 Hz), 6.90−6.89 (2H, m), 6.74 (d, 1H, J=8.1 Hz), 5.88 (1H, d, J=5.8 Hz), 5.52 (1H, d, J=6.1 Hz), 5.38 (1H, d, J=5.1 Hz), 4.80 (1H, q, J=5.6 Hz), 4.19 (1H, q, J=3.8 Hz), 4.01−3.97 (1H, m), 3.70 (s, 3H), 3.43 (1H, dd, J=13.9 and 5.8 Hz), 3.29 (1H, dd, J=14.2 and 7.1 Hz). LRMS (m/z) 390 (M+H)⁺. Anal. (C₁₇H₁₉N₅O₄S-0.50 H₂O) C, H, N, S.

### EXAMPLE 2(F)(11)

(2R,3R,4S,5S) -2-(6-amino-9H-purin-9-yl)-5-{[(4-methoxyphenyl)thio]methyl}tetrahydrofuran-3,4-diol.

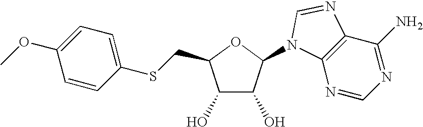

¹H NMR (DMSO-d₆) δ:8.33(1H, s), 8.14 (1H, s), 7.31 (2H, d, J=8.8 Hz), 7.29 (2H, s), 6.87 (2H, d, J=8.8 Hz), 5.86 (1H, d, J=6.1 Hz), 5.48 (1H, d, J=6.1 Hz), 5.33 (1H, d, J=4.8 Hz), 4.80 (1H, q, J=5.3 Hz), 4.14 (1H, q, J=4.8 Hz), 3.94−3.90 (1H, m), 3.72 (s, 3H), 3.27 (1H, dd, J=13.9 and 6.1 Hz), 3.10 (1H, dd, J=13.9 and 7.1 Hz). LRMS (m/z) 390 (M+H)⁺. Anal. (C₁₇H₁₉N₅O₄S-0.50 H₂O) C, H, N, S.

### EXAMPLE 2(F)(12)

(2R,3R,4S,5S)-2-(6-amino-9H-purin-9-yl)-5-{[(2-methylbenzyl)thio]methyl}tetrahydrofuran-3,4-diol.

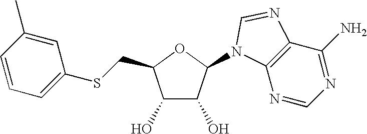

¹H NMR (DMSO-d₆) δ:8.35(1H, s), 8.14 (1H, s), 7.30 (2H, s), 7.14−7.02 (4H, m), 5.89 (1H, d, J=5.5 Hz), 5.51 (1H, d, J=6.0 Hz), 5.32 (1H, d, J=5.3 Hz), 4.76 (1H, q, J=4.3 Hz), 4.17 (1H, q, J=4.7 Hz), 4.05−4.00 (1H, m), 3.73 (s, 2H), 2.87 (1H, dd, J=13.8 and 5.8 Hz), 2.73 (1H, dd, J=13.9 and 7.0 Hz), 2.28 (s, 3H). LRMS (m/z) 388 (M+H)⁺. Anal. (C₁₈H₂₁N₅O₃S-0.40 H₂O) C, H, N, S.

### EXAMPLE 2(F)(13)

(2R,3R,4S,5S)-2-(6-amino-9H-purin-9-yl)-5-{[(3-methylbenzyl)thio]methyl}tetrahydrofuran-3,4-diol.

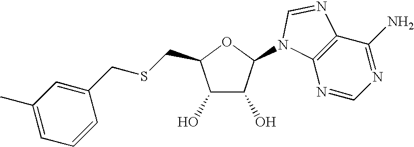

¹H NMR (DMSO-d₆) δ:8.34(1H, s), 8.13 (1H, s), 7.30 (2H, s), 7.15 (1H, t, J=7.4 Hz), 7.04−7.00 (3H, m), 5.88 (1H, d, J=5.5 Hz), 5.51 (1H, d, J=5.8 Hz), 5.31 (1H, d, J=5.3 Hz), 4.73 (1H, q, J=5.3 Hz), 4.17 (1H, q, J=4.7 Hz), 4.04−3.98 (1H, m), 3.69 (s, 2H), 2.83 (1H, dd, J=13.9 and 5.8 Hz), 2.68 (1H, dd, J=13.8 and 7.0 Hz), 2.25 (s, 3H). LRMS (m/z) 388 (M+H)⁺. (C₁₈H₂₁N₅O₃S-0.50 H₂O) C, H, N, S.

### EXAMPLE 2(F)(14)

(2R,3R,4S,5S)-2-(6-amino-9H-purin-9-yl)-5-({[3-(trifluoromethyl)phenyl]thio}methyl)tetrahydrofuran-3,4-diol.

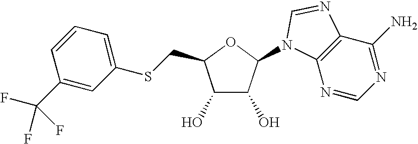

¹H NMR (DMSO-d₆) δ:8.33(1H, s), 8.14 (1H, s), 7.66−7.59 (2H, m), 7.51−7.47 (2H, m), 7.31 (2H, s), 5.90 (1H, d, J=5.7 Hz), 5.56 (1H, d, J=6.0 Hz), 5.42 (1H, d, J=4.5 Hz), 4.84−4.77 (1H, m), 4.25−4.18 (1H, m), 4.05−3.99 (1H, m), 3.53 (1H, dd, J=13.8 and 5.8 Hz), 3.44 (1H, dd, J=14.3 and 7.5 Hz). LRMS (m/z) 428 (M+H)⁺. Anal. (C₁₇H₁₆F₃N₅O₃S) C, H, N, S.

### EXAMPLE 2(F)(15)

(2R,3R,4S,5S)-2-(6-amino-9H-purin-9-yl)-5-({[4-(trifluoromethyl)phenyl]thio}methyl)tetrahydrofuran-3,4-diol.

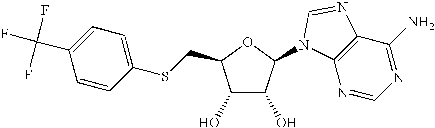

¹H NMR (DMSO-d₆) δ:8.36(1H, s), 8.15 (1H, s), 7.60 (2H, d, J=8.3 Hz), 7.51 (2H, d, J=8.3 Hz), 7.31 (2H, s), 5.90 (1H, d, J=5.8 Hz), 5.57 (1H, d, J 5.8 Hz), 5.41 (1H, d, J=5.1 Hz), 4.83 (1H, q, J=5.3 Hz), 4.25−4.19 (1H, m), 4.08−4.00 (1H, m), 3.54 (1H, dd, J=13.8 and 5.5 Hz), 3.44 (1H, dd, J=13.6 and 7.0 Hz). LRMS (m/z) 428 (M+H)⁺. (C₁₇H₁₆F₃N₅O₃S-0.50 H₂O) C, H, N, S.

### EXAMPLE 2(F)(16)

(2R,3R,4S,5S)-2-(6-amino-9H-purin-9-yl)-5-{[(2-pyridin-ylethyl)thio]methyl}tetrahydrofuran-3,4-diol

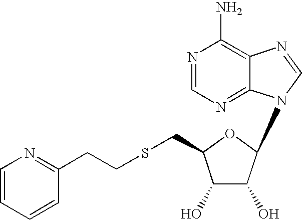

¹HNMR (300 MHz, DMSO-D₆) δ ppm 2.57 (t, 2H, J=6.0 Hz) 2.87 (m, 2H) 3.49 (q, 2H, J=6.0 Hz) 4.01 (m, J=3.58 Hz, 1 H) 4.13 (m, 1 H) 5.32 (s, 1 H) 5.50 (s, 1 H) 5.87 (d, J=5.65 Hz, 1 H) 7.20 (m, 2 H) 7.36 (s, 2 H) 7.68 (td, J=7.68, 1.79 Hz, 1 H) 8.15 (s, 1 H) 8.36 (s, 1 H) 8.46 (d, J=4.14 Hz, 1 H). Anal. Calcd for C₁₇H₂₀N₆O₃S.1H₂O C: 50.24, H: 5.46, N: 20.68, S: 7.89. Found C: 50.18, H: 5.29, N: 20.60, S: 7.80.

### EXAMPLE 2(F)(17)

(2S,3R,4R,5R)-2-(6-amino-9H-purin-9-yl)-5-[(pyridin-4-ylthio)methyl]tetrahydrofuran-3,4-diol

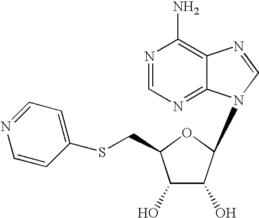

¹H NMR (400 MHz, DMSO-d₆) δ ppm 3.37 (dd, J=14.3, 7.5 Hz, 1 H) 3.48 (m, 1 H) 4.00 (s, 1 H) 4.17 (d, J=3.54 Hz, 1 H) 4.76 (d, J=5.6 Hz, 1 H) 5.38 (d, J=4.8 Hz, 1 H) 5.51 (d, J=6.1 Hz, 1 H) 5.84 (d, J=5.6 Hz, 1 H) 7.23 (m, 4 H) 8.08 (s, 1 H) 8.26 (m, 3 H). Anal. Calcd for C₁₅H₁₆N₆O₃S.0.5H₂O C: 48.77, H: 4.64, N: 22.75, S: 8.68. Found C: 48.81 H: 4.57, N: 22.71, S: 8.74.

### EXAMPLE 2(F)(18)

(2R,3R,4R,5S)-2-(6-amino-9H-purin-9-yl)-5-{[(2-hydroxyethyl)thio]methyl}tetrahydrofuran-3,4-diol

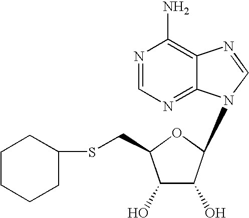

¹H NMR (400 MHz, DMSO-d₆) δ ppm 1.14 (m, 5 H) 1.48 (m, 1 H) 1.61 (m, 2 H) 1.84 (m, 2 H) 2.65 (m, 1H) 2.79 (dd, J=14.0, 7.0 Hz, 1 H) 2.91 (dd, J=12.0, 4.0 Hz, 1 H) 3.96 (m, 1 H) 4.14 (m, 1 H) 4.77 (q, J=5.6 Hz, 1 H) 5.28 (d, J=5.1 Hz, 1 H) 5.47 (d, J=6.1 Hz, 1 H) 5.86 (d, J=5.8 Hz, 1H) 7.28 (s, 1 H) 8.13 (s, 1 H) 8.34 (s, 1 H). Anal. Calcd for C₁₆H₂₃N₅O₃S.0.75H₂O C: 50.71, H: 6.52, N: 18.48, S: 8.46. Found C: 51.02 H: 6.29, N: 18.55, S: 8.37.

### EXAMPLE 2(F)(19)

(2R,3R,4S,5S)-2-(6-amino-9H-purin-9-yl)-5-[(pyridin-2-ylthio)methyl]tetrahydrofuran-3,4-diol

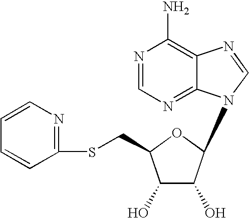

¹H NMR (400 MHz, DMSO-D6) δ ppm 3.16 (d, J=4.8 Hz, 1 H) 3.48 (dd, J=13.8, 7.0 Hz, 1 H) 3.61 (dd, J=12.0, 6.0 Hz, 1 H) 4.07 (m, 1 H) 4.17 (m, 1 H) 4.84 (q, J=6.0 Hz, 1 H) 5.36 (d, J=4.8 Hz, 1 H) 5.50 (d, J=6.3 Hz, 1 H) 5.88 (d, J=6.3 Hz, 1 H) 7.10 (dd, J=6.7, 4.9 Hz, 1 H) 7.30 (s, 1 H) 7.61 (d, J=7.7, 1.8 Hz, 1 H) 8.14 (s, 1 H) 8.35 (s, 1 H) 8.42 (d, J=4.0 Hz, 1 H). Anal. Calcd for C₁₅H₁₆N₆O₃S.0.25HCl.1.0H₂O.0.5CH₃OH C: 46.13, H: 5.06, N: 20.83, S: 7.95. Found C: 46.18 H: 5.16, N: 20.75, S: 7.93.

### EXAMPLE 2(F)(20)

(2S,3R,4R,5R)-ethyl-3-({[5-(6-amino-9H-purin-9-yl)-3,4-dihydroxytetrahydrofuran-2- yl]methyl}thio)propanoate

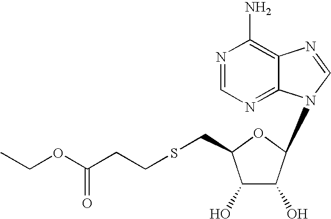

¹H NMR (300 MHz, CD₃OD) δ ppm 1.20 (t, J=4.0 Hz, 3 H) 2.55 (m, 2 H) 2.78 (m, 2 H) 2.97 (m, 2 H) 4.07 (q, J=4.0 Hz, 2 H) 4.20 (d, J=4.9 Hz, 1 H) 4.32 (d, J=4.9 Hz, 1 H) 4.79 (d, J=4.9 Hz, 1 H) 5.99 (d, J=4.9 Hz, 1 H) 8.21 (s, 1 H) 8.31 (s, 1 H). Anal. Calcd for C₁₅H₂₁N₅O₅S.0.2CH₃COOH.0.5HCl C: 44.71, H: 5.43, N: 16.93, S: 7.75. Found C: 44.49 H: 5.60, N. 16.66, S: 8.16.

### EXAMPLE 2(F)(21)

(2S,3R,4R,5R)-2-(6-amino-9H-purin-9-yl)-5-{[(2-furylmethyl)thio]methyl}tetrahydrofuran-3,4-diol

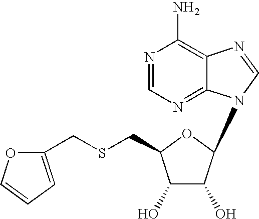

¹H NMR (400 MHz, DMSO-d₆) δ ppm 2.75 (dd, J=13.9, 7.1 Hz, 1H) 2.89 (m, 1 H) 3.16 (d, J=4.8 Hz, 1 H) 3.76 (s, 2 H) 3.97 (m, 1 H) 4.12 (m, 1 H) 4.73 (q, J=5.7 Hz, 1 H) 5.30 (d, J=5.3 Hz, 1H) 5.49 (d, J=6.1 Hz, 1 H) 5.87 (d, J=5.8 Hz, 1 H) 6.18 (d, J=3.0 Hz, 1 H) 6.34 (dd, J=3.0, 1.8 Hz, 1 H) 7.29 (s, 2 H) 7.55 (d, J=2.0 Hz, 1 H) 8.13 (s, 1 H) 8.33 (s, 1 H). Anal. Calcd for C₁₅H₁₇N₅O₄S.0.5H₂O C: 48.38, H: 4.87, N: 18.81, S: 8.61. Found C: 48.25, H: 4.72, N: 18.53, S: 8.69.

### EXAMPLE 2(F)(22)

(2S,3R,4R,5R)-2-(6-Amino-purin-9-yl)-5-(1H-imidazole-2-ylsulfanylmethyl)-tetrahydro-furan-3,4-diol

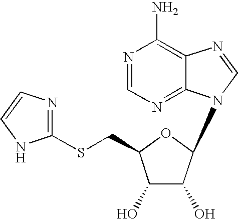

¹H NMR (400 MHz, MeOD) δ ppm 3.26 (m, 2 H) 3.69 (s, 1 H) 4.07 (m, J=4.04 Hz, 1 H) 4.18 (m, 1 H) 5.86 (d, J=5.56 Hz, 1 H) 6.91 (s, 2 H) 8.10 (d, J=7.33 Hz, 2 H). MS for C₁₃H₁₅N₇O₃S (MW:349), m/e 350 (MH⁺). Anal. Calcd for C₁₃H₁₅N₇O₃S.1.0H₂O.0.35 hexane C: 45.62, H: 5.55, N: 24.65. Found C: 45.84, H: 5.20, N: 24.27.

### EXAMPLE 2(F)(23)

(2S,3R,4R,5R)-2-(6-Amino-purin-9-yl)-5-(thiazol-2-ylsulfanylmethyl)-tetrahydro-furan-3,4-diol

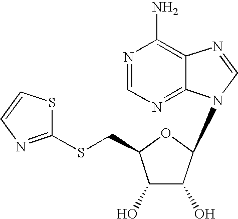

¹H NMR (400 MHz, MeOD) δ ppm 3.66 (m, 2 H) 4.29 (m, 1 H) 4.35 (m, 1 H) 5.95 (d, J=5.05 Hz, 1 H) 7.41 (d, J=3.28 Hz, 1 H) 7.61 (d, J=3.54 Hz, 1 H) 8.16 (s, 1 H) 8.21 (s, 1H). HRMS for C₁₃H₁₄N₆O₃S₂ (MW:366.425), m/e 367.0647 (MH⁺). Anal. Calcd for C₁₃H₁₄N₆O₃S₂.0.4H₂O C: 41.79, H: 3.99, N: 22.49. Found C: 41.96, H: 4.03, N: 22.10.

### EXAMPLE 2(F)(24)

(2S,3R,4R,5R)-2-(6-Amino-purin-9-yl)-5-(4-fluoro-benzylsulfanylmethyl)-tetrahydro-furan-3,4-diol

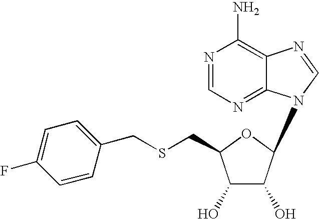

¹H NMR (400 MHz, MeOD) δ ppm 2.67 (m, 1 H) 3.63 (m, 2 H) 4.08 (m, 1 H) 4.24 (m, J=5.18, 5.18 Hz, 1 H) 4.66 (m, J=4.93, 4.93 Hz, 1 H) 5.90 (d, J=4.55 Hz, 1 H) 6.85 (t, J=8.72 Hz, 2 H) 7.13 (m, 2 H) 7.88 (s, 1 H) 8.09 (s, 1 H) 8.19 (s, 1 H). MS for C₁₇H₁₈FN₅O₃S (MW:391), m/e 392 (MH⁺). Anal. Calcd for C₁₇H₁₉FN₅O₃S.0.6MeOH C: 51.47, H: 5.01, N: 17.06. Found C: 51.56, H: 5.50, N: 17.21.

### EXAMPLE 2(F)(25)

(2S,3R,4R,5R)-2-(6-Amino-purin-9-yl)-5-(thiophen-2-ylmethylsulfanylmethyl)-tetrahydro-furan-3,4-diol

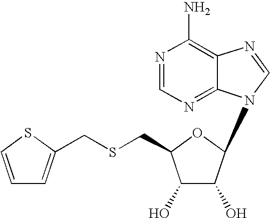

¹H NMR (400 MHz, CD₃OD) δ ppm 1.08 (t, J=7.1 Hz, 1 H) 2.74 (dd, J=14.3, 6.2 Hz, 1 H) 2.83 (m, 1 H) 3.51 (q, J=7.1 Hz, 1 H) 3.88 (q, J=14.4 Hz, 2 H) 4.10 (q, J=5.3 Hz, 1 H) 4.23 (t, J=5.2 Hz, 1 H) 4.66 (t, J=5.1 Hz, 1 H) 5.89 (d, J=4.8 Hz, 1 H) 6.75 (m, 2 H) 7.14 (dd, J=4.7, 1.6 Hz, 1 H) 8.09 (s, 1 H) 8.19 (s, 1 H). HRMS for C₁₅H₁₇N₅O₃S (MW:379.46), m/e 380.086 (MH⁺). Anal. Calcd for C₁₅H₁₇N₅O₃S.0.4H₂O.0.4HOAc C: 46.21, H: 4.76, N: 17.05. Found C: 46.19, H: 4.51, N: 16.92.

### EXAMPLE 2(F)(26)

(2S,3R,4R,5R)-2-(6-Amino-purin-9-yl)-5-cyclopentylsulfanylmethyl-tetrahydro-furan-3,4-diol

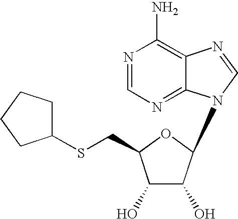

¹H NMR (400 MHz, DMSO-d₆) δ ppm 1.41 (m, 2 H) 1.47 (m, 2 H) 1.63 (m, 2 H) 1.89 (m, 2 H) 2.82 (dd, J=13.8, 7.0 Hz, 1 H) 2.93 (m, 1 H) 3.13 (m, 1 H) 4.02 (m, 1 H) 4.15 (m, 1 H) 4.77 (q, J=5.7 Hz, 1 H) 5.32 (d, J=5.1 Hz, 1 H) 5.50 (d, J=6.3 Hz, 1 H) 5.89 (d, J=5.8 Hz, 1 H) 7.30 (s, 2 H) 8.15 (s, 1 H) 8.36 (s, 1 H) MS for C₁₅H₂₁N₅O₃S (MW:351), m/e 352 (MH⁺). Anal. Calcd for C₁₅H₂₁N₅O₃S.0.3H₂O C: 50.49, H: 6.10, N: 19.63. Found C: 50.46, H: 6.17, N: 19.50.

### EXAMPLE 2(F)(27)

(2S,3R,4R,5R)-2-(6-Amino-purin-9-yl)-5-(3-phenyl-propylsufanylmethyl-tetrahydro-furan-3,4-diol

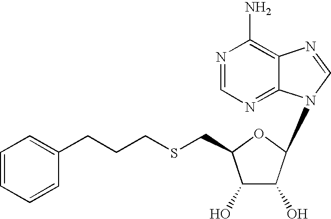

¹H NMR (400 MHz, CD₃OD) δ ppm 1.74 (m, 2 H) 2.44 (m, 2 H) 2.52 (m, 2 H) 2.83 (m, 4 H) 4.09 (q, J=5.5 Hz, 1 H) 4.23 (t, J=5.1 Hz, 1 H) 4.69 (t, J=5.2 Hz, 1 H) 5.89 (d, J=5.1 Hz, 1 H) 7.01 (m, 3 H) 7.11 (t, J=7.3 Hz, 2 H) 8.10 (s, 1 H) 8.21 (s, 1 H). HRMS for C₁₉H₂₃N₅O₃S (MW:401.15) m/e 402.1617 (MH⁺). Anal. Calcd for C₁₉H₂₃N₅O₃S.0.1CH₃COOH C: 56.59, H: 5.78, N: 17.19. Found C: 56.50, H: 5.76, N: 17.22.

### EXAMPLE 2(F)(28)

(2R,3R,4S,5S)-2-(6-amino-9H-purin-9-yl)-5-{[(2-methylphenyl)thio]methyl}tetrahydrofuran-3,4-diol

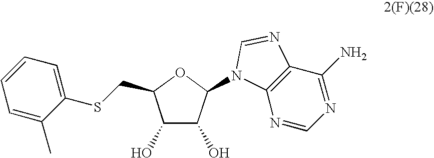

¹H NR (DMSO-d₆) δ: 8.16 (1H, s), 7.95 (1H, s), 7.15 (1H, d, J=6.82 Hz), 7.11 (2H s) 7.01−6.88 (3H, m) 5.70 (1H, d, J=6.1 Hz), 5.34 (1H, d, J=6.1 Hz), 5.20 (1H, d, J=5.1 Hz), 4.64 (1H, J=5.8 Hz), 4.02 (1H, q, J=4.8 Hz), 3.83−3.78 (1H, m), 3.20 (1H, dd, J=13.6 and 6.1 Hz), 3.08 (1H, dd, J=13.6 and 7.3 Hz), 2.08 (3H, s). LRMS (m/z) 374 (M+H)⁺.

### EXAMPLE 2(G)

Combinatorial Libraries of MTAP Substrates

Combinatorial libraries of thiol derivatives off the 5′ position of the adenosine were made as follows.

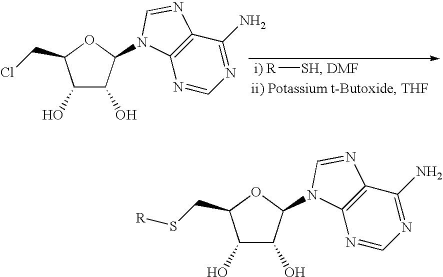

To a solution of the thiol in DMF (1.5 equiv.) was added a solution of alkyl mercaptan in DMF (1.0 equiv.) followed by the addition of a potassium t-butoxide solution in THF (1.5 equiv.). The mixture was heated to 55° C. for 12 h. The solvents were removed, and the residues were reconstituted in DMSO. Purification by HPLC afforded purified products (3-68% yield) as shown in Table 9 below.

TABLE 9
Library compounds of thiol derivatives off the 5′
position of the adenosine ring.

| | | | | m/z | MT- | MT- |
|---|---|---|---|---|---|---|
| Example | | | | [MW + | A_{—} | A_{—} |
| Number | Name | Stucture | MW | 1] | 10 | 50 |
| 2(G)(1) | (2R, 3R, 4S, 5S)-2-(6- amino-9H-purin-9-yl)-5- ({[2-(1, 4, 5, 6- tetrahydropyrimidin-2- yl)phenyl]thio}methyl)- tetrahydrofuran-3,4-diol | 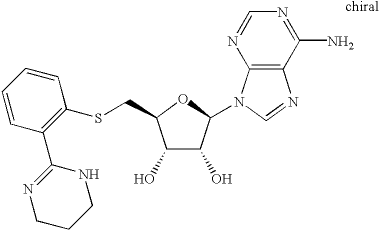 | 441.51 | 443 | 8 | 23 |
| 2(G)(2) | (2R, 3R, 4S, 5S)-2-(6- amino-9H-purin-9-yl)-5- {[(2-amino- phenyl)thio]methyl}tetrahydrofuran-3,4-diol | 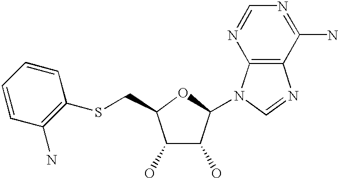 | 374.42 | 375 | 3 | 5 |
| 2(G)(3) | (2R, 3R, 4S, 5S)-2-(6- amino-9H-purin-9-yl)-5- {[(2-amino-7H-purin-6- yl)thio]methyl}tetrahydro- furan-3,4-diol | 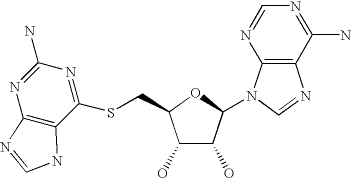 | 416.42 | 417 | 46 | 45 |
| 2(G)(4) | 2-({[(2S, 3S, 4R, 5R)-5-(6- amino-9H-purin-9-yl)- dihydroxytetrahydrofuran- 2-yl]methyl}thio)-5- ethylpyrimidin-4(3H)-one | 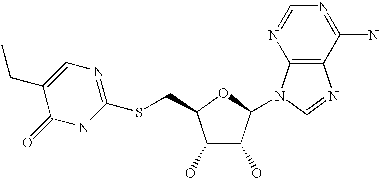 | 405.44 | 406 | 38 | 49 |
| 2(G)(5) | (2R, 3R, 4S, 5S)-2-(6- amino-9H-purin-9-yl)-5- {[(5-chloro-1H- yl)thio]methyl}tetrahydro- furan-3,4-diol | 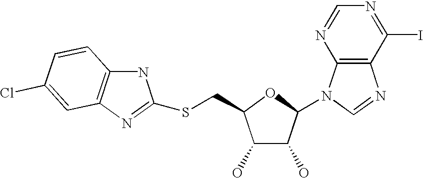 | 433.88 | 434/ 436 | 5 | 2 |
| 2(G)(6) | (2R, 3R, 4S, 5S)-2-(6- amino-9H-purin-9-yl)-5- {[(1-methyl-1H-tetrazol- 5-yl)thio]methyl}tetra- hydrofuran-3,4-diol | 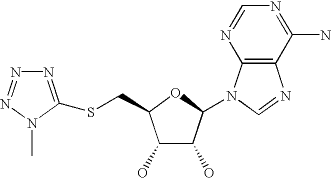 | 365.38 | 366 | 46 | 47 |
| 2(G)(7) | (2R, 3R, 4S, 5S)-2-(6- amino-9H-purin-9-yl)-5- ({[5-(propylthio)-1H- benzimidazol-2- yl]thio}methyl)tetrahydro- furan-3,4-diol | 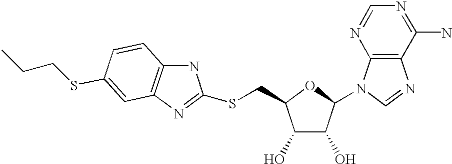 | 473.58 | 475 | 3 | 0 |
| 2(G)(8) | (2R, 3R, 4S, 5S)-2-(6- amino-9H-purin-9-yl)-5- [(pyrimidin-2- ylthio)methyl]tetrahydro- furan-3,4-diol | 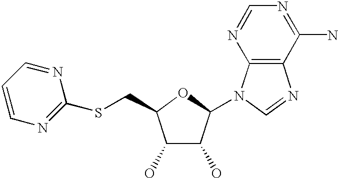 | 361.38 | 362 | 54 | 59 |
| 2(G)(9) | (2R, 3R, 4S, 5S)-2-(6- amino-9H-purin-9-yl)-5- {[(5-amino-1,3,4- thiadiazol-2- yl)thio]methyl}tetrahydro- furan-3,4-diol | 382.43 | 383 | 34 | 47 | |
| 2(G)(10) | (2R, 3R, 4S, 5S)-2-(6- amino-9H-purin-9-yl)-5- {[(4- aminophenyl)thio]methyl]- tetrahydrofuran-3,4- diol | 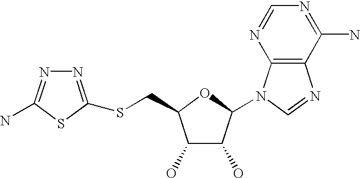 | 374.42 | 375 | 20 | 19 |
| 2(G)(11) | (2R, 3R, 4S, 5S)-2-(6- amino-9H-purin-9-yl)-5- {[(5-chloro-1,3- benzothiazol-2- yl)thio]methyl}tetrahydro- furan-3,4-diol | 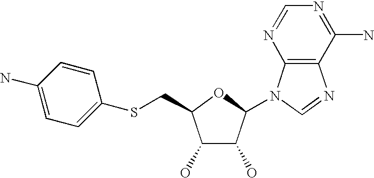 | 450.93 | 451/ 453 | 22 | 25 |
| 2(G)(12) | (2R, 3R, 4S, 5S)-2-(6- amino-9H-purin-9-yl)-5- [(1,3-benzothiazol-2- ylthio)methyl]tetrahydro- furan-3,4-diol | 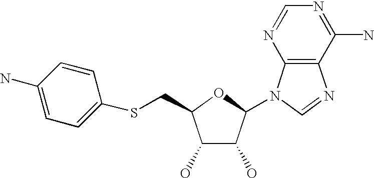 | 416.48 | 417 | 24 | 25 |
| 2(G)(13) | N-[4-({[(2S, 3S, 4R, 5R)-5- (6-amino-9H-purin-9-yl)- dihydroxytetrahydro- furan-2-yl]methyl}thio)phenyl]acetamide | 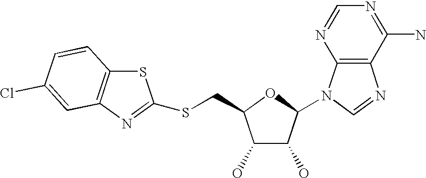 | 416.46 | 417 | 19 | 17 |
| 2(G)(14) | (2R, 3R, 4S, 5S)-2-(6- amino-9H-purin-9-yl)-5- {[(4-hydroxyphenyl)-thio]- methyl}tetrahydrofuran- 3,4-diol | 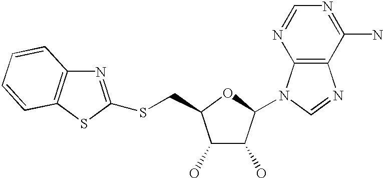 | 375.41 | 376 | 16 | 51 |
| 2(G)(15) | (2R, 3R, 4S, 5S)-2-(6- amino-9H-purin-9-yl)-5- {[(2-naphthylthio)methyl]- tetrahydrofuran-3,4-diol | 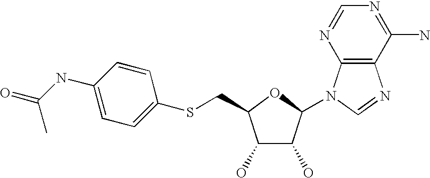 | 409.47 | 410 | 29 | 25 |
| 2(G)(16) | (2R, 3R, 4S, 5S)-2-(6- amino-9H-purin-9-yl)-5- {[(4-methoxybenzyl)- thio]methyl}tetrahydro- furan-3,4-diol | 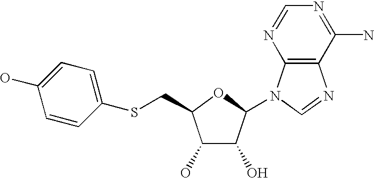 | 403.46 | 404 | 59 | 60 |
| 2(G)(17) | (2R, 3R, 4S, 5S)-2-(6- amino-9H-purin-9-yl)-5- {[(4-bromophenyl)- thio]methyl}tetrahydro- furan-3,4-diol | 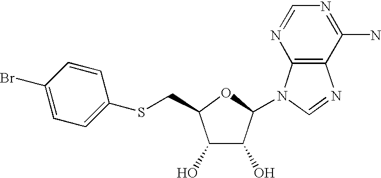 | 438.30 | 438/ 440 | 21 | 17 |
| 2(G)(18) | (2R, 3R, 4S, 5S)-2-(6- amino-9H-purin-9-yl)-5- [(1-naphthylthio)methyl]tetrahydrofuran-3,4-diol | 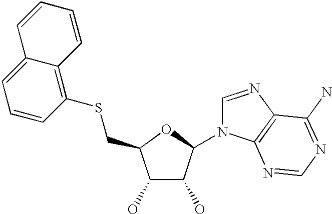 | 409.47 | 410 | 5 | 4 |
| 2(G)(19) | (2R, 3R, 4S, 5S)-2-(6- amino-9H-purin-9-yl)-5- {[(4-chlorophenyl)thio]- methyl}tetrahydro- furan-3,4-diol | 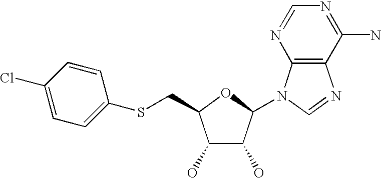 | 393.85 | 394/ 396 | 19 | 17 |
| 2(G)(20) | methyl 4- ({[(2S, 3S, 4R, 5R)-5-(6- amino-9H-purin-9-yl)-5- 3,4-dihydroxytetrahydro- furan-2-yl]methyl}thio)benzoate | 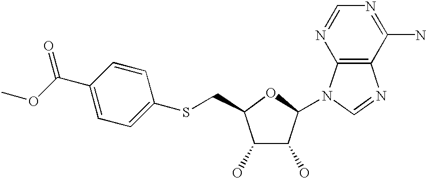 | 417.44 | 418 | 7 | 5 |
| 2(G)(21) | (2R, 3R, 4S, 5S)-2-(6- amino-9H-purin-9-yl)-5- {[(4-tert- butylphenyl)thio]methyl}tetrahydrofuran-3,4-diol | 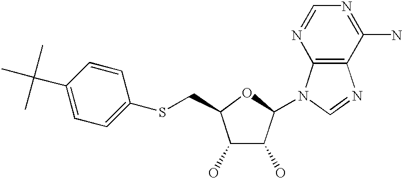 | 415.52 | 417 | 12 | 9 |
| 2(G)(22) | (2R, 3R, 4S, 5S)-2-(6- amino-9H-purin-9-yl)-5- {[(2,6-dimethylphenyl)- thio]methyl}tetrahydro- furan-3,4-diol | 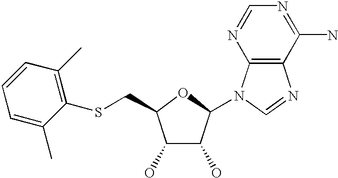 | 387.46 | 388 | 3 | 15 |
| 2(G)(23) | (2R, 3R, 4S, 5S)-2-(6- amino-9H-purin-9-yl)-5- {[(4-fluorophenyl)- thio]methyl}tetra- hydrafuran-3,4-diol | 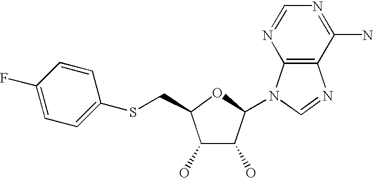 | 377.40 | 378 | 21 | 31 |
| 2(G)(24) | (2R, 3R, 4S, 5S)-2-(6- amino-9H-purin-9-yl)-5- {[(2,5-dimethoxy- phenyl)-thio]methyl}tetrahydrofuran- 3,4-diol | 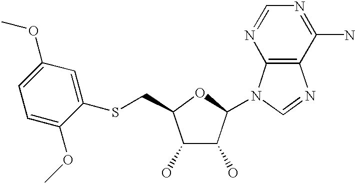 | 419.46 | 420 | 4 | 23 |
| 2(G)(25) | (2R, 3R, 4S, 5S)-2-(6- amino-9H-purin-9-yl)-5- {[(3,4-dimethoxyphenyl)- thio]methyl}tetrahydro- furan-3,4-diol | 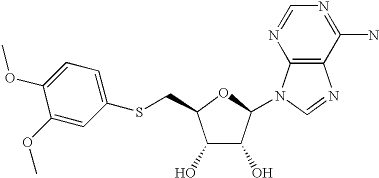 | 419.46 | 420 | 5 | 30 |
| 2(G)(26) | (2R, 3R, 4S, 5S)-2-(6- amino-9H-purin-9-yl)-5- {[(2-ethylphenyl)- thio]methyl}tetrahydro- furan-3,4-diol | 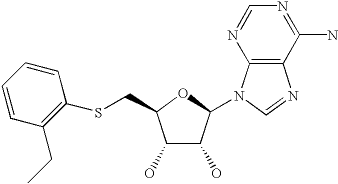 | 387.46 | 388 | 6 | 7 |
| 2(G)(27) | (2R, 3R, 4S, 5S)-2-(6- amino-9H-purin-9-yl)-5- {[(2-hydroxyphenyl)- thio]methyl}tetrahydro- furan-3,4-diol | 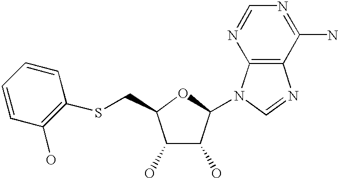 | 375.41 | 376 | 7 | 23 |
| 2(G)(28) | (2R, 3R, 4S, 5S)-2-(6- amino-9H-purin-9-yl)-5- {[2,5-dimethylphenyl)- thio]methyl}tetrahydro- furan-3,4-diol | 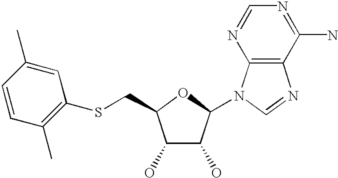 | 387.46 | 388 | 6 | 4 |
| 2(G)(29) | (2R, 3R, 4S, 5S)-2-(6- amino-9H-purin-9-yl)-5- {[(3-bromophenyl)- thio]methyl}tetrahydro- furan-3,4-diol | 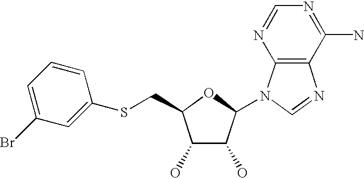 | 438.30 | 438/ 440 | 21 | 19 |
| 2(G)(30) | (2R, 3R, 4S, 5S)-2-(6- amino-9H-purin-9-yl)-5- ({[5-(prop-2-yn-1-ylthio)- 1,3,4-thiodiazol-2- yl]thio}methyl)tetra- hydrofuran-3,4-diol | 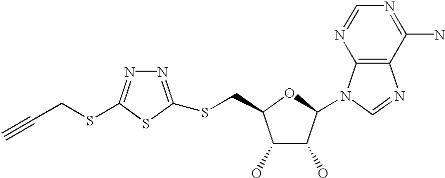 | 437.53 | 439 | 11 | 12 |
| 2(G)(31) | (2R, 3R, 4S, 5S)-2-(6- amino-9H-purin-9-yl)-5- {[(5-hydroxy-4-methyl- 4H-1,2,4-triazol-3-yl)- thio]methyl}tetrahydro- furan-3,4-diol | 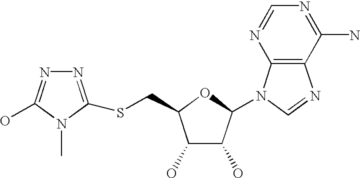 | 380.39 | 381 | 46 | 50 |
| 2(G)(32) | (2R, 3R, 4S, 5S)-2-(6- amino-9H-purin-9-yl)-5- {[(5,7-dimethyl[1,2,4]- triazolo[1,5-a]pyrimidin- 2-yl)thio]methyl}tetra- hydrofuran-3,4-diol | 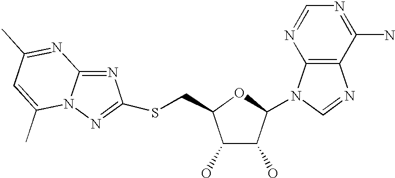 | 429.46 | 430 | 6 | 7 |
| 2(G)(33) | (2R, 3R, 4S, 5S)-2-(6- amino-9H-purin-9-yl)-5- ({[4-(trifluoromethyl)-pyr- imidin-2-yl]thio}methyl)- tetrahydrofuran-3,4-diol | 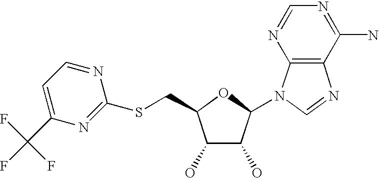 | 429.38 | 430 | 28 | 36 |
| 2(G)(34) | (2R, 3R, 4S, 5S)-2-(6- amino-9H-purin-9-yl)-5- {[(5-tert-butyl-2-methyl- phenyl)thio]methyl}- tetrahydrofuran-3,4-diol | 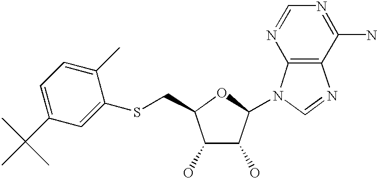 | 429.54 | 431 | 2 | 3 |
| 2(G)(35) | (2R, 3R, 4S, 5S)-2-(6- amino-9H-purin-9-yl)-5- {[(4-isopropylphenyl)- thio]methyl}tetrahydro- furan-3,4-diol | 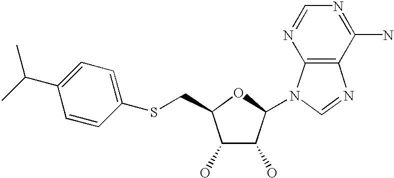 | 401.49 | 402 | 15 | 11 |
| 2(G)(36) | ethyl 4-amino-2- ({](2S, 3S, 4R, 5R)-5-(6- amino-9H-purin-9-yl)- 3-4-dihydroxytetrahydro- furan-2-yl]methyl}thio)- pyrimidine-5-carboxylate | 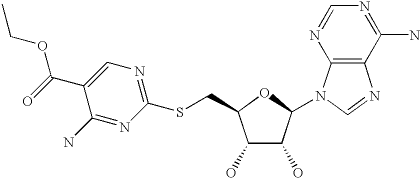 | 448.46 | 449 | 35 | 40 |
| 2(G)(37) | (2R, 3R, 4S, 5S)-2-(6- amino-9H-purin-9-yl)-5- {[(2-methyl-3- furyl)thio]methyl}tetra- hydrofuran-3,4-diol | 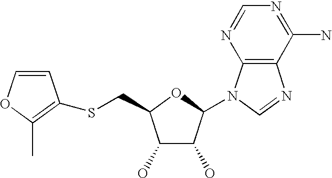 | 363.40 | 364 | 10 | 26 |
| 2(G)(38) | (2R, 3R, 4S, 5S)-2-(6- amino-9H-purin-9-yl)-5- {[(2,2,2-trifluoroethyl)- thio]methyl}tetrahydro- furan-3,4-diol | 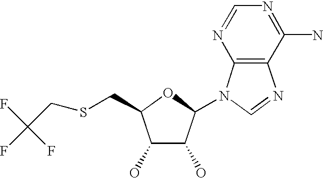 | 365.34 | 366 | 30 | 32 |
| 2(G)(39) | tert-butyl [2- ({[(2S, 3S, 4R, 5R)-5-(6- amino-9H-purin-9-yl)- 3,4-dihydroxytetrahydro- furan-2-yl]methyl}thio)- ethyl]carbamate | 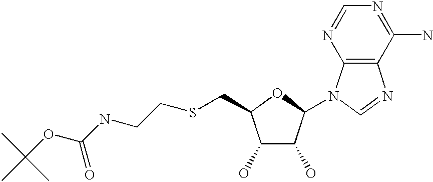 | 426.50 | 427 | 7 | 8 |
| 2(G)(40) | 7-({[(2S, 3S, 4R, 5R)-5-(6- amino-9H-purin-9-yl)- 3,4-dihydroxytetrahydro- furan-2-yl]methyl}thio)- 4-methyl-2H-chromen- 2-one | 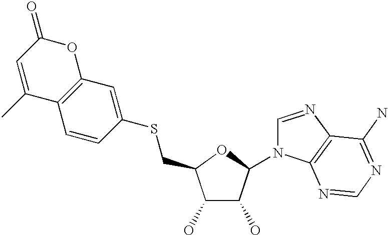 | 441.47 | 442 | 6 | 10 |
| 2(G)(41) | (2R, 3R, 4S, 5S)-2-(6- amino-9H-purin-9-yl)-5- ({[3-chloro-5- (trifluoromethyl)pyridin- yl]thio}methyl)tetra- hydrofuran-3,4-diol | 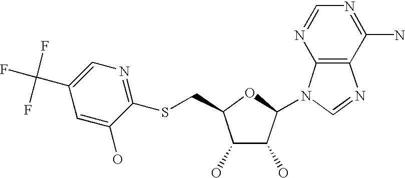 | 462.84 | 463/ 465 | 7 | 7 |
| 2(G)(42) | (2R, 3R, 4S, 5S)-2-(6- amino-9H-purin-9-yl)-5- [(quinolin-2-ylthio)methyl]- tetrahydrofuran-3,4-diol | 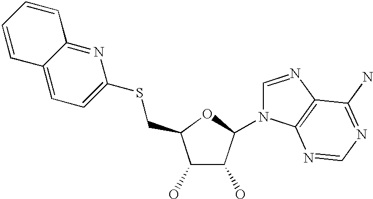 | 410.46 | 411 | 38 | 47 |
| 2(G)(43) | 2-({[(2S, 3S, 4R, 5R)-5-(6- amino-9H-purin-9-yl)- dihydroxytetrahydro- furan-2-yl]methyl}thio)- 4,6-dimethylnicotino- nitrile | 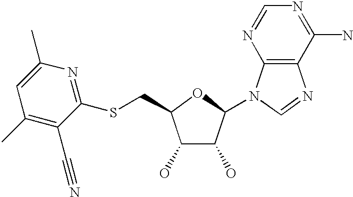 | 413.46 | 414 | 5 | 7 |
| 2(G)(44) | (2R, 3R, 4S, 5S)-2-(6- amino-9H-purin-9-yl)-5- [(allythio)methyl]tetra- hydrofuran-3,4-diol | 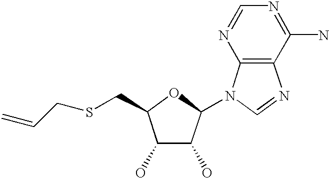 | 323.38 | 324 | 77 | 82 |
| 2(G)(45) | (2R, 3R, 4S, 5S)-2-(6- amino-9H-purin-9-yl)-5- [(isopropylthio)methyl]- tetrahydrofuran-3,4-diol | 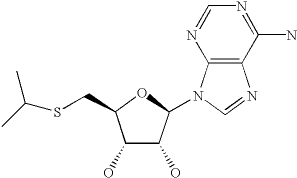 | 325.39 | 326 | 53 | 57 |
| 2(G)(46) | (2R, 3R, 4S, 5S)-2-(6- amino-9H-purin-9-yl)-5- {[(4-methyl-1H- benzimidazol-2-yl)- thio]methyl}tetrahydro- furan-3,4-diol | 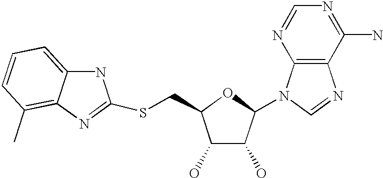 | 413.46 | 414 | 42 | 45 |
| 2(G)(47) | (2R, 3R, 4S, 5S)-2-(6- amino-9H-purin-9-yl)-5- [(1H-imidazo[4,5-c]- pyridin-2-ylthio)methyl]- tetrahydrofuran-3,4-diol | 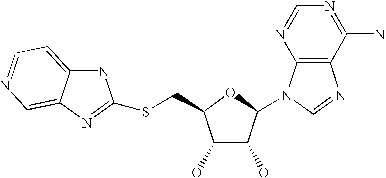 | 400.42 | 401 | 49 | 50 |
| 2(G)(48) | (2R, 3R, 4S, 5S)-2-(6- amino-9H-purin-9-yl)-5- {[(5-methyl-1H- benzimidazol-2-yl)thio]- methyl}tetrahydro- furan-3,4-diol | 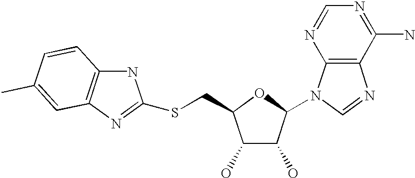 | 413.46 | 414 | 3 | 5 |
| 2(G)(49) | (2R, 3R, 4S, 5S)-2-(6- amino-9H-purin-9-yl)-5- {[(4-hydroxy-1H- pyrazolo[3,4-d]pyrimidin-6- yl)thio]methyl}tetra- hydrofuran-3,4-diol | 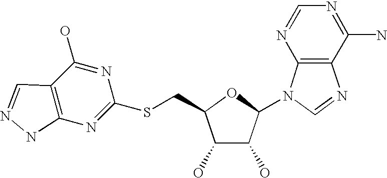 | 417.41 | 418 | 52 | 46 |
| 2(G)(50) | 2-({[(2S, 3S, 4R, 5R)-2-(6- amino-9H-purin-9-yl)- dihydroxytetrahydro- furan-2-yl]methyl}thio)- quinazolin-4-(3H)-one | 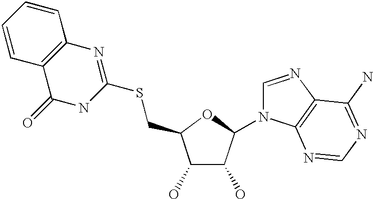 | 427.44 | 428 | 9 | 37 |
| 2(G)(51) | (2R, 3R, 4S, 5S)-2-(6- amino-9H-purin-9-yl)-5- {[(5-amino-1H- benzoimidazol-2- yl)thio]methyl}tetrahydro- furan-3,4-diol | 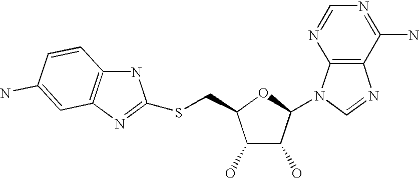 | 414.45 | 415 | 16 | 36 |
| 2(G)(52) | (2R, 3R, 4S, 5S)-2-(6- amino-9H-purin-9-yl)-5- {[(5-methyl-1,3,4- thiadiazol-2- yl)thio]methyl}tetrahydro- furan-3,4-diol | 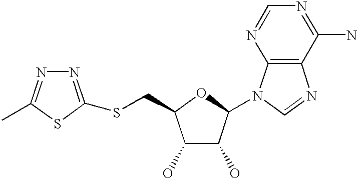 | 381.44 | 382 | 19 | 23 |
| 2(G)(53) | (2R, 3R, 4S, 5S)-2-(6- amino-9H-purin-9-yl)-5- [(1H-1,2,4-triazol-3- ylthio)methyl]tetrahydro- furan-3,4-diol | 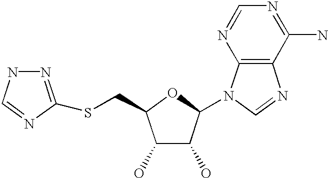 | 350.36 | 351 | 58 | 57 |
| 2(G)(54) | methyl ({[(2S, 3S, 4R, 5R)- 5-(6-amino-9H-purin-9- yl)-3,4-dihydroxy- tetrahydrofuran-2- yl]methyl}thio)acetate | 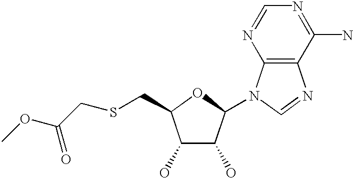 | 355.37 | 356 | 36 | 44 |
| 2(G)(55) | (2R, 3R, 4S, 5S)-2-(6- amino-9H-purin-9-yl)-5- {[(4-amino-1,3,5-triazin- 2-yl)thio]methyl}tetra- hydrofuran-3,4-diol | 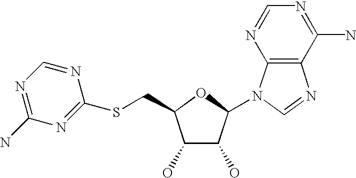 | 377.39 | 378 | 43 | 47 |
| 2(G)(56) | 2-({[(2S, 3S, 4R, 5R)-5-(6- amino-9H-purin-9-yl)- dihydroxytetrahydro furan-2-yl]methyl}thio)- N-methylacetamide | 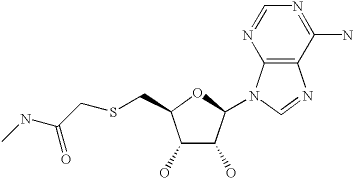 | 354.39 | 355 | 6 | 10 |
| 2(G)(57) | (2R, 3R, 4S, 5S)-2-(6- amino-9H-purin-9-yl)-5- hydroybutyl)thio]methyl}tetrahydrofuran-3,4- diol | 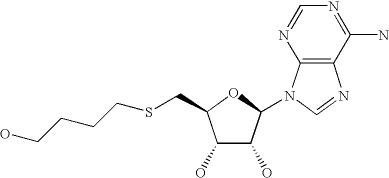 | 355.42 | 356 | 31 | 45 |
| 2(G)(58) | (2R, 3R, 4S, 5S)-2-(6- amino-9H-purin-9-yl)-5- {[(2-pyridin-4- ylethyl)thio]methyl}tetra- hydrofuran-3,4-diol | 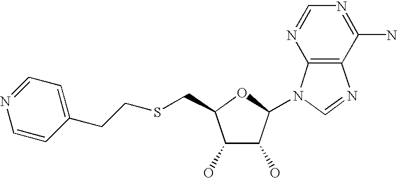 | 388.45 | 389 | 38 | 47 |
| 2(G)(59) | (2R, 3R, 4S, 5S)-2-(6- amino-9H-purin-9-yl)-5- {[(3-aminopyridin-2- yl)thio]methyl}tetra- hydrofuran-3,4-diol | 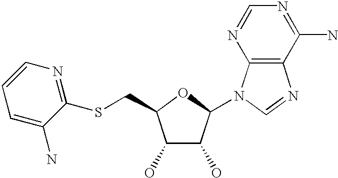 | 375.41 | 376 | 18 | 47 |
| 2(G)(60) | 2-({[(2S, 3S, 4R, 5R)-5-(6- amino-9H-purin-9-yl)- 3,4-dihydroxytetrahydro- furan-2-yl]methyl}thio)- nicotinamide | 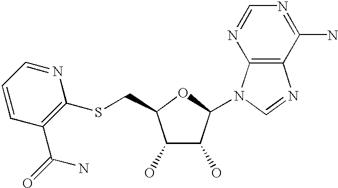 | 403.42 | 404 | 4 | 8 |
| 2(G)(61) | (2R, 3R, 4S, 5S)-2-(6- amino-9H-purin-9-yl)-5- {](2-pyrazin-2- ylethyl)thio]methyl}tetra- hydrofuran-3,4-diol | 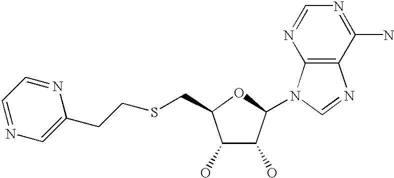 | 389.44 | 390 | 15 | 20 |
| 2(G)(62) | (2R, 3R, 4S, 5S)-2-(6- amino-9H-purin-9-yl)-5- {[(2-methyltetra- hydrofuran-3-yl)thio]methyl}tetrahydrofuran- 3,4-diol | 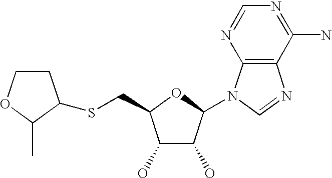 | 367.43 | 368 | 6 | 7 |
| 2(G)(63) | (2R, 3R, 4S, 5S)-2-(6- amino-9H-purin-9-yl)-5- ({[5-(hydroxymethyl)-1- methyl-1H-imidazol-2- yl]thio}methyl)tetra- hydrofuran-3,4-diol | 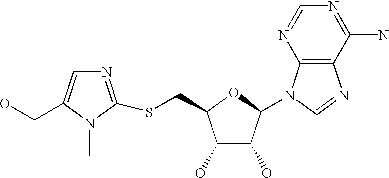 | 393.43 | 394 | 5 | 5 |
| 2(G)(64) | (2R, 3R, 4S, 5S)-2-(6- amino-9H-purin-9-yl)-5- {[(4-hydroxy-7H- pyrrolo[2,3-d]pyrimidin- 2-yl)thio]methyl}tetra- hydrofuran-3,4-diol | 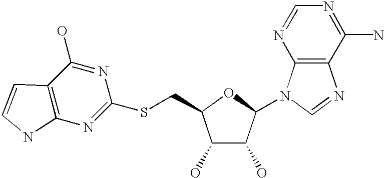 | 416.62 | 417 | 48 | 48 |
| 2(G)(65) | (2R, 3R, 4S, 5S)-2-(6- amino-9H-purin-9-yl)-5- {[(5-hydroxy-4- isopropyl-4H-1,2,4- triazol-3-yl)thio]methyl}tetrahydrofuran-3,4-diol | 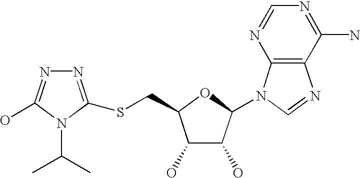 | 408.44 | 409 | 5 | 4 |
| 2(G)(66) | (2R, 3R, 4S, 5S)-2-(6- amino-9H-purin-9-yl)-5- [({5-[(dimethylamino)- methyl]-4-methyl-4H- 1,2,4-triazol-3-yl}thio)methyl]tetrahydro- furan-3,4-diol | 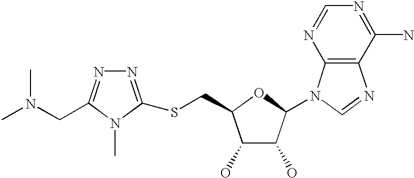 | 421.48 | 422 | 6 | 6 |
| 2(G)(67) | (2R, 3R, 4S, 5S)-2-(6- amino-9H-purin-9-yl)-5- {[(4,5-dimethyl-4H- 1,2,4-triazol-3- yl)thio]methyl}tetra- hydrofuran-3,4-diol | 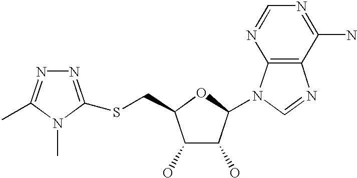 | 378.42 | 379 | 45 | 47 |
| 2(G)(68) | (2R, 3R, 4S, 5S)-2-(6- amino-9H-purin-9-yl)-5- [(sec- butylthio)methyl]tetra- hydrofuran-3,4-diol | 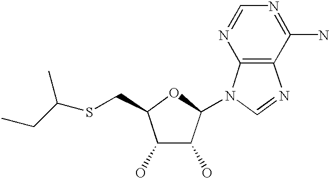 | 339.42 | 340 | 42 | 45 |
| 2(G)(69) | (2R, 3R, 4S, 5S)-2-(6- amino-9H-purin-9-yl)-5- [(pyrazin-2- ylthio)methyl]tetrahydro- furan-3,4-diol | 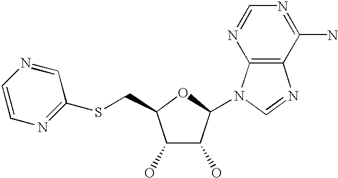 | 361.38 | 362 | 31 | 40 |
| 2(G)(70) | (2R, 3R, 4S, 5S)-2-(6- amino-9H-purin-9-yl)-5- {[(2-bromophenyl)- thio]methyl}tetrahydro- furan-3,4-diol | 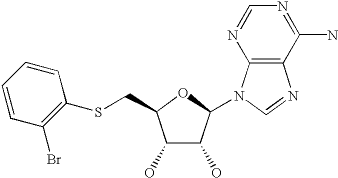 | 438.30 | 438/ 440 | 6 | 3 |
| 2(G)(71) | (2R, 3R, 4S, 5S)-2-(6- amino-9H-purin-9-yl)-5- {[(2-methylbutyl)- thio]methyl}tetrahydro- furan-3,4-diol | 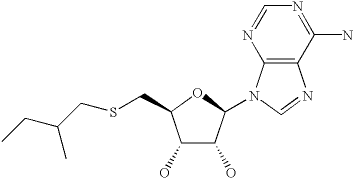 | 353.45 | 354 | 77 | 73 |
| 2(G)(72) | (2R, 3R, 4S, 5S)-2-(6- amino-9H-purin-9-yl)-5- {[(3-aminophenyl)thio]- methyl}tetrahydrofuran- 3,4-diol | 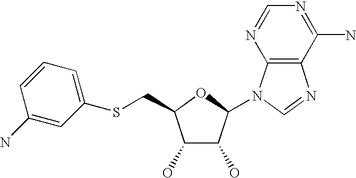 | 374.42 | 375 | 33 | 38 |
| 2(G)(73) | (2R, 3R, 4S, 5S)-2-(6- amino-9H-purin-9-yl)-5- {[(2-chlorobenzyl)thio]- methyl}tetrahydrofuran- 3,4-diol | 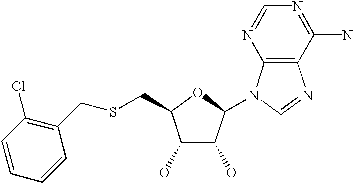 | 407.88 | 408/ 410 | 30 | 21 |
| 2(G)(74) | (2R, 3R, 4S, 5S)-2-(6- amino-9H-purin-9-yl)-5- ({[3-(trifluoromethyl)- benzylthio}methyl)tetra- hydrofuran-3,4-diol | 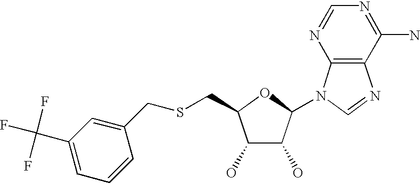 | 441.43 | 442 | 23 | 22 |
| 2(G)(75) | (2R, 3R, 4S, 5S)-2-(6- amino-9H-purin-9-yl)-5- hydroxypropyl)thio]- methyl}tetrahydrofuran- 3,4-diol | 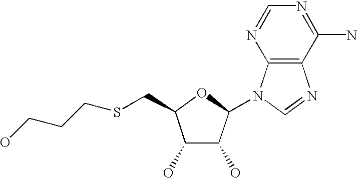 | 341.39 | 342 | 32 | 39 |
| 2(G)(76) | (2R, 3R, 4S, 5S)-2-(6- amino-9H-purin-9-yl)-5- {[(2,4-dichlorobenzyl)- thio]methyl}tetrahydro- furan-3,4-diol | 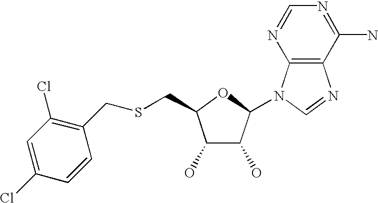 | 442.33 | 442/ 444/ 446 | 14 | 11 |
| 2(G)(77) | (2R, 3R, 4S, 5S)-2-(6- amino-9H-purin-9-yl)-5- hydroxyethyl)butyl]thio}methyl)tetrahydrofuran- 3,4-diol | 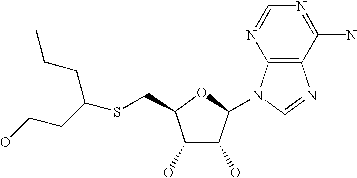 | 383.47 | 384 | 3 | 6 |
| 2(G)(78) | (2R, 3P, 4S, 5S)-2-(6- amino-9H-purin-9-yl)-5- hydroxyhexyl)thio]methyl}tetrahydrofuran-3,4-diol | 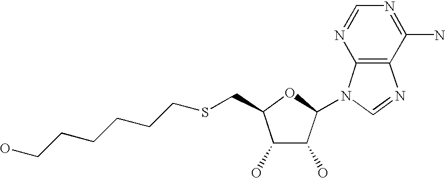 | 383.47 | 384 | 3 | 6 |
| 2(G)(79) | (2R, 3R, 4S, 5S)-2-(6- amino-9H-purin-9-yl)-5- {[(4-methyl-1,3-thiozol- 2-yl)thio]methyl}tetrahydrofuran-3,4-diol | 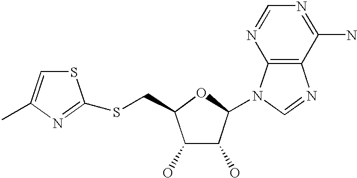 | 380.45 | 381 | 38 | 45 |
| 2(G)(80) | (2R, 3R, 4S, 5S)-2-(6- amino-9H-purin-9-yl)-5- ethylphenyl)thio]methyl}tetrahydrofuran-3,4-diol | 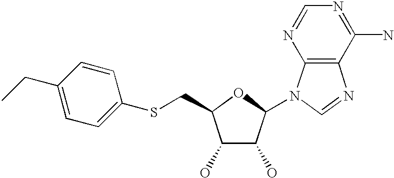 | 387.46 | 388 | 13 | 15 |
| 2(G)(81) | (2R, 3R, 4S, 5S)-2-(6- amino-9H-purin-9-yl)-5- ({[2-(1H-indol-3- yl)ethyl]thio}methyl)- tetrahydrofuran-3,4-diol | 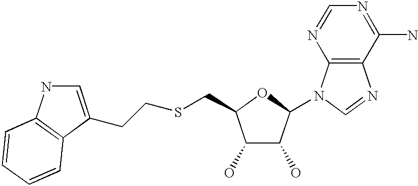 | 426.50 | 427 | 18 | 18 |
| 2(G)(82) | (2R, 3R, 4S, 5S)-2-(6- amino-9H-purin-9-yl)-5- ({[2-(trifluoromethyl)- phenyl]thio}methyl)tetra- hydrofuran-3,4-diol | 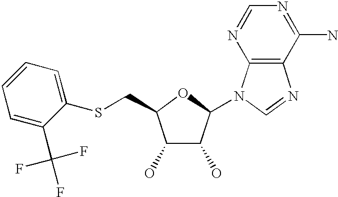 | 427.41 | 428 | 1 | 1 |
| 2(G)(83) | (2R, 3R, 4S, 5S)-2-(6- amino-9H-purin-9-yl)-5- {[(2,4-dimethoxybenzyl)- thio]methyl}tetrahydro- furan-3,4-diol | 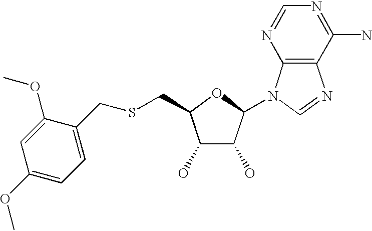 | 433.49 | 434 | 5 | 8 |
| 2(G)(84) | (2R, 3R, 4S, 5S)-2-(6- amino-9H-purin-9-yl)-5- dimethylphenyl)thio]- methyl}tetrahydrofuran- 3,4-diol | 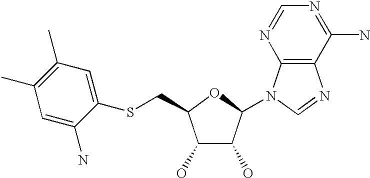 | 402.48 | 403 | 4 | 5 |
| 2(G)(85) | (2R, 3R, 4S, 5S)-2-(6- amino-9H-purin-9-yl)-5- [([1,3]thiozolo[5,4- b]pyridin-2-ylthio)- methyl]tetrahydrofuran- 3,4-diol | 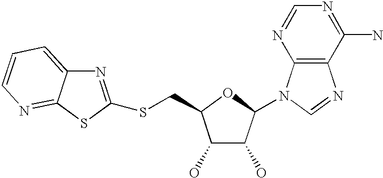 | 417.47 | 418 | 10 | 2 |
| 2(G)(86) | (2R, 3R, 4S, 5S)-2-(6- amino-9H-purin-9-yl)-5- {[(5-methoxy-1,3- benzothiozol-2-yl)thio]- methyl}tetrahydro- furan-3,4-diol | 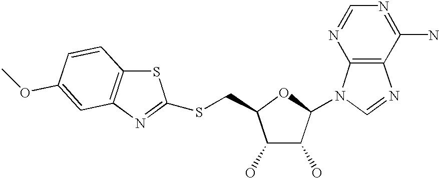 | 446.51 | 448 | 31 | 33 |
| 2(G)(87) | (2R, 3R, 4S, 5S)-2-(6- amino-9H-purin-9-yl)-5- [(2-thienylthio)methyl]- tetrahydrofuran-3,4-diol | 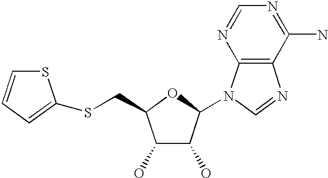 | 365.44 | 366 | 36 | 33 |
| 2(G)(88) | ethyl ({[(2S, 3S, 4R, 5R)-5- (6-amino-9H-purin-9-yl)- 3,4-dihydroxytetrahydro- furan-2-yl]methyl}thio)acetate | 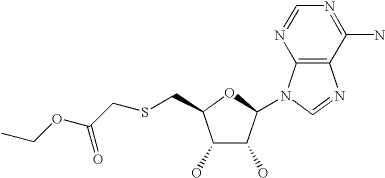 | 369.40 | 370 | 25 | 33 |
| 2(G)(89) | 2-({[(2S, 3S, 4R, 5R)-5-(6- amino-9H-purin-9-yl)- 3,4-dihydroxytetrahydro- furan-2-yl]methyl}thio)nicotinonitrile | 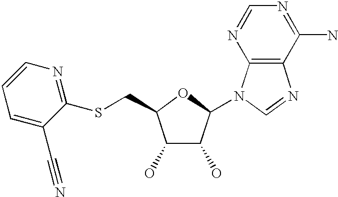 | 385.41 | 386 | 3 | 5 |
| 2(G)(90) | 3-({[(2S, 3S, 4R, 5R)-5-(6- amino-9H-purin-9-yl)- 3,4-dihydroxytetrahydro- furan-2-yl]methyl}thio)benzoic acid | 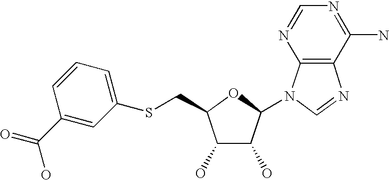 | 403.42 | 404 | 3 | 8 |
| 2(G)(91) | (2R, 3R, 4S, 5S)-2-(6- amino-9H-purin-9-yl)-5- {[(2-nitrophenyl)thio]- methyl}tetrahydrofuran- 3,4-diol | 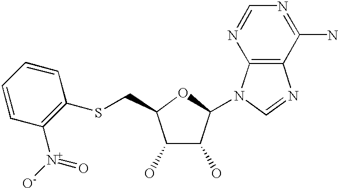 | 404.41 | 405 | 5 | 5 |
| 2(G)(92) | methyl 3-({[(2S, 3S, 4R, 5R)-5-(6-amino-9H-purin- 9-yl)-dihydroxytetra- hydrofuran-2-yl]- methyl}thio)propanoate | 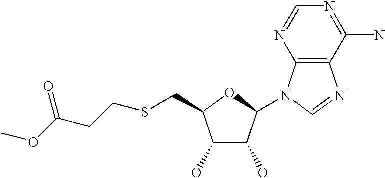 | 369.40 | 370 | 27 | 36 |
| 2(G)(93) | (2R, 3R, 4S, 5S)-2-(6- amino-9H-purin-9-yl)-5- {[(1-benzothien-3- ylmethyl)thio]methyl}tetrahydrofuran-3,4-diol | 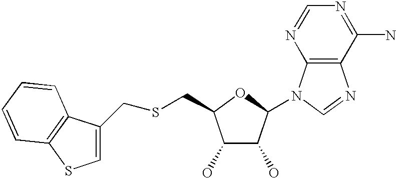 | 429.52 | 431 | 18 | 17 |
| 2(G)(94) | (2R, 3R, 4S, 5S)-2-(6- amino-9H-purin-9-yl)-5- ({[3-(2-phenylethyl)- pyrazin-2-yl]thio}methyl)tetrahydrofuran- 3,4-diol | 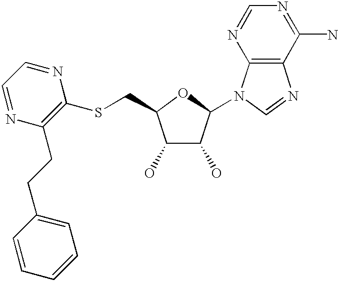 | 465.54 | 467 | 5 | 5 |
| 2(G)(95) | 4-({[(2S, 3S, 4R, 5R)-5-(6- amino-9H-purin-9-yl)- 3,4-dihydroxytetrahydro- furan-2-yl]methyl}thio)- benzoic acid | 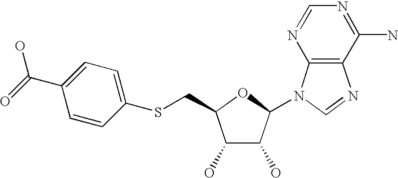 | 403.42 | 404 | 7 | 7 |
| 2(G)(96) | (2R, 3R, 4S, 5S)-2-(6- amino-9H-purin-9-yl)-5- {((2-chlorophenyl)thio]- methyl}tetrahydrofuran- 3,4-diol | 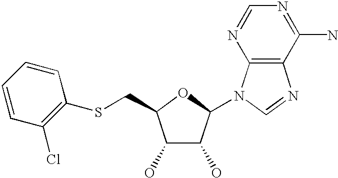 | 393.85 | 394/ 396 | 5 | 6 |
| 2(G)(97) | (2R, 3R, 4S, 5S)-2-(6- amino-9H-purin-9-yl)-5- {[(2,5-dichlorophenyl)- thio]methyl}tetrahydro- furan-3,4-diol | 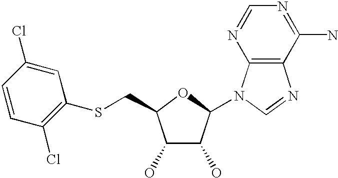 | 428.30 | 428/ 430/ 432 | 5 | 6 |
| 2(G)(98) | (2R, 3R, 4S, 5S)-2-(6- amino-9H-purin-9-yl)-5- {[(3-chlorophenyl)thio]- methyl}tetrahydrofuran- 3,4-diol | 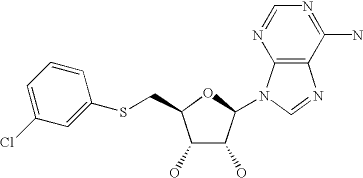 | 393.85 | 394/ 396 | 20 | 18 |
| 2(G)(99) | (2R, 3R,4S, 5S)-2-(6- amino-9H-purin-9-yl)-5- ({[3-(trifluoromethyl)- phenyl]thio}methyl)- tetrahydrofuran-3,4-diol | 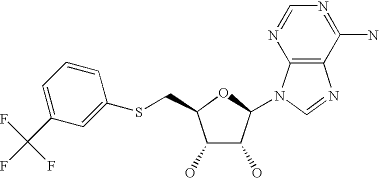 | 427.41 | 428 | 17 | 18 |
| 2(G)(100) | (2R, 3R, 4S, 5S)-2-(6- amino-9H-purin-9-yl)-5- {[(3-methylpyrazin-2- yl)thio]methyl}tetrahydro- furan-3,4-diol | 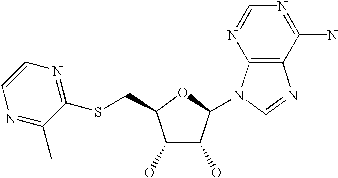 | 375.41 | 376 | 7 | 10 |
| 2(G)(101) | (2R, 3R, 4S, 5S)-2-(6- amino-9H-purin-9-yl)-5- {[(3-hydroxyphenyl)- thio]methyl}tetrahydro- furan-3,4-diol | 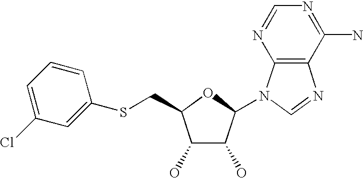 | 375.41 | 376 | 36 | 38 |
| 2(G)(102) | (2R, 3R, 4S, 5S)-2-(6- amino-9H-purin-9-yl)-5- {[(2,6-dichIorophenyl)- thio]methyl}tetrahydro- furan-3,4-diol | 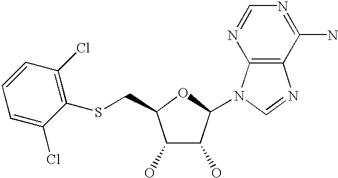 | 428.30 | 428/ 430/ 432 | 2 | 3 |
| 2(G)(103) | (2R, 3R, 4S, 5S)-2-(6- amino-9H-purin-9-yl)-5- ({[2-nitro-4- (trifluoromethyl)phenyl]thio}methyl)tetrahydro- furan-3,4-diol | 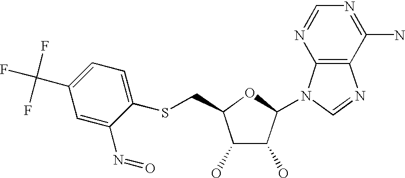 | 472.40 | 473 | 3 | 4 |
| 2(G)(104) | 2-({[(2S, 3S, 4R, 5R)-5- (6-amino-9H-purin-9-yl)- 3,4-dihydroxytetrahydro- furan-2-yl]methyl}thio)- N-phenylacetamide | 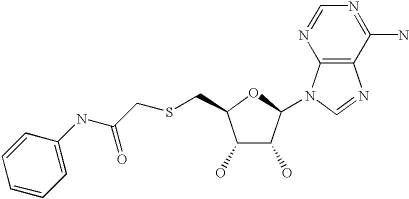 | 416.46 | 417 | 5 | 15 |
| 2(G)(105) | (2R, 3R, 4S, 5S)-2-(6- amino-9H-purin-9-yl)-5- {[(5-nitropyridin-2- yl)thio]methyl}tetrahydro- furan-3,4-diol | 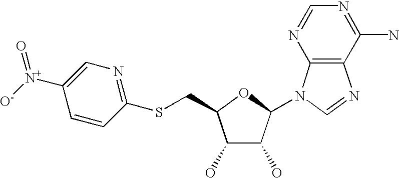 | 405.39 | 406 | 17 | 21 |
| 2(G)(106) | (2R, 3R, 4S, 5S)-2-(6- amino-9H-purin-9-yl)-5- [(1H-indol-3-ylthio)- methyl]-tetrahydrofuran- 3,4-diol | 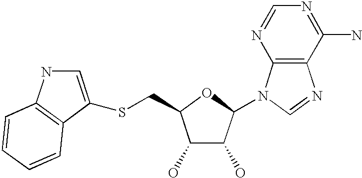 | 398.45 | 399 | 6 | 3 |
| 2(G)(107) | methyl 2-({[(2S, 3S, 4R, 5R)-5-(6-amino-9H-purin- 9-yl)-3,4-dihydroxy- tetrahydrofuran-2-yl]- methyl}thio)- benzoate | 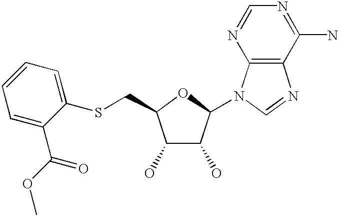 | 417.44 | 418 | 4 | 2 |
| 2(G)(108) | (2E)-3-[4-({[(2S, 3S, 4R, 5R)-5-(6-amino-9H-purin- 9-yl)-3,4-dihydroxytetra- hydrofuran-2-yl]methyl}thio)phenyl]acrylic acid | 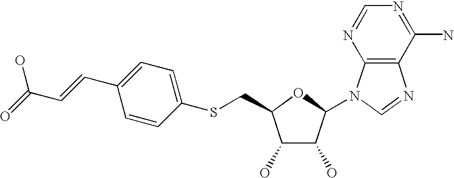 | 429.46 | 430 | 8 | 19 |
| 2(G)(109) | methyl 3-({[(2S, 3S, 4R, 5R)-5-(6-amino-9H-purin- 9-yl)-1,3,4-dihydroxytetra- hydrofuran-2-yl]methyl}thio)benzoate | 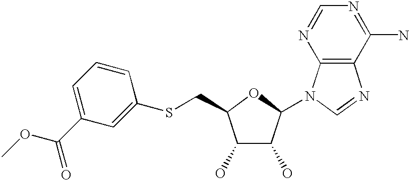 | 417.44 | 418 | 8 | 8 |
| 2(G)(110) | methyl (2E)-3-[4-({[(2S, 3S, 4R, 5R)-5-(6-amino- 9H-purin-9-yl)-dihydroxy- tetrahydrofuran-2-yl]- methyl}thio)phenyl]- acrylate | 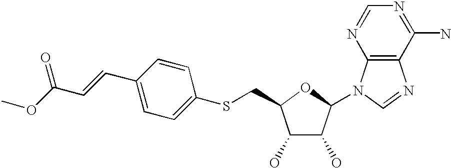 | 443.48 | 444 | 15 | 9 |
| 2(G)(111) | (2R, 3R, 4S, 5S)-2-(6- amino-9H-purin-9-yl)-5- ({[5-(3-methoxyphenyl)- 4-methyl-4H-1,2,4- triazol-3-yl]thio}- methyl)tetrahydrofuran- 3,4-diol | 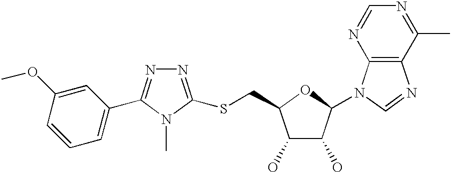 | 470.51 | 472 | 8 | 4 |
| 2(G)(112) | (2R, 3R, 4S, 5S)-2-(6- amino-9H-purin-9-yl)-5- ({[4-(2-furyl)pyrimidin-2- yl]thio}methyl)tetrahydro- furan-3,4-diol | 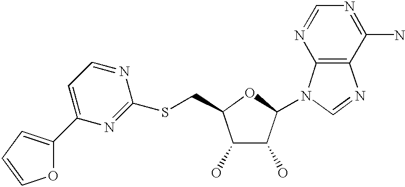 | 427.44 | 428 | 17 | 10 |
| 2(G)(113) | (2R, 3R, 4S, 5S)-2-(6- amino-9H-purin-9-yl)-5- {[(1-methyl-1H- benzimidazol-2-yl)thio]- methyl}tetrahydro- furan-3,4-diol | 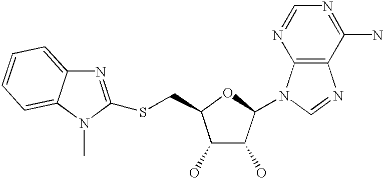 | 413.46 | 414 | 48 | 43 |
| 2(G)(114) | N-[2-({[(2S, 3S, 4R, 5R)-5-(6-amino-9H-purin- 9-yl)-3,4-dihydroxytetra- hydrofuran-2-yl]methyl}thio)ethyl]acetamide | 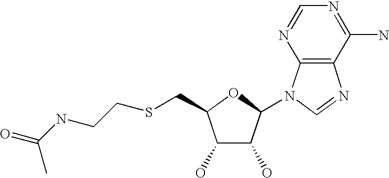 | 368.42 | 369 | 29 | 11 |
| 2(G)(115) | (2R, 3R, 4S, 5S)-2-(6- amino-9H-purin-9-yl)-5- ({[4-(methylthio)phenyl]- thio}methyl)tetrahydro- furan-3,4-diol | 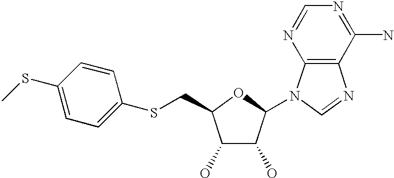 | 405.50 | 407 | 12 | 15 |
| 2(G)(116) | (2R, 3R, 4S, 5S)-2-(6- amino-9H-purin-9-yl)-5- ({[2-(trifluoromethoxy)- phenyl]thio}methyl)tetra- hydrofuran-3,4-diol | 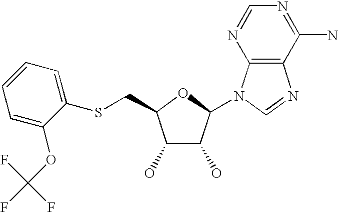 | 443.40 | 444 | 3 | 7 |
| 2(G)(117) | (2R, 3R, 4S, 5S)-2-(6- amino-9H-purin-9-yl)- 5-{[(2-fluorophenyl)thio)- methyl}tetrahydrofuran- 3,4-diol | 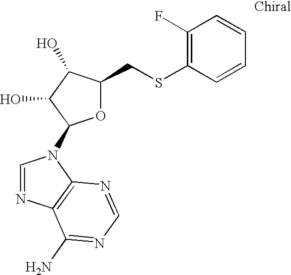 | 377.41 | 378 | 25 | 28 |
| 2(G)(118) | (2R, 3R, 4S, 5S)-2-(6- amino-9H-purin-9-yl)- 5-{[(5-methoxy-1H- benzimidazol-2-yl)thio]methyl}tetrahydrofuran- 3,4-diol | 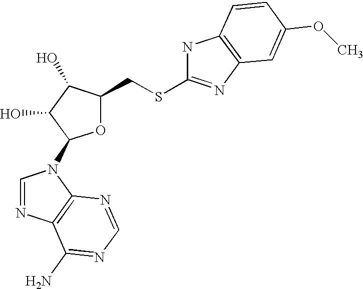 | 429.47 | 430 | 2.5 | 2.5 |
| 2(G)(119) | (2R, 3R, 4S, 5S)-2-(6- amino-9H-purin-9-yl)- 5-[(1H-benzimidazol-2- ylthio)methyl)tetrahydro- furan-3,4-diol | 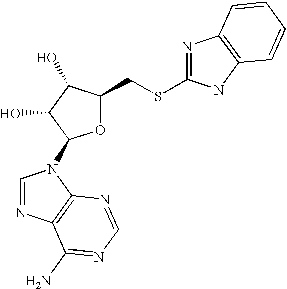 | 399.44 | 400 | 12 | 26 |
| 2(G)(120) | (2R, 3R, 4S, 5S)-2-(6- amino-9H-purin-9-yl)- 5-{[(1-methyl-1H-imidazol- 2-yl)thio)methyl}tetra- hydrofuran-3,4-diol | 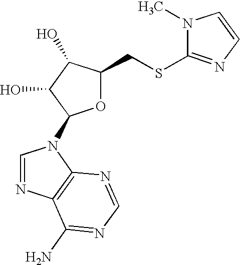 | 363.41 | 364 | 1 | 3 |
| 2(G)(121) | (2R, 3R, 4S, 5S)-2-(6- amino-9H-purin-9-yl)-5- [(nonylthio)methyl]tetra- hydrofuran-3,4-diol | 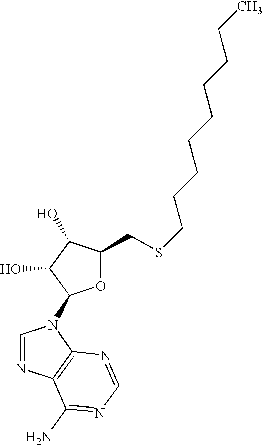 | 409.56 | 411 | 64.5 | 54.5 |
| 2(G)(122) | (2R, 3R, 4S, 5S)-2-(6- amino-9H-purin-9-yl)-5- [(1,3-benzoxazol-2-ylthio)- methyl]tetrahydrofuran- 3,4-diol | 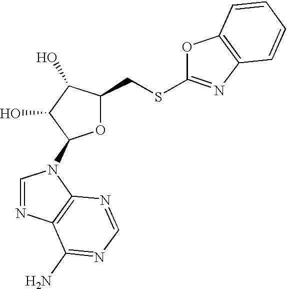 | 400.43 | 401 | 30.5 | 37 |
| 2(G)(123) | (5R)-5-[({[(2S, 3S, 4R, 5R)-5-(6-amino-9H-purin- 9-yl)-3,4-dihydroxytetra- hydrofuran-2-yl)methyl}thio)methyl]imidazo- lidine-2,4-dione | 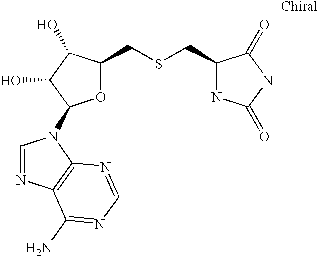 | 395.41 | 396 | 23 | 22 |
| 2(G)(124) | (2R, 3R, 4S, 5S)-2-(6- amino-9H-purin-9-yl)- 5-[{(4-chlorobenzyl)thio]- methyl}tetrahydrofuran- 3,4-diol | 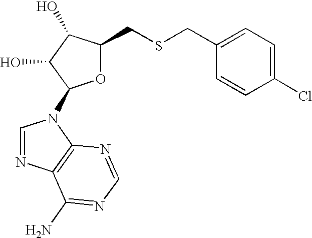 | 407.89 | 408/ 410 | 47 | 51 |
| 2(G)(125) | (2R, 3R, 4S, 5S)-2-(6- amino-9H-purin-9-yl)- 5-[(heptylthio)methyl]- tetrahydrofuran-3,4-diol | 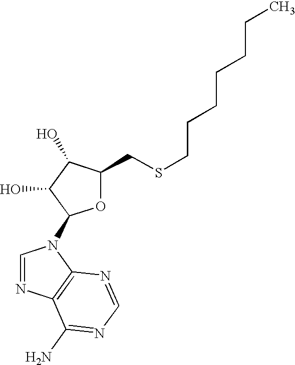 | 381.51 | 383 | 101 | 62.5 |
| 2(G)(126) | (2R, 3R, 4S, 5S)-2-(6- amino-9H-purin-9-yl)-5- [(hexylthio)methyl]tetra- hydrofuran-3,4-diol | 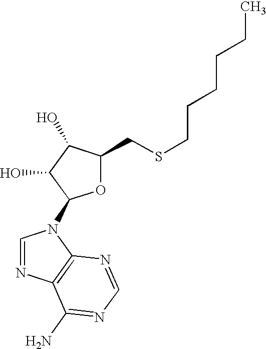 | 367.48 | 368 | 72 | 67.5 |
| 2(G)(127) | (2R, 3R, 4S, 5S)-2-(6- amino-9H-purin-9-yl)- 5-{[(2-fluorobenzyl)- thio]methyl}tetrahydro- furan-3,4-diol | 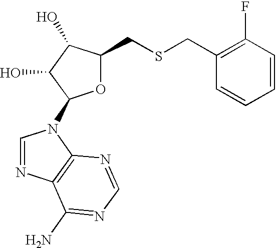 | 391.44 | 392 | 56 | 58.5 |
| 2(G)(128) | (2R, 3R, 4S, 5S)-2-(6- amino-9H-purin-9-yl)- 5{[(3,4-dichlorophenyl)- thio]methyl}tetrahydro- furan-3,4-diol | 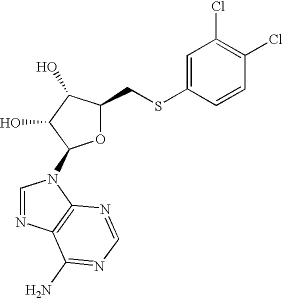 | 428.31 | 428/ 430/ 432 | 11 | 10.5 |
| 2(G)(129) | (2R, 3R, 4S, 5S)-2-(6- amino-9H-purin-9-yl)-5- [(decylthio)methyl]tetra- hydrofuran-3,4-diol | 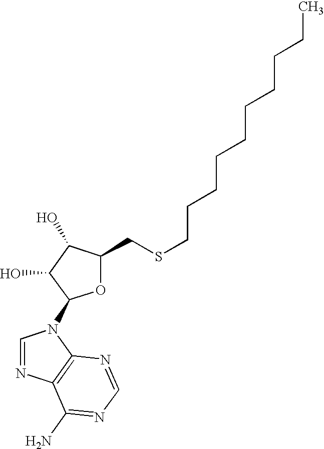 | 423.59 | 425 | 46 | 41.5 |
| 2(G)(130) | (2R, 3R, 4S, 5S)-2-(6- amino-9H-purin-9-yl)-5- {[(2,4-dichlorophenyl)- thio]methyl}tetrahydro- furan-3,4-diol | 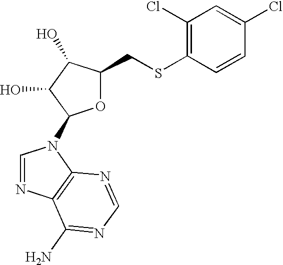 | 428.31 | 428/ 430/ 432 | −2 | 4.5 |
| 2(G)(131) | (2R, 3R, 4S, 5S)-2-(6- amino-9H-purin-9-yl)-5- {[(3,5-dichlorophenyl)- thio]methyl}tetrahydro- furan-3,4-diol | 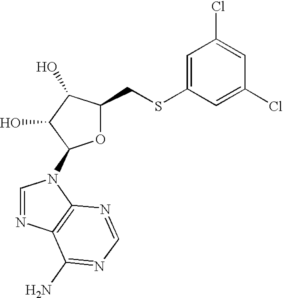 | 428.31 | 428/ 430/ 432 | 11.5 | 11 |
| 2(G)(132) | Ethyl 2-({[(2S, 3S, 4R, 5R)-5-(6-amino-9H-purin- 9-yl)-3,4-dihydroxytetra- hydrofuran-2-yl]methyl}thio)-1H-imidazole-4- carboxylate | 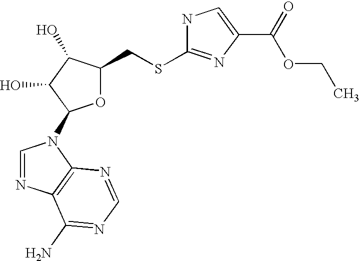 | 421.45 | 22 | 0 | 1.5 |
| 2(G)(133) | Butyl ({[(2S, 3S, 4R, 5R)- 5-(6-amino-9H-purin-9- yl)-3,4-dihydroxytetra- hydrofuran-2-yl]methyl}- thio)acetate | 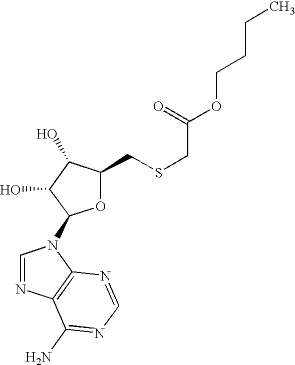 | 397.47 | 398 | 22.5 | 31.5 |
| 2(G)(134) | (2R, 3R, 4S, 5S)-2-(6- amino-9H-purin-9-yl)- 5-[(7H-purin-6-ylthio)- methyl)tetrahydrofuran- 3,4-diol | 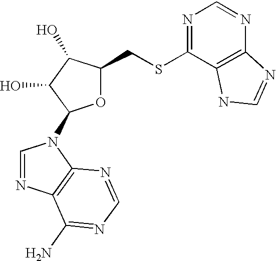 | 401.42 | 402 | | |
| 2(G)(135) | (2R, 3R, 4S, 5S)-2-(6- amino-9H-purin-9-yl)- 5-{[(5-methyl-1H- benzimidazol-2-yl)thio]- methyl}tetrahydro- furan-3,4-diol | 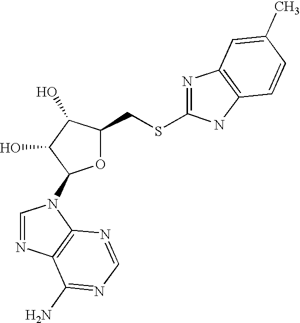 | 413.47 | 414 | | |
| 2(G)(136) | (2R, 3R, 4S, 5S)-2-(6- amino-9H-purin-9-yl)- 5-({[2-(butylamino)- ethyl]thio}methyl)- tetrahydrofuran-3,4-diol | 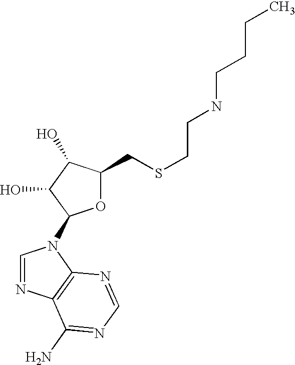 | 382.51 | 384 | 18 | 37.5 |
| 2(G)(137) | (2R, 3R, 4S, 5S)-2-(6- amino-9H-purin-9-yl)- 5-{[(mesitylmethyl)thio]- methyl}tetrahydrofuran- 3,4-diol | 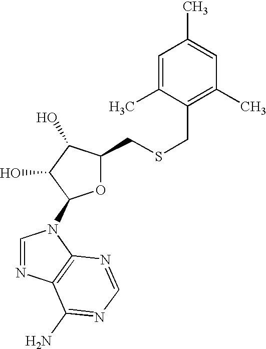 | 415.53 | 417 | 3.5 | 2 |
| 2(G)(138) | (2R, 3R, 4S, 5S)-2-(6- amino-9H-purin-9-yl)- 5-{[(4-phenyl-1,3- thiazol-2-yl)thio]methyl}- tetrahydrofuran-3,4-diol | 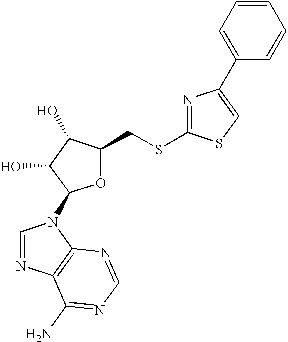 | 442.53 | 444 | 9 | 12.5 |
| 2(G)(139) | Butyl 3-({[(2S, 3S, 4R, 5R)-5-(6-amino-9H-purin- 9-yl)-dihydroxytetrahydro- furan-2-yl]methyl}thio)- propanoate | 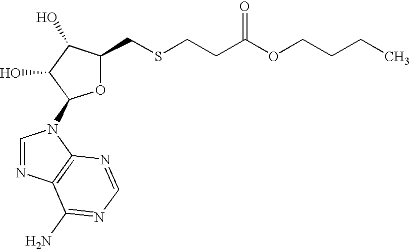 | 411.49 | 412 | 26.5 | 30 |
| 2(G)(140) | Ethyl 2-({[(2S, 3S, 4R, 5R)-5-(6-amino-9H-purin- 9-yl)-3,4-dihydroxytetra- hydrofuran-2-yl]methyl}- thio)-propanoate | 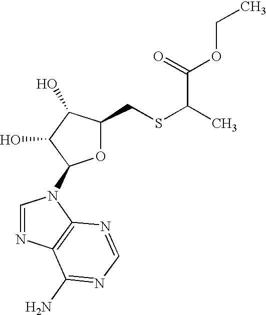 | 383.44 | 384 | 3 | 7.5 |
| 2(G)(141) | (2R, 3R, 4S, 5S)-2-(6- amino-9H-purin-9-yl)- 5-{[(2-hydroxypropyl)- thio]methyl}tetrahydro- furan-3,4-diol | 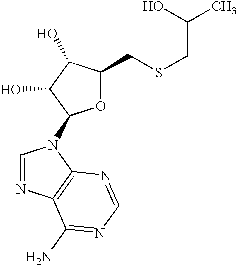 | 341.40 | 342 | 10 | 27 |
| 2(G)(142) | (2R, 3R, 4S, 5S)-2-(6- amino-9H-purin-9-yl)- 5-[(octylthio)methyl]tetra- hydrofuran-3,4-diol | 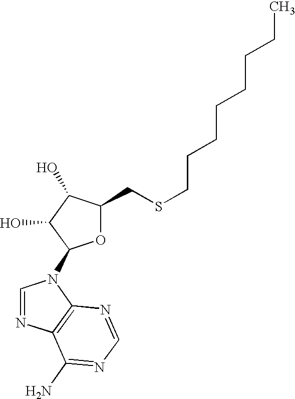 | 395.54 | 397 | 1.5 | 58 |
| 2(G)(143) | (2R, 3R, 4S, 5S)-2-(6- amino-9H-purin-9-yl)- 5-{[(2,3-dihydroxypropyl)- thio]methyl}tetrahydro- furan-3,4-diol | 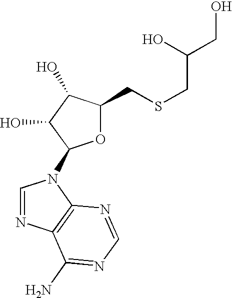 | 357.40 | 358 | 12 | 3 |
| 2(G)(143) | (2R, 3R, 4S, 5S)-2-(6- amino-9H-purin-9-yl)- 5-{[(2-chloro-6- fluorobenzyl)thio]methyl}- tetrahydrofuran-3,4-diol | 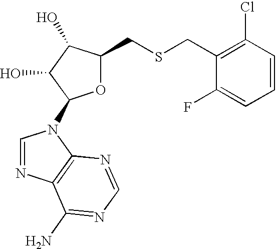 | 425.88 | 426/ 428 | 3 | 10.5 |
| 2(G)(144) | (2R, 3R, 4S, 5S)-2-(6- amino-9H-purin-9-yl)- 5-{[(2-hydroxy-1-methyl- propyl)thio]methyl}tetra- hydrofuran-3,4-diol | 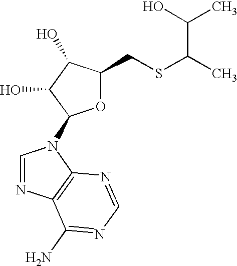 | 355.43 | 356 | 18 | 7.5 |
| 2(G)(145) | (2R, 3R, 4S, 5S)-2-(6- amino-9H-purin-9-yl)- 5-{[(3,4-dichlorobenzyl)- thio]methyl}tetrahydro- furan-3,4-diol | 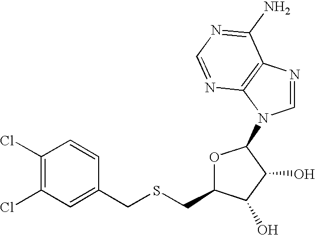 | 442.34 | 443 | 3.5 | 16 |
| 2(G)(146) | (2R, 3R, 4S, 5S)-2-(6- amino-9H-purin-9-yl)- 5-{[(2-isopropylphenyl)- thio]methyl}tetrahydro- furan-3,4-diol | 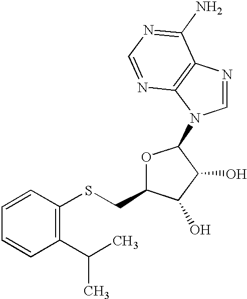 | 401.50 | 403 | 28 | 2 |
| 2(G)(147) | (2R, 3R, 4S, 5S)-2-(6- amino-9H-purin-9-yl)- 5-{[(3-fluorophenyl)- thio]methyl}tetrahydro- furan-3,4-diol | 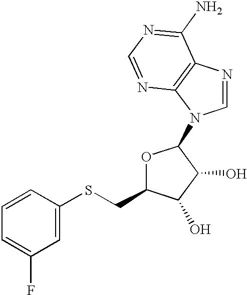 | 377.41 | 378 | 18.5 | 25.5 |
| 2(G)(148) | (2R, 3R, 4S, 5S)-2-(6- amino-9H-purin-9-yl)- 5-{[(3,5-dimethylphenyl)- thio]methyl}tetrahydro- furan-3,4-diol | 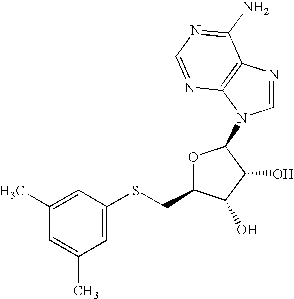 | 387.47 | 388 | 2 | 15 |
| 2(G)(149) | (2R, 3R, 4S, 5S)-2-(6- amino-9H-purin-9-yl)- 5-{[(2,4-dimethylphenyl)- thio]methyl}tetrahydro- furan-3,4-diol | 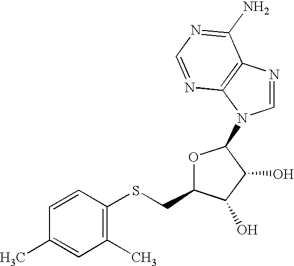 | 387.47 | 388 | 35.5 | 2.5 |
| 2(G)(150) | (2R, 3R, 4S, 5S)-2-(6- amino-9H-purin-9-yl)- 5-{[(3,4-dimethylphenyl)- thio]methyl}tetrahydro- furan-3,4-diol | 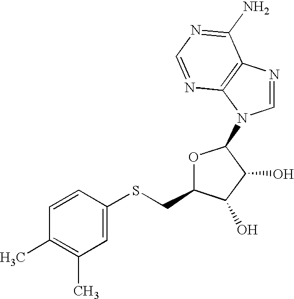 | 387.47 | 388 | 2 | 33.5 |
| 2(G)(151) | (2R, 3R, 4S, 5S)-2-(6- amino-9H-purin-9-yl)- 5-{[(2,3-dichlorophenyl)- thio]methyl}tetrahydro- furan-3,4-diol | 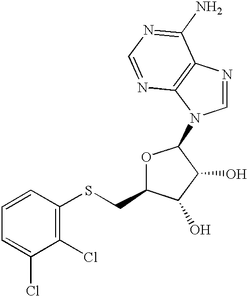 | 428.31 | 429 | 13.5 | 2 |
| 2(G)(152) | (2R, 3R, 4S, 5S)-2-(6- amino-9H-purin-9-yl)- 5-({[3-(methylthio)- 1,2,4-thiodiazol-5-yl)- thio}methyl)tetrahydro- furan-3,4-diol | 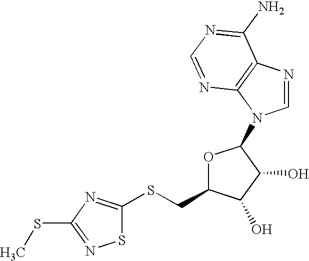 | 413.51 | 415 | 19.5 | 17 |
| 2(G)(153) | (2R, 3R, 4S, 5S)-2-(6- amino-9H-purin-9-yl)- 5-{[(6-chloro-1,3-benz- oxazol-2-yl)thio]methyl}tetrahydrofuran-3,4-diol | 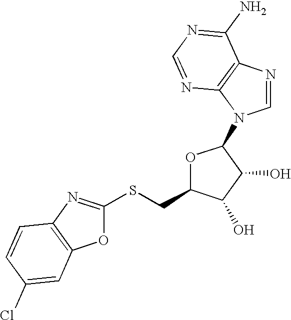 | 434.87 | 435/ 437 | 10 | 22.5 |
| 2(G)(154) | (2R, 3R, 4S, 5S)-2-(6- amino-9H-purin-9-yl)- 5-{[(4,6-dimethyl- pyrimidin-2-yl)thio]methyl}tetrahydrofuran-3,4-diol | 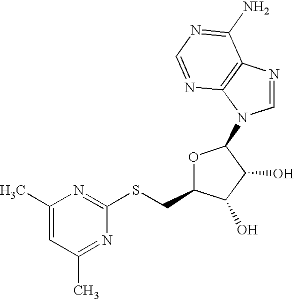 | 389.45 | 390 | 39 | 26 |
| 2(G)(155) | (2R, 3R, 4S, 5S)-2-(6- amino-9H-purin-9-yl)- 5-{[(4-hydroxy-5-methyl- pyrimidin-2-yl)thio]- methyl}tetrahydrofuran- 3,4-diol | 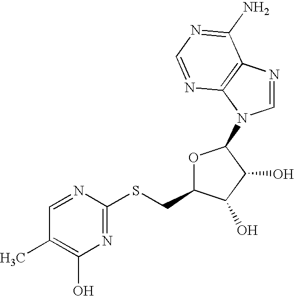 | 391.42 | 392 | 22.5 | 39 |
| 2(G)(156) | (2R, 3R, 4S, 5S)-2-(6- amino-9H-purin-9-yl)- 5-{[(1-phenylethyl)thio]- methyl}tetrahydrofuran- 3,4-diol | 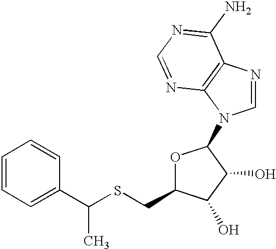 | 387.47 | 388 | 6 | 33 |
| 2(G)(157) | (2R, 3R, 4S, 5S)-2-(6- amino-9H-purin-9-yl)- 5-({[2-(hydroxymethyl)- phenyl]thio}methyl)tetra- hydrofuran-3,4-diol | 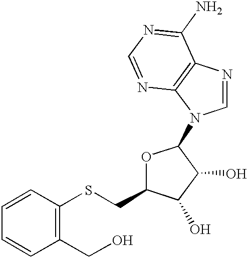 | 389.45 | 390 | 32.5 | 15.5 |
| 2(G)(158) | (2R, 3R, 4S, 5S)-2-(6- amino-9H-purin-9-yl)- 5-{[(4-hydroxy-5,6- dimethylpyrimidin-2-yl)- thio]methyl}tetrahydro- furan-3,4-diol | 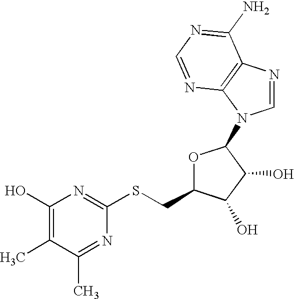 | 405.45 | 406 | 7 | 43.5 |
| 2(G)(159) | 2-({[(2S, 3S, 4R, 5R)-5-(6- amino-9H-purin-9-yl)- 3,4-dihydroxytetrahydro- furan-2-yl)methyl}thio)- acetamide | 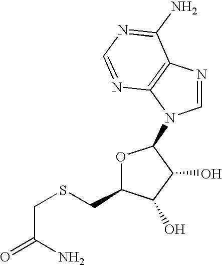 | 340.37 | 341 | 7.5 | 28 |
| 2(G)(160) | (2R, 3R, 4S, 5S)-2-(6- amino-9H-purin-9-yl)- 5-{[(1-benzyl-1H-imidazol- 2-yl)thio]methyl}tetra- hydrofuran-3,4-diol | 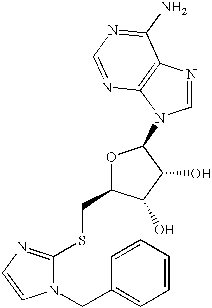 | 439.51 | 441 | 12 | 17 |
| 2(G)(161) | 2-({[(2S, 3S, 4R, 5R)-5-(6- amino-9H-purin-9-yl)- 3,4-dihydroxytetrahydro- furan-2-yl]methyl}thio)- N-methylbenzamide | 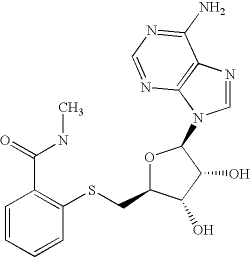 | 416.47 | 417 | 14.5 | 28 |
| 2(G)(162) | (2R, 3R, 4S, 5S)-2-(6- amino-9H-purin-9-yl)- 5-{[(4-hydroxy-6- propylpyrimidin-2-yl)- thio]methyl}tetrahydro- furan-3,4-diol | 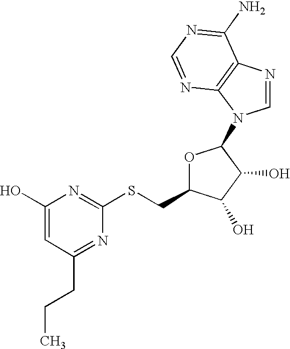 | 419.48 | 420 | 29 | 39.5 |
| 2(G)(163) | (2R, 3R, 4S, 5S)-2-(6- amino-9H-purin-9-yl)- 5-{[(5-chloro-1,3-benz- oxazol-2-yl)thio]methyl}- tetrahydrofuran-3,4-diol | 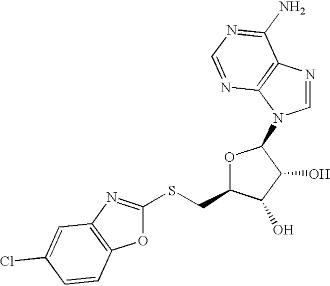 | 434.87 | 436 | 12 | 26.5 |
| 2(G)(164) | Methyl 2-({[(2S, 3S, 4R, 5R)-5-(6-amino-9H-purin- 9-yl)-3,4-dihydroxy- tetrahydrofuran-2-yl]methyl}thio)-1-methyl-1H- imidazole-5-carboxylate | 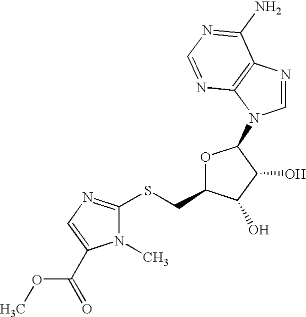 | 421.45 | 422 | 13.5 | 29 |
| 2(G)(165) | (2R, 3R, 4S, 5S)-2-(6- amino-9H-purin-9-yl)- 5-{[(4-tert-butyl-6- hydroxypyrimidin-2- yl)thio]methyl}tetrahydro- furan-3,4-diol | 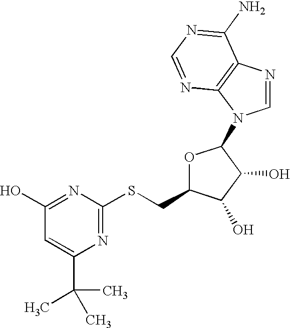 | 433.50 | 435 | 25 | 33.5 |

Biochemical and Biological Evaluation

An enzymatic assay to determine the activity of MTAP against a given substrate was performed. Human MTAP containing an N-terminal six-histidine tag was expressed in E. coli BL21 DE3 cells. The protein was purified to homogeneity by Ni2+ affinity chromatography. Enzymatic activity was measured using a coupled spectrophotometric assay designed to monitor the reaction product adenine (Savarese, T. M., Crabtree, G. W., and Parks, R. E. Jr., (1980) Biochem. Pharmacol. 30, 189-199). Various concentrations of the indicated 5′-deoxymethylthio adenosine (MTA) or substrate were incubated in assay buffer (40 mM potassium phosphate buffer, 1 mM, and DTT 0.8 units/ml xanthine oxidase coupling enzyme) for 5 minutes at 37° C. The reaction was initiated by the addition of MTAP. The exact concentration of enzyme used varied for each substrate tested and ranged from 2 nM to 500 nM. Activity as a function of enzyme concentration was determined for each substrate tested to ensure that the appropriate enzyme concentration was used. Activity was detected by continuous monitoring of absorbance at 305 nm for 10 minutes (ΔE=15,500 M⁻¹). Initial velocities were calculated by linear regression. kcat and Km values were determined by fitting initial velocity data to the Henri-Michaelis-Menton equation and are listed for some of the example compounds in Table 10 below.

Library compounds (10 and 50 uM) were tested using the assay described above with 2 nM MTAP enzyme. The resultant initial velocities are reported as a percentage of the initial velocity observed when MTA is the substrate. MTA controls, 10 and 50 uM concentrations, were run on each plate alongside the library compounds. The relative initial velocities, as compared to MTA at 10 and 50 uM, are listed in Table 9 above.

TABLE 10
Kcat and Km values for select Examples.

| Example No. | Structure | kcat (/s) | Km (uM) |
|---|---|---|---|
| 2(F)(17) | 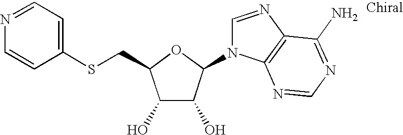 | 0.23 | 0.88 |
| 2(B)(16) | 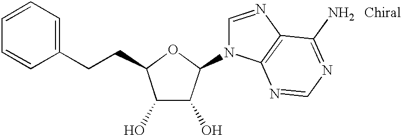 | 4.6 | 1.3 |
| 2(F)(8) | 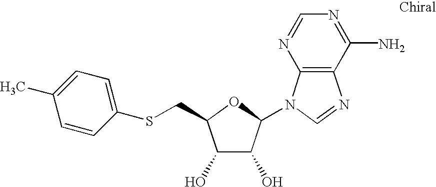 | 1.44 | 1.5 |
| 2(F)(15) | 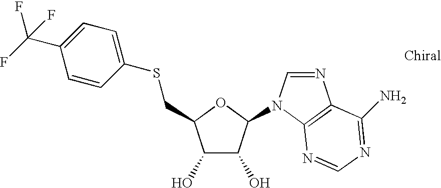 | 0.29 | 1.7 |
| Known* | 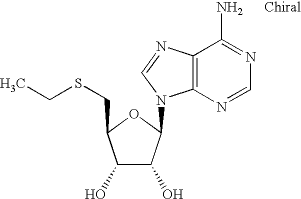 | 2.9 | 1.8 |
| 2(F)(7) | 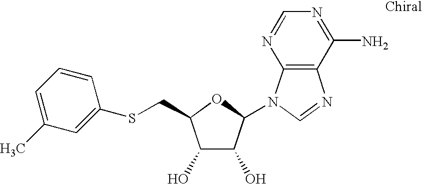 | 2.4 | 2 |
| 2(F)(10) | 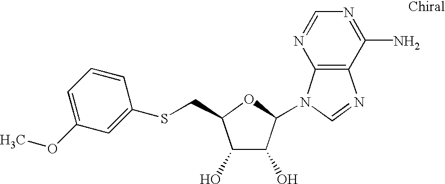 | 1.4 | 2.2 |
| MTA (Compd AA) | 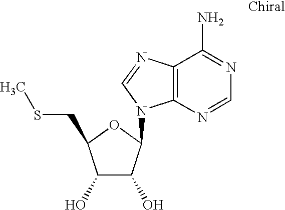 | 3.967 | 2.233 |
| 2(F)(27) | 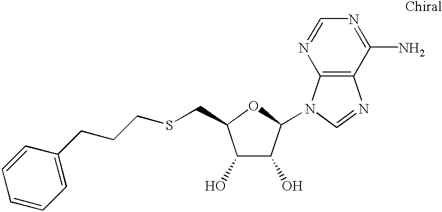 | 2.16 | 2.8 |
| known | 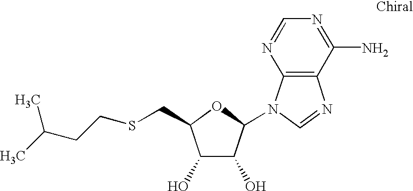 | 5.5 | 2.8 |
| known | 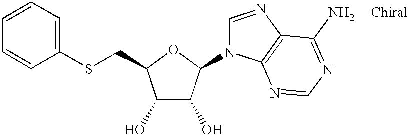 | 1.5 | 3 |
| known | 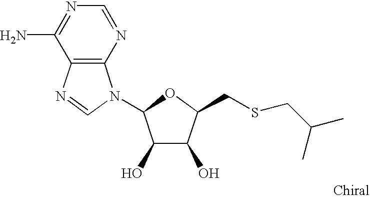 | 2.3 | 3.1 |
| 2(F)(26) | 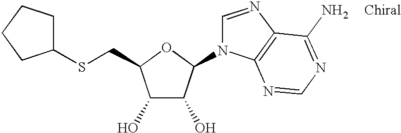 | 1.5 | 3.2 |
| known | 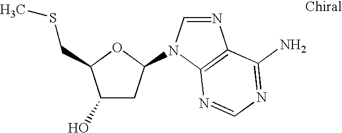 | 0.76 | 3.3 |
| known | 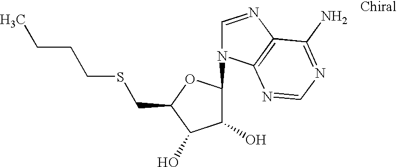 | 5.4 | 3.3 |
| 2(F)(23) | 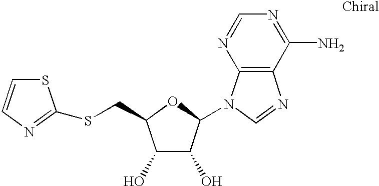 | 2.49 | 3.4 |
| 2(F)(18) | 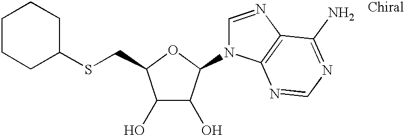 | 1.57 | 3.5 |
| 2(F)(5) | 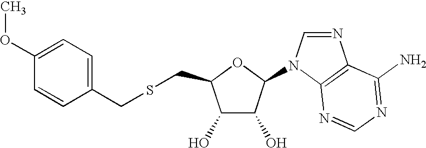 | 3.8 | 3.7 |
| known | 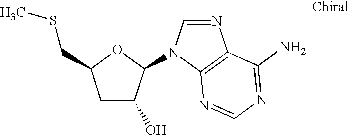 | 0.004 | 3.9 |
| 2(F)(1) | 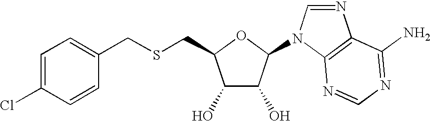 | 3.3 | 3.9 |
| 2(F)(13) | 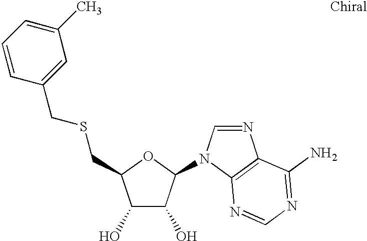 | 1.82 | 4 |
| 2(F)(20) | 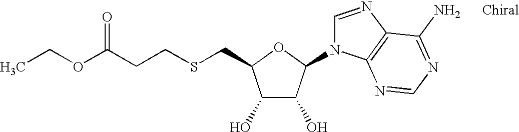 | 1.54 | 4.3 |
| 2(F)(21) | 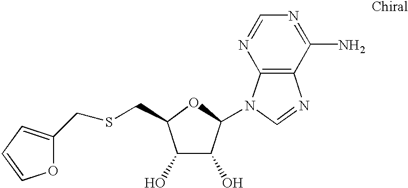 | 6.15 | 4.45 |
| known | 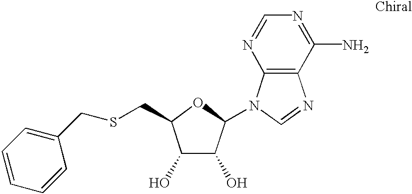 | 2.5 | 4.65 |
| 2(F)(14) | 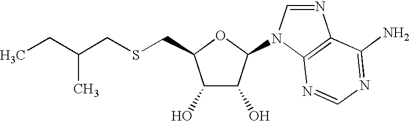 | 4.2 | 5 |
| known | 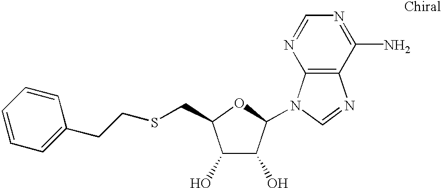 | 2.14 | 5 |
| 2(F)(19) | 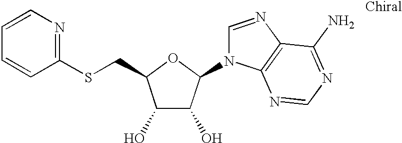 | 3.44 | 5.2 |
| 2(F)(24) | 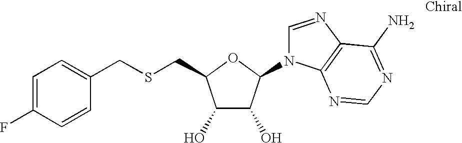 | 2.24 | 5.4 |
| 2(F)(28) | 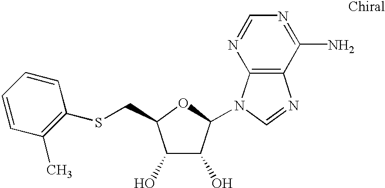 | 0.175 | 5.6 |
| known | 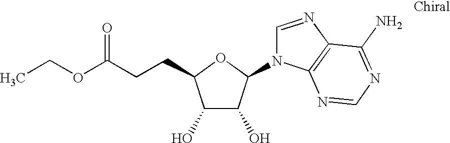 | 4.115 | 5.95 |
| 2(F)(25) | 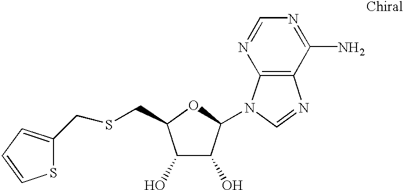 | 4.6 | 6 |
| known | 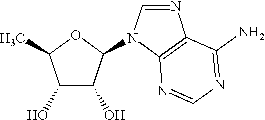 | 4.8 | 6 |
| 2(F)(6) | 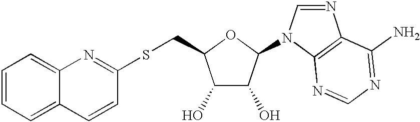 | 3.16 | 6.9 |
| 2(F)(3) | 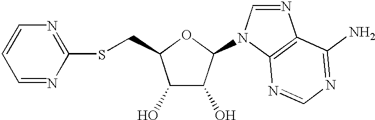 | 4.1 | 7 |
| 2(F)(11) | 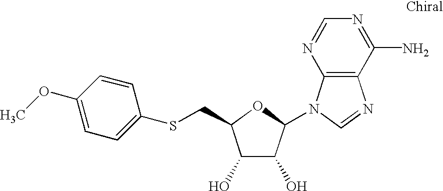 | 0.8 | 7 |
| 2(B)(4) | 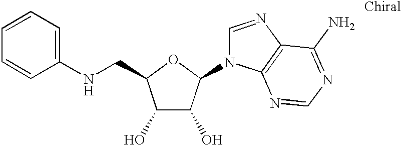 | 2.02 | 8.5 |
| 2(F)(22) | 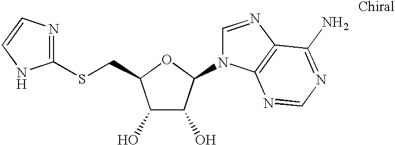 | 3.8 | 9 |
| 2(B)(15) | 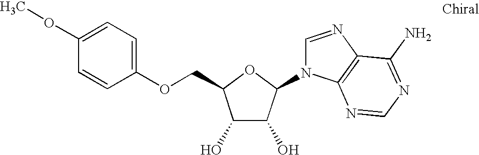 | 0.54 | 10 |
| 2(F)(12) | 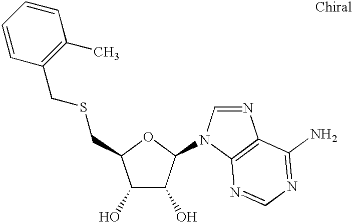 | 0.79 | 10 |
| 2(F)(16) | 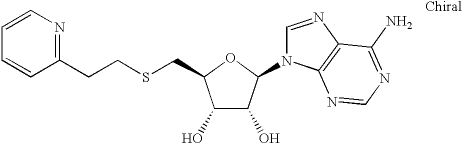 | 1.01 | 10.2 |
| 2(B)(12) | 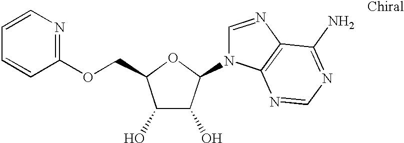 | 1.11 | 12 |
| 2(F)(9) | 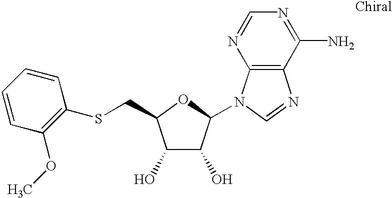 | 0.13 | 13 |
| known | 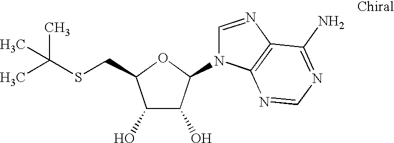 | 0.85 | 17 |
| 2(E)(2) | 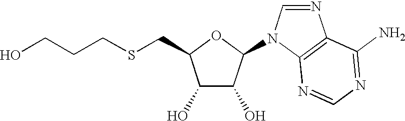 | 3.1 | 21 |
| known | 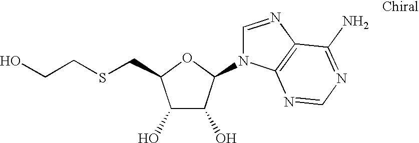 | 1.46 | 25 |
| 2(B)(14) | 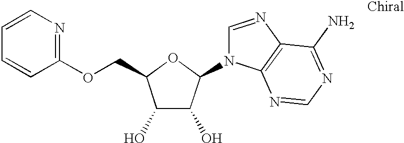 | 3.82 | 29 |
| 2(C)(11) | 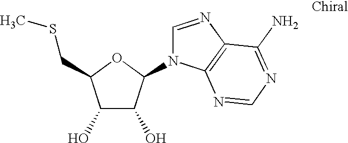 | 0.67 | 30 |
| 2(B)(13) | 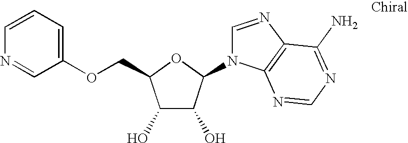 | 0.126 | 33 |
| known | 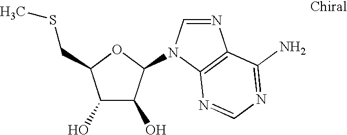 | 0.006 | 106 |
| 2(B)(7) | 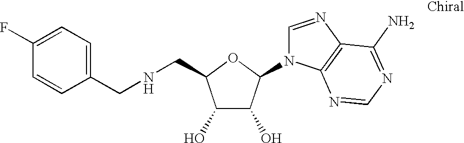 | 0.089 | 145 |
| known | 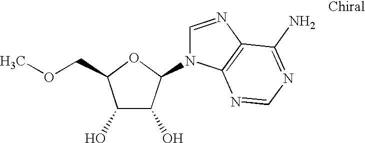 | 0.006 | 250 |
| 2(B)(1) | 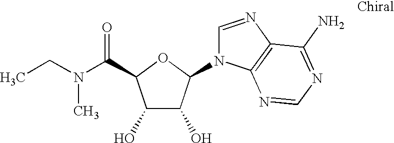 | 0.8 | 300 |
| known | 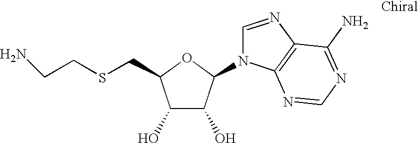 | 0.141 | 390 |
| 2(B)(11) | 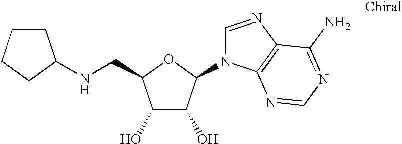 | 0.3 | 600 |
| 2(B)(8) | 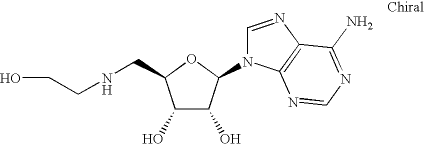 | 0.029 | 758 |
| 2(B)(19) | 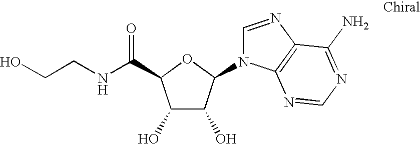 | 3 | 1000 |
| 2(B)(6) | 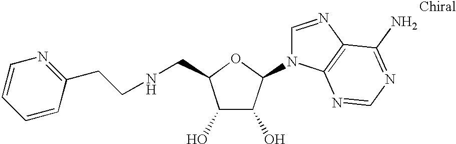 | 0.018 | 1300 |
| 2(C)(10) | 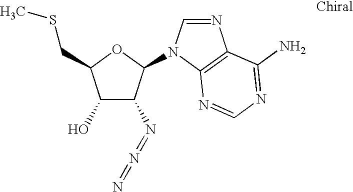 | 0.04 | 3600 |

*“Compounds that have been previously cited in the literature are indicated as known.”

### EXAMPLE 3

In Vitro Studies

### EXAMPLE 3(A)

Growth Inhibition Effect of Compound 7 In Vitro On MTAP-Competent and MTAP-Deficient Cells with and Without Methylthio-Adenosine as Anti-Toxicity Agent

The effect of combination therapy using Compound 7 and MTA was performed in vitro on both MTAP-deficient and MTAP-competent cells. Compound 7 is a GARFT inhibitor having a Kᵢ of 0.5 nM, and a K_{d} of 290 nM to mFBP (binds about 1400-fold less tightly than lometrexol; Bartlett et al. Proc AACR 40 (1999)) and can by synthesized by methods provided in Example 1 above.

The growth inhibition of Compound 7, both with and without MTA, was analyzed using 5 MTAP-competent and 3 MTAP-deficient human lung, colon, pancreatic, muscle, leukemic and melanoma cell lines, as listed in Table 4. All cell lines were purchased from the American Type Culture Collection. The growth conditions and media requirements of each cell line are summarized in Table 5. All cultures were maintained at 37° C., in 5% air-CO₂ atmosphere in a humidified incubator.

TABLE 4

| | MTAP | |
|---|---|---|
| Cell Line | Competent? | Origin |
| NCI-H460 | Yes | Human, large cell lung carcinoma |
| SK-MES-1 | Yes | Human, lung squamous cell carcinoma |
| HCT-8 | Yes | Human, ileocecal colorectal adenocarcinoma |
| HCT-116 | Yes | Human, colorectal carcinoma |
| A2058 | Yes | Human, melanoma |
| PANC-1 | No | Human, pancreatic epithelial carcinoma |
| BxPC-3 | No | Human, pancreatic adenocarcinoma |
| HT-1080 | No | Human, fibrosarcoma |

Cells were plated in columns 2-12 of a 96-well microtiter plate, with column 2 designated as the vehicle control. The same volume of medium was added to column 1. Column 1 was designated as the media control. After a 4-hour incubation, the cells were treated with Compound 7, with or without a non-growth inhibitory concentration of MTA, in quadruplicate wells. Cells were incubated with compound 7 for 72 hours or 168 hours, as indicated in Table 5 below, i.e., cells were exposed to Compound 7 and/or MTA continuously for ˜2.5-3 cell doublings. MTT (4,5-dimethylthiazol-2-yl)-2,5-diphenyl tetrazolium bromide; Sigma, St. Louis, Mo.) was added to a final concentration of 0.25-1 mg/ml in each well, and the plates were incubated for 4 hours. The liquid was removed from each well. DMSO was added to each well, then the plates were vortexed slowly in the dark for 7-20 minutes. The formazin product was quantified spectrophotometrically at 540 run on a Molecular Devices Vmax™ kinetic microplate reader.

TABLE 5

| | | | Plating | |
|---|---|---|---|---|
| | | Optional | Density | Incubation |
| Cell Line | Medium* | Supplements | (cells/well) | Time (hrs) |
| NCI-H460 | MEM** | None | 1500 | 72 |
| SK-MES-1 | MEM** | 5% | 1500 | 168 |
| | | nonessential | | |
| | | amino acids, | | |
| | | 5% sodium | | |
| | | pyruvate | | |
| HCT-8 | Iscove's** | 5% | 900 | 72 |
| | | nonessential | | |
| | | amino acids, | | |
| | | 5% sodium | | |
| | | pyruvate | | |
| HCT-116 | Iscove's** | 5% | 1000 | 168 |
| | | nonessential | | |
| | | amino acids, | | |
| | | 5% sodium | | |
| | | pyruvate | | |
| A2058 | Iscove's** | 5% | 2000 | 72 |
| | | nonessential | | |
| | | amino acids, | | |
| | | 5% sodium | | |
| | | pyruvate | | |
| PANC-1 | DMEM*** | None | 1000 | 168 |
| BxPC-3 | RPMI- | None | 1500 | 168 |
| | 1640*** | | | |
| HT-1080 | Iscove's** | 5% | 1000 | 72 |
| | | nonessential | | |
| | | amino acids, | | |
| | | 5% sodium | | |
| | | pyruvate | | |

*Supplemented with 10% dialyzed horse serum concentration (dHS), commercially available from Gibco Life Technologies, Gaitherburg, MD.
**MEM and Iscove's medium are commercially available from Gibco Life Technologies.
***DMEM and RPMI-1640 medium are commercially available from Mediatech, Washington, D.C.

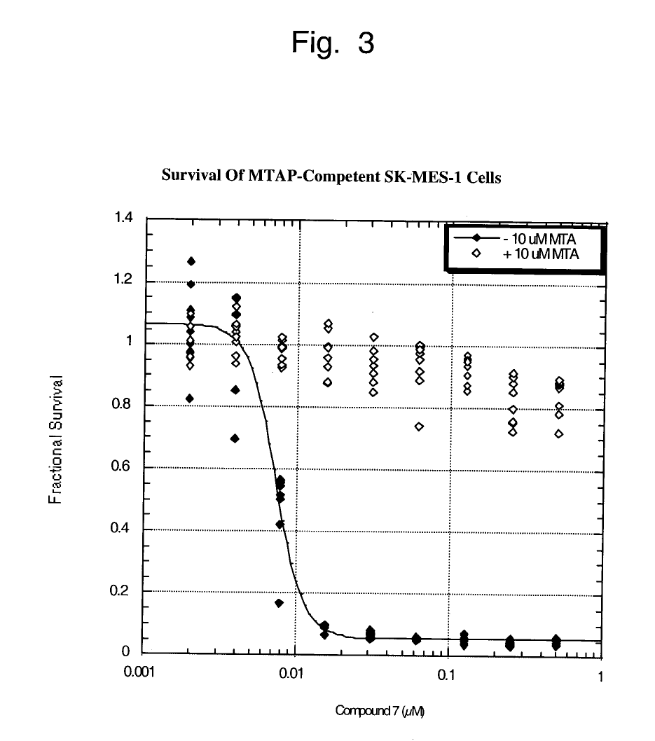

The effect of Compound 7 on SK-MES-1 cells, with and without MTA, is shown in FIG. 3. FIG. 3 indicates that Compound 7 fully inhibited cell growth as a single agent, with a background of approximately 5%. However, addition of 10 μM MTA to up to approximately 60 times the IC₅₀ concentration of Compound 7 decreased the induction of growth inhibition dramatically, causing the cell number to increase to about 75% of control at the highest concentration of Compound 7 tested.

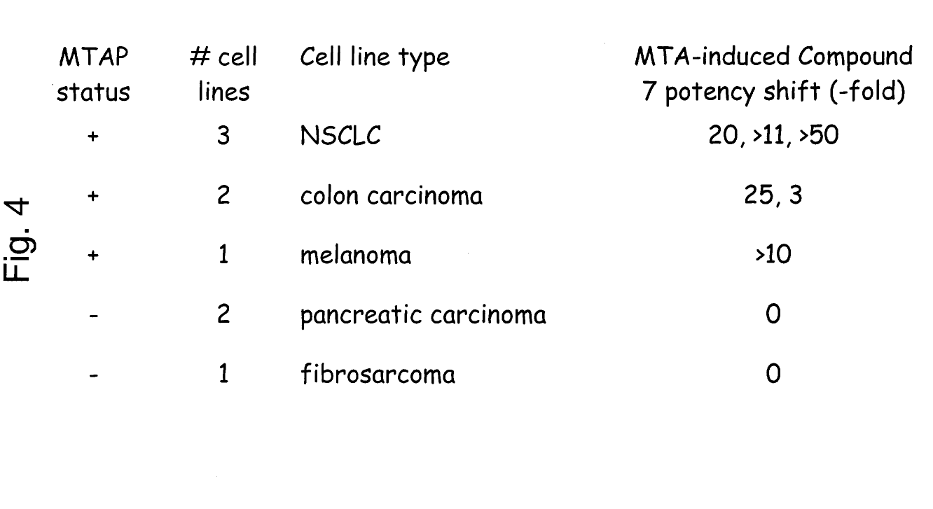

With regard to the growth inhibitory effect of Compound 7 on all 9 cell lines, FIG. 4 indicates that MTA reduced the growth inhibitory activity of Compound 7 in the 5 MTAP-competent human lung, colon and melanoma cell lines (3- to >50-fold shift in the IC₅₀ of Compound 7) but not in the 3 MTAP-deficient human cell lines.

### EXAMPLE 3(B)

Cytotoxicity of Compound 7 in vitro on MTAP- And Sham-Transfected BXPC-3, PANC-1 and HT-1080 Cells with and Without Methylthioadenosine or dcSAMe as Anti-Toxicity Agent

The efficacy of combination therapy of Compound 7 with MTA or dcSAMe on toxicity was evaluated using isogenic pairs of cell lines, i.e. BxPC-3, PANC-1, and HT-1080, which were either MTAP-deficient, or were made MTAP-competent by transfection of a plasmid carrying the MTAP-encoding gene.

Transfection

The coding region of the MTAP cDNA was PCR amplified from a placental cDNA library using the forward primer, GCAGACATGGCCTCTGGCACC (SEQ ID: 2), and reverse primer AGCCATGCTACTTTAATGTCTTGG (SEQ ID: 3). The amplified product was cloned to pCR-2.1-TOPO (Invitrogen, Carlsbad, Calif.) and sequenced (SEQ ID: 1). The MTAP cDNA was subcloned to the retroviral vector pCLNCX for production of recombinant retrovirus.

Retroviral production was conducted by transfecting the pCLNCX/MTAP vector into the PT67 amphotrophic retrovirus packaging cell line (Clontech, Palo Alto, USA) using calcium phosphate mediated transfection according to the suppliers protocol. Supernatants from the transfected packaging cells were collected at 48 hours post transfection and filtered through 0.45 μm filters before infection of target cells.

Transduction of target cell lines and isolation of MTAP expressing clonal cell lines was conducted by plating target cells at low density in 10 cm dishes and growing for 24 hours. Retroviral supernatants were diluted 1:2 with fresh medium containing polybrene at 8 μg/ml. Medium from target cells was removed and replaced with the prepared retroviral supernatant and cells were incubated for 24 hours. Retroviral supernatant was then removed and replaced with fresh medium and incubated another 24 hours. Infected target cells were then harvested and replated onto 10 cm dishes at a range of densities into medium containing geneticin at 400 ug/ml to select for transduced cells. After 2-3 weeks, isolated colonies were picked and expanded as individual clonal cell lines. Expression of the MTAP cDNA within individual clonal line s determined through RT-PCR analysis using the Advantage One Step RT-PCR kit (Clontech, Palo Alto, USA) according to the manufacturer's protocol.

Cytotoxicity

Cytoxicity data was collected using BxPC-3, PANC-1 and HT-1080 cells which were cultured in Iscove's medium supplemented with 10% dialyzed, horse serum, 5% nonessential amino acids and 5% sodium pyruvate.

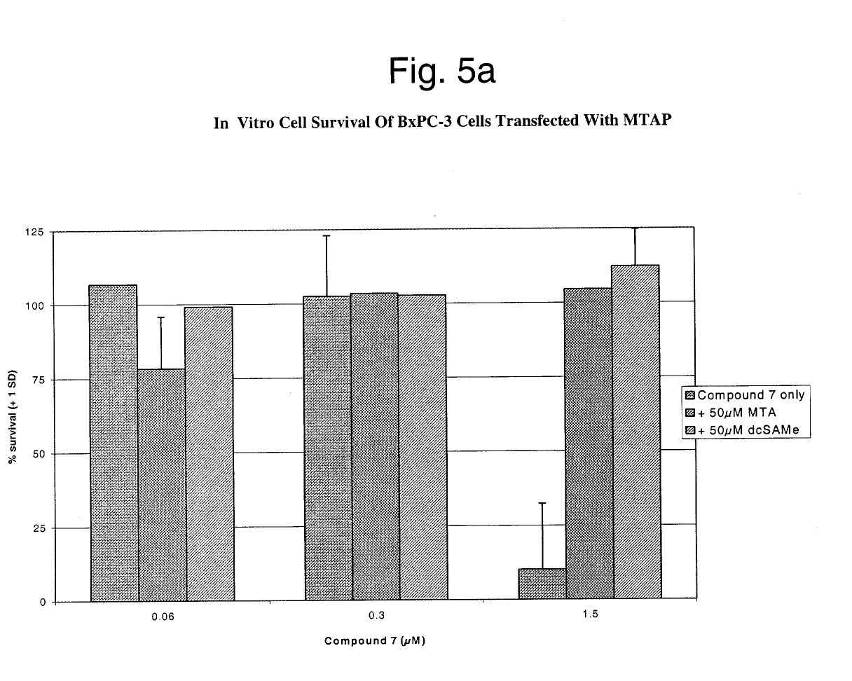

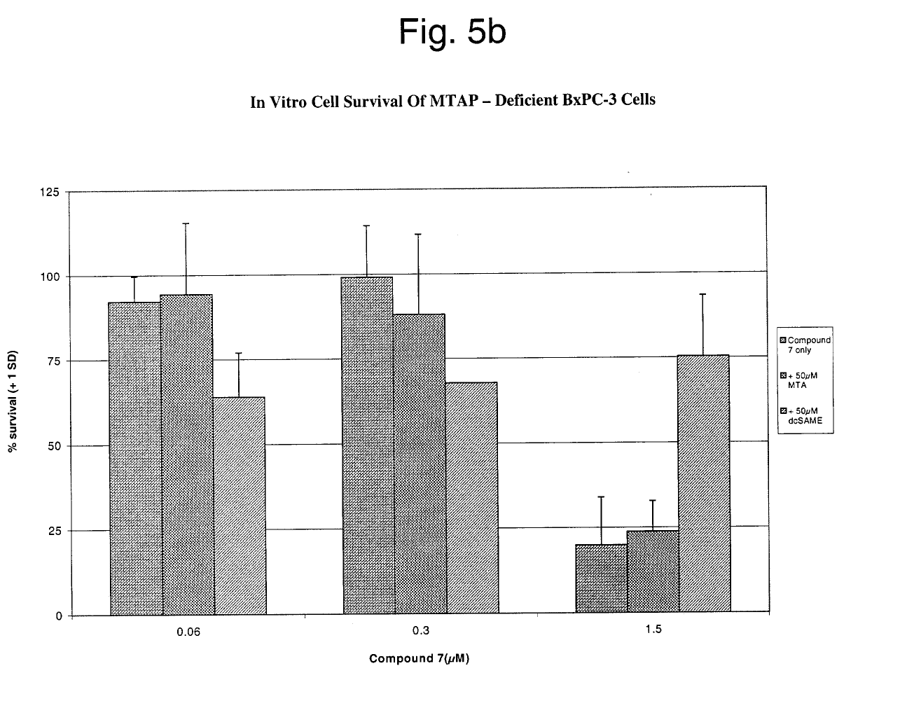

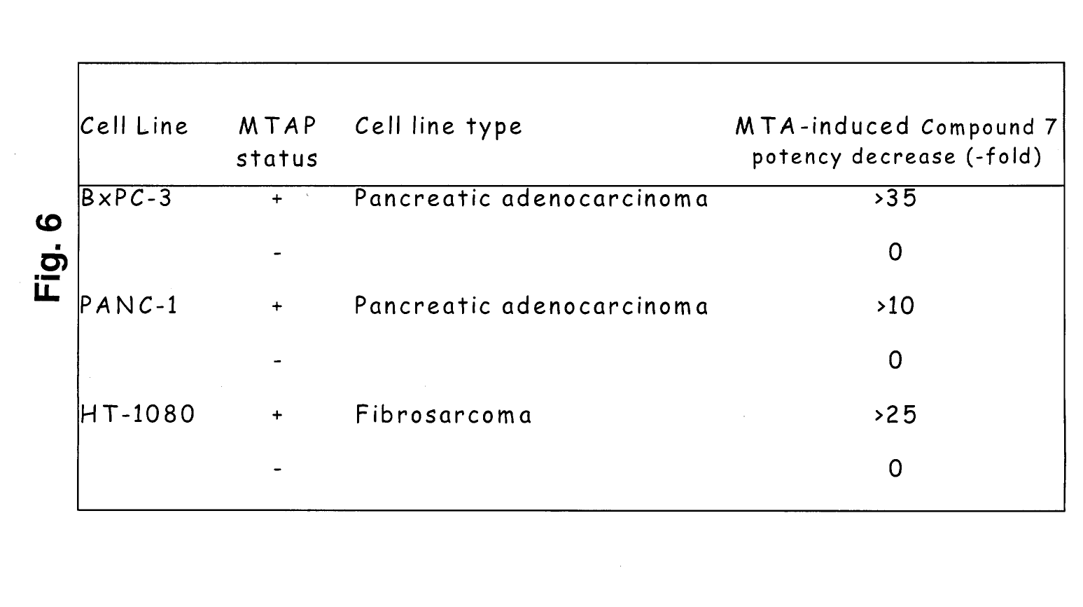

Mid-log-phase cells were trypsinized and placed in 60 mm tissue culture dishes at 200 or 250 cells per dish. Cells from each cell line were left to attach for 4 hours and then were treated with Compound 7, with or without MTA or dcSAMe, in 5-fold serial dilutions for 6 or 24 hours. For data shown in FIGS. 5a and 5b, cells were exposed to drug(s) for 6 hours only. For data shown in FIG. 6, cells were exposed to Compound 7 for 24 hours and to MTA continuously for the duration of colony growth (i.e. 24 hours and thereafter). Cells were incubated until visible colonies formed in the control dishes, as indicated in Table 6 below. Cells were next washed with PBS, and then fixed and stained with 1% w/v crystal violet in 25% methanol (Sigma, St. Louis, Mo.). After washing the dishes 2-3 times. with deionized water, the colonies were counted. Triplicate dishes were used for each drug concentration.

TABLE 6

| Cell Line | Medium | Incubation Time (days) |
|---|---|---|
| BxPC-3 | Iscove's medium* | 13-14 |
| HT-1080 | Iscove's medium* | 6-7 |
| PANC-1 | Iscove's medium* | 14 |

*Iscove's medium was supplemented with 10% dialyzed horse serum, 5% nonessential amino acid, 5% sodium pyruvate, and 1% monothioglycerol.

The cytotoxicity data for 6 hours of simultaneous drug exposure with Compound 7 with or without dcSAMe or MTA is summarized in FIGS. 5a and 5b. FIG. 5a indicates that cell survival of MTAP-competent cells increased to 100% at 1.5 μM Compound 7 with either 50 μM MTA or dcSAMe. By contrast, as indicated in FIG. 5b, the same concentrations of MTA and dcSAMe in MTAP-deficient cells either did not increase cell survival (MTA) or increased cell survival by less than observed for the MTAP competent cells (dcSAMe).

FIG. 6 summarizes selective reduction of cytotoxicity of Compound 7 by the introduction of MTA. Exposure of Compound 7 for 24 hours, with exposure to MTA for those 24 hours and continuously thereafter, achieved a >10- to >35-fold shift in the MTAP-competent cell lines versus their MTAP-deficient counterparts.

### EXAMPLE 3(C)

Growth Inhibition Effect of Compounds 1 and 3 in vitro on MTAP-Competent Cells with and Without Methylthioadenosine as an Anti-Toxicity Agent

The growth inhibition effect of combination therapy using Compound 1 or Compound 3 in combination with MTA was performed in vitro on MTAP-competent NCI-H460 cells. Compound 1 is a specific inhibitor of AICARFT having a micromolar Kᵢ and a K_{d} of 83 nM to mFBP. Compound 3 is a GARFT inhibitor having a Kᵢ of 2.8 nM and a K_{d} 0.0042 nM to mFBP. (Bartlett et al. Proc AACR 40 (1999)). Compounds 1 and 3 have the following chemical structures, respectively, and can be synthesized by methods described in U.S. Pat. Nos. 5,739,141 and 5,639,747, which are incorporated herein by reference in their entirety:

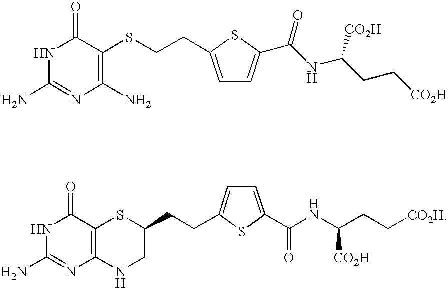

The growth inhibition of Compound 1 and Compound 3, each with and without MTA, was analyzed using an MTAP-competent human lung carcinoma cell line. NCI-H460 cells were grown,, plated and treated with varying concentrations of Compound 1 or Compound 3 in combination with MTA, in the same manner as described in Example 3 (A) above.

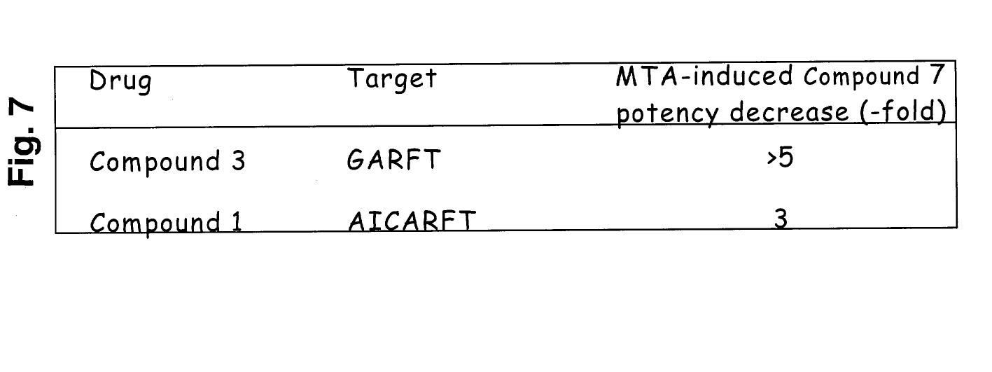

With regard to the growth inhibitory effect of Compound 1 on the MTAP-competent cell line, FIG. 7 indicates that exposure of Compound 1 with MTA reduced the growth inhibitory activity of Compound 1 in the MTAP-competent human lung by a factor of 3. Similarly, exposure of Compound 3 with MTA reduced the growth inhibitory activity of Compound 3 in the MTAP-competent cell line by a factor of greater than 5.

### EXAMPLE 3(1))

Cytotoxicity of Compound 7 in vitro on MTAP-Competent Cells when Administered with MTA During and After Administration of Compound 7.

Cytoxicity data for combination therapy of Compound 7 with MTA was collected using MTAP-competent NCI-H1460 cells. NCI-H460 cells were cultured, incubated and stained as described in Example 3(B) above, but with an incubation time of up to eight days.

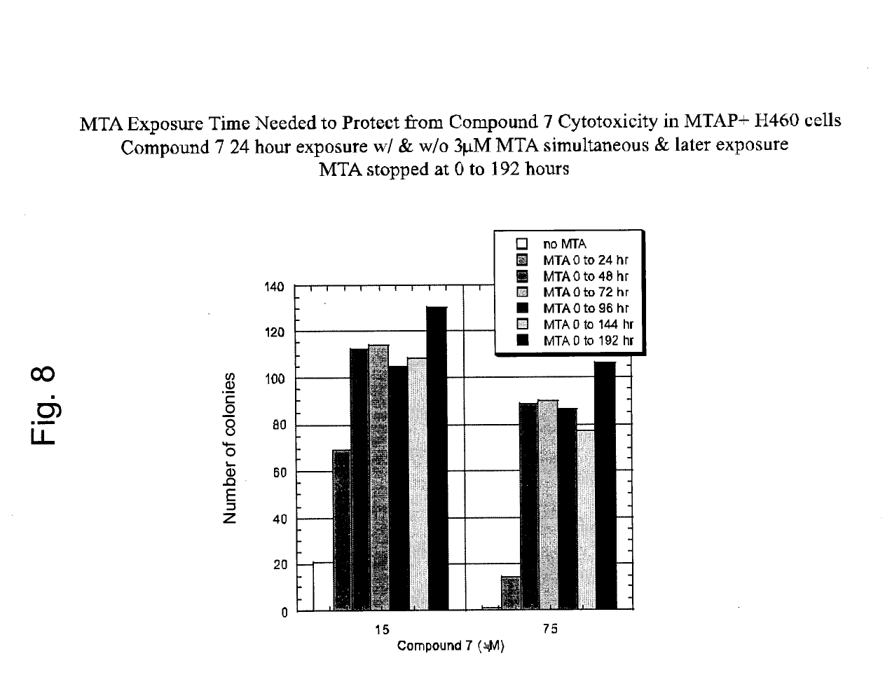

As shown in FIG. 8, increasing the duration of MTA exposure increased the number of surviving colonies treated with cytotoxic concentrations of Compound 7. In particular, extending MTA administration to at least 48 hours, i.e. for at least 1 day subsequent to exposure with Compound 7, fully protected cells from Compound 7-induced cytotoxicity.

### EXAMPLE 4

Effect of Compound 7 in vivo in MTAP-Deficient Xenograft Model with and Without Methylthioadenosine as an Anti-Toxicity Agent

To evaluate the in vivo effect of combination therapy on known human MTAP-deficient tumors, an MTAP-deficient cell line was introduced to mice to produce xenograft MTAP-deficient tumors. 108 BALB/c/nu/nu female mice bearing subcutaneous tumor fragments produced from the MTAP-deficent BxPC-3 cell line were housed 3 per cage with free access to food and water. Mice were fed a folate-deficient chow (#Td84052, Harlan Teklad, Madison, Wis.) beginning 14 days prior to initiation of drug treatment and continuing throughout the study. After randomization by tumor volume into 8 treatment groups and assigning the remaining 12 mice to group 7, beginning on the twenty-first day after tumor implant mice were dosed with Compound 7 daily for 4 days, and with MTA or vehicle twice-a-day for 8 days, in the amounts indicated in Table 7 below. The vehicle for both compounds was 0.75% sodium bicarbonate in water (7.5% NaHCO₃ solution (Cellgro #25-035-4, Mediatech, Herndon, Va.) diluted 1:10 in sterile water for injection (Butler, Columbus, Ohio)) under pH adjusted to 7.0-7.4. Solutions were sterilized by filtration through 0.22 micron polycarboniate filters (Cameo 25GAS, Micron Separations Inc., Westboro, Mass.). Tumor volumes and animal weight loss, which is an indicator of toxicity, were recorded daily for 14 days at the same time of day, then on a Monday, Wednesday, Friday schedule for the remainder of the study.

TABLE 7

| Group | Compound 7 (mg/kg) | MTA (mg/kg) |
|---|---|---|
| 1 | 0 | 0 |
| 2 | 0 | 50 |
| 3 | 20 | 0 |
| 4 | 10 | 0 |
| 5 | 5 | 0 |
| 6 | 2.5 | 0 |
| 7 | 40 | 50 |
| 8 | 20 | 50 |
| 9 | 10 | 50 |

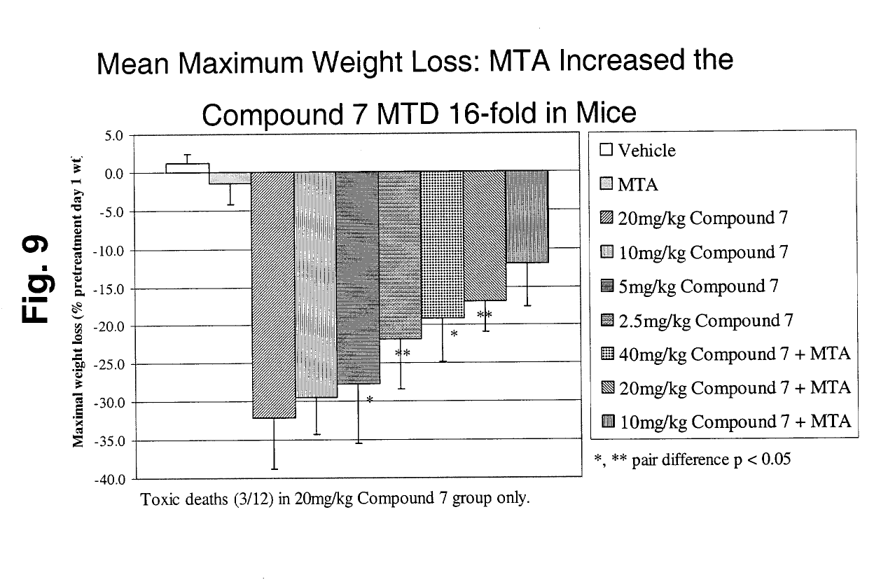

A graphic representation of the magnitude of animal weight loss of the subject animals, induced by varying doses of Compound 7 and MTA, is provided in FIG. 9. The similarities in weight loss between mice treated with 2.5 mg/kg Compound 7 alone versus mice treated with 40 mg/kg Compound 7 plus 50 mg/kg MTA, indicate a 16-fold reduction in toxicity.

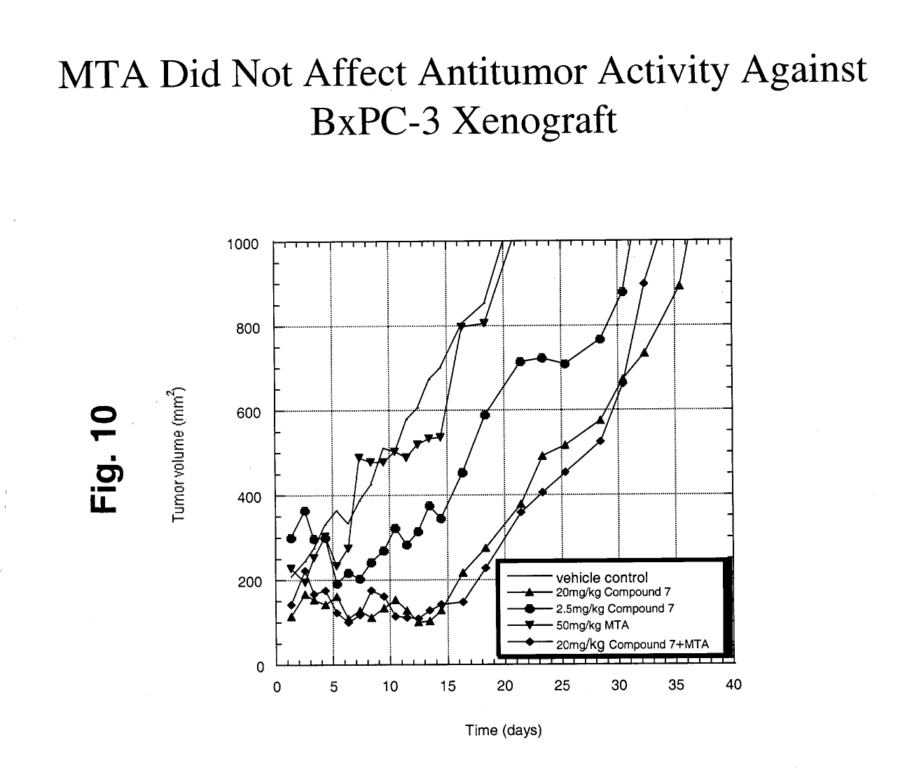

The BxPC-3 xenograft experiments further indicate that MTA lessened the toxicity of Compound 7 without adversely affecting its antitumor activity. As shown in FIG. 10 and in Table 8 below, there was no significant difference in the antitumour data for Compound 7, based on the mean time for tumours to grow to a volume of 1000 mm³ (approximately 35.2 days for 20 mg/kg Compound 7 alone versus 35.3 days for 20 mg/kg Compound 7+MTA).

TABLE 8
The activity of Compound 7 qd daily x4 with and
without 50 mg/kg MTA bid daily x8 against the
human pancreatic BxPC-3 tumor

| | | Time to 1000 | | | p-values^{b} | | | |
|---|---|---|---|---|---|---|---|---|
| | | mm³ (days) | | | Compound 7 (mg/kg) | | | Vehicle |
| Treatment | n^{a} | Mean | SD | Median | 20 | 5 | 2.5 | control |
| Vehicle control | 12 | 20.8 | 4.9 | 20.4 | | | | |
| 20 mg/kg Compound 7 | 9 | 35.2 | 6.6 | 36.4 | | 0.290 | 0.329 | |
| 10 mg/kg Compound 7 | 11 | 34.0 | 6.0 | 33.4 | | | | |
| 5 mg/kg Compound 7 | 12 | 32.1 | 6.4 | 32.4 | | | | |
| 2.5 mg/kg Compound 7 | 10 | 32.3 | 5.9 | 32.4 | | | | <0.0001 |
| 50 mg/kg MTA | 11 | 22.6 | 6.8 | 21.4 | | | | |
| 20 mg/kg Compound | 12 | 35.3 | 3.4 | 34.9 | 0.957 | 0.135 | 0.170 | 0.462 |
| 7 + MTA | | | | | | | | |
| 10 mg/kg Compound | 12 | 37.7 | 4.9 | 37.9 | | | | |
| 7 + MTA | | | | | | | | |

^{a}Number of evaluable tumors.
^{b}2-sided p-values calculated in Excel.

Thus, adding MTA twice a day for 8 days to the daily administration of Compound 7 for 4 days in nu/nu tumor-bearing mice on a folate-deficient diet increased the therapeutic window of Compound 7 by 16- fold.

### EXAMPLE 5

In vivo Effect of Extended Dosing Schedule of MTA on Maximally Tolerated Dose of Compound 7

A series of experiments were undertaken in order to evaluate the in vivo effect of schedule of administration of MTA on reduction of toxicity induced by toxicity. BALB/c/nu/nu female mice were housed 3 per cage with free access to food and water. Mice were fed a folate-deficient chow (#Td84052, Harlan-Teklad, Madison, Wis.) for at least 14 days prior to initiation of drug treatment and continuing throughout the study. Mice were dosed with Compound 7 daily for 4 days, and with MTA or vehicle twice daily on the schedule indicated in Table 11. Animal weight loss, which is a measure of toxicity, was recorded at least daily for 18 days at the same time of day. Table 11 presents a summary of data from multiple experiments, i.e., at least two experiments for each schedule. These data indicate that coadministration of MTA can increase the maximum tolerated dose of Compound 7. To produce this effect, MTA must be administered at the beginning of treatment with Compound 7 and continuing until after treatment with Compound 7. Further, since the activity of Compound 7 continues for at least a few days after the last dose was administered, to produce an effect MTA must be administered during this period of activity, i.e. for at least 2 days after the last dose of the cytotoxic was administered.

TABLE 11

| Compound 7 | MTA | Increase in Compound 7 maximum |
|---|---|---|
| (days) | (days) | tolerated dose (-fold dose) |
| 1-4 | 3-8 | None |
| 1-4 | 1-6 | 4 |
| 1-4 | 1-5 | None |
| 1-4 | 5-7 | None |
| 1-4 | 3-8 | None |

What is claimed is:

1. A method for selectively killing MTAP-deficient cells of a mammal, the method comprising:
(a) administering to the mammal an inhibitor of glycinamide ribonucleotide formyltransferase, aminoimidazolecarboximide ribonucleotide formyltransferase: or both in a therapeutically effective amount; and
(b) administering to the mammal an anti-toxicity agent in an amount effective to increase the maximally tolerated dose of the inhibitor; wherein the anti-toxicity agent is administered during and after administration of the inhibitor.

2. The method of claim 1, wherein the anti-toxicity agent is an MTAP substrate or a prodrug of an MTAP substrate.

3. The method of claim 2, wherein the anti-toxicity agent has Formula X: 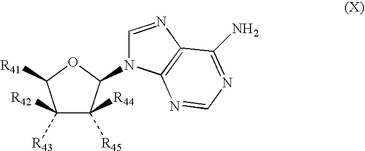
R₄₁ is selected from the group consisting of:
(a) —R_{g} wherein R_{g} represents a C₁-C₅ alkyl, C₂-C₅ alkenylene or alkynylene radical, unsubstituted or substituted by one or more substitutents independently selected from C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl;
(b) —R_{g}(Y)RₕRᵢ wherein R_{g} is as defined above, Y represents O, NH, S, or methylene; and Rₕ and Rᵢ represent, independently, (i) H; (ii) a C₁-C₉ alkyl, or a C₂-C₆ alkenyl or alkynyl, unsubstituted or substituted by one or more substitutents independently selected from C₁ to C₆ alkoxy; C₁ to C₆ alkoxy(C₁ to C₆)alkyl; C₂ to C₆ alkynyl; acyl; halo; amino; hydroxyl; nitro; mercapto; —NCOORₒ; —CONH₂; C(O)N(Rₒ)₂; C(O)Rₒ; or C(O)ORₒ, wherein Rₒ is selected from the group consisting of H, C₁-C₆ alkyl, C₂-C₆ heterocycloalkyl, cycloalkyl, heteroaryl, aryl, and amino, unsubstituted or substituted with C₁-C₆ alkyl, 2- to 6-membered heteroalkyl, heterocycloalkyl, cycloalkyl, C₁-C₆ boc-aminoalkyl; cycloalkyl, heterocycloalkyl, aryl or heteroaryl; or (iii) a monocyclic or bicyclic cycloalkyl, heterocycloalkyl, aryl or heteroaryl, unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mereapto, cycloalkyl, heterocycloalkyl, aryl heteroaryl, —COORₒ, —NCORₒ wherein Rₒ is as defined above, 2 to 6 membered heteroalkyl, C₁ to C₆ alkyl-cycloalkyl, C₁ to C₆ alkylheterocycloalkyl, C₁ to C₆ alkyl-aryl or C₁ to C₆ alkyl-aryl;
(c) C(O)NRⱼRₖ wherein Rⱼ and Rₖ represent, independently, (i) H; or (ii) a C₁-C₆ alkyl, amino, C₁-C₆ haloalkyl, C₁-C₆ aminoalkyl, C₁-C₆ boc-aminoalkyl, C₁-C₆ cycloalkyl, C₁-C₆ alkenyl, C₂-C₆ alkenylene, C₂-C₆ alkynylene radical, wherein Rⱼ and Rₖ are optionally joined together to form, together with the nitrogen to which they are bound, a heterocycloalkyl or heteroaryl ring containing two to five carbon atoms and wherein the C(O)NRⱼRₖ group is further unsubstituted or substituted by one or more substitutents independently selected from —C(O)Rₒ, —C(O)ORₒ wherein Rₒ is as defined above, C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl; or
(d) C(O)ORₕ wherein Rₕ is as defined above;
R₄₂ and R₄₄ represent, independently, H or OH; and
R₄₃ and R₄₅ represent, independently, H, OH, amino or halo;
where any of the cycloalkyl, heterocycloalkyl, aryl, heteroaryl moieties present in the above may be further substituted with one or more additional substituents independently selected from the group consisting of nitro, amino, —(CH₂)_{z}—CN where z is 0-4, halo, haloalkyl, haloaryl, hydroxyl, keto, C₁ to C₆ alkyl, C₂ to C₆ alkenyl, C₂ to C₆ alkynyl, heteroalkyl, unsubstituted cycloalkyl, unsubstituted heterocycloalkyl, unsubstituted aryl or unsubstituted heteroaryl;
and salts or solvates thereof.

4. The method of claim 3, wherein the anti -toxicity agent has a Kcat/Km ratio that is greater than 0.05 s⁻¹ μM⁻¹.

5. The method of claim 2, wherein the anti-toxicity agent has Formula XI: 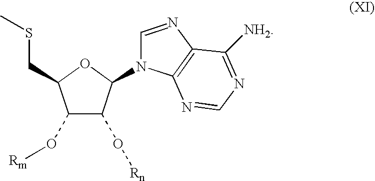
wherein
Rₘ and Rₙ are, independently, selected from the group consisting of H; a phosphate or a sodium salt thereof; C(O)N(Rₒ)₂; C(O)Rₒ; or C(O)ORₒ, wherein Rₒ is selected from the group consisting of H, C₁-C₆ alkyl, C₂-C₆ heterocycloalkyl, cycloalkyl, heteroaryl, aryl, and amino, unsubstituted or substituted with C₁-C₆ alkyl, C₁-C₆ heteroalkyl, C₂-C₆ heterocycloalkyl, cycloalkyl, C₁-C₆ boc-aminoalkyl, and
solvates or salts thereof.

6. The method of claim 5, wherein Rₘ and Rₙ independently represent:

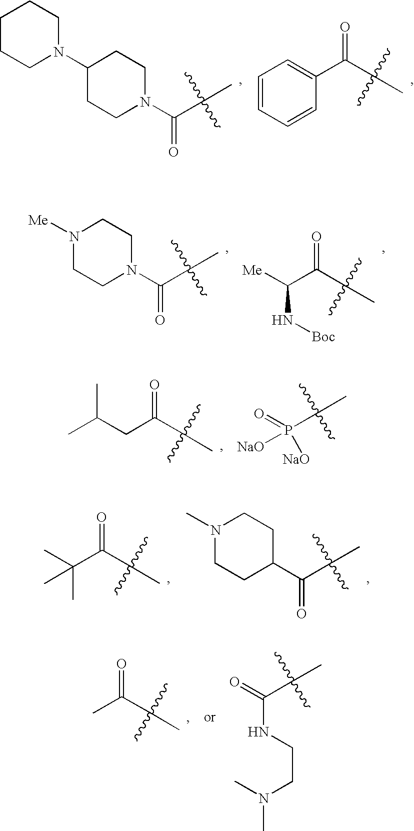

7. The method of claim 1, wherein the inhibitor is a compound of Formula I: 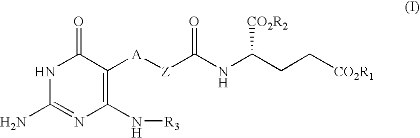
wherein:
A represents sulfur or selenium;
Z represents: a) a noncyclic spacer which separates A from the carbonyl carbon of the amido group by 1 to 10 atoms, said atoms being independently selected from carbon, oxygen, sulfur, nitrogen and phosphorus, said spacer being unsubstituted or substituted with one or more substituents selected from the group consisting of alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—OR_{c}, —(CH₂)_{z}—CN where z is 0-4, halo, —OH, —O—Rₐ—O—R_{b}, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NRₑ, —O—CO—NR_{d}Rₑ, —NR_{c}—CO—NR_{d}Rₑ, —NRₑ—CO—Rₑ, —NR_{c}—CO₂—ORₑ, —CO—NR_{c}—CO—R_{d}, —O—SO₂—R_{c}, —O—SO—R_{c}, —O—S—R_{c}, —S—CO—R_{c}, —SO—CO—OR_{c}, —SO₂—CO—OR_{c}, —O—SO₃, —NR_{c}—SR_{d}, NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}—CSO₂—R_{c}, —NR_{c}—CS—R_{d}, —O—CS—R_{c}, —O—CSO—R_{c}, —SO₂—R_{c}, —SO₂—NR_{d}Rₑ, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, hydroxyl, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group; and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —(CH₂)_{z}—CN where z is 0-4, halo, haloalkyl, haloaryl, —OH, keto (═O), —N—OH, NR_{c}—OR_{c}, —NR_{d}R_{c}, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d}, and Rₑ are as defined above; b) a cycloalkyl, heterocycloalkyl, aryl or heteroaryl didiradical being unsubstituted or substituted with one or more substituents from those substituents recited in a); or c) a combination of at least one of said non-cyclic spacer and at least one of said diradicals, wherein when said noncyclic spacer is bonded directly to A, said non-cyclic spacer separates A from one of said diradicals by 1 to about 10 atoms and further wherein when said non-cyclic spacer is bonded directly to the carbonyl carbon of the amido group, said noncyclic spacer separates the carbonyl carbon of the amido group from one of said diradicals by 1 to about 10 atoms;
R₁ and R₂ represent, independently, hydro, C₁ to C₆ alkyl, or a hydrolyzable group; and
R₃ represents hydro or a C₁ to C₆ alkyl or cycloalkyl group unsubstituted or substituted by one or more halo, hydroxyl or amino.

8. The method of claim 7, wherein Z represents a moiety of formula Q-X—Ar wherein:
Q represents a C₁-C₅ alkenyl, or a C₂-C₅ alkenylene or alkynylene radical, unsubstituted or substituted by one or more substitutents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring;
X represents a diradical of methylene, monocyclic cycloalkyl, heterocycloalkyl, aryl or heteroaryl ring, sulfur, oxygen or amino radicaal, unsubstituted or substituted by one or more substituents independently selected from C₁ to C₆ alkyl, C₂ to C₆ alkenyl, C₁ to C₆ alkoxy C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring; and
Ar represents a monocyclic or bicyclic cycloalkyl, heterocycloalkyl, aryl or heteroaryl ring, wherein Ar may be fused to the monocyclic cycloalkyl, heterocycloalkyl, aryl or heteroaryl ring of X, said Ar is unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring.

9. The method of claim 1, wherein the inhibitor is a compound of Formula II: 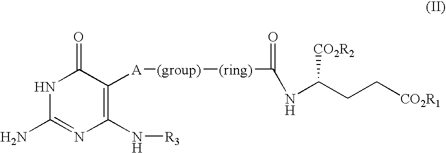
wherein:
A represents sulfur or selenium;
(group) represents a non-cyclic spacer which separates A from (ring) by 1 to 5 atoms, said atoms being independently selected from carbon, oxygen, sulfur, nitrogen and phosphorus, said spacer being unsubstituted or substituted by one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring;
(ring) represents at least one cycloalkyl, heterocycloalkyl, aryl or heteroaryl ring, unsubstituted or substituted with or more substituents selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring;
R₁ and R₂ represent, independently, hydro, C₁ to C₆ alkyl, or a readily hydrolyzable group; and
R₃ represents hydro or a C₁ to C₆ alkyl or cycloalkyl group unsubstituted or substituted by one or more halo, hydroxyl or amino.

10. The method of claim 9, wherein the inhibitor has the chemical structure:

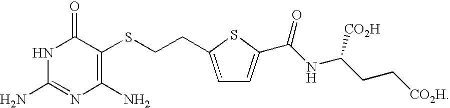

11. The method of claim 9, wherein the inhibitor has the chemical structure:

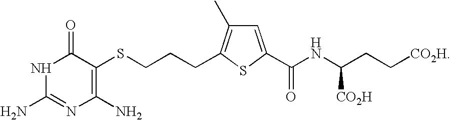

12. The method of claim 7, wherein the inhibitor is a compound of Formula III: 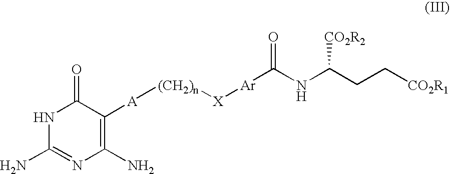
wherein:
n is an integer from 0 to 5;
A represents sulfur or selenium;
X represents a diradical of methylene, a monocyclic cycloalkyl, heterocycloalkyl, aryl or heteroaryl ring, oxygen, sulfur or an amine;
Ar represents an aromatic diradical wherein Ar can form a fused bicyclic ring system with said ring of X; and
R₁ and R₂, represent, independently, hydro or C₁-C₆ alkyl.

13. The method of claim 1, wherein the inhibitor is an inhibitor specific to glycinamide ribonucleotide formyltransferase.

14. The method of claim 13, wherein the inhibitor is a compound having the Formula VII: 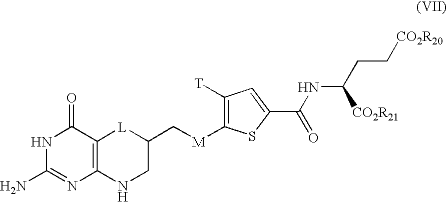
wherein L represents sulfur, CH₂ or selenium;
M represents a sulfur, oxygen, or a diradical of C₁-C₃ alkane, C₂-C₃ alkene, C₂-C₃ alkyne, or amine, wherein M is unsubstituted or substituted by one or more substituents selected from the group consisting ofalkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—OR_{c}, —(CH₂)_{z}—CN where z is 0-4, halo, —OH, —O—Rₐ—O—R_{b}, —OR_{b}, —CO—R_{c}, —O—CO—R_{c—, —CO—OR}_{c}OR_{c}, —O—CO—O—CO—R_{c}, —O—R_{c}, keto (═O), thioketo (═S), —SO₂—R_{c}, —SO—R_{c}, —NR_{d}Rₑ—CO—NR_{d}Rₑ, —O—CO—NR_{d}Rₑ, —NR_{c}—CO—NR_{d}Rₑ, —NR_{c}—CO—Rₑ, —NR_{c}—CO₂—ORₑ, —CO—NR_{c}—CO—R_{d}, —O—SO₂—R_{c}, —O—SO-R_{c}, —O—S—R_{c}, —S—CO—R_{c}, —SO—CO—OR_{c}, —SO₂—CO—OR_{c}, —O—SO₃, —NR_{c}—SR_{d}, —NR_{c}—SO—R_{d}, —NR_{c}—SO₂—R^{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂—R_{c}, —SO₂—NR_{d}Rₑ, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, hydroxyl, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group; and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —(CH₂)_{z}—CN where z is 0-4, halo, haloalkyl, haloaryl, —OH, keto (═O), —N—OH, NR_{c}—OR_{c}, —NR_{d}Rₑ, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d}, and Rₑ are as defined above;
T represents C₁-C₆ alkyl; C₂-C₆ alkenyl; C₂-C₆ alkynyl; —C(O)E, wherein E represents hydro, C₁-C₃ alkyl, C₂-C₃ alkenyl, C₂-C₃ alkynyl, O—(C₁-C₃) alkoxy, or NR₁₀R₁₁, wherein R₁₀ and R₁₁ represent independently hydro, C₁-C₃ alkyl, C₂-C₃ alkenyl, C₂-C₃ alkynyl; hydroxyl; nitro; SR₁₂, wherein R₁₂ is hydro, C₁-C₆ alkyl, C₂-C₆ alkenyl, C₂-C₆ alkynyl, cyano; or O(C₁-C₃) alkyl; and
R₂₀ and R₂₁ are each independently hydro or a moiety that forms, together with the attached CO₂, a readily hydrolyzable ester group.

15. The method of claim 14, wherein the inhibitor does not have a high affinity to a membrane binding folate protein.

16. The method of claim 15, wherein the inhibitor has the chemical structure:

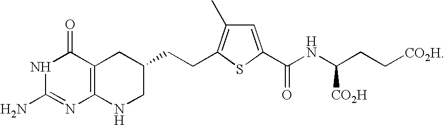

17. The method of claim 15, wherein the inhibitor has the chemical structure:

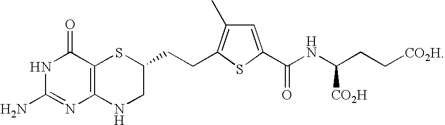

18. The method of claim 15, wherein the inhibitor has the chemical structure:

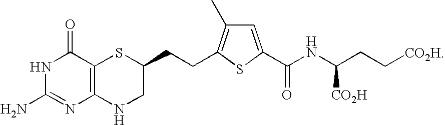

19. The method of claim 15, wherein the inhibitor has the chemical structure:

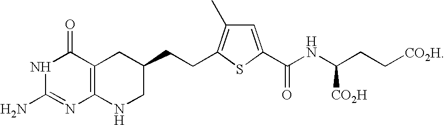

20. The method of claim 13, wherein the inhibitor is a compound having the Formula IV: 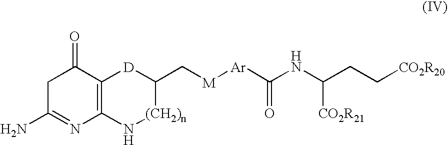
wherein:
n represents an integer from 0 to 2;
D represents sulfur, CH₂, oxygen, NH or selenium, provided that when n is 0, D is not CH₂, and when n is; 1, D is not CH₂ or NH;
M represents sulfur, oxygen, or a diradical of C₁-C₃ alkane, C₂-C₃ alkene, C₂-C₃ alkyne, or amine, wherein M is unsubstituted or substituted by one or more substituents selected from the group consisting of alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—OR_{c}, —(CH₂)_{z}—CN where z is 0-4, halo, —OH, —O—Rₐ—O—R_{b}, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NR_{d}Rₑ, —O—CO—NR_{d}Rₑ, —NR—CO—NR_{d}Rₑ, —NR_{c}—CO—Rₑ, —NR_{c}—CO₂—ORₑ, —CO—NR—CO—R_{d}, —O—SO₂—R_{c}, —O—SO—R_{c}, —O—S—R_{c}, —S—CO—R_{c}, —SO—CO—OR_{c}, —SO₂—CO—OR_{c}, —O—SO₃, —NR_{c}—SR_{d}, —NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂-R_{c}, —SO₂—NR_{d}R_{c}, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, hydroxyl, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group, and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —(CH₂)_{z}—CN where z is 0-4, halo, haloalkyl, haloaryl, —OH, keto (═O), —N—OH, NR_{c}—ORₑ, —NR_{d}Rₑ, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl; and unsubstituted heteroaryl, where Rₐ, R_{b}, R_{c}, R_{d}, and Rₑ are as defined above;
Ar represents a diradical of a cycloalkyl, heterocycloalkyl, aryl or heteroaryl ring system, said Ar is unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring; and
R₂₀ and R₂₁ represent, independently, hydro or a moiety that forms, together with the attached CO₂, a readily hydrolyzable ester group.

21. The method of claim 20, wherein the inhibitor is a compound having the Formula V: 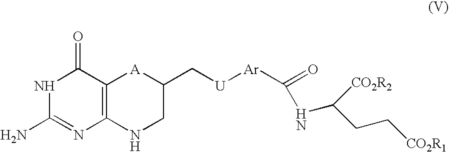
wherein:
A represents sulfur or selenium;
U represents CH₂, sulfur, oxygen or NH;
Ar represents a diradical of a cycloalkyl, heterocycloalkyl, aryl or heteroaryl ring system, said Ar is unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl ring; and
R₂₀ and R₂₁ represent, independently, hydro or a moiety that forms, together with the attached CO₂, a readily hydrolyzable ester group.

22. The method of claim 20, wherein the inhibitor is a compound having the Formula VI: 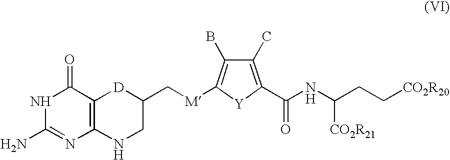
wherein:
D′ represents oxygen, sulfur or selenium;
M′ represents a sulfur, oxygen, or a diradical of C₁-C₃ alkane, C₂-C₃ alkene, C₂-C₃ alkyne, or amine, said M′ is unsubstituted or substituted by one or more substituents selected from the group consisting of alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—OR_{c}, —(CH₂)_{z}—CN where z is 0-4, halo, —OH, —O—Rₐ—O—R_{b}, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NR_{d}Rₑ, —O—CO—NR_{d}Rₑ, —NR_{c}—CO—NR_{d}Rₑ, —NR_{c}—CO—Rₑ, —NR_{c}—CO₂—ORₑ, —CO—NR_{c}—CO—R_{d}, —O—SO₂—R_{c}, —O—SO—R_{c}, —O—S—R_{c}, —S—CO—R_{c}, —SO—CO—OR_{c}, —SO₂—CO—OR_{c}, —O—SO₃, —NR_{c}—SR_{d}, —NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂—R_{c}, —SO₂—NR_{d}Rₑ, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, hydroxyl, halo, alkyl, heteroalkyl, haloalkyl, alkenyl, alkynyl —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group; and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —(CH₂)_{z}—CN where z is 0-4, halo, haloalkyl, haloaryl, —OH, keto (═O), —N—OH, NR_{c}—OR_{c}, —NR_{d}Rₑ, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d}, and Rₑ are as defined above;
Y represents O, S or NH;
B represents hydro or halo;
C represents hydro or halo or an unsubstituted or substituted C₁-C₆ alkyl; and
R₂₀ and R₂₁ represent independently hydro or a moiety that forms, together with the attached CO₂, a readily hydrozyable ester group.

23. The method of claim 22, wherein the inhibitor has the chemical structure:

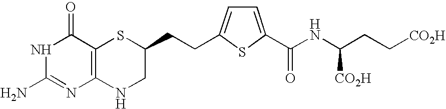

24. The method of claim 1, wherein the inhibitor is an inhibitor specific to aminoimidazolecarboximide ribonucleotide formyltransferase.

25. The method of claim 24, wherein the inhibitor is a compound having the Formula VIII: 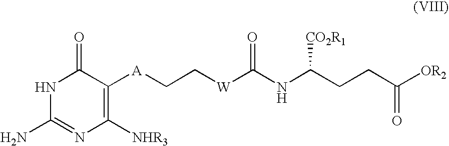
wherein:
A represents sulfur or selenium;
W represents an unsubstituted phenylene or thinylene diradical;
R₁ and R₂ represent, independently, hydro, C₁ to C₆ alkyl, or other readily hydrolyzable group; and
R₃ represents hydro or a C₁-C₆ alkyl or cycloalkyl group, unsubstituted or substituted by one or more halogen, hydroxyl or amino groups.

26. The method of claim 24, wherein the inhibitor is a compound having the Formula IX: 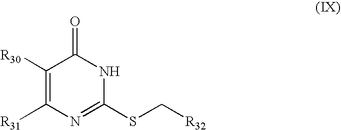
wherein:
R₃₀ represents hydro or CN;
R₃₁ represent phenyl or thienyl, unsubstituted or substituted with phenyl, phenoxy, thienyl, tetrazolyl, or 4-morpholinyl; and
R₃₂ is phenyl substituted with —SO₂NR₃₃R₃₄ or —NR₃₃SO₂R₃₄, unsubstituted or substituted with C₁-C₄ alkyl, C₁-C₄ alkoxy, or halo, wherein R₃₃ is H or C₁-C₄ alkyl and R₃₄ is C₁-C₄ alkyl, unsubstituted or substituted with heteroalkyl, aryl, heteroaryl, indolyl, or is 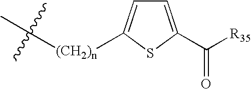
wherein n is an integer of from 1 to 4, R₃₅ is hydroxyl, C₁-C₄ alkoxy, or a glutamic-acid or glutamate-ester moiety linked through the amine functional group.

27. The method of claim 26, wherein the inhibitor is selected from the group consisting of:

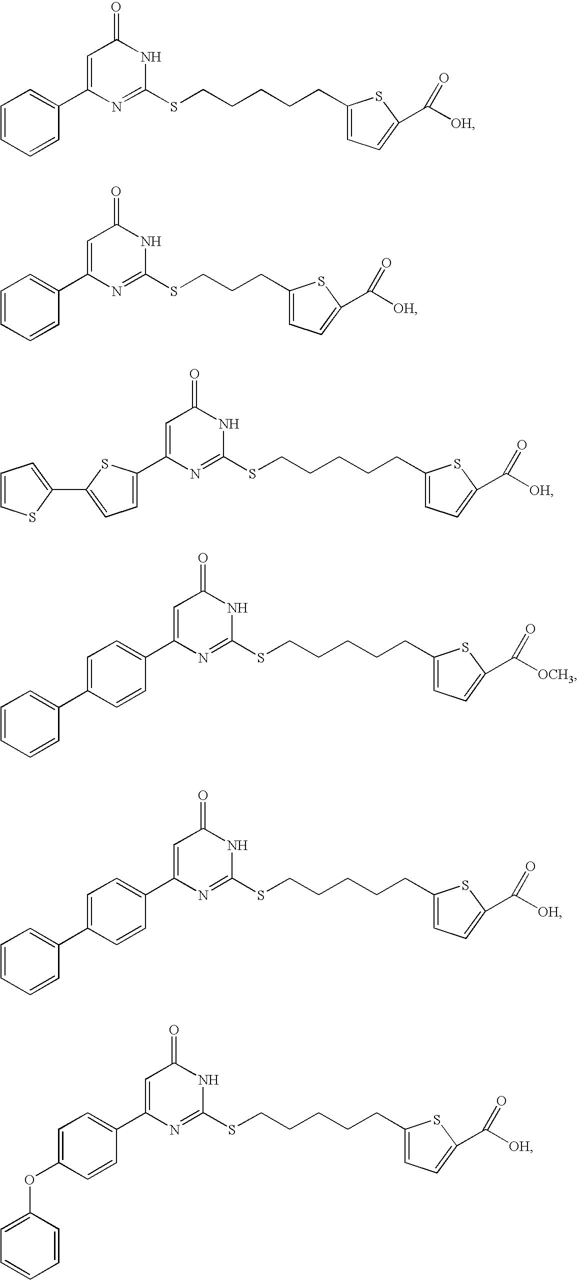

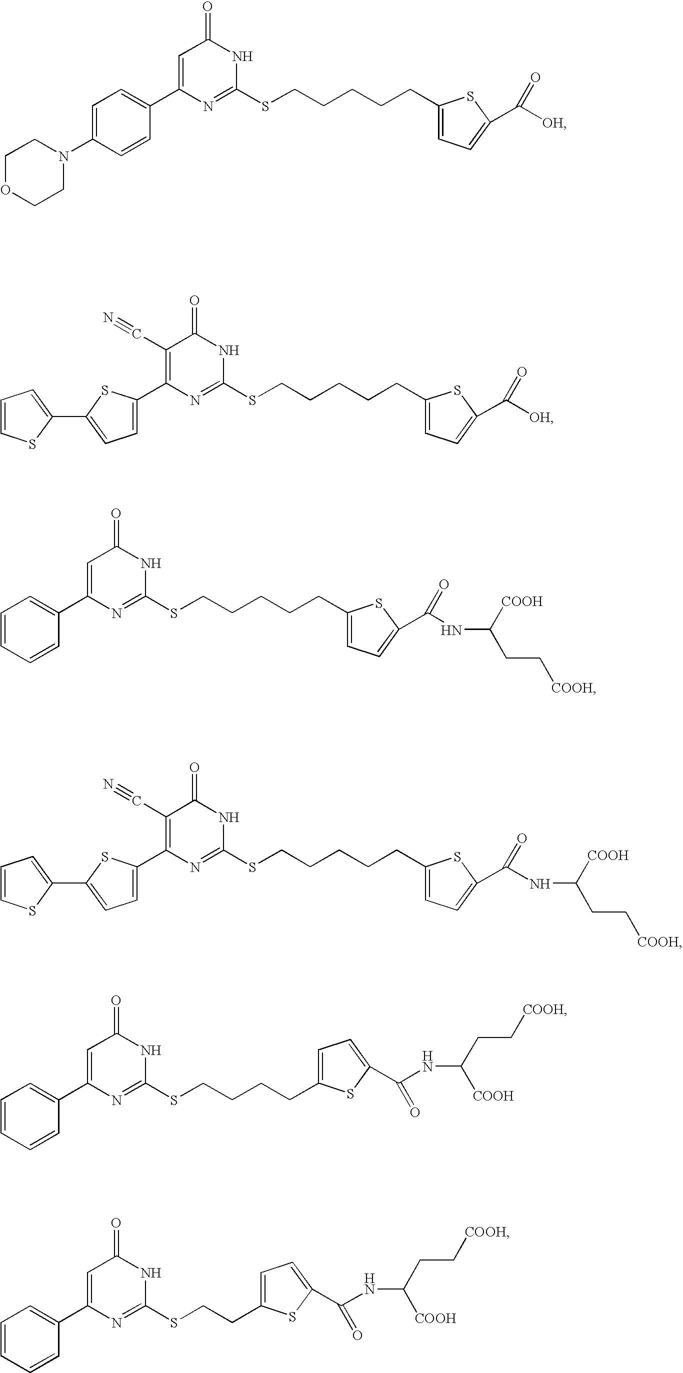

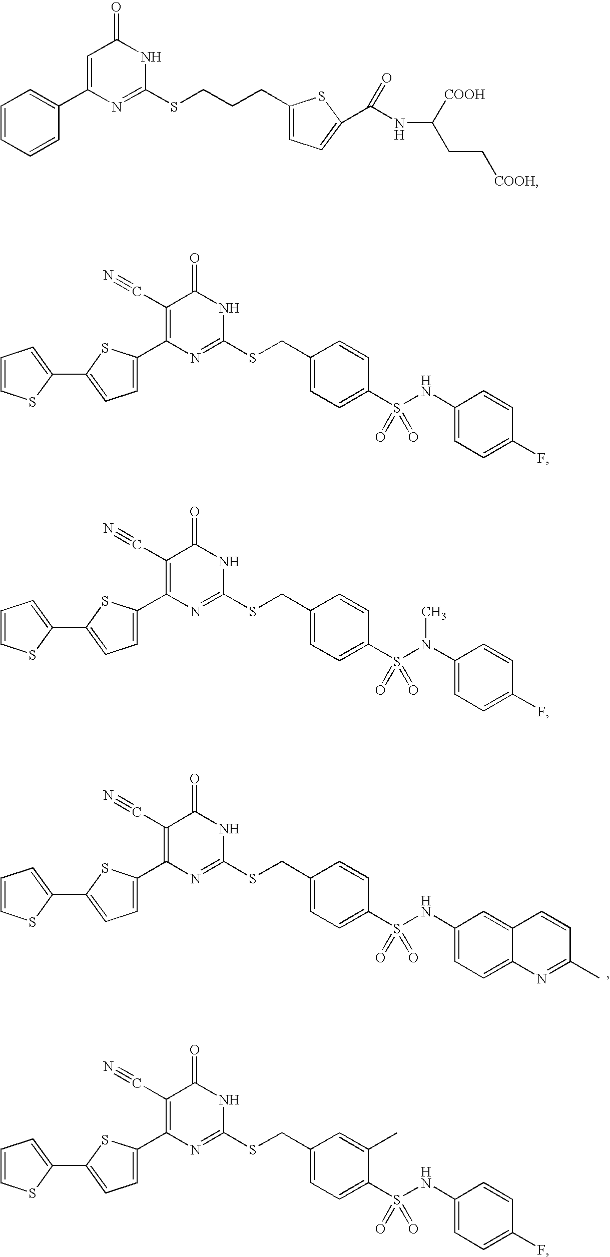

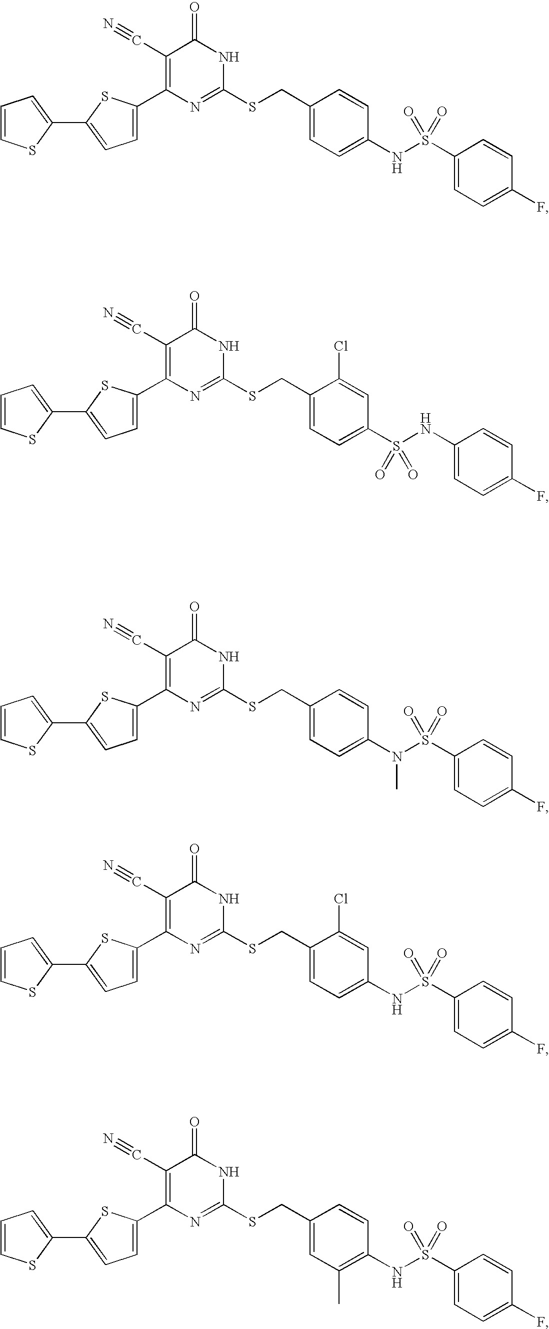

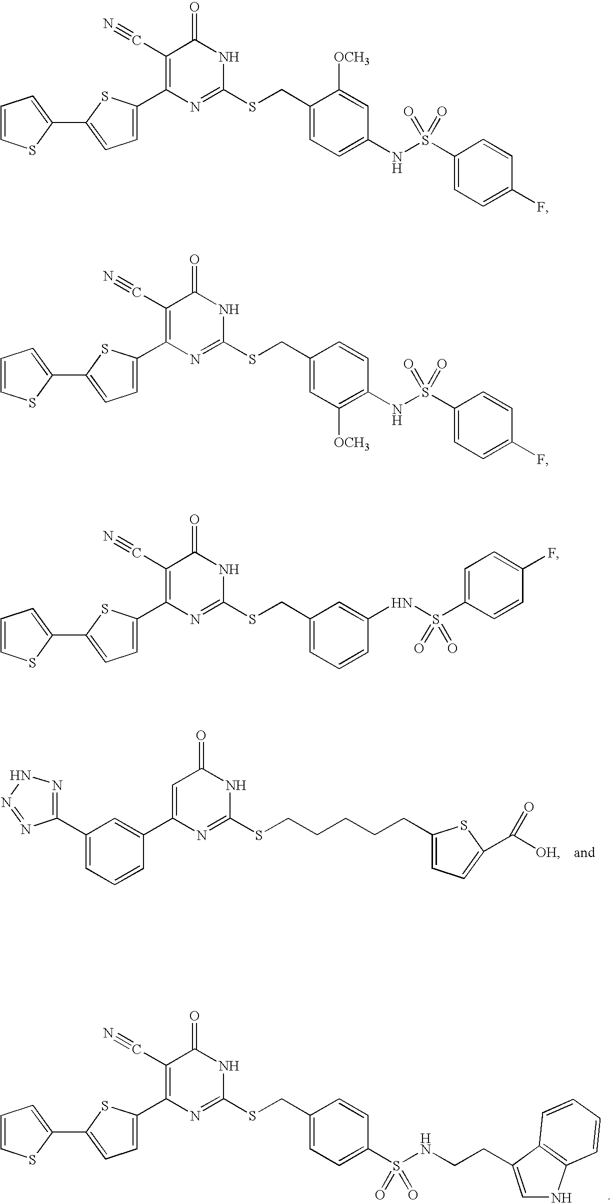

28. The method according to claim 1, wherein the mammal is a human.

29. The method according to claim 1, wherein the anti-toxicity agent is administered to the mammal parenterally orally.

30. The method according to claim 1, wherein the anti toxicity agent is administered during and after each dose of the inhibitor.

31. The method according to claim 1, wherein the anti-toxicity agent is administered to the mammal by multiple bolus or pump dosing, or by slow release formulations.

32. The method according to claim 1, wherein the method is used to treat a cell proliferative disorder selected from the group comprising lung cancer, leukemia, glioma, urothelial cancer, colon cancer, breast cancer, prostate cancer, pancreatic cancer, skin cancer, head and neck cancer.

33. A method for selectively killing MTAP-deficient cells of a mammal, the method comprising:
(c) administering to the mammal an inhibitor of glycinamide ribonucleotide formyltransferase (“GARFT”), aminoimidazolecarboximide ribonucleotide formyltransferase (“AICARFT”) or both in a therapeutically effective amount; and
(d) administering to the mammal an anti-toxicity agent in an amount effective to increase the maximally tolerated dose of the inhibitor;
wherein the inhibitor does not have high affinity to a membrane binding folate protein.

34. The method of claim 33, wherein the inhibitor is predominantly transported into cells by a reduced folate carrier protein.

35. The method of claim 33, wherein the anti-toxicity agent is an MTAP substrate or a prodrug of an MTAP substrate.

36. The method of claim 35, wherein the anti-toxicity agent has Formula X 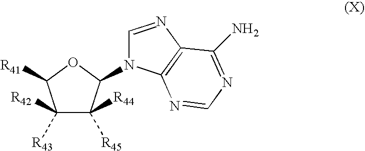
R₄₁ is selected from the group consisting of:
(a) —R_{g} wherein R_{g} represents a C₁-C₅ alkyl, C₂-C₅ alkenylene or alkynylene radical, unsubstituted or substituted by one or more substitutents independently selected from C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl;
(b) —R_{g}(Y)RₕRᵢ wherein R_{g} is as defined above, Y represents O, NH, S, or methylene; and Rₕ and Rᵢ represent, independently, (i) H;: (ii), a C₁-C₉ alkyl, or a C₂-C₆ alkenyl or alkynyl, unsubstituted or substituted by one or more substitutents independently selected from C₁ to C₆ alkoxy; C₁ to C₆ alkoxy(C₁ to C₆)alkyl; C₂ to C₆ alkynyl; acyl; halo; amino; hydroxyl; nitro; mercapto; —NCOORₒ; —CONH₂; C(O)N(Rₒ)₂; C(O)Rₒ; or C(O)ORₒ, wherein Rₒ is selected from the group consisting of H, C₁-C₆ alkyl, C₂-C₆ heterocycloalkyl, cycloalkyl, heteroaryl, aryl, and amino, unsubstituted or substituted with C₁-C₆ alkyl, 2- to 6-membered heteroalkyl, heterocycloalkyl, cycloalkyl, C₁-C₆ boc-aminoalkyl; cycloalkyl, heterocycloalkyl, aryl or heteroaryl; or (iii) a monocyclic or bicyclic cycloalkyl, heterocycloalkyl, aryl or heteroaryl, unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl heteroaryl, —COORₒ, —NCORₒ wherein Rₒ is as defined above, 2 to 6 membered heteroalkyl, C₁ to C₆ alkyl-cycloalkyl, C₁ to C₆ alkyl-heterocycloalkyl, C₁ to C₆ alkyl-aryl or C₁ to C₆ alkyl-aryl;
(c) C(O)NRⱼRₖ wherein Rⱼ and Rₖ represent, independently, (i) H; or (ii) a C₁-C₆ alkyl, amino, C₁-C₆ haloalkyl, C₁-C₆ aminoalkyl, C₁-C₆ boc-aminoalkyl, C₁-C₆ cycloalkyl, C₁-C₆ alkenyl, C₂-C₆ alkenylene, C₂-C₆ alkynylene radical, wherein Rⱼ and Rₖ are optionally joined together to form, together with the nitrogen to which they are bound; a heterocycloalkyl or heteroaryl ring containing two to five carbon atoms and wherein the C(O)NRⱼRₖ group is further unsubstituted or substituted by one or more substitutents independently selected from —C(O)Rₒ, —C(O)ORₒ wherein Rₒ is as defined above, C₁ to C₆ alkyl, C₂ to C₆ alkenyl, C₁ to C₆ alkoxy, C₁ to C₆ alkoxy(C₁ to C₆)alkyl C₂ to C₆ alkynyl, acyl, halo, amino, hydroxyl, nitro, mercapto, cycloalkyl, heterocycloalkyl, aryl or heteroaryl; or
(d) C(O)ORₕ wherein Rₕ is as defined above;
R₄₂ and R₄₄ represent, independently, H or OH; and
R₄₃ and R₄₅ represent, independently, H, OH, amino or halo;
where any of the cycloalkyl, heterocycloalkyl, aryl, heteroaryl moieties present in the above may be further substituted with one or more additional substituents independently selected from the group consisting of nitro, amino, —(CH₂)_{z}—CN where z is 0-4, halo, haloalkyl, haloaryl, hydroxyl, keto, C₁ to C₆ alkyl, C₂ to C₆ alkenyl, C₂ to C₆ alkynyl, heteroalkyl, unsubstituted cycloalkyl, unsubstituted heterocycloalkyl, unsubstituted aryl or unsubstituted heteroaryl;
and salts or solvates thereof.

37. The method of claim 36, wherein the anti-toxicity agent has a Kcat/Km ratio that is greater than 0.05 s⁻¹ μM⁻¹.

38. The method of claim 35, wherein the anti-toxicity agent has Formula XI: 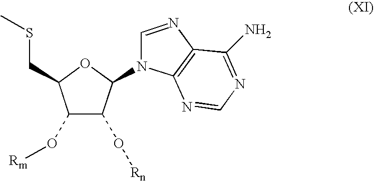
wherein
Rₘ and Rₙ are, independently, selected from the group consisting of H; a phosphate or a sodium salt thereof; C(O)N(Rₒ)₂; C(O)Rₒ; or C(O)ORₒ, wherein Rₒ is selected from the group consisting of H, C₁-C₆ alkyl, C₂-C₆ heterocycloalkyl, cycloalkyl, heteroaryl, aryl, and amino, unsubstituted or substituted with C₁-C₆ alkyl, C₁-C₆ heteroalkyl, C₂-C₆ heterocycloalkyl cycloalkyl, C₁-C₆ boc-aminoalkyl, and solvates or salts thereof.

39. The method of claim 38, wherein Rₘ and Rₙ independently represent

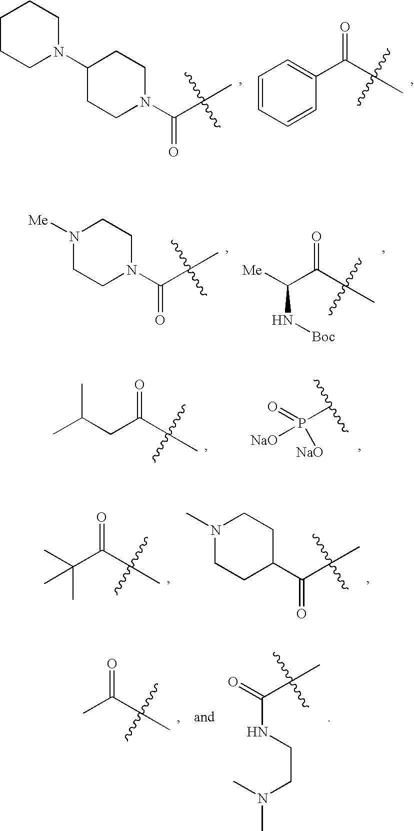

40. The method of claim 33, wherein the inhibitor is an inhibitor specific to glycinamide ribonucleotide formyltransferase.

41. The method of claim 40, wherein the inhibitor is a compound having the Formula VII: 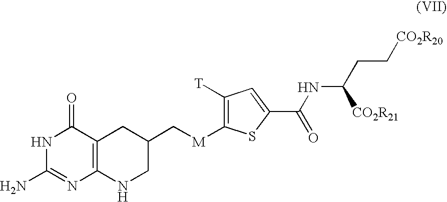
wherein L represents sulfur, CH₂ or selenium;
M represents a sulfur, oxygen, or a diradical of C₁-C₃ alkane, C₂-C₃ alkene, C₂-C₃ alkyne, or amine, wherein M is unsubstituted or substituted by one or more substituents selected from the group consisting of alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—OR_{c}, —(CH₂)_{z}—CN where z is 0-4, halo, —OH, —O—Rₐ—O—R_{b}, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NR_{d}Rₑ, —O—CO—NR_{d}Rₑ, —NR_{c}—CO—NR_{d}Rₑ, —NR_{c}—CO—Rₑ, —NR_{c}—CO₂—ORₑ, —CO—NR_{c}—CO—R_{d}, —O—SO₂—R_{c}, —O—SO—R_{c}, —O—S—R_{c}, —S—CO—R_{c}, —SO—CO—OR_{c}, —SO₂—CO—OR_{c}, —O—SO₃, —NR_{c}—SR_{d}, —NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂—R_{c}, —SO₂NR_{d}Rₑ, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, hydroxyl, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group; and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —(CH₂)_{z}—CN where z is 0-4, halo, haloalkyl, haloaryl, —OH, keto (═O), —N—OH, NR_{c}—OR_{c}, —NR_{d}Rₑ, —CO—NR_{d}Rₑ, —CO—ORₑ, —CO—R_{c}, NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d} and Rₑ are as defined above;
T represents C₁-C₆ alkyl; C₂-C₆ alkenyl; C₂-C₆ alkynyl; —C(O)E, wherein E represents hydro, C₁-C₃ alkyl, C₂-C₃ alkenyl, C₂-C₃ alkynyl, O—(C₁-C₃) alkoxy, or NR₁₀R₁₁, wherein R₁₀ and R₁₁ represent independently hydro, C₁-C₃ alkyl, C₂-C₃ alkenyl, C₂-C₃ alkynyl; hydroxyl; nitro; SR₁₂, wherein R₁₂ is hydro, C₁-C₆ alkyl, C₂-C₆ alkenyl, C₂-C₆ alkynyl, cyano; or O(C₁-C₃) alkyl; and
R₂₀ and R₂₁ are each independently hydro or a moiety that forms, together with the attached CO₂, a readily hydrolyzable ester group.

42. The method of claim 41, wherein the inhibitor has the chemical structure:

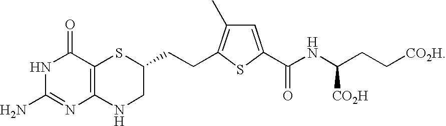

43. The method of claim 41, wherein the inhibitor has the chemical structure:

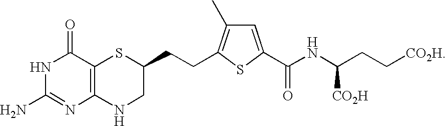

44. The method of claim 41, wherein the inhibitor has the chemical structure:

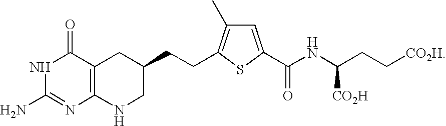

45. The method of claim 41 wherein the inhibitor has the chemical structure:

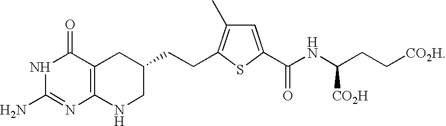

46. The method of claim 33 wherein the inhibitor has the chemical structure:

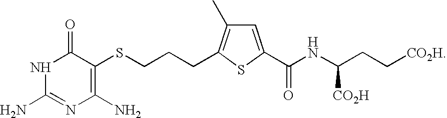

47. The method according to claim 33, wherein the mammal is a human.

48. The method according to claim 33, wherein the anti-toxicity agent is administered to the mammal parenterally or orally.

49. The method according to claim 33, wherein the anti-toxicity agent is administered during and after each dose of the inhibitor.

50. The method according to claim 33, wherein the anti-toxicity agent is administered to the mammal by multiple bolus or pump dosing, or by slow release formulations.

51. The method according to claim 33, wherein the method is used to treat a cell proliferative disorder selected from the group comprising lung cancer, leukemia, glioma, urothelial cancer, colon cancer, breast cancer, prostate cancer, pancreatic cancer, skin cancer, head and neck cancer.

52. A method for selectively killing MTAP-deficient cells of a mammal, the method comprising:
(a) administering to the mammal an inhibitor of glycinamide ribonucleotide formyltransferase (“GARFT”) in a therapeutically effective amount, the inhibitor having the formula: 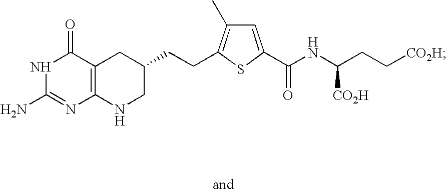
(b) administering to the mammal an anti-toxicity agent in an amount effective to increase the maximally tolerated dose of the inhibitor;
wherein the anti-toxicity agent is administered during and after administration of the inhibitor.

53. The method of claim 52, wherein the anti-toxicity agent has a Kcat/Km ratio that is greater than 0.05 s⁻¹ μM⁻¹.

54. The method of claim 2, wherein the anti-toxicity agent has Formula XII: 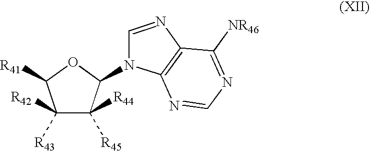
R₄₁ is selected from the group consisting of:
(a) —R_{g} wherein R_{g} represents a C₁-C₅ alkyl, C₂-C₅ alkenylene or alkynylene radical, unsubstituted or substituted by one or more substitutents independently selected from C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl;
(b) —R_{g}(Y)RₕRᵢ wherein R_{g} is as defined above, Y represents O, NH, S, or methylene; and Rₕ and Rᵢ represent, independently, (i) H; (ii) a C₁-C₉ alkyl, or a C₂-C₆ alkenyl or alkynyl, unsubstituted or substituted by one or more substitutents independently selected from C₁ to C₆ alkoxy; C₁ to C₆ alkoxy(C₁ to C₆)alkyl; C₂ to C₆ alkynyl; acyl; halo; amino hydroxyl; nitro; mercapto; —NCOORₒ; —CONH₂; C(O)N(Rₒ)₂; C(O)Rₒ; or C(O)ORₒ, wherein Rₒ is selected from the group consisting of H, C₁-C₆ alkyl, C₁-C₆ heterocycloalkyl, cycloalkyl, heteroaryl, aryl, and amino, unsubstituted or substituted with C₁-C₆ alkyl, 2- to 6-membered heteroalkyl, heterocycloalkyl, cycloalkyl, C₁-C₆ boc-aminoalkyl; cycloalkyl, heterocycloalkyl, aryl or heteroaryl; or (iii) a monocyclic or bicyclic cycloalkyl, heterocycloalkyl, aryl or heteroaryl, unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl heteroaryl, —COORₒ, —NCORₒ wherein Rₒ is as defined above, 2 to 6 membered heteroalkyl, C₁ to C₆ alkyl-cycloalkyl, C₁ to C₆ alkyl-heterocycloalkyl, C₁ to C₆ alkyl-aryl or C₁ to C₆ alkyl-aryl;
(c) C(O)NRⱼRₖ wherein Rⱼ and Rₖ represent, independently, (i) H; or (ii) a C₁-C₆ alkyl, amino, C₁-C₆ haloalkyl, C₁-C₆ aminoalkyl, C₁-C₆ boc-aminoalkyl, C₁-C₆ cycloalkyl, C₁-C₆ alkenyl, C₂-C₆ alkenylene, C₂-C₆ alkynylene radical, wherein Rⱼ and Rₖ are optionally joined together to form, together with the nitrogen to which they are bound, a heterocycloalkyl or heteroaryl ring containing two to five carbon atoms and wherein the C(O)NRⱼRₖ group is further unsubstituted or substituted by one or more substitutents independently selected from —C(O)Rₒ, —C(O)ORₒ wherein Rₒ is as defined above, C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or, heteroaryl; or
(d) C(O)ORₕ wherein Rₕ is as defined above;
R₄₂ and R₄₄ represent, independently, H or OH; and
R₄₃ and R₄₅ represent, independently, H, OH, amino or halo;
where any of the cycloalkyl, heterocycloalkyl, aryl, heteroaryl moieties present in the above may be further substituted with one or more additional substituents independently selected from the group consisting of nitro, amino, —(CH₂)_{z}—CN where z is 0-4, halo, haloalkyl, haloaryl, hydroxyl, keto, C₁ to C₆ alkyl, C₂ to C₆ alkenyl, C₂ to C₆ alkynyl, heteroalkyl, unsubstituted cycloalkyl, unsubstituted heterocycloalkyl, unsubstituted aryl or unsubstituted heteroaryl;
and R₄₆ represents (i) H; (ii) a C₁-C₉ alkyl, or a C₂-C₆ alkenyl or alkynyl, unsubstituted or substituted by one or more substitutents independently selected from C₁ to C₆ alkoxy; C₁ to C₆ alkoxy(C₁ to C₆)alkyl; C₂ to C₆ alkynyl; acyl; halo; amino; hydroxyl; nitro; mercapto; cycloalkyl, heterocycloalkyl, aryl or heteroaryl; or (iii) a monocyclic or bicyclic cycloalkyl, heterocycloalkyl, aryl or heteroaryl, unsubstituted or substituted with one or more substituents independently selected from C₁ to C₆ alkyl, C₂ to C₆ alkenyl, C₁ to C₆ alkoxy, C₁ to C₆ alkoxy(C₁ to C₆)alkyl, C₂ to C₆ alkynyl, acyl, halo, amino, hydroxyl, nitro, mercapto, cycloalkyl, heterocycloalkyl, aryl or heteroaryl;
and salts or solvates thereof.


SEQUENCE LISTING (3 sequences)

SEQ ID NO: 1 - DNA, 870 bp, Artificial
  Cloned MTAP cDNA
gcagacatggcctctggcaccaccaccaccgccgtgaagattggaataattggtggaacaggcctggatgatccagaaattttagaaggaagaactgaaaaatatgtggatactccatttggcaagccatctgatgccttaattttggggaagataaaaaatgttgattgcgtcctccttgcaaggcatggaaggcagcacaccatcatgccttcaaaggtcaactaccaggcgaacatctgggctttgaaggaagagggctgtacacatgtcatagtgaccacagcttgtggctccttgagggaggagattcagcccggcgatattgtcattattgatcagttcattgacaggaccactatgagacctcagtccttctatgatggaagtcattcttgtgccagaggagtgtgccatattccaatggctgagccgttttgccccaaaacgagagaggttcttatagagactgctaagaagctaggactccggtgccactcaaaggggacaatggtcacaatcgagggacctcgttttagctcccgggcagaaagcttcatgttccgcacctggggggcggatgttatcaacatgaccacagttccagaggtggttcttgctaaggaggctggaatttgttacgcaagtatcgccatggcgacagattatgactgctggaaggagcacgaggaagcagtttcggtggaccgggtcttaaagaccctgaaagaaaacgctaataaagccaaaagcttactgctcactaccatacctcagatagggtccacagaatggtcagaaaccctccataacctgaagaatatggcccagttttctgttttattaccaagacattaaagtagcatggct

SEQ ID NO: 2 - DNA, 21 bp, Artificial
  Forward Primer
gcagacatggcctctggcacc

SEQ ID NO: 3 - DNA, 24 bp, Artificial
  Reverse Primer
agccatgctactttaatgtcttgg